(19) United States
(12) Patent Application Publication
(10) Pub. No.: US 2026/0102962 A1
(43) Pub. Date: Apr. 16, 2026

(54) Dynamic Covalent Chemistry of Spiropyrans for Synthesis of Photoswitchable Photoinitiators and Uses Thereof, and Process for Locally Polymerizing a Starting Material by Dual Color Photopolymerization

(71) Applicant: xolo GmbH, Berlin (DE)

(72) Inventor: Yves Garmshausen, Berlin (DE)

(21) Appl. No.: 19/422,039

(22) Filed: Dec. 16, 2025

Related U.S. Application Data
(63) Continuation-in-part of application No. PCT/EP2024/066792, filed on Jun. 17, 2024.

(30) Foreign Application Priority Data
Jun. 19, 2023 (DE) 102023115968.2

(51) Int. Cl. B29C 64/124; B29K 33/00; B29K 105/00; B33Y 10/00; B33Y 70/00; C07D 491/107; C08F 22/22

(52) U.S. Cl. CPC B29C 64/124; B33Y 10/00; B33Y 70/00; C07D 491/107; C08F 22/22; B29K 2033/08; B29K 2105/0005

(57) ABSTRACT

The present invention relates to dynamic covalent chemistry of spiropyrans and application thereof. In particular, a process is provided to synthesize spiropyrans via an exchange reaction. Thereby, substitution patterns at spiropyrans may be firstly synthetically accessible. The provided spiropyrans contain specific substituents and substitution patterns and are used as dual color photoinitiators, which result in improved dual color volumetric printing (xolography). The present invention relates further to a process for locally polymerizing a starting material by dual color photopolymerization. In particular, photoinitiators are provided, which cause efficient curing of photopolymerizable formulations upon irradiation with two different wavelengths, and which may be used for volumetric printing (xolocure initiators).

### CROSS-REFERENCE TO RELATED APPLICATIONS

This application is a continuation-in-part of PCT/EP2024/066792, filed Jun. 17, 2024, which claims priority to German Patent Application No. 10 2023 115 968.2, filed Jun. 19, 2023. The content of these applications is incorporated by reference herein.

### FIELD OF INVENTION

The present invention relates to dynamic covalent chemistry of spiropyrans and application thereof. In particular, a process is provided to synthesize spiropyrans via an exchange reaction. Thereby, substitution patterns at spiropyrans may be firstly synthetically accessible. The provided spiropyrans contain specific substituents and substitution patterns and are used as dual color photoinitiators, which result in improved dual color volumetric printing (xolography).

The present invention relates further to a process for locally polymerizing a starting material by dual color photopolymerization. In particular, photoinitiators are provided, which cause efficient curing of photopolymerizable formulations upon irradiation with two different wavelengths, and which may be used for volumetric printing (xolocure initiators).

### BACKGROUND OF THE ART

### Spiropyran, and Merocyanine Used as Photoswitches

A definition of photoswitches, structures which are photochromic, and terms used in the field can be found in [Bouas-Laurent, Dürr, Pure and Applied Chemistry, 2001, 73, 639-665]. In general, a photoswitch is a photochromic compound which can undergo a reversible structural change in at least one direction via a photochemical path. A typical example is the spiropyran/merocyanine system, as illustrated below:

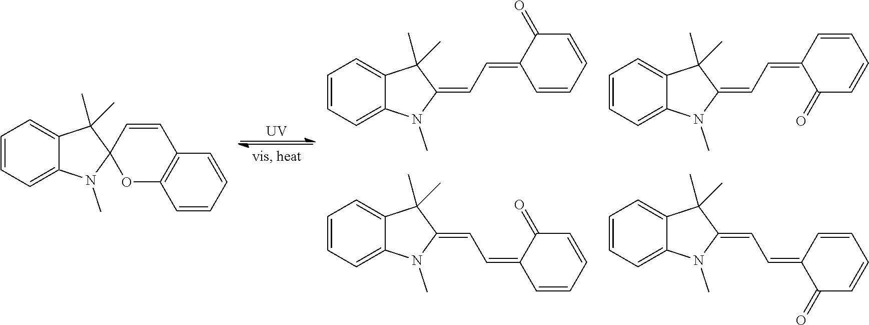

The colorless spiropyran is the thermodynamically favored form and undergoes a ring opening reaction upon irradiation with UV light to the metastable colored merocyanine form (positive photochromism). The merocyanine form can be a mixture of different isomers, where the bonds between the five-membered and six-membered ring are either E or Z configurated. Within this invention the term merocyanine refers to one or more of the different E/Z isomers. The reverse reaction from merocyanine to spiropyran can be conducted either by light or thermally (T-type photochromism).

Spiropyrans are generally synthesized by the condensation of a salicylaldehyde and a 2-methyleneindoline, which may be obtained by deprotonation of an indolenium salt. The spiropyran may be functionalized afterwards, especially if it contains suitable functional groups:

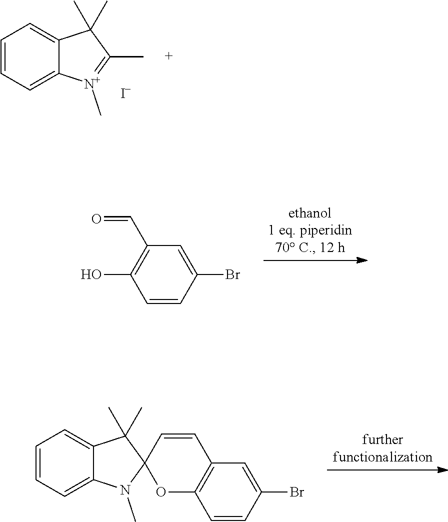 [SMILES: CC1=[N+](C)c2ccccc2C1(C)C ; [I-] ; O=Cc1cc(Br)ccc1O ; CN1c2ccccc2C(C)(C)C12C=Cc1cc(Br)ccc1O2]

Due to the free hydroxy group and the free aldehyde group, the direct functionalization of salicylaldehydes is limited. There exists no direct protecting group for a salicylaldehyde. Salicylaldehydes are sensitive to nucleophiles, oxidative and reductive conditions. Typical reactions like the halide-metal exchange of a bromine with n-butyl lithium are not possible, since the hydroxy group is deprotonated, instead of undergoing the desired halide-metal exchange. Furthermore, the hydroxy group has a strong directing effect and limits the direct functionalization to the position para to the hydroxy group. Also, the aldehyde as a strong electron withdrawing group deactivates the aromatic system, which allows only very reactive reagents for the direct functionalization, such as acetic anhydride with aluminum chloride in a Friedl-Crafts-acylation. Other typical reactions, like the reduction of a nitro group are not possible in presence of the formyl group of the salicylaldehyde as the formyl group is reduced under similar conditions. Thus, functionalization of the salicylaldehyde is limited.

### Dynamic Covalent Chemistry

An overview on dynamic covalent chemistry with terms and definitions can be found in [Yinghua Jin, Chao Yu, Ryan J. Denman, Wei Zhang, Chemical Society Reviews, 2013, 42, 6634-6654, Peter T. Corbett, Julien Leclaire, Laurent Vial, Kevin R. West, Jean-Luc Wietor, Jeremy K. M. Sanders, Sijbren Otto Chemical Reviews, 2006, 106, 3652-3711 and Rowan et al. Angewandte Chemie International Edition, 2002, 41, 898-952.]. Dynamic covalent chemistry describes reversible chemical reactions which are under equilibrium control. The final product distribution in the thermodynamic minimum depends on the relative stabilities of the final products. Applications of dynamic covalent chemistry can be found in many self-organizing systems, such as host-guest interactions, covalent organic frameworks, dynamic combinatorial libraries, drug discovery, polymeric materials. To give an example, two imines can react in an exchange reaction. Therefore, imines have been widely applied in dynamic covalent chemistry:

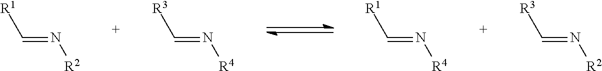

Currently, dynamic covalent chemistry is limited to the few reactions which are suited for the exchange. These are mainly: Aldol reaction, Diels-Alder reaction, Metathesis of C—C double and triple bonds, and the exchange of boronic and carboxylic esters, imines, aminals, disulfides, and boronic acid anhydrides. Importantly, dynamic covalent chemistry has not focused on spiropyrans yet.

### Photoinitiators

An overview on photoinitiators with terms and definitions can be found in [Green, Industrial Photoinitiators A Technical Guide, CRC Press, 2010; Fouassier and Lalevée, Photoinitiators Structures, Reactivity and Applications in Polymerization, Wiley VCH, 2021]. Curing of a material refers to the polymerization of monomers. Depending on the mechanism of the polymerization several functional groups have been found to be suitable monomers, which include but are not limited to acrylates, methacrylates, thiol+ene, epoxides, oxiranes, oxetanes, or vinylethers. Photoinitiators start the polymerization upon irradiation with light. The polymerization can be conducted in a radical fashion, cationic, anionic, or metal catalyzed. Photoinitiators are either type 1, where a single compound can start the polymerization or type 2, where a co-initiator is required.

Type 1 photoinitiators can undergo homolytic (radical) or heterolytic bond cleavage (cationic/anionic) to form the reactive species. In the following reactions further species can be produced which can cause a different nature of the polymerization, e.g. an amine radical is initially produced by homolytic bond cleavage, which than abstracts a hydrogen atom from the environment to form an amine and start a base mediated polymerization. Type 2 photoinitiators can start the polymerization by two different mechanisms, where the first is hydrogen abstraction from a co-initiator, which produces two radicals and can cause further reactive species from follow up reactions. The second mechanism is a photoredox reaction, where an electron is transferred between co-initiator and photoinitiator. The photoredox reaction can be proceeded by further proton transfer reactions to produce radicals or other follow up reactions which result in further reactive species such as acids, bases or radicals. Several structural motives are known to initiate a polymerization and examples of the most important ones are shown below.

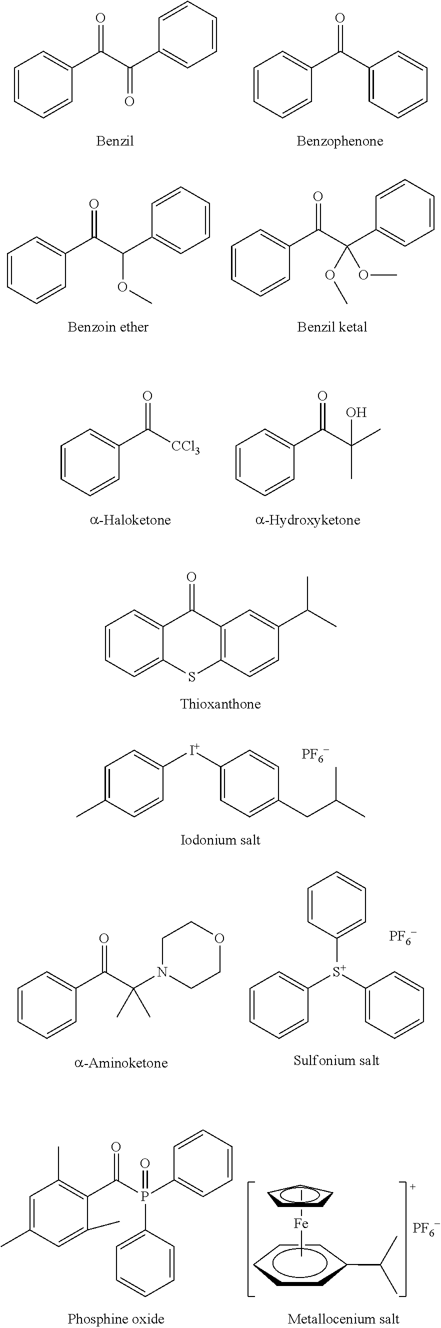

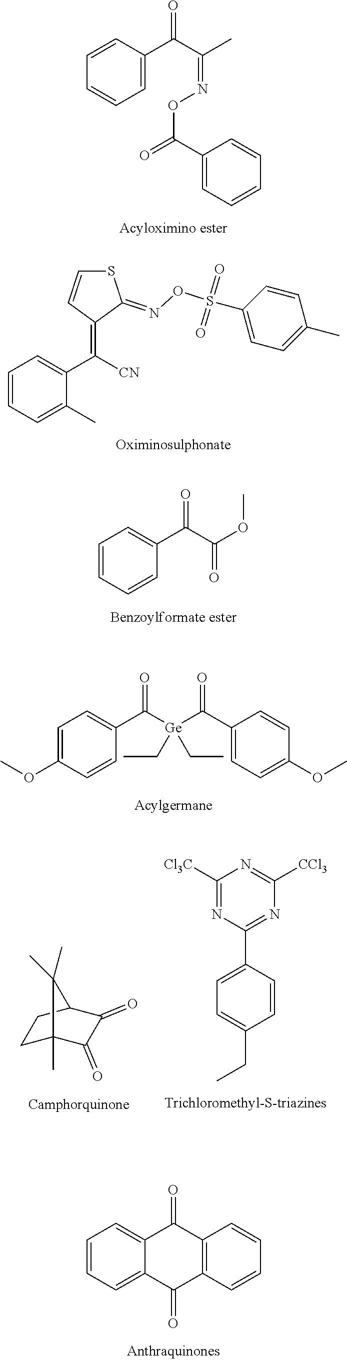

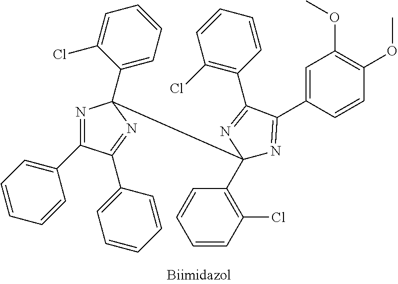 [SMILES: COc1ccc(C2=NC(c3ccccc3Cl)(C3(c4ccccc4Cl)N=C(c4ccccc4)C(c4ccccc4)=N3)N=C2c2ccccc2Cl)cc1OC]

### Additive Manufacturing/3D Printing

Additive manufacturing or 3D printing can be performed with various techniques and materials and allow to produce customized parts. Compared to injection molding, additive manufacturing enables the formation of more complex structures, saving material and giving the opportunity to construct specialized parts for machines with superior performance. However, most of the additive manufacturing techniques rely on a layer by layer build sequence. Therefore, the object is sliced into many layers and one layer is printed after the other. Consequently, certain limitations are inherent, which is the necessity for support structures to prevent overhanging parts from falling apart. Usually, several mechanical steps are involved between printing of the layers, which lead to long production times. The starting material for a print must be in a form to allow for the mechanical operations without destruction of the object, which restricts starting materials to certain powder sizes or viscosities.

To overcome the limitations of classic additive manufacturing, volumetric printing has been proposed as a solution. In such an approach the resin is in a container and by the action of light curing does not happen on the walls of the container but inside the volume. This removes the necessity of support structures, allows for resins of low and high viscosity, allows the formation of soft objects, and does not suffer from the inhibition by oxygen or water at the surface.

To overcome the issues of two photon polymerization and tomographic reconstruction, a dual color photoinitiating system has been developed (xolography). This requires a photoinitiator which upon irradiation with light of a first wavelength λ1 is transformed from its thermodynamically stable ground state form A into a metastable ground state species B. When species B absorbs light of the second wavelength λ2, it can initiate a polymerization reaction via form C. Form C is an excited state of B, which produces radicals, cations or anions by further reaction with or without a co-initiator. In the sense of this invention the terms first wavelength, second wavelength, and third wavelength can refer to a range of wavelengths. For usage in a volumetric printing approach a back reaction from B to A must be possible to avoid hardening of unwanted areas. The back reaction from B to A can be triggered thermally or by irradiation with a third wavelength λ3.

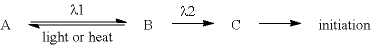

The dual color volumetric printing technology described above is known as xolography. Dual color photoinitiating systems suitable for this approach can be based on spiropyrans, which bear a carbonyl moiety [Garmshausen et al., WO2020245456A1]. The dual color photoinitiators described therein are synthesized by the reaction of a 2-methyleneindoline and a salicylaldehyde which maybe post-functionalized in subsequent steps.

JP H0375127A by Kenji and Ichiro claimed three photoswitches for volumetric printing. The molecules described therein are known for their photochromic properties, but do not show selective curing, where beams of both wavelengths would intersect as has been shown by Neckers [U.S. Pat. No. 5,230,986A, column 6, line 27-35]. Even if they were able to initiate with dual color irradiation, they show a slow thermal reverse reaction from B to A, which makes them unsuitable in volumetric printing applications. The spiropyran described therein is synthesized from a 2-methyleneindoline and a salicylaldehyde.

U.S. Pat. No. 5,230,986A by Neckers disclosed iodinated benzospiropyrans with suitable co-initiators as two photon radical photoinitiators with the following structure, where at least one of the two substituents X₁ and X₃ is an iodine:

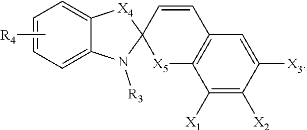

The disclosed molecules are of limited efficiency for photopolymerization, which makes high concentrations necessary [Lee, Neckers, Chem. Mater. 1991, 3, 852-858 and Lee, Neckers, Chem. Mater. 1991, 3, 858-864]. Consequently, the penetration depth of the light is limited and the volume suitable for printing cannot exceed 2 mm as has been shown by the same authors [Lee, Neckers, Chem. Mater. 1991, 3, 858-864, FIGS. 12 and 13]. The iodo substituent acts as a triplet sensitizer on the spiropyran form, so that the unwanted side reaction from irradiation using the first wavelength alone is dominant, where the spiropyran causes the photo redox reaction and hence formation of radicals, without isomerizing to the merocyanine. Iodinated benzospiropyrans are further limited by a slow thermal back reaction in more viscous media which makes them unsuitable for volumetric printing. The disclosed molecules are all synthesized via condensation of a 2-methyleneindoline and a salicylaldehyde.

Two other spiropyran-based two component systems have been reported for recording holograms [Jeudy, Robillard, Opt. Commun. 1975, 13, 25-28; Ichimura, Sakuragi, J. Polym. Sci. Polym. Lett, 1988, 26 185-189]. However, both are not suited for volumetric printing, since on the one hand the thermal back reaction in both cases is too slow for a commercially successful application and on the other hand both cause substantial polymerization with the first wavelength alone [Lee, Neckers, Chem. Mater. 1991, 3, 858-864]. In both publications the molecules are synthesized from the condensation of a 2-methyleneindoline and a salicylaldehyde.

The reaction of a spiropyran with 3,5-dinitrosalicylaldehyde is proposed to exchange the salicylaldehyde moiety without analytical proof [Bertelson, Techniques of Chemistry, Volume III, Photochromism, Chapter 3]. The reaction is further proposed to serve as a protecting strategy for salicylaldehydes. When the reaction is carried out as described by Bertelson, precipitation occurs accordingly. While this precipitate is claimed to be the exchanged spiropyran, analysis reveals, that 3,5-dinitrosalicylaldehyde acts as an acid in the mixture, protonating the original spiropyran and leading to precipitation of the protonated spiropyran without any exchange. Furthermore, the acid is found to catalyze an Aldol condensation, in cases where the spiropyran carries an acyl functionality capable of undergoing this reaction, such as acetyl. Other acid sensitive functionalities show decomposition when heated with 3,5-dinitrosalicylaldehyde.

Spiropyran has been proposed as a protecting group for salicylaldehydes, which can be cleaved using strong oxidants, such as potassium permanganate, sodium periodate, and ozone [Young Jin Cho et al. Tetrahedron Letters, 2000, 41, 3915-3917]. The oxidant causes the formation of salicylic acid derivatives and other side products, resulting in limited applicability. Furthermore, the protecting group cannot be recovered using this approach.

Among the many known techniques for additive manufacturing or 3D-printing, volumetric printing techniques offer several advantages, especially when the printed object is supposed to be used in a biological context (bioprinting). Volumetric printing techniques allow the formation of constructs with increased complexity compared to conventional 3D printing techniques, as they intrinsically don't require support structures and allow for printing of soft hydrogel objects. Light based volumetric printing techniques comprise the solidification of specific voxels within a volume of a photopolymerizable resin. Volumetric printing technologies include, but are not limited to: xolography, computed axial lithography/tomographic reconstruction, multiphotonpolymerisation, or cone beam lithography.

Another implementation of volumetric printing relies on two photon polymerization, where a common photoinitiator is excited by the simultaneous uptake of two photons, causing polymerization in the focus of a laser beam [S. Maruo et al., Opt Lett., 1997, 22, 132-134]. Two photon polymerization requires costly and delicate setups with intense pulsed laser sources. Furthermore, the technique is inherently slow due to the nonlinearity of the process, reducing the achievable object size considerably.

Furthermore, the volumetric printing method of computed axial lithography or tomographic reconstruction has been developed, where the resin is illuminated with different light patterns from various angles. Due to the overlaying light an intensity distribution in the volume is generated, which causes curing wherever a certain threshold is exceeded. The light patterns which are required to form a three-dimensional object are calculated before in a similar way to a reverse process of computer tomographic analysis [Kelly et al., US20180326666A1, 2018; Shusteff et al., US20180015672A1, 2018; Delrot et al. WO2019043529A1, 2019].

In a similar approach to the method of computed axial lithography or tomographic reconstruction, cone beam lithography or parallax manufacturing has been disclosed in WO2024163474A1. In parallax manufacturing an image is projected, wherein the image size increases with increasing distance from the light source. More specifically the light path can be in a cone shape or truncated cone shape or in a shape which resembles a pyramid or truncated pyramid with a quadratic or rectangular base. By moving the light source relative to the resin, each voxel of the resin can experience irradiation from different angles. By changing the image for each relative position of irradiation and resin, each voxel can absorb a specific light dose leading to solidification of the resin in the shape of the voxels which have absorbed a light dose above a threshold light dose. As such the method of cone beam lithography can be similar to the reverse process of cone beam reconstruction.

In xolography, light of two different wavelengths is intersected in a volume of resin. At the intersection voxels a polymerization is induced, which causes the resin to solidify. By changing the positions of intersection in the volume a shaped body with a specific geometry can be solidified. The process can involve a photoswitchable photoinitiator and a co-initiator which have been disclosed in WO2020245456A1, WO2021089090A1, U.S. Pat. No. 5,230,986A, WO2023034398A1, WO2023220461A1, and WO2024186326.

The dual color photoinitiators disclosed in the prior art suffer from various problems including limited reactivity, slow back reactions, and residual coloring in formed objects.

### SUMMARY OF THE INVENTION

### Problem to be Solved

The problems to be solved and shortcomings are stated in the background of the art. Especially, spiropyrans which may be of interest for the application as photoinitiators, may not be accessible by the conventional synthetical methods stated above. Furthermore, some methods of the prior art may provide spiropyrans, in only low yield, low conversion, with high impurities and, thus, with only low efficiency. As a consequence, spiropyrans that can be used as photoinitiators, especially dual color photoinitiators, are only limited accessible, so that the provision of new photoinitiators, especially dual color photoinitiators which are crucial for the development of volumetric printing, is hampered and needs to be overcome. Another problem to be solved is to overcome the limitations of functionalizing a salicylaldehyde or 2-methyleneindoline efficiently, so that new substitution patterns of salicylaldehyde may become accessible for the synthesis of photoinitiators.

### Solution to the Problem

### Part A

The problem of the prior art is solved by the provision, the preparation, and the use of dual color photoinitiators according to the present disclosure, especially by the process for the manufacture of a spiropyran according to independent claim 1 and by the spiropyran according to independent claim 16, the process for the manufacture of a spiropyran according to dependent claim 6, or 12, the manufacture of a precursor of formula (2) according to claim 14, and a process for locally polymerizing a starting material according to claims 30 to 32.

Especially, it may be the object of the present invention to provide a synthetic method for the formation of a spiropyran structural motif. The provided technology shall be applicable to the fields of spiropyrans and/or dynamic covalent chemistry in general and to dual color photoinitiators in particular. More particular, it may be the object of the present invention to overcome limitations of the currently available dual color photoinitiators by providing spiropyrans. More particular a method for the synthesis of photoinitiators, which are spiropyrans, may be provided, which are not or only with difficulty accessible in the previously described synthetic pathways. The thereby provided photoinitiators cause curing of photopolymerizable formulations upon irradiation with two different wavelengths and may be used for volumetric printing (xolography).

The first aspect of the invention is a process for the manufacture of a spiropyran represented by the following formula (1):

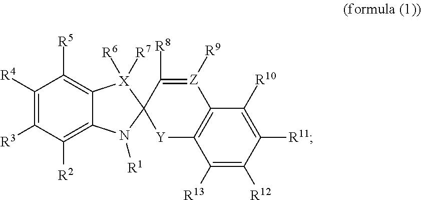

the process comprising the steps of
   providing a precursor, wherein the precursor is a spiropyran represented by the following formula (2):

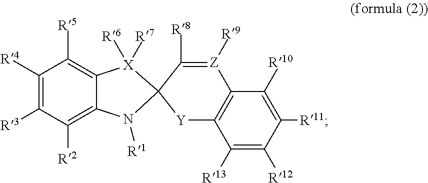

providing a reactant, wherein the reactant is
an indolenium salt represented by the following formula (3):

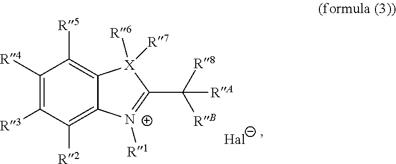

or the corresponding 2-methyleneindoline compound; or
a salicylaldehyde represented by the following formula (4):

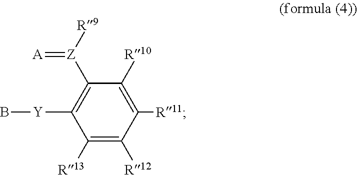

or
a spiropyran represented by the following formula 5:

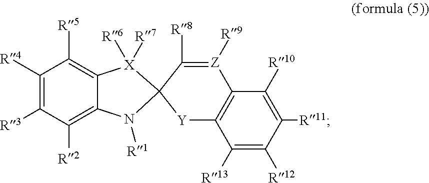

optionally pre-activating the precursor
   providing a reaction mixture comprising the precursor or optionally a preactivated precursor, and the reactant to obtain said spiropyran of formula (1);
wherein the spiropyran, the precursor, and the reactant are different from each other;
wherein if the reactant is an indolenium salt of formula (3), the obtained spiropyran of formula (1) is represented by the following formula (1A):

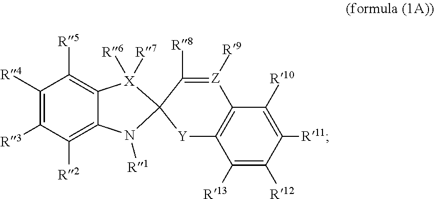

wherein R^{″1} to R^{″8} of formula (1A) are independently the same as R^{″1} to R^{″8} of formula (3) and R^{′9} to R^{′13} of formula (1A) are independently the same as R^{′9} to R^{′13} of formula (2);
wherein if the reactant is a salicylaldehyde of formula (4), the obtained spiropyran of formula (1) is represented by the following formula (1B):

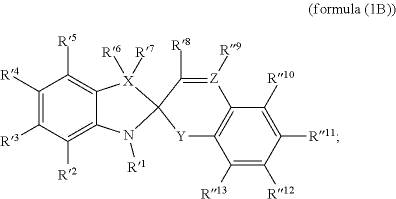

wherein R^{′1} to R^{′8} of formula (1B) are independently the same as R^{′1} to R^{′8} of formula (2) and R^{″9} to R^{″13} of formula (1B) are independently the same as R^{″9} to R^{″13} of formula (4);
wherein if the reactant is a spiropyran of formula (5), the obtained spiropyran of formula (1) is represented by formula (1A), wherein R^{″1} to R^{″8} of formula (1A) are independently the same as R^{″1} to R^{″8} of formula (5) and R^{′9} to R^{′13} of formula (1A) are independently the same as R^{′9} to R^{′13} of formula (2), or
the obtained spiropyran of formula (1) is represented by formula (1B), wherein R^{′1} to R^{′8} of formula (1B) are independently the same as R^{′1} to R^{′8} of formula (2) and R^{″9} to R^{″13} of formula (1B) are independently the same as R^{″9} to R^{″13} of formula (5);
wherein X is selected from S, C, or N; if X is S, then R⁶, R⁷, R^{′6}, R^{′7}, R^{″6}, R^{″7} may not be present accordingly; if X is N, then R⁷, R^{′7}, R^{″7} may not be present accordingly,
wherein Y is selected from O, S, or N; where Y is N, the substituent contains the atoms necessary to complete a cyclic structure with R¹³ selected from the group consisting of benzimidazole, indoline, indole, dihydroquinoline, and tetrahydroquinoline;
wherein Z is selected from N or C;
wherein if present A is selected from O, S, or Se;
wherein if present B is selected from H or D;
wherein if present Hal- is a halogen anion or an anionic compound;
wherein if present R¹ to R¹³, R^{′1} to R^{′13}, and R^{″1} to R^{″13} are independently selected from the group consisting of H; D; halogen; NO₂; CN; OH; SH; substituted or unsubstituted C₁-C₂₀-alkyl; substituted or unsubstituted C₃-C₂₀-cycloalkyl; substituted or unsubstituted C₆-C₄₈-aryl; substituted or unsubstituted C₂-C₄₂-heteroaryl; substituted or unsubstituted C₂-C₄₉-alkyl acyl; substituted or unsubstituted C₂-C₄₉-aryl acyl; substituted or unsubstituted C₁-C₂₀-alkoxy; substituted or unsubstituted C₆-C₄₈-aryloxy; NH₂; substituted or unsubstituted C₁-C₂₀-alkyl ester; substituted or unsubstituted C₆-C₄₈-aryl ester; substituted or unsubstituted C₁-C₂₀ alkyl amide; substituted or unsubstituted C₆-C₄₈-aryl amide; NR′₂; SiR′₃; —O—SiR′₃, wherein R′ is independently selected from the group consisting of substituted or unsubstituted C₁-C₂₀-alkyl and substituted or unsubstituted C₆-C₄₈-aryl, two R′ may form a ring structure; substituted or unsubstituted carboxylic acids and salts thereof; substituted or unsubstituted sulfonic acids and salts thereof; substituted or unsubstituted sulfonic esters; substituted or unsubstituted sulfonic amides; formyl; ether, thioether; carbonate; carbonate ester; sulfates; boronic acids; boronic esters; phosphonic acids; phosphonic esters; phosphines; phosphates; peroxycarbonic acids; thiocarbonic acids; sulfinic acids; sulfinic esters; sulfonates; thiolesters, sulfoxides; sulfones; alkylsulfones; hydrazides; thioaldehydes; ketones; thioketones; oximes; hydrazines; nitroso; azo; diazo; diazonium; isocyanides; cyanate; isocyanate; thiocyanate; isothiocyanate; hydroperoxide; peroxide; acetals; ketal; orthoester; orthocarbonate esters; ammonium; imines; imides; azide; nitrate; isonitrile; nitrosoxy; substituted or unsubstituted carbamates; substituted or unsubstituted ethers; substituted or unsubstituted polyether carbamates; substituted or unsubstituted arylazo; substituted or unsubstituted C₂-C₂₀-alkynyl and substituted or unsubstituted C₂-C₂₀-alkenyl;
wherein the one or more substituents if present in one or more of R¹ to R¹³, are independently selected from the group consisting of D; halogen; NO₂; CN, C₂-C₄₉-alkyl acyl; substituted or unsubstituted C₁-C₂₀-alkoxy; substituted or unsubstituted C₆-C₄₈-aryloxy; substituted or unsubstituted C₂-C₄₉-aryl acyl; (meth)acrylate; tosyl; sulfonic acid or salts thereof; carboxylic acid or salts thereof; boronic acid or salts thereof; phosphonic acid or salts thereof; NR′₃⁺, wherein R′ is independently selected from the group consisting of H, D, substituted or unsubstituted C₁-C₂₀-alkyl and substituted or unsubstituted C₆-C₄₈-aryl, two R′ may form a ring structure; NH₂; and OH;
wherein if present two adjacent groups of R² to R⁵, R^{′2} to R^{′5}, R^{″2} to R^{″5}, R¹⁰ to R¹³, R^{′10} to R^{′13} and R^{″10} to R^{″13} may be independently linked to each other to form a fused ring structure; and
wherein if present R^{″A} to R^{″B} are independently selected from H and D.

A second aspect of the embodiment is a process for the manufacture of a precursor represented by the following formula (2):

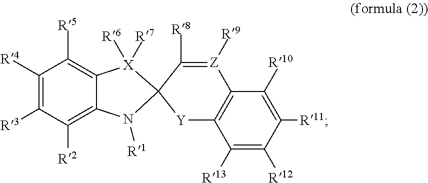

wherein R^{′1}, R^{′6} to R^{′9} are independently selected from the group consisting of H, D, substituted or unsubstituted C₁-C₁₀-alkyl, preferably methyl; substituted or unsubstituted C₆-C₃₂-aryl, preferably phenyl; substituted or unsubstituted C₂-C₂₀-alkynyl and substituted or unsubstituted C₂-C₂₀-alkenyl and benzyl;
X is C;
Z is C;
Y is O;
R^{′2} to R^{′5} and R^{′10} to R^{′13} are independently selected from the group consisting of H, D, F, Cl, Br, substituted or unsubstituted C₁-C₁₀-alkyl, substituted or unsubstituted C₆-C₃₂-aryl, substituted or unsubstituted C₁-C₂₀-alkoxy, substituted or unsubstituted C₆-C₄₈-aryloxy, CF₃, CN; wherein two adjacent groups of may be linked to each other to form a fused ring structure, preferably, a fused aromatic C₆-ring; and a substituent of the following formula:

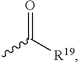

wherein R¹⁹ is defined as it is defined in the description;
wherein at least one of R^{′2} to R^{′5} and R^{′10} to R^{′13} is a substituent of the following formula:

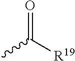

the process comprising the steps of:
   providing a reactant, wherein the reactant is a spiropyran represented by the following formula (2A):

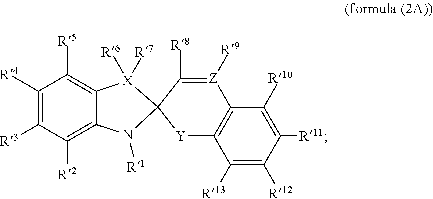

wherein at least one of R^{′2} to R^{′5} and R^{′10} to R^{′13} is a halogen atom selected from the group consisting of Cl, Br and I; and the rest of the substituents are the same as in the precursor of formula (2);
   reacting the halogen atom of the reactant in a metal-halogen exchange reaction, preferably in a metal-halogen exchange reaction with an organolithium reagent or Grignard reagent, to obtain a metal-spiropyran species,
   subsequently reacting the metal-spiropyran species with a Weinreb-amide of the following formula:

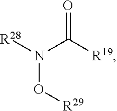

wherein R²⁸ and R²⁹ are selected from the group consisting of substituted or unsubstituted C₁-C₁₀-alkyl; substituted or unsubstituted C₆-C₃₂-aryl; substituted or unsubstituted C₂-C₂₀-alkynyl and substituted or unsubstituted C₂-C₂₀-alkenyl, preferably are R²⁸ and R²⁹ are each methyl,
to obtain the precursor of formula (2), wherein preferably the precursor of formula (2) is obtained after an acidic aqueous work-up.

A third aspect of the invention is a spiropyran represented by the following formula (1):

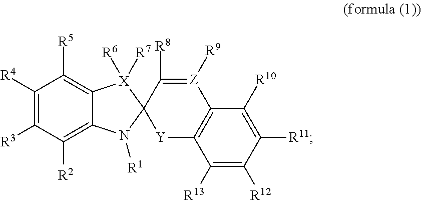

wherein X is selected from S, C, or N; if X is S, then R⁶, R⁷, R^{′6}, R^{′7}, R^{″6}, R^{″7} may not be present accordingly; if X is N, then R⁷, R^{′7}, R^{″7} may not be present accordingly,
wherein Y is selected from O, S, or N; where Y is N, the substituent contains the atoms necessary to complete a cyclic structure with R¹³ selected from the group consisting of benzimidazole, indoline, indole, dihydroquinoline, and tetrahydroquinoline;
wherein Z is selected from N or C;
wherein R¹ to R¹³ are independently selected from the group consisting of H; D; halogen; NO₂; CN; OH; SH; CF₃; substituted or unsubstituted C₁-C₂₀-alkyl; substituted or unsubstituted C₃-C₂₀-cycloalkyl; substituted or unsubstituted C₆-C₄₈-aryl; substituted or unsubstituted C₂-C₄₂-heteroaryl; substituted or unsubstituted C₂-C₄₉-alkyl acyl; substituted or unsubstituted C₂-C₄₉-aryl acyl; substituted or unsubstituted C₁-C₂₀-alkoxy; substituted or unsubstituted C₆-C₄₈-aryloxy; NH₂; substituted or unsubstituted C₁-C₂₀-alkyl ester; substituted or unsubstituted C₆-C₄₈-aryl ester; substituted or unsubstituted C₁-C₂₀ alkyl amide; substituted or unsubstituted C₆-C₄₈-aryl amide; NR′₂; SiR′₃; —O—SiR′₃, wherein R′ is independently selected from the group consisting of substituted or unsubstituted C₁-C₂₀-alkyl and substituted or unsubstituted C₆-C₄₈-aryl, two R′ may form a ring structure; substituted or unsubstituted carboxylic acids and salts thereof; substituted or unsubstituted sulfonic acids and salts thereof; substituted or unsubstituted sulfonic esters; substituted or unsubstituted sulfonic amides; formyl; ether; thioether; carbonate; carbonate ester; sulfates; boronic acids; boronic esters; phosphonic acids; phosphonic esters; phosphines; phosphates; peroxycarbonic acids; thiocarbonic acids; sulfinic acids; sulfinic esters; sulfonates; thiolesters, sulfoxides; sulfones; alkylsulfones; hydrazides; thioaldehydes; ketones; thioketones; oximes; hydrazines; nitroso; azo; diazo; diazonium; isocyanides; cyanate; isocyanate; thiocyanate; isothiocyanate; hydroperoxide; peroxide; acetals; ketal; orthoester; orthocarbonate esters; ammonium; imines; imides; azide; nitrate; isonitrile; nitrosoxy; substituted or unsubstituted carbamates; substituted or unsubstituted ethers; substituted or unsubstituted polyether carbamates; substituted or unsubstituted arylazo; substituted or unsubstituted C₂-C₂₀-alkynyl and substituted or unsubstituted C₂-C₂₀-alkenyl; wherein two adjacent groups of may be linked to each other to form a fused ring structure, preferably, a fused aromatic C₆-ring;
wherein the one or more substituents, if present in one or more of R¹ to R¹³, are independently selected from the group consisting of D; halogen; NO₂; CN, C₂-C₄₉-alkyl acyl; substituted or unsubstituted C₁-C₂₀-alkoxy; substituted or unsubstituted C₆-C₄₈-aryloxy; substituted or unsubstituted C₂-C₄₉-aryl acyl; (meth)acrylate; tosyl; sulfonic acid or salts thereof; carboxylic acid or salts thereof; boronic acid or salts thereof; phosphonic acid or salts thereof; NR′₃, wherein R′ is independently selected from the group consisting of H, D, substituted or unsubstituted C₁-C₂₀-alkyl and substituted or unsubstituted C₆-C₄₈-aryl, two R′ may form a ring structure; NH₂; and OH; wherein if present two adjacent groups of R¹⁰ to R¹³, and R² to R⁵ may be independently linked to each other to form a fused ring structure;
and
wherein at least one substituent for R² to R⁵ and R¹⁰ to R¹³ is selected from one of the groups consisting of chlorine; bromine; iodine; formyl; carbonate; carbonate ester; ester; amide; CF₃; substituted or unsubstituted C₂-C₄₉-alkyl acyl; substituted or unsubstituted C₂-C₄₉-aryl acyl; ketone; acyl; oxime; aldehyde, NO₂; CN; (meth)acrylate; sulfones; alkylsulfones; sulfonamides; SO₂Me; SO₂NH₂; methoxy; and tosyl; or of the following formulae:

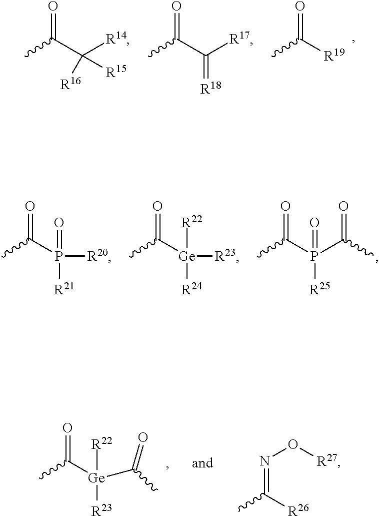

wherein R¹⁴ to R²⁷ are independently selected from the group consisting of H; D; halogen; NO₂; CN; OH; SH; CF₃; substituted or unsubstituted C₁-C₂₀-alkyl; substituted or unsubstituted C₃-C₂₀-cycloalkyl; substituted or unsubstituted C₆-C₄₈-aryl; substituted or unsubstituted C₂-C₄₂-heteroaryl; substituted or unsubstituted C₂-C₄₉-alkyl acyl; substituted or unsubstituted C₂-C₄₉-aryl acyl; substituted or unsubstituted C₁-C₂₀-alkoxy; substituted or unsubstituted C₆-C₄₈-aryloxy, and NH₂; substituted or unsubstituted C₁-C₂₀-alkyl ester; substituted or unsubstituted C₆-C₄₈-aryl ester; substituted or unsubstituted C₁-C₂₀ alkyl amide; substituted or unsubstituted C₆-C₄₈-aryl amide; NR′₂, SiR′₃, —O—SiR′₃ wherein R′ is independently selected from the group consisting of substituted or unsubstituted C₁-C₂₀-alkyl and substituted or unsubstituted C₆-C₄₈-aryl, two R′ may form a ring structure; substituted or unsubstituted carboxylic acids and salts thereof; substituted or unsubstituted sulfonic acids and salts thereof; substituted or unsubstituted sulfonic esters; substituted or unsubstituted sulfonic amides; formyl; ether, thioether; carbonate; carbonate ester; sulfates; boronic acids; boronic esters; phosphonic acids; phosphonic esters; phosphines; phosphates; peroxycarbonic acids; thiocarbonic acids; sulfinic acids; sulfinic esters; sulfonates; thiolesters, sulfoxides; sulfones; alkylsulfones; hydrazides; thioaldehydes; ketones; thioketones; oximes; hydrazines; nitroso; azo; diazo; diazonium; isocyanides; cyanate; isocyanate; thiocyanate; isothiocyanate; hydroperoxide; peroxide; acetals; ketal; orthoester; orthocarbonate esters; ammonium; imines; imides; azide; nitrate; isonitrile; nitrosoxy; substituted or unsubstituted carbamates; substituted or unsubstituted ethers; substituted or unsubstituted polyether carbamates; substituted or unsubstituted arylazo; substituted or unsubstituted C₂-C₂₀-alkynyl and substituted or unsubstituted C₂-C₂₀-alkenyl;
wherein the one or more substituents, if present in one or more of R¹⁴-R²⁷, are independently selected from the group consisting of D; halogen; NO₂; CN, C₂-C₄₉-alkyl acyl; substituted or unsubstituted C₁-C₂₀-alkoxy; substituted or unsubstituted C₆-C₄₈-aryloxy; substituted or unsubstituted C₂-C₄₉-aryl acyl; (meth)acrylate; tosyl; sulfonic acid or salts thereof, carboxylic acid or salts thereof, boronic acid or salts thereof, phosphonic acid or salts thereof, NR′₃, wherein R′ is independently selected from the group consisting of H, D, substituted or unsubstituted C₁-C₂₀-alkyl and substituted or unsubstituted C₆-C₄₈-aryl, two R′ may form a ring structure; NH₂; and OH; and R¹⁵ and R¹⁶ may be linked to each other to form a unsubstituted or substituted ring structure,
and
wherein at least one other substituent for R² to R⁵ and R¹⁰ to R¹³ is selected from the group consisting of chlorine; bromine; iodine; formyl; carbonate; carbonate ester; ester; amide; CF₃; substituted or unsubstituted C₂-C₄₉-alkyl acyl; substituted or unsubstituted C₂-C₄₉-aryl acyl; ketone; acyl; oxime; aldehyde, NO₂; CN; (meth)acrylate; sulfones; alkylsulfones; sulfonamides; SO₂Me; SO₂NH₂; methoxy; and tosyl;
and/or
wherein at least one other substituent for R² to R⁵ and R¹⁰ to R¹³ is selected from sulfonic acid or salts thereof; carboxylic acid or salts thereof; boronic acid or salts thereof; phosphonic acid or salts thereof; NR′₃⁺, wherein R′ is independently selected from the group consisting of H, D, substituted or unsubstituted C₁-C₂₀-alkyl and substituted or unsubstituted C₆-C₄₈-aryl, two R′ may form a ring structure; substituted or unsubstituted sulfonic esters; substituted or unsubstituted sulfonic amides; phosphonic esters; phosphines; phosphates; sulfinic acids; sulfinic esters; sulfonates; sulfoxides; sulfones; alkylsulfones; oximes; isocyanides; cyanate; isocyanate; thiocyanate; isothiocyanate; ammonium; substituted or unsubstituted carbamates; (meth)acrylate; tosyl; NH₂; and OH.

Furthermore, the spiropyrans of the present invention may be photoinitiators, preferably dual color photoinitiators, or used as such. Further embodiments are subject to dependent claims.

In a further aspect of the present invention, a formulation may contain the spiropyran of formula (1), which may be a photoinitiator.

Another aspect of the present invention is a process for locally polymerizing a starting material by using a spiropyran according to the present invention as a photoinitiator and irradiating the spiropyran with two light sources of different wavelengths.

One aspect of the present invention is a process for locally polymerizing a starting material by dual color photopolymerization, comprising:
providing a polymerizable starting material containing photoinitiator molecules wherein the photoinitiator molecule is a spiropyran according to the present invention, which can be converted by sequential optical excitation into a reactive state in which the photoinitiator molecules locally trigger polymerization of the starting material; and
photopolymerizing the starting material in a local volume by irradiating light of a first wavelength and light of a second wavelength, different from the first wavelength, into the local volume, whereby in the local volume
the photoinitiator molecules are converted, due to the absorption of the light of the first wavelength, from an initial state in which the photoinitiator molecules substantially do not absorb the light of the second wavelength, into an intermediate state with changed optical properties compared to the initial state, such that the photoinitiator molecules in the intermediate state absorb the light of the second wavelength; and
the photoinitiator molecules are transferred from the intermediate state to the reactive state due to the absorption of the light of the second wavelength, which triggers the polymerization locally; and/or
the photoinitiator molecules may be transferred from the intermediate state to the initial state spontaneously in a thermal reaction.
   One further aspect of the present invention is a process for the formation of shaped body by dual color photopolymerization, comprising:
providing a polymerizable starting material containing photoinitiator molecules wherein the photoinitiator molecule is a spiropyran as it is defined in the present invention, which can be converted by sequential optical excitation into a reactive state in which the photoinitiator molecules locally trigger polymerization of the starting material; and
photopolymerizing the starting material in a local volume by irradiating light of a first wavelength and light of a second wavelength, different from the first wavelength, into the local volume, whereby in the local volume
the photoinitiator molecules are converted, due to the absorption of the light of the first wavelength, from an initial state in which the photoinitiator molecules substantially do not absorb the light of the second wavelength, into an intermediate state with changed optical properties compared to the initial state, such that the photoinitiator molecules in the intermediate state absorb the light of the second wavelength; and
the photoinitiator molecules are transferred from the intermediate state to the reactive state due to the absorption of the light of the second wavelength, which triggers the polymerization locally; and
the photoinitiator molecules may be transferred from the intermediate state to the initial state spontaneously in a thermal reaction.
   Yet another aspect of the present invention is a process for locally polymerizing a starting material by dual color photopolymerization and forming a shaped body, comprising the following steps:
providing a container which is at least partially filled with a polymerizable starting material containing photoinitiator molecules, which can be converted by sequential optical excitation into a reactive state in which the photoinitiator molecules locally trigger polymerization of the starting material; and
photopolymerizing the starting material in the container by irradiating light of a first wavelength and light of a second wavelength, different from the first wavelength, into the container, whereby in the container
the photoinitiator molecules are converted, due to the absorption of the light of the first wavelength, from an initial state in which the photoinitiator molecules substantially do not absorb the light of the second wavelength, into an intermediate state with changed optical properties compared to the initial state, such that the photoinitiator molecules in the intermediate state absorb the light of the second wavelength; and
the photoinitiator molecules are transferred from the intermediate state to the reactive state due to the absorption of the light of the second wavelength, which triggers the polymerization locally to form the shaped body; and
   wherein the initial state of the photoinitiator molecules in the photopolymerizable material has an extinction coefficient which is lower than 5000 L mol⁻¹ cm⁻¹.

In yet another aspect, the present invention discloses a process for locally polymerizing a starting material by dual color photopolymerization and forming a shaped body, comprising the following steps:
providing a container which is at least partially filled with a polymerizable starting material containing photoinitiator molecules, which can be converted by sequential optical excitation into a reactive state in which the photoinitiator molecules locally trigger polymerization of the starting material; and
photopolymerizing the starting material in a container by irradiating light of a first wavelength and light of a second wavelength, different from the first wavelength, into the container, whereby in the container
the photoinitiator molecules are converted, due to the absorption of the light of the first wavelength, from an initial state in which the photoinitiator molecules substantially do not absorb the light of the second wavelength, into an intermediate state with changed optical properties compared to the initial state, such that the photoinitiator molecules in the intermediate state absorb the light of the second wavelength; and
the photoinitiator molecules are transferred from the intermediate state to the reactive state due to the absorption of the light of the second wavelength, which triggers the polymerization locally to form a shaped body;
   wherein the photopolymerizable material has an absorbance at the first wavelength in a range between 1 and 0.07 absorbance units.

### DETAILED DESCRIPTION

### 1. General Process for the Manufacture of Spiropyran of Formula (1)

The spiropyran of formula (1) may be obtained by a reversible exchange reaction.

Without being bound by any theory, the inventors provide a plausible mechanism of the present reversible exchange reaction in the following outlined scheme which may occur in the reaction mixture: A spiropyran opens to the merocyanine form. Water can undergo a Michael Addition to the merocyanine, which reacts further to the free salicylaldehyde and the 2-methyleneindoline. The 2-methyleneindoline is a strong nucleophile which can add to another merocyanine in a reversible fashion. In further steps, one 2-methyleneindoline is released from the adduct, leaving a new merocyanine which can close to the respective spiropyran. The thereby released 2-methyleneindoline can undergo a spiropyran condensation with salicylaldehyde which has been liberated earlier from a spiropyran (shown in the scheme below) or a different salicylaldehyde which has been added to the reaction mixture (not shown in the scheme below). Vice versa, the released salicylaldehyde can undergo a spiropyran condensation with 2-methyleneindoline which has been liberated earlier from a spiropyran (shown in the scheme below) or a different 2-methyleneindoline which has been added to the reaction mixture (not shown in the scheme below).

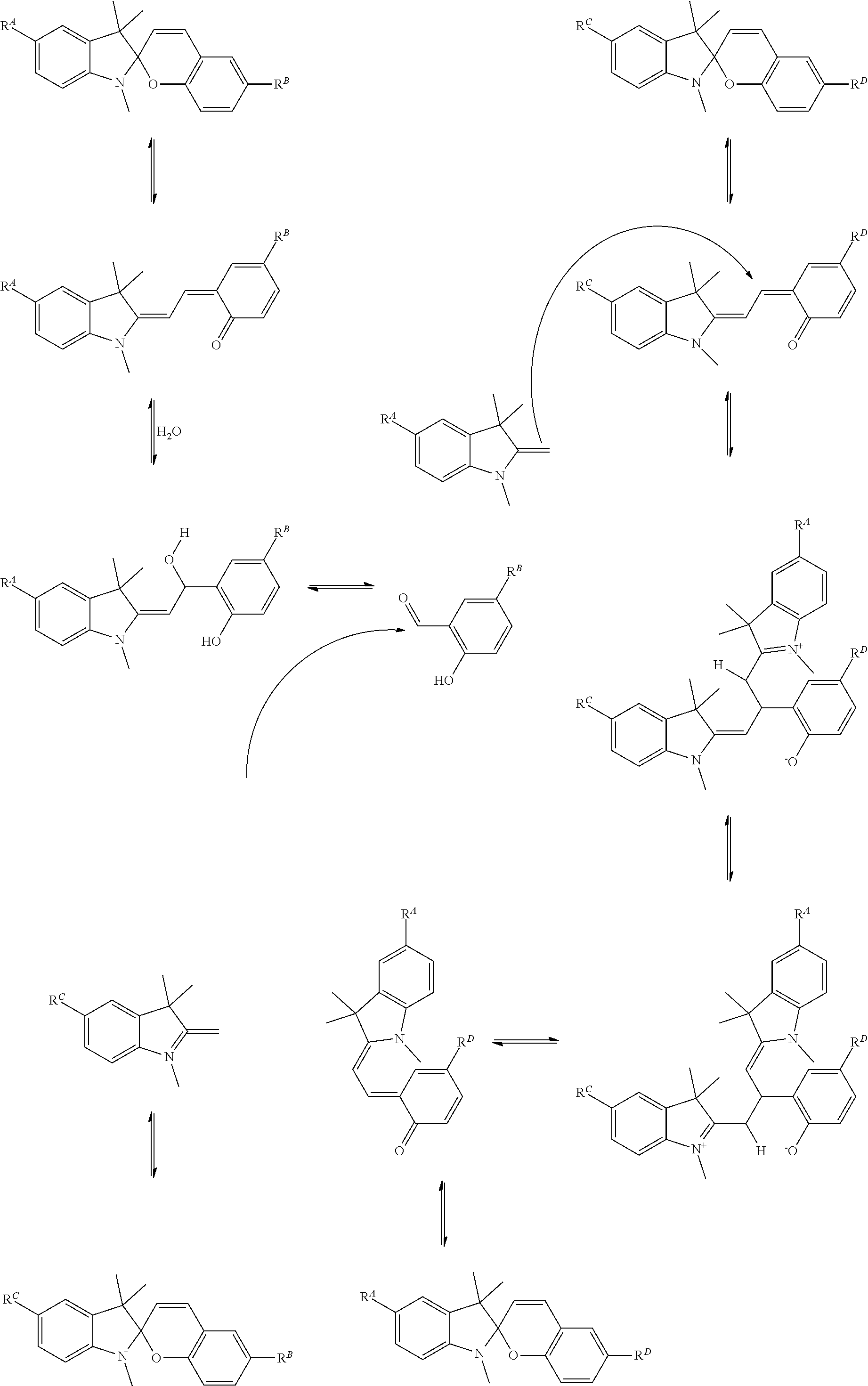

Many nucleophiles could undergo the described Michael Addition on the merocyanine. In some cases, it may be advantageous to treat the precursor spiropyran with a nucleophile, such as methylamine, and pre-activate it for the reaction (shown in scheme below). This activation may lead to the formation of the respective imine/iminium/salicylaldehyde and 2-methyleneindoline derivatives and may be more efficient than with water. The respective salicylaldehyde and 2-methyleneindoline may be separated, e.g. by basic extraction, prior to addition of the reactant.

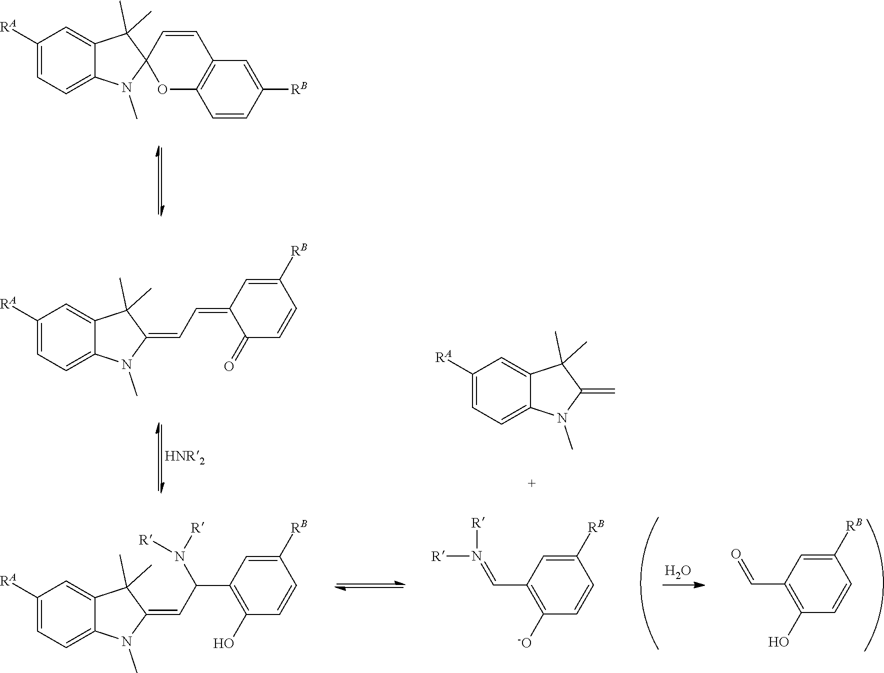

Another option to accelerate the exchange of two spiropyrans may be to add a catalytic amount of a 2-methyleneindoline, so that there may always be some nucleophile in slight excess present. In some embodiments, the efficiency of the reaction may be enhanced by substitution of the indole with an electron donating moiety and/or by substitution of the salicylaldehyde with an electron withdrawing moiety. Such a substitution pattern may facilitate the opening to the merocyanine and the attack of the 2-methyleneindoline on the merocyanine. In another embodiment, the equilibrium may be shifted towards the product side by substitution of R^{8′} in the precursor of formula (2) with an electron donating moiety, such as methyl. Such substituent may influence the basicity und nucleophilicity of the corresponding 2-methyleneindoline derivative.

Accordingly, a mixture of two or more spiropyrans may exchange their respective components which originate from the 2-methyleneindoline and salicylaldehyde, respectively. The spiropyrans may be dissolved in a suitable solvent and heated until the equilibrium may be reached. Starting from two spiropyrans, with different 2-methyleneindoline and salicylaldehyde components, four different spiropyrans in the mixture may be found, when the thermodynamic equilibrium may be reached. The number of possible spiropyrans may increase according to the possible combinations. From the number of starting spiropyrans originating from different 2-methyleneindolines I and different salicylaldehydes S, the maximum number of different derivatives in the mixture P may be deduced according to the formula P=S*I.

As stated above, in one aspect, a process for the manufacture of a spiropyran represented by the following formula (1):

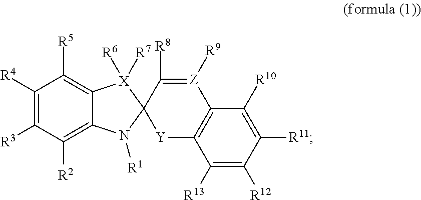

is provided. The process comprises the steps of
   providing a precursor, wherein the precursor is a spiropyran represented by the following formula (2):

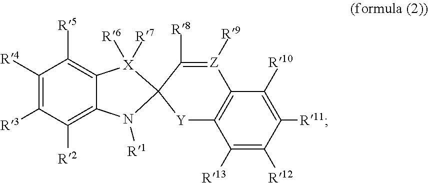

providing a reactant, wherein the reactant is
an indolenium salt represented by the following formula (3):

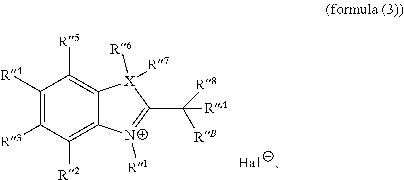

or the corresponding 2-methyleneindoline compound; or as an alternative
a salicylaldehyde represented by the following formula (4):

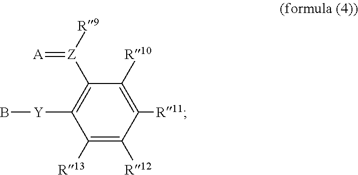

or as another alternative
a spiropyran represented by the following formula 5:

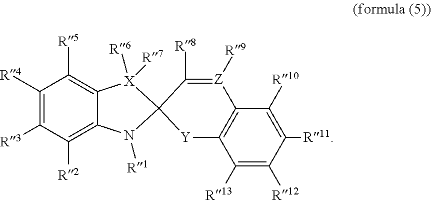

Optionally, the precursor is pre-activated. A reaction mixture comprising the precursor or optionally a preactivated precursor is provided, as well as the reactant to obtain said spiropyran of formula (1). Thereby, the spiropyran, the precursor, and the reactant are different from each other. If the reactant is an indolenium salt of formula (3), the obtained spiropyran of formula (1) is represented by the following formula (1A):

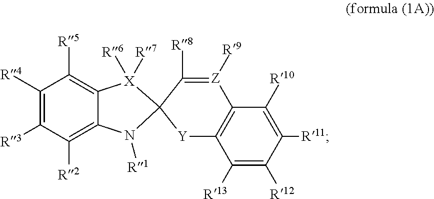

wherein R^{″1} to R^{″8} of formula (1A) are independently the same as R^{″1} to R^{″8} of formula (3) and R^{′9} to R^{′13} of formula (1A) are independently the same as R^{′9} to R^{′13} of formula (2);
If the reactant is a salicylaldehyde of formula (4), the obtained spiropyran of formula (1) is represented by the following formula (1B):

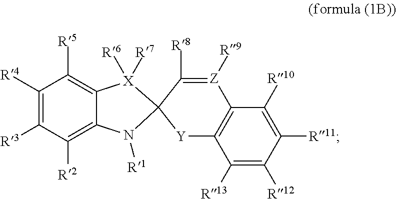

wherein R^{′1} to R^{′8} of formula (1B) are independently the same as R^{′1} to R^{′8} of formula (2) and R^{″9} to R^{″13} of formula (1B) are independently the same as R^{″9} to R^{″13} of formula (4).

In a further aspect of the invention, the present invention solves the problem of providing a methyleneindolin.

The skilled person is aware of that a side product of the spiropyran of formula (2) and a methyleneindoline of formula (3) may be a methyleneindoline of the following formula (3B):

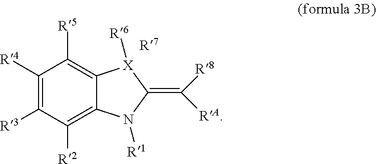

Therein, the substituents of R^{′1}, R^{′2}, R^{′3}, R^{′4}, R^{′5}, R^{′6}, R^{′7} and R^{′8}, as well as X, are the same as in formula (2), from which the methyleneindoline of formula (3B) derives from. The methyleneindoline of (3B) may be protonated as the corresponding indolenium salt. In a preferred embodiment of methyleneindoline of formula (3B), X is C and/or R′A is H. The skilled person will realize that the process for the manufacture of a spiropyran of the present invention may be also used to provide methyleneindolines, like methyleneindolines of formula (3B), that may have been challenging to access or may not have been accessible at all by alternative conventional synthetic routes. The methyleneindoline of formula (3B) may be used as a precursor for a different reaction, for example for the synthesis of another spiropyran.

In a further aspect of the invention, the present invention solves the problem of providing salicylaldehyde. The salicylaldehyde may contain substituents and/or a substituent pattern that may not be accessible by conventional synthetic routes.

The skilled person is aware of that a side product of the spiropyran of formula (2) and salicylaldehyde of formula (4) may be a salicylaldehyde of the following formula (4B):

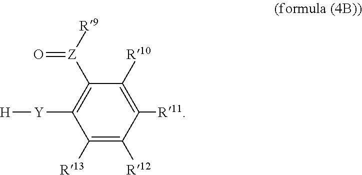

The salicylaldehyde of formula (4b) may be a side product, which may be obtained as a side product of spiropyran of formula (2) which may was pre-activated with a nucleophile, like an amine, as it is described further below.

Therein, the substituents of R^{′9}, R^{′10}, R^{′11}, R^{′12} and R^{′13}, as well as Y and Z, are the same as in formula (2), from which the salicylaldehyde of formula (4B) derives from. In a preferred embodiment of salicylaldehyde of formula (4B), Y is O, Z is C and/or R^{′9} is H. The salicylaldehyde of formula (4B) may be a compound wherein at least one of the substituents R^{′10}, R^{′11}, R^{′12} and R^{′13} are not directly accessible from salicylaldehyde. Consequently, the skilled person will realize that the process for the manufacture of a spiropyran of the present invention may be also used to provide salicylaldehydes, like salicylaldehydes of formula (4B), that may have been challenging to access or may not have been accessible at all by alternative conventional synthetic routes.

Such salicylaldehydes may be accessed by utilizing the spiropyran of formula (2) as a protecting group for the salicylaldehyde of formula (4B) through which substituents R^{′10}, R^{′11}, R^{′12} and R^{′13} can be modified by the methods known in the art. Subsequently, the desired salicylaldehyde of formula (4B) may be “deprotected” by reacting the spiropyran of formula (2) with a nucleophile, for example an amine as it is further specified under pre-activation of the precursor of formula (2) or salicylaldehyde of formula (4) by using the method to manufacture a spiropyran of formula (1B). The salicylaldehyde of formula (4B) may be used as a precursor for a different reaction, for example for the synthesis of another spiropyran.

If the reactant is a spiropyran of formula (5), the obtained spiropyran of formula (1) is represented by formula (1A), wherein R^{″1} to R^{″8} of formula (1A) are independently the same as R^{″1} to R^{″8} of formula (5) and R^{′9} to R^{′13} of formula (1A) are independently the same as R^{′9} to R^{′13} of formula (2), or
the obtained spiropyran of formula (1) is represented by formula (1B), wherein R^{′1} to R^{′8} of formula (1B) are independently the same as R^{′1} to R^{′8} of formula (2) and R^{″9} to R^{″13} of formula (1B) are independently the same as R^{″9} to R^{″13} of formula (5);
X is selected from S, C, or N; if X is S, then R⁶. R⁷, R^{′6}, R^{′7}, R^{″6}, R^{″7} may not be present accordingly; if X is N, then R⁷, R^{′7}, R^{″7} may not be present accordingly preferably X is C.

Y is selected from O, S, or N; where Y is N, the substituent contains the atoms necessary to complete a cyclic structure with R13 selected from the group consisting of benzimidazole, indoline, indole, dihydroquinoline, and tetrahydroquinoline, preferably Y is O.

Z is selected from N or C, preferably Z is C.

If present A is selected from O, S, or Se, preferably A is O.

If present B is selected from H or D, preferably B is H.

If present Hal- is a halogen anion or an anionic compound, preferably Hal- is Cl—, Br—, I—, more preferably Hal- is I—.

If present R¹ to R¹³, R^{′1} to R^{′13}, and R^{″1} to R^{″13} are independently selected from the group consisting of H; D; halogen; NO₂; CN; OH; SH; substituted or unsubstituted C₁-C₂₀-alkyl; substituted or unsubstituted C₃-C₂₀-cycloalkyl; substituted or unsubstituted C₆-C₄₈-aryl; substituted or unsubstituted C₂-C₄₂-heteroaryl; substituted or unsubstituted C₂-C₄₉-alkyl acyl; substituted or unsubstituted C₂-C₄₉-aryl acyl; substituted or unsubstituted C₁-C₂₀-alkoxy; substituted or unsubstituted C₆-C₄₈-aryloxy; NH₂; substituted or unsubstituted C₁-C₂₀-alkyl ester; substituted or unsubstituted C₆-C₄₈-aryl ester; substituted or unsubstituted C₁-C₂₀ alkyl amide; substituted or unsubstituted C₆-C₄₈-aryl amide; NR′₂; SiR′₃; —O—SiR′₃, wherein R′ is independently selected from the group consisting of substituted or unsubstituted C₁-C₂₀-alkyl and substituted or unsubstituted C₆-C₄₈-aryl, two R′ may form a ring structure; substituted or unsubstituted carboxylic acids and salts thereof; substituted or unsubstituted sulfonic acids and salts thereof; substituted or unsubstituted sulfonic esters; substituted or unsubstituted sulfonic amides; formyl; ether, thioether; carbonate; carbonate ester; sulfates; boronic acids; boronic esters; phosphonic acids; phosphonic esters; phosphines; phosphates; peroxycarbonic acids; thiocarbonic acids; sulfinic acids; sulfinic esters; sulfonates; thiolesters, sulfoxides; sulfones; alkylsulfones; hydrazides; thioaldehydes; ketones; thioketones; oximes; hydrazines; nitroso; azo; diazo; diazonium; isocyanides; cyanate; isocyanate; thiocyanate; isothiocyanate; hydroperoxide; peroxide; acetals; ketal; orthoester; orthocarbonate esters; ammonium; imines; imides; azide; nitrate; isonitrile; nitrosoxy; substituted or unsubstituted carbamates; substituted or unsubstituted ethers; substituted or unsubstituted polyether carbamates; substituted or unsubstituted arylazo; substituted or unsubstituted C₂-C₂₀-alkynyl and substituted or unsubstituted C₂-C₂₀-alkenyl;

If in one or more of R¹ to R¹³ one or more substituents are present, they are independently selected from the group consisting of D; halogen; NO₂; CN, C₂-C₄₉-alkyl acyl; substituted or unsubstituted C₁-C₂₀-alkoxy; substituted or unsubstituted C₆-C₄₈-aryloxy; substituted or unsubstituted C₂-C₄₉-aryl acyl; (meth)acrylate; tosyl; sulfonic acid or salts thereof; carboxylic acid or salts thereof; boronic acid or salts thereof; phosphonic acid or salts thereof; NR′₃, wherein R′ is independently selected from the group consisting of H, D, substituted or unsubstituted C₁-C₂₀-alkyl and substituted or unsubstituted C₆-C₄₈-aryl, two R′ may form a ring structure; NH₂; and OH.

If two adjacent groups of R² to R⁵, R^{′2} to R^{′5}, R^{″2} to R^{″5}, R¹⁰ to R¹³, R^{′10} to R^{′13} and R^{″10} to R^{″13} are present, they may be independently linked to each other to form a fused ring structure; and if R^{″A} to R^{″B} are present, the may be independently selected from H and D.

Preferably, at least one of R² to R⁵ and R¹⁰ to R¹³ in formula (1) is a substituent selected from the group consisting of chlorine; bromine; iodine; formyl; carbonate; carbonate ester; ester; amide; CF₃; substituted or unsubstituted C₂-C₄₉-alkyl acyl; substituted or unsubstituted C₂-C₄₉-aryl acyl; ketone; acyl; oxime; aldehyde, NO₂; CN; (meth)acrylate; sulfones; alkylsulfones; sulfonamides; SO₂Me; SO₂NH₂; methoxy; and tosyl; or one of the following formulae:

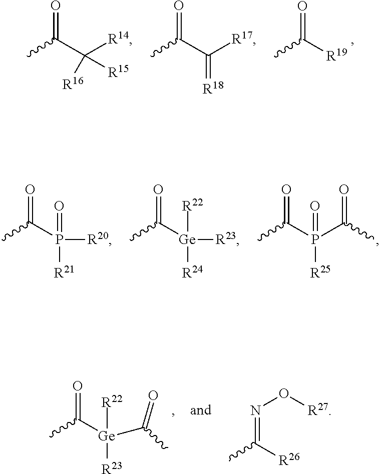

More preferably, at least one of R¹⁰ and R¹² to R¹³ is a substituent selected from the group consisting of chlorine; bromine; iodine; formyl; carbonate; carbonate ester; ester; amide; CF₃; substituted or unsubstituted C₂-C₄₉-alkyl acyl; substituted or unsubstituted C₂-C₄₉-aryl acyl; ketone; acyl; oxime; aldehyde, NO₂; CN; (meth)acrylate; sulfones; alkylsulfones; sulfonamides; SO₂Me; SO₂NH₂; methoxy; and tosyl; or one of the following formulae:

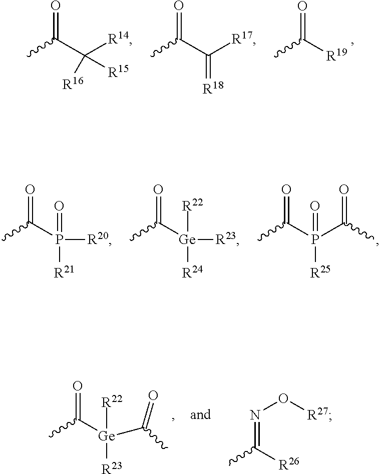

wherein R¹⁴ to R²⁷ are defined above.

More preferably, at least one of R² to R⁵ and additionally at least one of R¹⁰ to R¹³, like preferably R¹⁰ and R¹² to R¹³, in formula (1) are substituents selected from the group consisting of carbonyl; chlorine; bromine; iodine; formyl; carbonate; carbonate ester; ester; amide; CF₃; substituted or unsubstituted C₂-C₄₉-alkyl acyl; substituted or unsubstituted C₂-C₄₉-aryl acyl; ketone; acyl; oxime; aldehyde, NO₂; CN; (meth)acrylate; sulfones; alkylsulfones; sulfonamides; SO₂Me; SO₂NH₂; methoxy; and tosyl; or one of the following formulae:

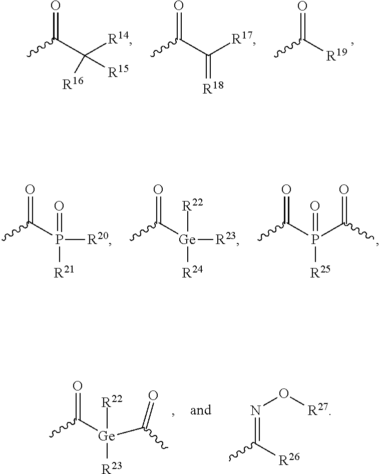

so that the spiropyran of formula (1) contains at least two of such substituents.

A spiropyran with one of the above substituents may be beneficial as a photoinitiator, preferably dual color photoinitiator. On the other hand, spiropyrans containing one of those substituents, may be obtained by a limited or none manufacturing methods, since organometal reagents, like organolithium reagents, may rather attack the carbonyl of the substituent leading to undesired side products. The process for the manufacture of spiropyrans of formula (1) may avoid the use of organometal reagents in the presence sensitive moieties, so that surprisingly the process may provide spiropyrans containing the above substituents, i.e. carbonyl moieties, with higher efficiency.

R¹⁴ to R²⁷ may be independently selected from the group consisting of H; D; halogen; NO₂; CN; OH; SH; substituted or unsubstituted C₁-C₂₀-alkyl; substituted or unsubstituted C₃-C₂₀-cycloalkyl; substituted or unsubstituted C₆-C₄₈-aryl; substituted or unsubstituted C₂-C₄₂-heteroaryl; substituted or unsubstituted C₂-C₄₉-alkyl acyl; substituted or unsubstituted C₂-C₄₉-aryl acyl; substituted or unsubstituted C₁-C₂₀-alkoxy; substituted or unsubstituted C₆-C₄₈-aryloxy; NH₂; substituted or unsubstituted C₁-C₂₀-alkyl ester; substituted or unsubstituted C₆-C₄₈-aryl ester; substituted or unsubstituted C₁-C₂₀ alkyl amide; substituted or unsubstituted C₆-C₄₈-aryl amide; NR′₂; SiR′₃; —O—SiR′₃, wherein R′ is independently selected from the group consisting of substituted or unsubstituted C₁-C₂₀-alkyl and substituted or unsubstituted C₆-C₄₈-aryl, two R′ may form a ring structure; substituted or unsubstituted carboxylic acids and salts thereof; substituted or unsubstituted sulfonic acids and salts thereof; substituted or unsubstituted sulfonic esters; substituted or unsubstituted sulfonic amides; formyl; ether; thioether; carbonate; carbonate ester; sulfates; boronic acids; boronic esters; phosphonic acids; phosphonic esters; phosphines; phosphates; peroxycarbonic acids; thiocarbonic acids; sulfinic acids; sulfinic esters; sulfonates; thiolesters, sulfoxides; sulfones; alkylsulfones; hydrazides; thioaldehydes; ketones; thioketones; oximes; hydrazines; nitroso; azo; diazo; diazonium; isocyanides; cyanate; isocyanate; thiocyanate; isothiocyanate; hydroperoxide; peroxide; acetals; ketal; orthoester; orthocarbonate esters; ammonium; imines; imides; azide; nitrate; isonitrile; nitrosoxy; substituted or unsubstituted carbamates; substituted or unsubstituted ethers; substituted or unsubstituted polyether carbamates; substituted or unsubstituted arylazo; substituted or unsubstituted C₂-C₂₀-alkynyl and substituted or unsubstituted C₂-C₂₀-alkenyl; wherein the one or more substituents, if present in one or more of R¹⁴-R²⁷, are independently selected from the group consisting of D; halogen; NO₂; CN; C₂-C₄₉-alkyl acyl; substituted or unsubstituted C₁-C₂₀-alkoxy; substituted or unsubstituted C₆-C₄₈-aryloxy; substituted or unsubstituted C₂-C₄₉-aryl acyl; (meth)acrylate; tosyl; sulfonic acid or salts thereof; carboxylic acid or salts thereof; boronic acid or salts thereof; phosphonic acid or salts thereof; NR′₃, wherein R′ is independently selected from the group consisting of H, D, substituted or unsubstituted C₁-C₂₀-alkyl and substituted or unsubstituted C₆-C₄₈-aryl, two R′ may form a ring structure; NH₂; and OH; and R¹⁵ and R¹⁶ may be linked to each other to form a unsubstituted or substituted ring structure.

Preferably, at least one of R² to R⁵ and R¹⁰ to R¹³ in formula (1) is a substituent selected from the group consisting of chlorine; bromine; iodine; formyl; carbonate; carbonate ester; ester; amide; CF₃; substituted or unsubstituted C₂-C₄₉-alkyl acyl; substituted or unsubstituted C₂-C₄₉-aryl acyl; ketone; acyl; oxime; aldehyde, NO₂; CN; (meth)acrylate; sulfone; alkylsulfone; sulfonamide; SO₂Me; SO₂NH₂; dicyanovinylene and tosyl. More preferably, at least one of R² to R⁵ and R¹⁰ to R¹³ in formula (1) is a substituent selected from the group consisting of chlorine; bromine; iodine, preferably bromine or iodine.

A halide such as chlorine, bromine or iodine may be transformed to a carbonyl functionality in a subsequent reaction, as outlined in the background of the art. Halides can be provided on every position of a salicylaldehyde of formula (4) and on every position of a 2-methyleneindoline of formula (3) allowing for transformation to a carbonyl group on every position. Thereby, bromides can also be introduced by reaction of N-bromosuccinimide with salicylaldehydes which carry electron withdrawing groups such as trifluoromethyl. Therefore, halogenated spiropyrans may be prepared as precursors for carbonyl substituted spiropyrans. To give an example, one way to introduce the carbonyl is by metal-halide exchange of a bromine on spiropyran using n-butyl lithium and reaction of the formed organo-lithium species with a Weinreb amide. The new spiropyran containing a carbonyl can be subjected to an exchange reaction as disclosed in the invention, allowing substitution patterns which may have not been accessible by conventional methods of the prior art.

More preferably, at least one, preferably at least two, of R² to R⁵ and R¹⁰ to R¹³ in formula (1) is a substituent selected from the group consisting of CN, F, Cl, OCF₃, NO₂, ester groups, ketone, formyl, acyl groups, SO₂R, such as SO₂CF₃, SO₂Me, SO₂Ph, or SO₂NH₂, SF₅, NR₃⁺, pyridinium, halogen, and fluorinated alkyls or aryls, such as CF₃. Even more preferably, a substituent selected from the group consisting of acyl, substituted or unsubstituted benzoyl, CN, F, Cl, OCF₃, NO₂, and CF₃, most preferably acyl, substituted or unsubstituted benzoyl, and CF₃.

In one embodiment R² to R⁵ are independently selected from the group consisting of H and electron withdrawing groups, and one of R¹⁰ to R¹³ is selected from unsubstituted or substituted C₆-C₄₉-aryl acyl; or unsubstituted or substituted C₂-C₄₉-alkyl acyl. Typical electron withdrawing groups may be CN, F, Cl, OCF₃, NO₂, ester groups, formyl, acetyl, benzoyl, substituted benzoyl, acyl groups, SO₂R, such as SO₂CF₃, SO₂Me, SO₂Ph, or SO₂NH₂, SF₅, NR₃⁺, pyridinium, halogen, and fluorinated alkyls or aryls, such as CF₃.

It has been surprisingly found, that in the spiropyran of formula (1), the electron withdrawing groups may be decoupled. Thus, if the spiropyran of formula (1) is used as a photoinitiator, preferably dual color photoinitiator, UV-light is absorbed, and the spiropyran of formula (1) opens to the corresponding merocyanine. The ring opening of the spiropyran to the corresponding merocyanine may be more efficient, the more electron withdrawing groups are present in R² to R⁵ and R¹⁰ to R¹³ in formula (1) as substituents. Hence, in a preferred embodiment multiple electron withdrawing groups in R² to R⁵ and R¹⁰ to R¹³, for example two or more than two CF₃ or other electron withdrawing groups are substituted at R² to R⁵ and R¹⁰ to R¹³. Without being bound by any theory, in the merocyanine form, the acceptors are in conjugation, decreasing the HOMO and LUMO of the merocyanine. When the electron deficient merocyanine absorbs visible light, the excited state is formed, which is strongly oxidizing and has a long lifetime.

In one embodiment at least one of R^{2′} to R^{5′} of spiropyran of formula (2) is independently selected from the group consisting of electron donating groups. Typical electron donating groups may be SH, SR, OH, OR, NH₂, NHR, and NR₂. Such a substitution pattern may facilitate the opening to the merocyanine and the attack of the 2-methyleneindoline on the merocyanine. Preferably, R⁴ is OR, more preferably methoxy. In a further embodiment it may be provided that R¹ is selected from the group consisting of C₁-C₂₀-alkyl and C₆-C₄₈-aryl, alternatively C₁-C₈-alkyl and C₆-C₁₈-aryl, alternatively C₁-C₄-alkyl and C₆-C₁₂-aryl, preferably methyl, benzyl and phenyl.

In an embodiment, the photoinitiator of formula (1) may be linked to a polymerizable group. The polymerizable group may be selected from the group consisting of (meth)acrylate, acrylamide, vinylether, and vinylester, preferably (meth)acrylate by any of R¹ to R¹³.

In a further embodiment, at least one of R¹ to R¹³ in the photoinitiator of formula (1) may comprise at least one structural motif selected from the group of thioxanthone, acenaphtylene-1,2-dione, thiochroman-4-on, 9-fluorenone, anthraquinone, benzanthrone, 9,10-phenanthrenequinone, xanthone, 1,3-indanedione, chromone, 1,4-naphthoquinone, coumarin, benzil, benzophenone, and acetophenone.

In a further embodiment, at least one of R¹ to R¹³ in the photoinitiator of formula (1) may be substituted or unsubstituted C₆-C₄₈-aryl or substituted or unsubstituted C₁-C₂₀-alkyl, wherein the substituent comprises at least one structural motif selected from the group consisting of thioxanthone, acenaphtylene-1,2-dione, thiochroman-4-on, 9-fluorenone, anthraquinone, benzanthrone, 9,10-phenanthrenequinone, xanthone, 1,3-indanedione, chromone, 1,4-naphthoquinone, coumarin, benzil, benzophenone, and acetophenone.

In another embodiment, two or more spiropyran of formula (1) may be linked to each other by a linker group. The chemical bond(s) to the linker group may be independently established by any of R¹ to R¹³, preferably by R¹.

Preferably, at least one of R² to R⁵ and R¹⁰ to R¹³ in formula (1) is a substituent selected from the group consisting of sulfonic acid or salts thereof; carboxylic acid or salts thereof; boronic acid or salts thereof; phosphonic acid or salts thereof; NR′₃, wherein R′ is independently selected from the group consisting of H, D, substituted or unsubstituted C₁-C₂₀-alkyl and substituted or unsubstituted C₆-C₄₈-aryl, two R′ may form a ring structure; substituted or unsubstituted sulfonic esters; substituted or unsubstituted sulfonic amides; phosphonic esters; phosphines; phosphates; sulfinic acids; sulfinic esters; sulfonates; sulfoxides; sulfones; alkylsulfones; oximes; isocyanides; cyanate; isocyanate; thiocyanate; isothiocyanate; ammonium; substituted or unsubstituted carbamates; (meth)acrylate; tosyl; NH₂ and OH.

Preferably, R⁶ and R⁷ are independently substituted or unsubstituted C₁-C₂₀-alkyl. Two adjacent groups of R⁶ and R⁷ may be linked to each other to form a fused cycloalkyl ring structure, preferably, a fused C₄-C₈-cycloalkyl, even more preferably cyclohexyl or cyclopentyl. Preferably, the substituted C₁-C₂₀-alkyl may be substituted with at least one substituent select of the group consisting of terminal sulfonic acids or salts thereof, carboxylic acid or salts thereof, and ammonium salts. or alternatively looped to form C₄-C₈ cycloalkyl ring.

Preferably, R¹ is selected from the group consisting of H, D, substituted or unsubstituted C₁-C₆-alkyl, —CH₂—CH₂—OH, —CH₂—COOH, —CH₂—CH₂—COOH, —CH₂—CH₂—CH₂—NMe₃, —CH₂—CH₂—CH₂—SO₃, phenyl and benzyl. More preferably, R¹ is methyl, —CH₂—CH₂—OH, phenyl or benzyl, —CH₂—COOH, —CH₂—CH₂—COOH, —CH₂—CH₂—CH₂—NMe₃ and —CH₂—CH₂—CH₂—SO₃.

Preferably, R⁸ may be selected from H, D, substituted or unsubstituted C₁-C₆-alkyl, and substituted or unsubstituted phenyl. More preferred, R⁸ is H or methyl.

Preferably, R² to R⁵ may be independently selected from H, D, substituted or unsubstituted C₁-C₆-alkyl, carboxylic acid and salts thereof, sulfonic acid and salts thereof, phosphonic acid and salts thereof, fluorine, bromine, chlorine, iodine, substituted or unsubstituted C₂-C₄₉-alkyl acyl; substituted or unsubstituted C₁-C₂₀-alkoxy; substituted or unsubstituted C₂-C₄₉-aryl acyl, CN, NO₂, aldehyde, ketone, sulfones, sulfonamides, SO₂Me, SO₂Ph, SO₂NH₂, CF₃.

More preferably, at least one of R², R³, R⁵ is substituted or unsubstituted C₂-C₄₉-alkyl acyl; substituted or unsubstituted C₂-C₄₉-aryl acyl, CN, aldehyde, or ketone.

More preferably, at least one of R² to R⁵ is substituted C₂-C₄₉-alkyl acyl; substituted C₂-C₄₉-aryl acyl, where the substituent is chosen from the group of electron withdrawing groups.

Preferably, R¹⁰ to R¹³ may be independently selected from H, D, substituted or unsubstituted C₁-C₆-alkyl, carboxylic acid and salts thereof, sulfonic acid and salts thereof, phosphonic acid and salts thereof, fluorine, bromine, chlorine, iodine, substituted or unsubstituted C₂-C₄₉-alkyl acyl; substituted or unsubstituted C₁-C₂₀-alkoxy; substituted or unsubstituted C₂-C₄₉-aryl acyl, CN, NO₂, aldehyde, ketone, sulfone, sulfonamide, SO₂Me, SO₂Ph, SO₂NH₂, CF₃, OCF₃.

More preferably, at least one of R¹⁰, R¹¹, R¹³ is substituted or unsubstituted C₂-C₄₉-alkyl acyl; substituted or unsubstituted C₂-C₄₉-aryl acyl, CN, aldehyde, or ketone.

In another embodiment, R¹¹ may be selected from the group consisting of CF₃, H, D, substituted or unsubstituted alkoxy, substituted or unsubstituted, more preferably alkyl, methyl, tert-butyl, methoxy.

In one preferred embodiment, R¹¹ may be H, D, substituted or unsubstituted alkoxy, or substituted or unsubstituted alkyl, more preferably methyl, tert-butyl or methoxy. The introduction of R¹¹ being one of the selected substituents has the advantage to provide improved dual color photoinitiators.

More preferably, at least one of R¹⁰ to R¹³ is substituted C₂-C₄₉-alkyl acyl; substituted C₂-C₄₉-aryl acyl, where the substituent is chosen from electron withdrawing groups.

In one embodiment, R¹² and R¹³, or R¹¹ and R¹² may be linked together to form a fused 5- or 6-membered ring. Preferably, the fused 5- or 6-membered ring may contain at least one heteroatom. The heteroatom may be selected from the group consisting of O, S and N. The formed fused 5- or 6-membered ring may be a derivative of flavone, xanthone, thioxanthone, coumarin, or naphthoquinone. Even more preferably, the spiropyran of formula (1) is selected from the group consisting of:

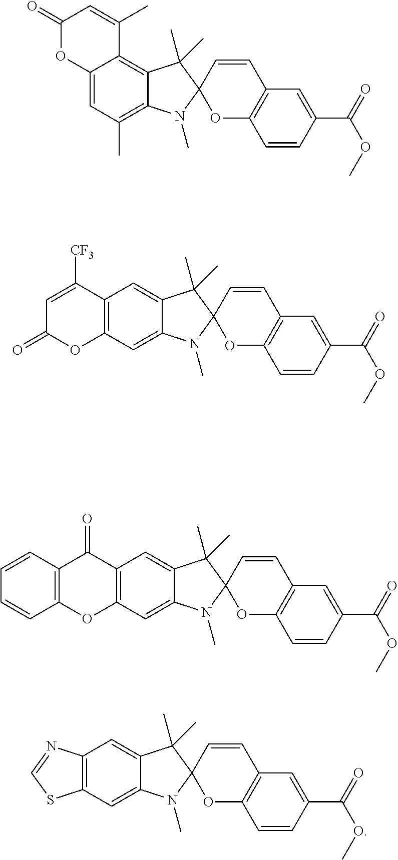 [SMILES: COC(=O)c1ccc2c(c1)C=CC1(O2)N(C)c2c(C)cc3oc(=O)cc(C)c3c2C1(C)C ; COC(=O)c1ccc2c(c1)C=CC1(O2)N(C)c2cc3oc(=O)cc(C(F)(F)F)c3cc2C1(C)C ; COC(=O)c1ccc2c(c1)C=CC1(O2)N(C)c2cc3oc4ccccc4c(=O)c3cc2C1(C)C ; COC(=O)c1ccc2c(c1)C=CC1(O2)N(C)c2cc3scnc3cc2C1(C)C]

Preferably, R¹⁴ may be selected from H, methyl, halogen, more preferably R¹⁴, R¹⁵, and R¹⁶ may be independently selected from H, methyl, halogen. More preferably R¹⁴, R¹⁵, and R¹⁶ are chlorine.

Preferably, R¹⁴, R¹⁵, and R¹⁶ may be independently selected from H, D, CN, substituted or unsubstituted C₁-C₁₀-alkyl; substituted or unsubstituted C₃-C₁₀-cycloalkyl; substituted or unsubstituted C₆-C₃₂-aryl; substituted or unsubstituted C₂-C₂₈-heteroaryl, more preferably, R¹⁴, R¹⁵, and R¹⁶ may be selected from methyl, phenyl, or substituted phenyl.

In a further preferred embodiment, R¹⁴ may be NR′₂, wherein R′ may be independently selected from the group consisting of H, D, substituted or unsubstituted C₁-C₁₀-alkyl and substituted or unsubstituted C₆-C₃₂-aryl, and two R′ may form a ring structure; R¹⁵ and R¹⁶ may be independently selected from H, D, CN, substituted or unsubstituted C₁-C₁₀-alkyl; substituted or unsubstituted C₃-C₁₀-cycloalkyl; substituted or unsubstituted C₆-C₃₂-aryl; substituted or unsubstituted C₂-C₂₈-heteroaryl. More preferably, R¹⁴ may be NR′₂, wherein R′ may be independently selected from the group consisting of substituted or unsubstituted C₁-C₁₀-alkyl, two R′ may form a ring structure; R¹⁵ and R¹⁶ may be independently selected from substituted or unsubstituted C₁-C₁₀-alkyl; substituted or unsubstituted C₃-C₁₀-cycloalkyl; substituted or unsubstituted C₆-C₃₂-aryl. Most preferably, R¹⁴ may be NR′₂, wherein R′ may be methyl, ethyl, or two R′ completing a morpholine; R¹⁵ and R¹⁶ are independently selected from methyl, ethyl, and benzyl.

In another preferred embodiment, R¹⁴ may be OR′, wherein R′ is selected from the group consisting of H, D, substituted or unsubstituted C₁-C₁₀-alkyl; substituted or unsubstituted C₃-C₁₀-cycloalkyl; substituted or unsubstituted C₆-C₃₂-aryl; substituted or unsubstituted C₂-C₂₈-heteroaryl, SiR″₃, wherein R″ is independently selected from the group consisting of substituted or unsubstituted C₁-C₁₀-alkyl and substituted or unsubstituted C₆-C₃₂-aryl, R¹⁵ and R¹⁶ may be independently selected from H, D, CN, substituted or unsubstituted C₁-C₁₀-alkyl; substituted or unsubstituted C₃-C₁₀-cycloalkyl; substituted or unsubstituted C₆-C₃₂-aryl; substituted or unsubstituted C₂-C₂₈-heteroaryl. More preferably, R¹⁴ may be OR′, wherein R′ is independently selected from the group consisting of H, D, substituted or unsubstituted C₁-C₁₀-alkyl, substituted or unsubstituted C₃-C₁₀-cycloalkyl, SiR″₃, wherein R″ is independently selected from the group consisting of substituted or unsubstituted C₁-C₁₀-alkyl and substituted or unsubstituted C₆-C₃₂-aryl. R¹⁵ and R¹⁶ may be independently selected from substituted or unsubstituted C₁-C₁₀-alkyl; substituted or unsubstituted C₃-C₁₀-cycloalkyl; substituted or unsubstituted C₆-C₃₂-aryl. Most preferably, R¹⁴ is OR′, wherein R′ is methyl, ethyl, benzyl, or trimethylsilyl; and R¹⁵ and R¹⁶ are independently selected from methyl, ethyl, phenyl, and benzyl.

In a further preferred embodiment, R¹⁴ and R¹⁵ may be OR′, wherein R′ is independently selected from the group consisting of H, D, substituted or unsubstituted C₁-C₁₀-alkyl; substituted or unsubstituted C₃-C₁₀-cycloalkyl; substituted or unsubstituted C₆-C₃₂-aryl; substituted or unsubstituted C₂-C₂₈-heteroaryl, R¹⁶ may be selected from H, D, CN, substituted or unsubstituted C₁-C₁₀-alkyl; substituted or unsubstituted C₃-C₁₀-cycloalkyl; substituted or unsubstituted C₆-C₃₂-aryl; substituted or unsubstituted C₂-C₂₈-heteroaryl. More preferably, R¹⁴ and R¹⁵ may be OR′, wherein R′ is independently selected from the group consisting of H, D, substituted or unsubstituted C₁-C₁₀-alkyl, substituted or unsubstituted C₃-C₁₀-cycloalkyl; and R¹⁶ may be selected from substituted or unsubstituted C₁-C₁₀-alkyl; substituted or unsubstituted C₃-C₁₀-cycloalkyl; substituted or unsubstituted C₆-C₃₂-aryl. Most preferably, R¹⁴ and R¹⁵ may be OR′, wherein R′ is H, methyl, ethyl, or benzyl; and R¹⁶ may be selected from methyl, ethyl, phenyl, and benzyl.

Preferably, R¹⁷ may be selected from substituted or unsubstituted C₁-C₁₀-alkyl; substituted or unsubstituted C₃-C₁₀-cycloalkyl; substituted or unsubstituted C₆-C₃₂-aryl; substituted or unsubstituted C₂-C₂₈-heteroaryl, substituted or unsubstituted C₂-C₂₀-alkynyl and substituted or unsubstituted C₂-C₂₀-alkenyl, OR′, wherein R′ is selected from the group consisting of H, D, substituted or unsubstituted C₁-C₁₀-alkyl; substituted or unsubstituted C₃-C₁₀-cycloalkyl; substituted or unsubstituted C₆-C₃₂-aryl; substituted or unsubstituted C₂-C₂₈-heteroaryl, the substituent may contain the atoms necessary to complete a cyclic structure with one of R⁵-R⁸ or R¹⁰-R¹³ forming a phenanthrenequinone. More preferably, R¹⁷ is selected from substituted or unsubstituted C₁-C₁₀-alkyl; substituted or unsubstituted C₆-C₃₂-aryl; OR′, wherein R′ is selected from the group consisting of H, substituted or unsubstituted C₁-C₁₀-alkyl. Most preferably, R¹⁷ may be methyl, ethyl, phenyl, methoxy, or ethoxy.

Preferably, R¹⁸ may be O or NR′ wherein R′ is selected from substituted or unsubstituted C₁-C₂₀-alkyl ester; substituted or unsubstituted C₆-C₄₈-aryl ester. More preferably, R¹⁸ may be O or NR′ wherein R′ is substituted or unsubstituted C₆-C₄₈-aryl ester. Most preferably, R¹⁸ may be O or NR′ wherein R′ is phenylester or tolylester.

Preferably, the substituent on any of R¹⁴ to R²⁷, preferably R¹⁹, may contain the atoms necessary to complete a cyclic structure with one of R⁵-R⁸ or R¹⁰-R¹³.

Preferably, R¹⁹ may be selected from substituted or unsubstituted C₁-C₁₀-alkyl, preferably methyl or ethyl; substituted or unsubstituted C₃-C₁₀-cycloalkyl; substituted or unsubstituted C₆-C₃₂-aryl; substituted or unsubstituted C₂-C₂₈-heteroaryl, substituted or unsubstituted C₂-C₂₀-alkynyl and substituted or unsubstituted C₂-C₂₀-alkenyl, the substituent may contain the atoms necessary to complete a cyclic structure with one of R⁵-R⁸ or R¹⁰-R¹³, more preferably the substituent may contain the atoms necessary to complete a cyclic structure with one of R⁵-R⁸ or R¹⁰-R¹³ forming an anthracene, thioxanthone, fluorenone, acenaphtylene-1,2-dione, thiochroman-4-on, 9-fluorenone, anthraquinone, benzanthrone, 9,10-phenanthrenequinone, xanthone, 1,3-indanedione, chromone, 1,4-naphthoquinone, coumarin, more preferably R¹⁹ may be selected from substituted or unsubstituted phenyl or naphthyl. More preferably, if the selected substituent of R¹⁹ is additionally substituted, the additional substituent may be selected from the group consisting of H, D, methyl, alkyl, phenyl, CN, Cl, Br, F, methoxy, NMe₂, CF₃, SO₂Me, and SO₂NH₂.

In one preferred embodiment the following formula:

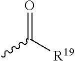

may be α-aminoacyl, α-N,N-dialkylamino-acyl, α-hydroxyacyl or α-alkoxyacyl.

If present, R^{′1} or R^{″1} may be the same as R¹ defined above, R^{′2} or R^{″2} may be the same as R² defined above, R^{′3} or R^{″3} may be the same as R³ defined above, R^{′4} or R^{″4} may be the same as R⁴ defined above, R^{′5} or R^{″5} may be the same as R⁵ defined above, R^{′6} or R^{″6} may be the same as R⁶ defined above, R^{′7} or R^{″7} may be the same as R⁷ defined above, R^{′8} or R^{″8} may be the same as R⁸ defined above, R^{′9} or R^{″9} may be the same as R⁹ defined above, R^{′10} or R^{″10} may be the same as R¹⁰ defined above, R^{′11} or R^{″11} may be the same as R¹¹ defined above, R^{′12} or R^{″12} may be the same as R¹² defined above, R^{′13} or R^{″13} may be the same as R¹³ defined above.

Here halogen may be fluorine, chlorine, bromine, iodine.

Here alkyl, alkenyl, and alkynyl may be cyclic, linear, or branched.

Here alkyl acyl has the following formula

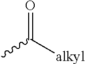

and aryl acyl has the following formula

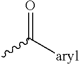

wherein the waved line represents the bond of the acyl group to the structure of formula (1).

In the case that one (or more) of the groups R¹-R¹³ is selected as amide, the bond can be made via the N as well as via the CO.

In the case that one (or more) of the groups R¹-R¹³ is selected as ester, the bond can be made via the O as well as via the CO.

In a further embodiment, it is provided that R³-R⁸ and R¹⁰-R¹³ are independently selected from the group consisting of H and electron withdrawing groups.

A spiropyran of formula (1) containing one or more preferred substituents above may be beneficial for the use as a photoinitiator, preferably dual color photoinitiator, or as an intermediate product to obtain a photoinitiator. On the other hand, spiropyrans containing one of those substituents, may be obtained by limited or none manufacturing methods, since organometallic reagents, such as organolithium reagents, which may be used in a conventional synthetic pathway may rather undergo metal-substituent exchange, like metal-halogen exchange leading to undesired side products. However, also other undesired side reactions are possible with the above moieties by conventional methods of the prior art. The process for the manufacture of spiropyrans of formula (1) of the present invention may avoid the use of organometal reagents and other harsh reagents in the presence of sensitive functional groups so that surprisingly the process may provide spiropyrans containing the above substituents with higher efficiency, or may be accessible for the first time.

Without being bound by any theory, the skilled person may be aware of that while the formylation of 4-hydroxybenzophenone in a Reimer-Tiemann reaction or a Casiraghi reaction may yield the respective 2-hydroxy-5-benzoyl-benzaldehyde, a similar reaction on other hydroxybenzophenone derivatives such as 2-hydroxybenzophenone or 3-hydroxybenzophenone may not be practically accessible. The process for the manufacture of spiropyrans of formula (1) of the present invention may circumvent the formylation of hydroxybenzophenone derivatives so that surprisingly the process may provide spiropyrans containing carbonyl substituents in other position than R¹¹ with higher efficiency, or may be accessible for the first time.

While electron-rich acid anhydrides can be used in Friedl-Crafts acylations to the 5-position of 2-hydroxy-benzaldehydes, this is not possible for other positions or electron deficient acid chlorides or anhydrides. It is further limited in that salicylaldehydes which are functionalized with electron withdrawing groups are not reactive enough for Friedl-Crafts acylations, thus it is very difficult to introduce more than one electron withdrawing group. In one aspect of the invention, the process above may be used to access spiropyrans of formula (1) with more than one electron withdrawing group, which additionally may also contain at least one carbonyl.

For that, one approach may be a process in which a halide such as chlorine, bromine or iodine may be transformed to a carbonyl functionality in a subsequent reaction. Halides can be provided on every position of a salicylaldehyde of formula (4) and on every position of a 2-methyleneindoline of formula (3) allowing for transformation to a carbonyl group on every position of a spiropyran. Thereby, bromides can also be introduced by reaction of N-bromosuccinimide with salicylaldehydes which carry electron withdrawing groups such as trifluoromethyl. Therefore, halogenated spiropyrans may be prepared as precursors for carbonyl substituted spiropyrans. The new spiropyran containing a carbonyl can be subjected to an exchange reaction as disclosed in the invention, allowing substitution patterns which may have not been accessible by conventional methods of the prior art. This process may also be applicable for the introduction of electron deficient carbonyl groups or carbonyl groups in positions which are not accessible by direct functionalization of salicylaldehydes or 2-methleneindolines. Thereby, the process may introduce two or more carbonyl functionalities when other electron withdrawing substituents are already present in the precursor of formula (2) or any reactant of formula (3), formula (4) or formula (5). Furthermore, the method of the present invention allows to introduce two identical carbonyl groups simultaneously, when a spiropyran is substituted with a halide each on the indole half and the salicylaldehyde half of the molecule. In this case two equivalents of n-butyl lithium and the respective Weinreb amide may be used.

The advantage of such a method is the simple simultaneous introduction of the necessary functional carbonyl groups leading to dual color photoinitiators with surprisingly improved properties regarding absorption spectra, switching and initiation efficiency. While methods according to the state of the art allow only the simultaneous functionalization in R⁴ and R¹¹ with electron rich carbonyls such as acyl or benzoyl, when no other electron withdrawing groups are present. The newly described method is neither restricted in the choice of positions nor in the electronic properties of the carbonyl substituent or further already present electron withdrawing substituents, particularly when R⁴ or R¹¹ is H. By installing multiple functional groups in one reaction a short synthetic approach to complex and highly functionalized spiropyrans is provided. When further functional groups which are compatible with the reaction conditions to implement the groups, such as CF₃, methyl, methoxy and others, are introduced beforehand, it is possible to introduce three or more functional groups with a surprisingly short synthetic route. These spiropyrans may be better suited as dual color photoinitiators than dual color initiator described in the state of the art.

The present invention may further be advantageous for introducing functional groups on the spiropyran, the 2-methyleneindoline or the salicylaldehyde which interfere with the reaction conditions for introducing a carbonyl functionality. This aspect may be of certain importance for the synthesis of spiropyrans which are functionalized with at least two different carbonyl groups or at least one carbonyl group and at least one interfering substituent. This includes substituents which cause insufficient solubility in solvents, such as tetrahydrofuran, like charged groups, such as sulfonate, sulfonic acid, carboxylate, carboxylic acid, quaternary ammonium salts, phenoxide, hydroxy, phosphonic acid, and phosphonate. This aspect further includes the halides, such as Cl, Br, and I which may undergo a metal-halogen exchange themselves, and functional groups which may react with organo-metallic species such as n-butyl lithium which include but are not limited to ketone, aldehyde, nitrile, ester, carboxamide, carbonate, cyanate, isocyanate, nitro, carbamate, oxime, sulfonyl, methyl sulfone, alkyl sulfones, and phosphine oxide. This aspect further includes aromatic systems which can be deprotonated by n-butyl lithium due to very electron withdrawing substituents such as two trifluoromethyl groups.

The present invention may further be advantageous for introducing different carbonyl groups on either side of the spiropyran motif with flexibility to the position, which leads to improved characteristics regarding the dual color photoinitiation properties.

### 1.1. Precursor, Spiropyran of Formula (2)

At least one of R^{′2} to R^{′5} and R^{′10} to R^{′13}, preferably at least one of R^{′10} to R^{′13}, more preferably at least one of R^{′10}, R^{′12} and R^{′13} of the precursor of formula (2) may be a substituent selected from the group consisting of chlorine; bromine; iodine; formyl; carbonate; carbonate ester; ester; amide; CF₃; substituted or unsubstituted C₂-C₄₉-alkyl acyl; substituted or unsubstituted C₂-C₄₉-aryl acyl; ketone; acyl; oxime; aldehyde, NO₂; CN; (meth)acrylate; sulfones; alkylsulfones; sulfonamides; SO₂Me; SO₂NH₂; methoxy; and tosyl; or one of the following formulae:

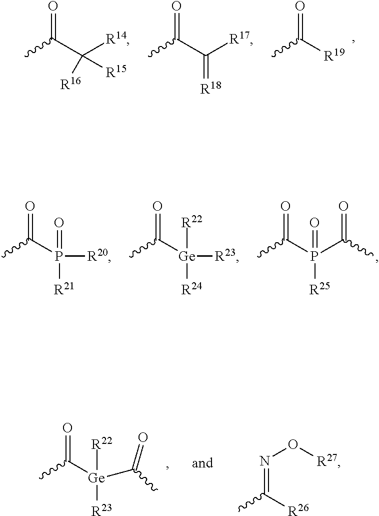

wherein R¹⁴ to R²⁷ are defined as above.

Preferably, if at least one of R^{′2} to R^{′5} is substituted with one of the above substituents, then R^{′10} to R^{′13} is also substituted with one of the above substituents, which are preferably electron withdrawing groups, more preferably CF₃.

Preferably, R⁸ is selected from the group consisting of H, D or C₁-C₈-alkyl, preferably methyl.

Hence, a spiropyran of formula (1) containing at least one of the above substituents may be efficiently obtained by a precursor containing one of the substituents above. Surprisingly, such spiropyran of formula (1) may not have been accessible by a conventional method reported in the prior art.

The precursor being the spiropyran of formula (2) may be provided with 1.0 equivalents in the reaction mixture.

The precursor may be preactivated with a nucleophile as it is disclosed above. Preferably, the nucleophile may be an amine base, preferably amine base, to obtain a pre-activated precursor prior to providing the reaction mixture. In one embodiment, the precursor may be in situ pre-activated in the reaction mixture by adding a catalytic amount of an amine base. The catalytic amount of amine base may be 0.02 to 0.2 equivalents, preferably 0.5 to 0.15 equivalents, most preferably 0.1 equivalents, based on the amount of the precursor. The formation of the pre-activated precursor may improve the formation of the spiropyran of formula (1).

In another embodiment, the precursor may be pre-activated before addition to the reaction mixture by reaction with water, an amine base and/or an acid. The amount of amine base and/or acid may be 0.5-100 equivalents, preferably 0.8 to 10 equivalents, most preferably 1 to 3 equivalents, based on the amount of the precursor. The formation of the pre-activated precursor may improve the formation of the spiropyran of formula (1). The amine base, may be preferably a primary or secondary amine base, more preferably a primary amine base. The primary amine base may be alkylamine, preferably methylamine, ethylamine, propyl amine or a mixture of those. Methylamine may be preferred, since methylamine can be easily removed from the reaction mixture. The acid may be an inorganic acid or an organic acid, preferably a carboxylic acid. More preferably, the carboxylic acid may be formic acid or acetic acid.

Alternatively, in a further embodiment a secondary amine base like piperidine may be preferred. Furthermore, the precursor may be functionalized with a group which can be reduced or oxidized. After the reduction or oxidation, the spiropyran is either subjected to an exchange reaction yielding another spiropyran or treated with a primary amine base to yield the corresponding salicylaldehyde and 2-methyleneindoline. In an embodiment R^{′2} to R^{′5} and R^{′10} to R^{′13} may contain a group, which can be reduced or oxidized. Groups which can be reduced or oxidized include but are not limited to formyl, hydroxy, ketone, carboxylic acid, cyano, nitro, and amino.

In a further embodiment, the present invention may be directed to a process for the manufacture of a precursor represented by the following formula (2):

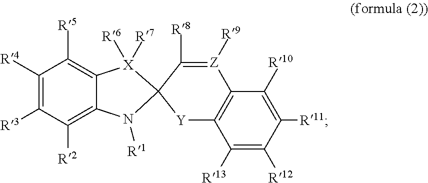

wherein R^{′1}, R^{′6} to R^{′9} are independently selected from the group consisting of H, D, substituted or unsubstituted C₁-C₁₀-alkyl, preferably methyl; substituted or unsubstituted C₆-C₃₂-aryl, preferably phenyl; substituted or unsubstituted C₂-C₂₀-alkynyl and substituted or unsubstituted C₂-C₂₀-alkenyl and benzyl;
X is C;
Z is C;
Y is O;
R^{′2} to R^{′5} and R^{′10}-R^{′13} are independently selected from the group consisting of H, D, substituted or unsubstituted C₁-C₁₀-alkyl, substituted or unsubstituted C₆-C₃₂-aryl, substituted or unsubstituted C₁-C₂₀-alkoxy, substituted or unsubstituted C₆-C₄₈-aryloxy, F, Cl, CF₃, CN; wherein two adjacent groups of may be linked to each other to form a fused ring structure, preferably, a fused aromatic C₆-ring; and a substituent of the following formula:

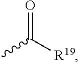

wherein R¹⁹ is defined as it is defined above in the description;
wherein at least one of R² to R⁵ and R¹⁰ to R¹³ is a substituent of the following formula:

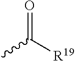

the process comprising the steps of:
   providing a reactant, wherein the reactant is a spiropyran represented by the following formula (2A):

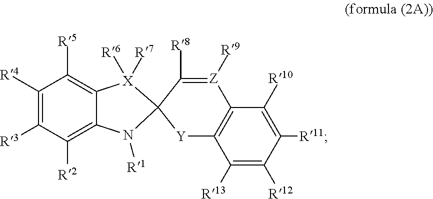

wherein at least one of R^{′2} to R^{′5} and R^{′10} to R^{′13} is a halogen atom selected from the group consisting of Cl, Br and I; and the rest of the substituents in the reactant of formula (2a) are the same as in the precursor of formula (2);
   reacting the halogen atom of the reactant in a metal-halogen exchange reaction with an organolithium reagent or Grignard reagent, preferably organolithium reagent, to obtain a metal-spiropyran species,
   subsequently reacting the metal-spiropyran species with a Weinreb-amide of the following formula:

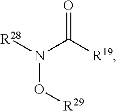

wherein R²⁸ and R²⁹ are selected from the group consisting of substituted or unsubstituted C₁-C₁₀-alkyl; substituted or unsubstituted C₆-C₃₂-aryl; substituted or unsubstituted C₂-C₂₀-alkynyl and substituted or unsubstituted C₂-C₂₀-alkenyl, preferably are R²⁸ and R²⁹ are each methyl, to obtain the precursor of formula (2), wherein preferably the precursor of formula (2) is obtained after an acidic aqueous work-up.

The acidic aqueous work-up may be performed after reacting the metal-spiropyran species with a Weinreb-amide to obtain the precursor of formula (2). The acidic aqueous work-up may be necessary to form the desired ketone moiety. The acidic aqueous work-up may contain at least water and an acid, like preferably an inorganic or organic acid. More preferably the acidic aqueous work-up may contain at least water and an acid selected from group consisting of hydrochloric acid, acetic acid or formic acid.

Preferably, at least one of R^{′2} to R^{′5} of the reactant of formula (2a) is a halogen atom selected from the group consisting of Cl, Br and I and at least one of R^{′10} to R^{′13} of the reactant of formula (2a) is also a halogen atom selected from the group consisting of Cl, Br and I, and wherein at least two or more halogen atoms react in a metal-halogen exchange reaction and with a Weinreb-amide to obtain a precursor according to formula (2). Hence, the obtained precursor of formula (2) may contain at least two, preferably two, substituents of the following formula:

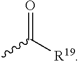

Preferably, the organolithium reagent is an alkyl organolithium reagent or an aryl organolithium reagent, more preferably, the organolithium reagent is an alkyl organolithium reagent. An alkyl organolithium reagent is preferably selected from the group consisting of n-BuLi, sec-BuLi, tert-BuLi, preferably n-BuLi.

Preferably, the halogen atom in the reactant of formula (2) is Br.

In an especially preferred embodiment of the present invention, a precursor of formula (2) may be obtained according to the following reaction scheme:

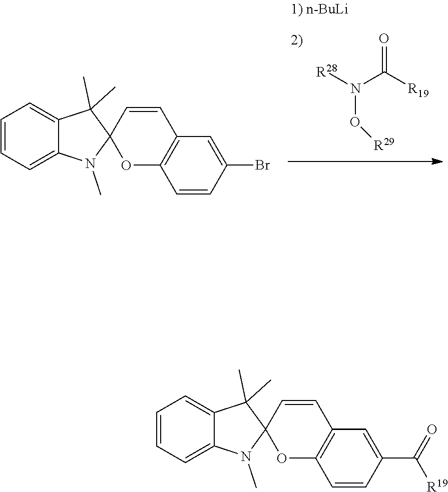

wherein the substitutents are the same as defined above. The skilled person knows that the spriopyran reactant and product in the reaction scheme may contain additional substitutents, which were left out in the reaction scheme for the sake of improved readability. Hence, if necessary, the skilled person also knows that the bromine substituent can be provided on any other position of the aryls as well, leading to the respective isomer, where also the carbonyl substituent is provided on the respective position.

Preferably, the process for the manufacture of the precursor of formula (2) is followed by the process for the manufacture of the spiropyran of formula (1).

For the process to manufacture the precursor of formula (2) the skilled person knows without being bound by any theory, that halogenated spiropyrans may be preferred for reactions with metal-organic reagents, since the spiropyran motif itself may be quite inert. Methods known from the state of the art may utilize either the combination of n-butyl lithium+acid chloride/anhydride or n-butyl lithium+nitrile. The combination of n-BuLi with acid chloride/anhydride may have the disadvantage that the carbonyl may be directly formed, so that a second lithiated spiropyran may add to the carbonyl. Furthermore, the reaction mixture may be strongly basic, which may promote aldol condensation of the already formed products. Nitriles with n-BuLi may have a lower reactivity compared to acid chlorides, which may have the disadvantage that they require prolonged reaction times or higher temperatures, than the typically applied −78° C. This may lead to unspecific side reactions and lower yields.

Hence, the present invention surprisingly solves the problem of the state of the art by providing a method of functionalizing a precursor spiropyran, which may then be used in the exchange reaction. A new method to introduce the carbonyl on the spiropyran is by metal-halide exchange of a halide on spiropyran using an organolithium species, like n-butyl lithium, and reacting the formed organo-lithium species with a Weinreb amide. The reaction works surprisingly well, with high yields, preferably quantitative yields. The only side products of the metal halogen exchange reaction and the subsequent carbonyl introduction may be a few percent, like less than 5 mol %, of de-halogenated spiropyran of the overall yield. The low amount of side products has the surprising effect, that a simplified purification of the desired spiropyran may be accessible so that the manufacture beyond lab scale, like industrial scale is possible. The new method may allow for the first time to introduce more than one carbonyl moiety to the spiropyran by providing a spiropyran precursor with two halogen atoms. The person skilled in the art will notice, that the reaction may be very efficient as the carbonyls cannot be introduced stepwise. The reaction may tolerate several functional groups substituted to spiropyran including alkyl, alkoxy, CF₃, and aryl groups substituted with these substituents, etc. as it is defined above.

### 1.1.A Indolenium Salt of Formula (3) and Spiropyran of Formula (2)

In a further aspect, the invention may provide a process for the manufacture of a spiropyran represented by the following formula (1):

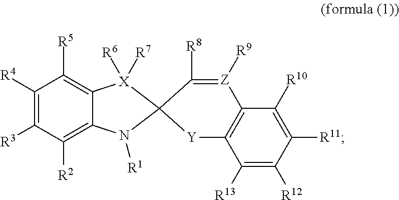

wherein the process comprises the steps of
   providing a precursor, wherein the precursor is a spiropyran represented by the following formula (2):

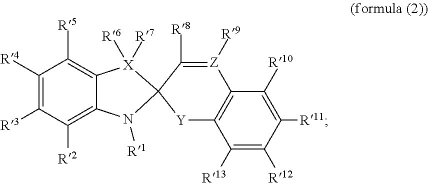

providing a reactant, wherein the reactant is
an indolenium salt represented by the following formula (3):

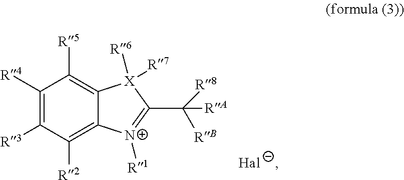

or the corresponding 2-methyleneindoline compound;
   optionally pre-activating the precursor
   providing a reaction mixture comprising the precursor or optionally a preactivated precursor, and the reactant to obtain said spiropyran of formula (1);
wherein the spiropyran, the precursor, and the reactant are different from each other;
wherein if the reactant is an indolenium salt of formula (3), the obtained spiropyran of formula (1) is represented by the following formula (1A):

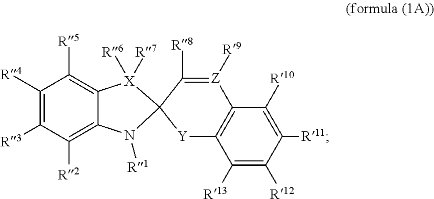

wherein R^{″1} to R^{″8} of formula (1A) are independently the same as R^{″1} to R^{″8} of formula (3) and R^{′9} to R^{′13} of formula (1A) are independently the same as R^{′9} to R^{′13} of formula (2);

Preferably, the reactant is the indolenium salt of formula (3) wherein X, Hal, R^{″A}, R^{″B} and R^{″1} to R^{″8} are substituents as defined above.

Thereby, R^{′9}, R^{′10}, R^{′11}, R^{′12} and R^{′13} of the precursor being the spiropyran of formula (2), and the spiropyran of formula (1A) correspond to R⁹, R¹⁰, R¹¹, R¹² and R¹³ of the spiropyran of formula (1). Hence, the precursor being the spiropyran of formula (2), and the spiropyran of formula (1A) may contain the same substituents for R^{′9}, R^{′10}, R^{′11}, R^{′12} and R^{′13}, as the spiropyran of formula (1) for R⁹, R¹⁰, R¹¹, R¹² and R¹³ above as preferred embodiments.

Thereby, R^{″1}, R^{″2}, R^{″3}, R^{″4}, R^{″5}, R^{″6}, R^{″7} and R^{″8} of the indolenium salt of formula (3), and the spiropyran of formula (1A) correspond to R¹, R², R³, R⁴, R⁵, R⁶, R⁷ and R⁸ of spiropyran of formula (1). Hence, indolenium salt of formula (3), and the spiropyran of formula (1A) may contain the same substituents for R^{″1}, R^{″2}, R^{″3}, R^{″4}, R^{″5}, R^{″6}, R^{″7} and R^{″8}, as the spiropyran of formula (1) for R¹, R², R³, R⁴, R⁵, R⁶, R⁷ and R⁸ above as preferred embodiments.

More preferably the amount of electron withdrawing substituents in R^{″2} to R^{″5} of formula (3) is higher than the amount of electron withdrawing substituents in R^{′2} to R^{′5} of formula (2).

Even more preferably, the substituent in R^{″4} of formula (3) is stronger electron withdrawing than the substituent in R^{′4} of formula (2), and/or the substituent in R^{″4} of formula (3) is chosen from the group consisting of carbonyl; chlorine; bromine; iodine; formyl; carbonate; carbonate ester; ester; amide; CF₃; substituted or unsubstituted C₂-C₄₉-alkyl acyl; substituted or unsubstituted C₂-C₄₉-aryl acyl; ketone; acyl; acetyl; benzoyl; oxime; aldehyde, NO₂; CN; (meth)acrylate; sulfones; alkylsulfones; sulfonamides; SO₂Me; SO₂NH₂.

The amount of electron withdrawing substituents may have an effect on the acidity of the compound. This may have the beneficial effect, that the newly formed 2-methyleneindoline is formed as a side product, which may be directly protonated to the corresponding indolenium salt. As a consequence, the newly formed 2-methyleneindoline may not be available for a second condensation reaction, so that the equilibrium is shifted to the formation of the desired spiropyran of formula (1) and the back reaction in the equilibrium is hindered.

Furthermore, if substituents in R^{′2} to R^{′5} of formula (2) render the newly formed 2-methyleneindole more basic, the acid-base equilibrium may force the reaction to the formation of the corresponding indolenium salt substituted with R^{′2} to R^{′5} as defined.

It has been surprisingly found that the equilibrium of the reaction may be independent of the nature of the salicylaldehyde, which makes the reaction very versatile and spiropyrans of formula (1) may be accessible by the present invention for the first time. Furthermore, the process in which the indolenium salt of formula (3) is used, has been found to be surprisingly mild, when the salicylaldehyde is substituted with electron withdrawing groups so that the process may be applicable in the presence of most substituents, which may be acid or base sensitive.

Preferably, the indolenium salt of formula (2) is formed from the corresponding 2-methyleneindoline compound and an acid, wherein preferably the acid is an organic or inorganic acid, more preferably the acid is selected from the group consisting of hydrochloric acid, acetic acid or formic acid.

Prior to mixing the precursor with the reactant in a reaction mixture, the reactant may be pre-activated by converting the indolenium salt of formula (3) into a corresponding 2-methyleneindoline compound, which is then added to the reaction mixture. The corresponding 2-metyhleneindoline compound may have the following formula (3A):

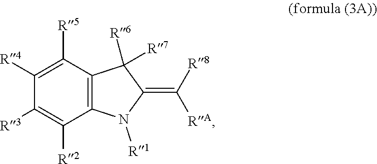

wherein the substituents are defined as in the indolenium salt of formula (3). Hence, instead of adding an indolenium salt of formula (3), the corresponding 2-methyleneindoline compound may be used.

The reactant, i.e. the indolenium salt of formula (3), may be activated with a base, more preferably the reactant is activated with an alkylamine, hydroxide, or carbonate. Hence, the indolenium salt may first be pre-activated by deprotonation of the indolenium salt to form 2-methyleneindoline as pre-activated reactant. The indolenium salt may be deprotonated with a base. The indolenium salt may be deprotonated in situ. Preferably, the indolenium salt is in situ deprotonated with a base to form a 2-methyleneindoline compound. The pre-activated reactant of formula (3), i.e. the corresponding 2-methyleneindoline compound, may be provided in the reaction mixture to obtain a spiropyran of formula (1A). Thereby, a different 2-methyleneindoline compound will be newly formed as a side product.

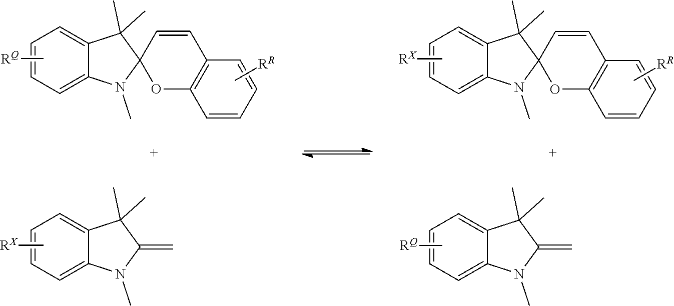

The indolenium salt of formula (3) may be provided to the reaction mixture with an excess of more than 1.1 equivalents, more preferably in range of 1.1 to 1.5 equivalents, more preferably in a range of 1.1 to 1.2 equivalents, most preferably 1.2 equivalents, based on the amount of the precursor of formula (2). If the indolenium salt of formula (3) or the corresponding 2-methyleneindoline is applied in excess, it may add to the spiropyran a second time and undergo another addition or condensation. Hence, the reaction may be carried out with an indolenium salt of formula (3) in slight excess.

Preferably, the concentration of acid or base may be adjusted to provide an overall amount 0.1 equivalents of deprotonated 2-methyleneindoline derivatives in the overall reaction mixture.

The following indolenium salts may be especially preferred:

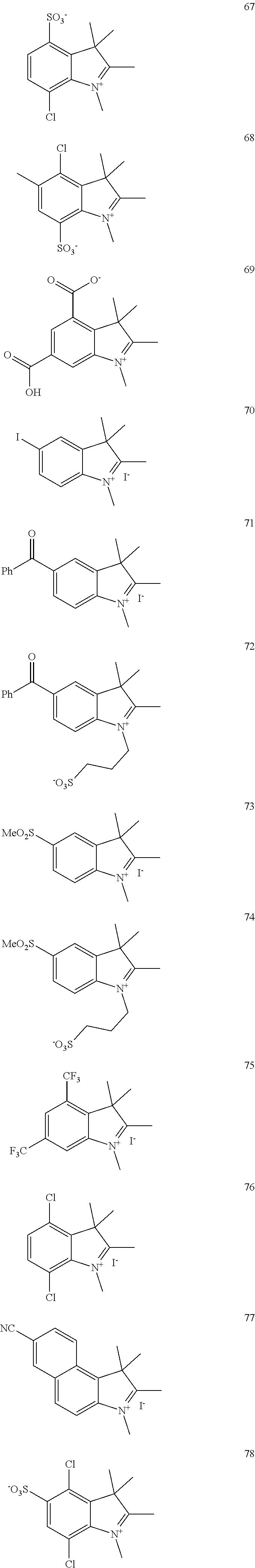

In a further aspect of the invention, the indolenium salt of formula (3) or the corresponding 2-methyleneindoline compound may be provided with an excess of at least 2 equivalents, preferably in the range of 2.5 to 100 equivalents, more preferably in the range of 3 to 10 equivalents, most preferably in the range of 3.5 to 5 equivalents to the reaction mixture containing a spiropyran of formula (2). Where an indolenium salt is applied an amine base may be provided to the reaction mixture in a range of 1 to 3 equivalents. Where a 2-methyleneindoline is applied, acid may be provided in a range of 1 to 2 equivalents. Given these conditions an adduct of the product spiropyran with the following formula

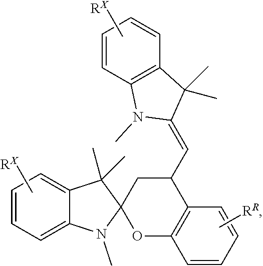

may be obtained from indolenium salt or 2-methyleneindoline compound as reactants. Therein, R^{x} may represent the corresponding substituents R^{′2} to R^{′5} of formula (2) independently from each other, which may be the same or different, as described above. R^{R} may represent substituents R^{′10} to R^{′13} of formula (2) independently from each other, which may be the same or different.

This adduct may be easily separated (purified) from the reaction mixture, preferably by precipitation. In a further reaction the adduct may be treated with an acid in a polar solvent to provide the product spiropyran. The acid is applied in the second reaction with 1 to 50%, preferably 5 to 20%, more preferably 10%. The acid may be an inorganic acid or an organic acid, preferably the acid is chosen from hydrochloric acid, sulfuric acid, a carboxylic acid, methane sulfonic acid, toluene sulfonic acid, or trifluoroacetic acid, more preferably the acid is formic acid or acetic acid. The polar solvent may be chosen from THF, nitromethane, MTBE, methanol, ethanol, 2-propanol, 1-butanol, ethylene glycol, preferably the solvent is ethanol or methanol. The temperature of the second reaction may be 0 to 150° C., preferably 15 to 100° C., more preferably 20 to 70° C., most preferably 25° C. Hence, the formation of the adduct may have the advantage that it can be surprisingly easily separated from the reaction mixture and can be further reacted to obtain the desired spiropyran of formula (1).

The reaction mixture may further comprise a catalytic amount of a base, preferably a secondary amine base. The secondary amine base may be preferably piperidine. The catalytic amount of base may preferably be in a range of 0.02 to 0.2 equivalents, more preferably in a range of 0.05 to 0.15 equivalents, most preferably 0.1 equivalents based on the precursor.

In one embodiment, piperidine may be used between and 1 and 1.3 equivalents to pre-activate the indolenium salt prior adding to the reaction mixture, so that the corresponding 2-methyleneindoline is formed, which is then added to the reaction mixture with the precursor of formula (2).

In one embodiment the reaction mixture comprising the indolenium salt of the corresponding 2-methyleneindoline may be free of base.

Preferably, at least one, more preferably each, of R^{′2} to R^{′5} of the precursor of formula (2) may be hydrogen or deuterium, and at least one of R^{″2} to R^{″5} of the indolenium salt of formula (3) and the corresponding 2-methyleneindoline may be an electron withdrawing group, preferably benzoyl or methyl sulfone (SO₂Me). Alternatively, if R^{″2} to R^{″5} of the indolenium salt of formula (3) and the corresponding 2-methyleneindoline may not be an electron withdrawing group, R^{′2} to R^{′5} of the precursor of formula (2) is an electron donating substituent as defined above, preferably methoxy. For the formation of the desired spiropyran of formula (1A) it may be advantageous to accelerate the reaction if at least one of R^{′10} to R^{′13} of formula (2) may be independently selected from substituted or unsubstituted C₁-C₆-alkyl, carboxylic acid and salts thereof, sulfonic acid and salts thereof, phosphonic acid and salts thereof, fluorine, bromine, chlorine, iodine, substituted or unsubstituted C₂-C₄₉-alkyl acyl; substituted or unsubstituted C₁-C₂₀-alkoxy; substituted or unsubstituted C₂-C₄₉-aryl acyl, CN, NO₂, aldehyde, ketone, sulfone, alkylsulfone; sulfonamide, SO₂Me, SO₂Ph, SO₂NH₂, CF₃, OCF₃. Preferably, R^{′11} of formula (2) is CF₃. By having an electron-withdrawing group for R^{′10} to R^{′13} of formula (2) it has been surprisingly found, that the reaction with the indolenium salt of formula (3) and the corresponding 2-methyleneindoline can facilitated, so that a higher variety of substituents at the indolenium salt of formula (3) and the corresponding 2-methyleneindoline is tolerated for the formation of spiropyran of formula (1A).

A polar solvent, preferably alcohol, most preferably n-butanol or ethanol, may be added to the reaction mixture. A polar solvent may be THF, water, acetic acid, nitromethane, formic acid, methanol, ethanol, isopropanol. Alcohol may be methanol, ethanol, isopropanol, n-butanol, ethylene glycol. The reaction mixture may be stirred at above 40° C., preferably in a range of 40° C. to 150° C., more preferably in range of 70° C. to 120° C. The reaction mixture may be stirred for more than 1 hour, preferably in a range of 0.5 hours to 48 hours, more preferably in a range of 1 hour to 12 hours, most preferably in the range of 1 hour to 2 hours. The process of the present invention may yield more than 80% yield, like 80% to 100% yield, preferably 90% to 99% yield of the spiropyran of formula (2), if an indolenium salt of formula (3) is used as a reactant.

In a preferred embodiment, R^{′2} to R^{′5} of the precursor of formula (2) may be hydrogen and at least one of R^{″2} to R^{″5} of the indolenium salt of formula (3) may be substituted or unsubstituted benzoyl, the indolenium salt of formula (3) may be used in 0.9 to 1.2 equivalents, preferably 1.0 to 1.1 equivalents, based on the amount of the precursor, piperidine may be used as a catalyst in 0.1 equivalents and ethanol may be used as a solvent. After stirring the reaction mixture at 70° C. for 1 to 2 hours, the desired product, i.e. spiropyran of formula (1) may be obtained with more than 80% yield next to a newly formed indolenium salt as a further product. If R^{″2} to R^{″5} of the indolenium salt of formula (3) is not substituted by a strong electron withdrawing group, preferably R^{′2} to R^{′5} of the precursor of formula (2) may be selected to be an alkoxy, preferably methoxy, so that a similar high yield of more than 80% may be obtained.

A scavenger, like a template, may be added to the reaction mixture. The scavenger may provide a favorable interaction with the product, i.e. the spiropyran of formula (1), or respective merocyanine or the newly formed 2-Methyleneindoline or the corresponding indolenium salt which may be obtained as a further product. Thereby, the equilibrium of the reaction may be shifted to the product side, so that more of the desired spiropyran of formula (1) may be obtained. A non-limiting example for a scavenger is a proton, which may trap the formed 2-Methyleneindoline as the corresponding indolenium salt. A proton may be obtained if the scavenger is an acid.

### 1.1.B Salicylaldehyde of Formula (4) and Spiropyran of Formula (2)

In a preferred aspect, the invention may provide a process for the manufacture of a spiropyran represented by the following formula (1):

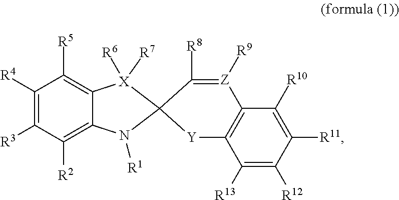

wherein the process comprises the steps of providing a precursor, wherein the precursor is a spiropyran represented by the following formula (2):

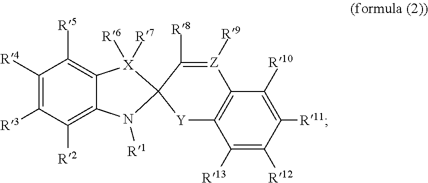

and providing a salicylaldehyde represented by the following formula (4):

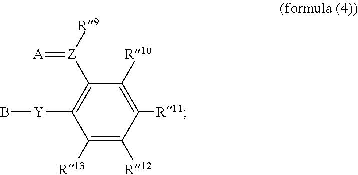

optionally pre-activating the precursor, and then
providing a reaction mixture comprising the precursor or optionally a preactivated precursor, and the reactant to obtain said spiropyran of formula (1);
wherein the spiropyran, the precursor, and the reactant are different from each other;
wherein if the reactant is a salicylaldehyde of formula (4), the obtained spiropyran of formula (1) is represented by the following formula (1B):

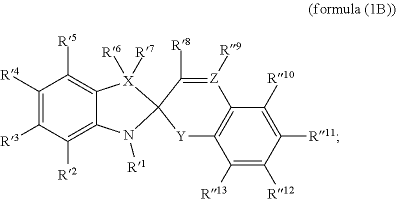

wherein R^{′1} to R^{′8} of formula (1B) are independently the same as R^{′1} to R^{′8} of formula (2) and R^{″9} to R^{″13} of formula (1B) are independently the same as R^{″9} to R^{″13} of formula (4);

Thereby, R^{′1}, R^{′2}, R^{′3}, R^{′4}, R^{′5}, R^{′6}, R^{′7} and R^{′8} of the precursor being spiropyran of formula (2), and the spiropyran of formula (1B) correspond to R¹, R², R³, R⁴, R⁵, R⁶, R⁷ and R⁸ of spiropyran of formula (1). Hence, the precursor being spiropyran of formula (2), and the spiropyran of formula (1B) may contain the same substituents for R^{′1}, R^{′2}, R^{′3}, R^{′4}, R^{′5}, R^{′6}, R^{′7} and R^{′8}, as the spiropyran of formula (1) for R¹, R², R³, R⁴, R⁵, R⁶, R⁷ and R⁸ above as preferred embodiments.

Thereby, additionally, R^{″9}, R^{″10}, R^{″11}, R^{″12} and R^{″13} of the salicylaldehyde of formula (4) and the spiropyran of formula (1B) correspond to R⁹, R¹⁰, R¹¹, R¹² and R¹³ of the spiropyran of formula (1). Hence, salicylaldehyde of formula (4) and the spiropyran of formula (1B) may contain the same substituents for R^{″9}, R^{″10}, R^{″11}, R^{″12} and R^{″13}, as the spiropyran of formula (1) for R⁹, R¹⁰, R¹¹, R¹² and R¹³ above as preferred embodiments.

Preferably, the reactant is a salicylaldehyde of formula (4), wherein A, B, Z, Y, and R^{″9} to R^{″13} are substituents as defined above. More preferably R^{″9} is H or D; and R^{″10} to R^{″13} are independently selected from the group consisting of H; D; halogen; tosyl; formyl; carbonate; carbonate ester; ester; amide; CF₃; substituted or unsubstituted C₂-C₄₉-alkyl acyl; substituted or unsubstituted C₂-C₄₉-aryl acyl; ketone; acyl; oxime; aldehyde, NO₂; CN, (meth)acrylate; sulfone; alkylsulfone; sulfonamide; SO₂Me; SO₂NH₂; sulfonic acid or salts thereof; carboxylic acid or salts thereof; boronic acid or salts thereof; phosphonic acid or salts thereof; NR′₃⁺, wherein R′ is independently selected from the group consisting of H, D, substituted or unsubstituted C₁-C₂₀-alkyl and substituted or unsubstituted C₆-C₄₈-aryl, two R′ may form a ring structure; substituted or unsubstituted sulfonic esters; substituted or unsubstituted sulfonic amides; phosphonic esters; phosphines; phosphates; sulfinic acids; sulfinic esters; sulfonates; sulfoxides; sulfones; alkylsulfones; oximes; isocyanides; cyanate; isocyanate; thiocyanate; isothiocyanate; ammonium; substituted or unsubstituted carbamates; NH₂; OH; substituted or unsubstituted C₁-C₂₀-alkyl; substituted or unsubstituted C₃-C₂₀-cycloalkyl; substituted or unsubstituted C₆-C₄₈-aryl; substituted or unsubstituted C₁-C₂₀-alkoxy; substituted or unsubstituted C₆-C₄₈-aryloxy; substituted or unsubstituted C₁-C₂₀-alkyl ester; substituted or unsubstituted C₆-C₄₈-aryl ester; SiR′₃, —O—SiR′₃ wherein R′ is independently selected from the group consisting of substituted or unsubstituted C₁-C₂₀-alkyl and substituted or unsubstituted C₆-C₄₈-aryl, two R′ may form a ring structure.

Thereby, it has been surprisingly found that a spiropyran of formula (2) may be treated with a salicylaldehyde of formula (4) of low acidity to exchange and thereby release a new salicylaldehyde from the spiropyran.

The salicylaldehyde of formula (4) may be used in excess, preferably in more than 1.1 equivalents, more preferably in a range of 1.1 to 2.0 equivalents based on the amount of the precursor. As consequence, the formation of a spiropyran of formula (1) which may have a low solubility, may be enhanced by shifting the equilibrium to the product side. Thereby, larger quantities of a new salicylaldehyde may be released as a side product.

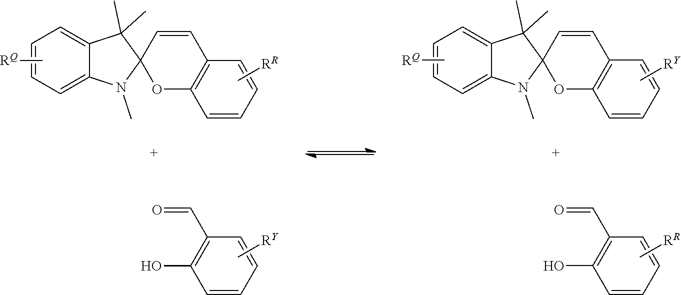

The reaction may be preferably performed in a polar solvent as defined above, more preferably alcohol as defined above, more preferably ethanol. The substituents RY is preferably selected from non-polar groups. RY, i.e. R^{″10}-R^{″13} in salicylaldehyde of formula (4), may be more preferably selected from H, tert-butyl, bromine, or chlorine.

Preferably the precursor of formula (2) has been pre-activated with a nucleophile. Preferably, the nucleophile is an amine base, as it is defined above in regard to the precursor.

A scavenger, like a template, may be added to the reaction mixture. The scavenger may provide a favorable interaction with the product, i.e. the spiropyran of formula (1), or respective merocyanine or the newly formed salicylaldehyde as a side product. Thereby the equilibrium of the reaction may be shifted to the product side, so that more of the desired spiropyran of formula (1) containing the substituents R^{Q} and R^{Y} may be obtained.

### 1.1.C Spiropyran of Formula (2) and Spiropyran of Formula (5) in an Exchange Reaction

In another aspect, the invention may provide a process for the manufacture of a spiropyran represented by the following formula (1):

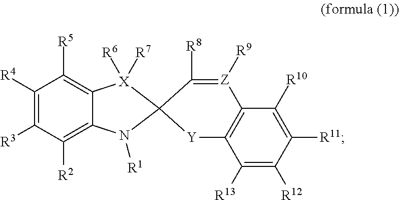

wherein the process comprises the steps of providing a precursor, wherein the precursor is a spiropyran represented by the following formula (2):

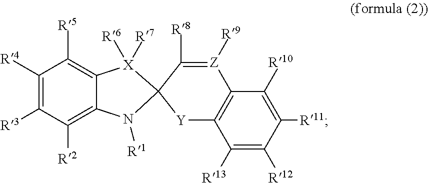

and a spiropyran represented by the following formula (5):

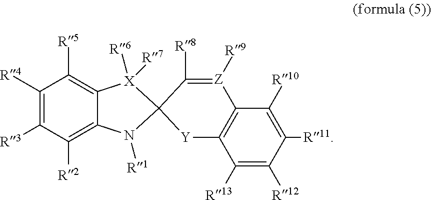

providing a reaction mixture comprising the precursor or optionally a preactivated precursor, and the reactant to obtain said spiropyran of formula (1), wherein the spiropyran, the precursor, and the reactant are different from each other;
wherein if the reactant is a spiropyran of formula (5),
the obtained spiropyran of formula (1) is represented by formula (1A), wherein R^{″1} to R^{″8} of formula (1A) are independently the same as R^{″1} to R^{″8} of formula (5) and R^{′9} to R^{′13} of formula (1A) are independently the same as R^{′9} to R^{′13} of formula (2), or
the obtained spiropyran of formula (1) is represented by formula (1B), wherein R^{′1} to R^{′8} of formula (1B) are independently the same as R^{′1} to R^{′8} of formula (2) and R^{″9} to R^{″13} of formula (1B) are independently the same as R^{″9} to R^{″13} of formula (5).

The preferred embodiments for the substituents of spiropyran of formula (1) may also apply for the substituents of the spiropyran of formula (1A) and spiropyran of formula (1B) as defined above.

Preferably, the reactant is a spiropyran of formula (5), wherein the reaction mixture may comprise a further catalyst. More preferably, the catalyst may be an amine base. Even more preferably the catalyst may be a primary or secondary amine base. Still more preferably, the catalyst may be a C1-C6 alkylamine, and most preferably, piperidine, ethylamine or methylamine. In an alternative preferred embodiment, the catalyst may be a 2-methyleneindoline. The catalyst may be added in a catalytic amount, like 0.02 to 0.2 equivalents, preferably 0.05 to 0.15, based on the amount of the precursor of formula (2). The catalyst may pre-activate the precursor.

The reaction mixture of the process may comprise a solvent, preferably a polar solvent as defined above, more preferably alcohol as defined above, even more preferably ethanol or n-butanol. The reaction may be carried out at a temperature from 0 to 200° C., 10 to 180° C., 20 to 170° C., 30 to 160° C., 40 to 140° C. or 60 to 100° C. The reaction may be carried out at a pressure from 0.01 to 100 atm, 0.1 to 10 atm or 0.5. to 1.5 atm, such as normal pressure (=1 atm corresponding to 101325 Pa). In a further embodiment, the reaction may be carried out in an alcohol at elevated temperature, preferably in a range of 40° C. to 140° C., more preferably in a range of 60° to 100° C. In a further preferred embodiment, the molar ratio of the precursor (that is, a precursor of formula (2)) to the reactant (that is, a reactant of formula (3) or of formula (4) or of formula (5)) may be from 1:100 to 100:1, from 1:50 to 50:1, from 1:20 to 20:1 or from 1:10 to 10:1. In a further preferred embodiment, the concentration of the precursor (that is, a precursor of formula (2)) in the reaction mixture may be from 0.00001 M to 100 M, from 0.001 to 1 M, or from 0.001 to 0.1 M. In a further preferred embodiment, the concentration of the reactant (that is, a reactant of formula (3) or of formula (4) or of formula (5)) in the reaction mixture may be from 0.00001 M to 100 M, from 0.001 to 1 M, or from 0.001 to 0.1 M. In an especially preferred embodiment, the solvent is ethanol and the elevated temperature may be 70° C., or n-butanol and the elevated temperature may be 120° C. In another preferred embodiment, after providing a reaction mixture comprising the precursor or optionally a preactivated precursor, and the reactant, said spiropyran of formula (1) may be obtained after 24 hours, preferably in a range of 24 hours and 10 days, more preferably in a range of 6 to 8 days, most preferably 7 days. Without being bound by any theory it is believed that the following equilibrium may be obtained following the above conditions. Thereby, it is desired, that the equilibrium is on the product side, i.e. right side of the equation.

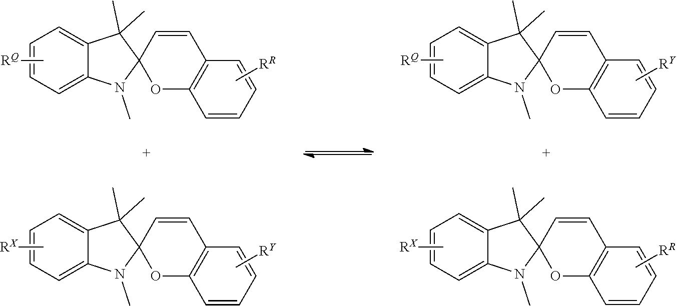

For obtaining the desired spiropyran, the desired spiropyran may have a lower solubility compared to the precursor in said solvent. Hence, the equilibrium may be on the product side. If the reaction mixture may contain a polar solvent, like alcohol, R^{R} and R^{Y}, i.e. R^{″2}-R^{″5} and R^{′10}-R^{′13}, or R²-R⁵ and R¹⁰-R¹³, respectively, may be one or more non-polar substituent, preferably aromatic systems, like substituted or unsubstituted C₆-C₄₈-aryl, alkyl groups, preferably substituted or unsubstituted C₁-C₂₀-alkyl; substituted or unsubstituted C₃-C₂₀-cycloalkyl; or halides, preferably bromine. Having one or more non-polar substituent in formula (I) in a polar solvent, may have the effect that the spiropyran of formula (I) may precipitate out in the reaction mixture, so that the equilibrium is shifted to the product side, giving control over the reaction. Furthermore, one of the formed spiropyrans may be thermodynamically strongly favored over the corresponding merocyanine. This may mainly lead to the formation of the stable spiropyrans and may also shift the equilibrium to the product side. For example, if the unsubstituted spiropyran contains R^{Q}, R^{Y}=H, the formation of higher substituted spiropyrans with R^{R} and R^{X} being substituents other than H, may be improved. Hence, the equilibrium may also be shifted to the product side.

On the other hand, the reaction mixture may further comprise a scavenger, i.e. template, and said scavenger may be able to bind to the obtained spiropyran, or to bind to an obtained side product in the reaction mixture so that the equilibrium may be shifted to the product side. In other words, this scavenger may be a template being added to the reaction mixture, providing favorable interaction with at least one of the product spiropyrans or respective merocyanines and thereby providing a shift of the equilibrium to the product side.

### 2. Spiropyran of Formula (1)

Another aspect of the present invention is a spiropyran represented by the following formula 1:

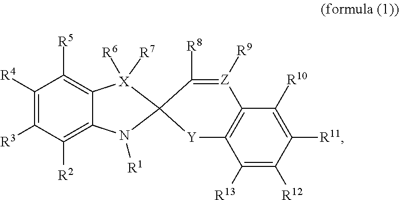

wherein X is selected from S, C, or N, X is selected from S, C, or N; if X is S, then R⁶, R⁷, R^{′6}, R^{′7}, R^{″6}, R^{″7} may not be present accordingly; if X is N, then R⁷, R^{′7}, R^{″7} may not be present accordingly,
wherein Y is selected from O, S, or N; where Y is N, the substituent contains the atoms necessary to complete a cyclic structure with R¹³ selected from the group consisting of benzimidazole, indoline, indole, dihydroquinoline, and tetrahydroquinoline, preferably Y is O;
wherein Z is selected from N or C, preferably Z is C;
wherein R¹ to R¹³ are independently selected from the group consisting of H; D; halogen; NO₂; CN; OH; SH; substituted or unsubstituted C₁-C₂₀-alkyl; substituted or unsubstituted C₃-C₂₀-cycloalkyl; substituted or unsubstituted C₆-C₄₈-aryl; substituted or unsubstituted C₂-C₄₂-heteroaryl; substituted or unsubstituted C₂-C₄₉-alkyl acyl; substituted or unsubstituted C₂-C₄₉-aryl acyl; substituted or unsubstituted C₁-C₂₀-alkoxy; substituted or unsubstituted C₆-C₄₈-aryloxy; NH₂; substituted or unsubstituted C₁-C₂₀-alkyl ester; substituted or unsubstituted C₆-C₄₈-aryl ester; substituted or unsubstituted C₁-C₂₀ alkyl amide; substituted or unsubstituted C₆-C₄₈-aryl amide; NR′₂; SiR′₃; —O—SiR′₃, wherein R′ is independently selected from the group consisting of substituted or unsubstituted C₁-C₂₀-alkyl and substituted or unsubstituted C₆-C₄₈-aryl, two R′ may form a ring structure; substituted or unsubstituted carboxylic acids and salts thereof; substituted or unsubstituted sulfonic acids and salts thereof; substituted or unsubstituted sulfonic esters; substituted or unsubstituted sulfonic amides; formyl; ether; thioether; carbonate; carbonate ester; sulfates; boronic acids; boronic esters; phosphonic acids; phosphonic esters; phosphines; phosphates; peroxycarbonic acids; thiocarbonic acids; sulfinic acids; sulfinic esters; sulfonates; thiolesters, sulfoxides; sulfones; hydrazides; thioaldehydes; ketones; thioketones; oximes; hydrazines; nitroso; azo; diazo; diazonium; isocyanides; cyanate; isocyanate; thiocyanate; isothiocyanate; hydroperoxide; peroxide; acetals; ketal; orthoester; orthocarbonate esters; ammonium; imines; imides; azide; nitrate; isonitrile; nitrosoxy; substituted or unsubstituted carbamates; substituted or unsubstituted ethers; substituted or unsubstituted polyether carbamates; substituted or unsubstituted arylazo; substituted or unsubstituted C₂-C₂₀-alkynyl and substituted or unsubstituted C₂-C₂₀-alkenyl; wherein two adjacent groups of may be linked to each other to form a fused ring structure, preferably, a fused aromatic C₆-ring;
wherein the one or more substituents, if present in one or more of R¹ to R¹³, are independently selected from the group consisting of D; halogen; NO₂; CN, C₂-C₄₉-alkyl acyl; substituted or unsubstituted C₁-C₂₀-alkoxy; substituted or unsubstituted C₆-C₄₈-aryloxy; substituted or unsubstituted C₂-C₄₉-aryl acyl; (meth)acrylate; tosyl; sulfonic acid or salts thereof; carboxylic acid or salts thereof; boronic acid or salts thereof; phosphonic acid or salts thereof; NR′₃⁺, wherein R′ is independently selected from the group consisting of H, D, substituted or unsubstituted C₁-C₂₀-alkyl and substituted or unsubstituted C₆-C₄₈-aryl, two R′ may form a ring structure; NH₂; and OH;
wherein if present two adjacent groups of R¹⁰ to R¹³, and R² to R⁵ may be independently linked to each other to form a fused ring structure;
and
wherein at least one substituent for R² to R⁵ and R¹⁰ to R¹³ is selected from the group consisting of chlorine; bromine; iodine; formyl; carbonate; carbonate ester; ester; amide; CF₃; substituted or unsubstituted C₂-C₄₉-alkyl acyl; substituted or unsubstituted C₂-C₄₉-aryl acyl; ketone; acyl; oxime; aldehyde, NO₂; CN; (meth)acrylate; sulfones; alkylsulfones; sulfonamides; SO₂Me; SO₂NH₂; methoxy; and tosyl; or one of the following formulae:

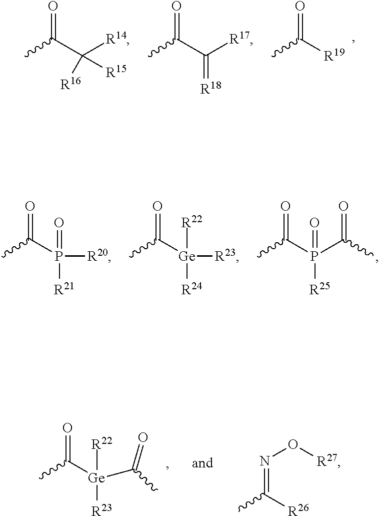

wherein R¹⁴ to R²⁷ are independently selected from the group consisting of H; D; halogen; NO₂; CN; OH; SH; substituted or unsubstituted C₁-C₂₀-alkyl; substituted or unsubstituted C₃-C₂₀-cycloalkyl; substituted or unsubstituted C₆-C₄₈-aryl; substituted or unsubstituted C₂-C₄₂-heteroaryl; substituted or unsubstituted C₂-C₄₉-alkyl acyl; substituted or unsubstituted C₂-C₄₉-aryl acyl; substituted or unsubstituted C₁-C₂₀-alkoxy; substituted or unsubstituted C₆-C₄₈-aryloxy, and NH₂; substituted or unsubstituted C₁-C₂₀-alkyl ester; substituted or unsubstituted C₆-C₄₈-aryl ester; substituted or unsubstituted C₁-C₂₀ alkyl amide; substituted or unsubstituted C₆-C₄₈-aryl amide; NR′₂, SiR′₃, —O—SiR′₃ wherein R′ is independently selected from the group consisting of substituted or unsubstituted C₁-C₂₀-alkyl and substituted or unsubstituted C₆-C₄₈-aryl, two R′ may form a ring structure; substituted or unsubstituted carboxylic acids and salts thereof; substituted or unsubstituted sulfonic acids and salts thereof; substituted or unsubstituted sulfonic esters; substituted or unsubstituted sulfonic amides; formyl; ether, thioether; carbonate; carbonate ester; sulfates; boronic acids; boronic esters; phosphonic acids; phosphonic esters; phosphines; phosphates; peroxycarbonic acids; thiocarbonic acids; sulfinic acids; sulfinic esters; sulfonates; thiolesters, sulfoxides; sulfones; alkylsulfones; hydrazides; thioaldehydes; ketones; thioketones; oximes; hydrazines; nitroso; azo; diazo; diazonium; isocyanides; cyanate; isocyanate; thiocyanate; isothiocyanate; hydroperoxide; peroxide; acetals; ketal; orthoester; orthocarbonate esters; ammonium; imines; imides; azide; nitrate; isonitrile; nitrosoxy; substituted or unsubstituted carbamates; substituted or unsubstituted ethers; substituted or unsubstituted polyether carbamates; substituted or unsubstituted arylazo; substituted or unsubstituted C₂-C₂₀-alkynyl and substituted or unsubstituted C₂-C₂₀-alkenyl;
wherein the one or more substituents, if present in one or more of R¹⁴-R²⁷, are independently selected from the group consisting of D; halogen; NO₂; CN, C₂-C₄₉-alkyl acyl; substituted or unsubstituted C₁-C₂₀-alkoxy; substituted or unsubstituted C₆-C₄₈-aryloxy; substituted or unsubstituted C₂-C₄₉-aryl acyl; (meth)acrylate; tosyl; sulfonic acid or salts thereof, carboxylic acid or salts thereof, boronic acid or salts thereof, phosphonic acid or salts thereof, NR′₃⁺, wherein R′ is independently selected from the group consisting of H, D, substituted or unsubstituted C₁-C₂₀-alkyl and substituted or unsubstituted C₆-C₄₈-aryl, two R′ may form a ring structure; NH₂; and OH; and R¹⁵ and R¹⁶ may be linked to each other to form a unsubstituted or substituted ring structure,
and
wherein at least one other substituent for R² to R⁵ and R¹⁰ to R¹³ is selected from the group consisting of chlorine; bromine; iodine; formyl; carbonate; carbonate ester; ester; amide; CF₃; substituted or unsubstituted C₂-C₄₉-alkyl acyl; substituted or unsubstituted C₂-C₄₉-aryl acyl; ketone; acyl; oxime; aldehyde, NO₂; CN; (meth)acrylate; sulfones; alkylsulfones; sulfonamides; SO₂Me; SO₂NH₂; methoxy; and tosyl;
and/or
wherein at least one other substituent for R² to R⁵ and R¹⁰ to R¹³ is selected from sulfonic acid or salts thereof; carboxylic acid or salts thereof; boronic acid or salts thereof; phosphonic acid or salts thereof; NR′₃⁺, wherein R′ is independently selected from the group consisting of H, D, substituted or unsubstituted C₁-C₂₀-alkyl and substituted or unsubstituted C₆-C₄₈-aryl, two R′ may form a ring structure; substituted or unsubstituted sulfonic esters; substituted or unsubstituted sulfonic amides; phosphonic esters; phosphines; phosphates; sulfinic acids; sulfinic esters; sulfonates; sulfoxides; sulfones; alkylsulfones; oximes; isocyanides; cyanate; isocyanate; thiocyanate; isothiocyanate; ammonium; substituted or unsubstituted carbamates; (meth)acrylate; tosyl; NH₂; and OH.

Preferably, at least one of R² to R⁵ is selected from one of the following formulae:

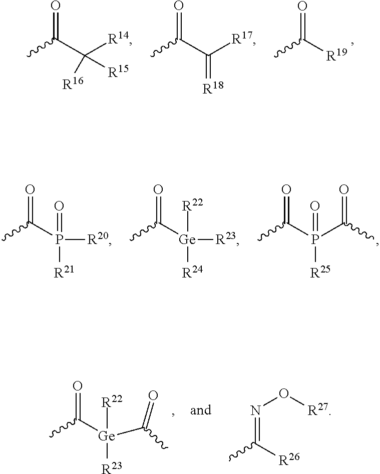

wherein R¹⁴ to R²⁷ are defined above. Additionally, at least one of R¹⁰ to R¹³ may be selected from the group consisting of chlorine; bromine; iodine; formyl; carbonate; carbonate ester; ester; amide; CF₃; substituted or unsubstituted C₂-C₄₉-alkyl acyl; substituted or unsubstituted C₂-C₄₉-aryl acyl; ketone; acyl; oxime; aldehyde; NO₂; CN; (meth)acrylate; sulfones; alkylsulfones; sulfonamides; SO₂Me; SO₂NH₂; methoxy; and tosyl.

On the other hand, at least one other substituent for R¹⁰ to R¹³ may be alternatively or additionally added, selected from sulfonic acid or salts thereof; carboxylic acid or salts thereof; boronic acid or salts thereof; phosphonic acid or salts thereof; NR′₃⁺, wherein R′ is independently selected from the group consisting of H, D, substituted or unsubstituted C₁-C₂₀-alkyl and substituted or unsubstituted C₆-C₄₈-aryl, two R′ may form a ring structure; substituted or unsubstituted sulfonic esters; substituted or unsubstituted sulfonic amides; phosphonic esters; phosphines; phosphates; sulfinic acids; sulfinic esters; sulfonates; sulfoxides; sulfones; alkylsulfones; oximes; isocyanides; cyanate; isocyanate; thiocyanate; isothiocyanate; ammonium; substituted or unsubstituted carbamates; (meth)acrylate; tosyl; NH₂; and OH.

Furthermore, preferably, at least one of R¹⁰ to R¹³; more preferably at least one of R¹⁰ and R¹² to R¹³, is selected from one of the following formulae:

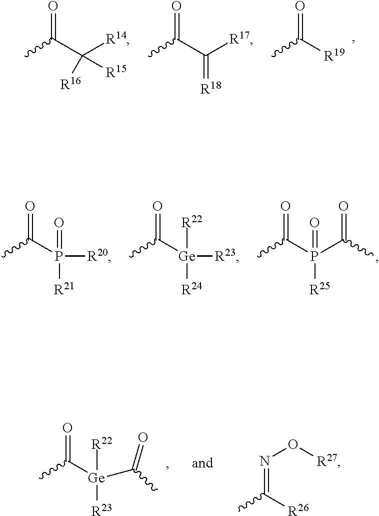

wherein R¹⁴ to R²⁷ are defined above. Additionally, at least one of R² to R⁵ may be selected from chlorine; bromine; iodine; formyl; carbonate; carbonate ester; ester; amide; CF₃; substituted or unsubstituted C₂-C₄₉-alkyl acyl; substituted or unsubstituted C₂-C₄₉-aryl acyl; ketone; acyl; oxime; aldehyde; NO₂; CN; (meth)acrylate; sulfones; alkylsulfones; sulfonamides; SO₂Me; SO₂NH₂; methoxy; and tosyl.

On the other hand, at least one other substituent for R² to R⁵ may be alternatively or additionally added, selected from sulfonic acid or salts thereof; carboxylic acid or salts thereof; boronic acid or salts thereof; phosphonic acid or salts thereof; NR′₃⁺, wherein R′ is independently selected from the group consisting of H, D, substituted or unsubstituted C₁-C₂₀-alkyl and substituted or unsubstituted C₆-C₄₈-aryl, two R′ may form a ring structure; substituted or unsubstituted sulfonic esters; substituted or unsubstituted sulfonic amides; phosphonic esters; phosphines; phosphates; sulfinic acids; sulfinic esters; sulfonates; sulfoxides; sulfones; alkylsulfones; oximes; isocyanides; cyanate; isocyanate; thiocyanate; isothiocyanate; ammonium; substituted or unsubstituted carbamates; (meth)acrylate; tosyl; NH₂; and OH.

Preferably, at least one of R² to R⁵ and additionally at least one of R¹⁰ to R¹³ are substituents selected from one of the following formulae:

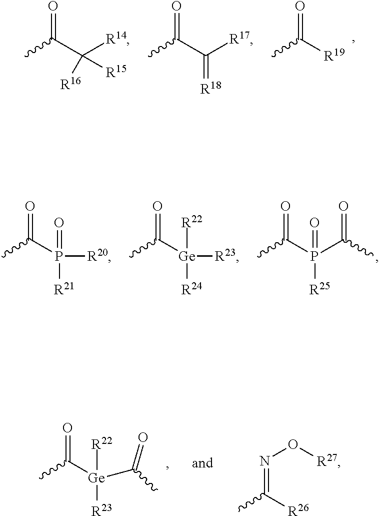

wherein R¹⁴ to R²⁷ are defined above.

Preferably, the selected substituent at one R² to R⁵ is different from the selected substituent at one of R¹⁰ to R¹³.

In one embodiment, at least two substituents of R², R³, R⁵, and R¹⁰-R¹³ are independently selected from the group consisting of

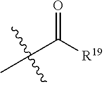

or an electron withdrawing group.

In a further embodiment, at least two substituents of R²-R⁵, R¹⁰, R¹², R¹³ are independently selected from the group consisting of

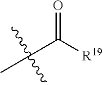

or an electron withdrawing group.

In yet another embodiment, one of R²-R⁵ is

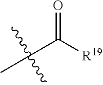

and one of R¹⁰-R¹³ is

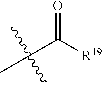

wherein R¹⁹ is the same.

In one embodiment, one of R²-R⁵ is

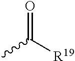

and one of R¹⁰-R¹³ is

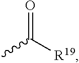

wherein R¹⁹ is different.

In one embodiment, at least one of R²-R⁵ is an electron withdrawing group, and/or at least one of R⁴, R¹⁰, R¹², R¹³ is

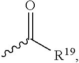

preferably at least one of R¹², R¹³ is

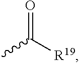

more preferably R¹³ is

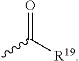

In one embodiment, at least one of R²-R⁵ and/or R¹⁰-R¹³ is a substituted arylacyl and contains an electron withdrawing group as a substituent, preferably the substituent is selected from the group consisting of CN, CF₃, F, Cl, Br, I, OCF₃, substituted or unsubstituted alkylester, substituted or unsubstituted aryl ester, SO₂Me and SO₂NH₂.

In one embodiment, at least one of R²-R⁵ is a substituted or unsubstituted arylacyl and at least one of R¹⁰-R¹³ is a substituted or unsubstituted alkylacyl.

In one embodiment, R⁴ is an electron withdrawing group and R¹³ is

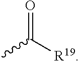

In one embodiment, R¹¹ is H or an electron withdrawing group.

In one embodiment, R¹³ is

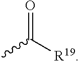

In one embodiment, at least one of R¹⁰-R¹³ is a substituted arylacyl and contains an electron withdrawing group as a substituent, preferably the substituent is selected from the group consisting of CN, CF₃, F, Cl, Br, I, OCF₃, substituted or unsubstituted alkylester, substituted or unsubstituted aryl ester, SO₂Me and SO₂NH₂.

In a preferred embodiment the substituted or unsubstituted aryl acyl as mentioned above is preferably selected from the group consisting of phenyl acyl having the following formula

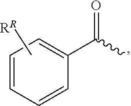

1-naphthyl acyl having the following formula

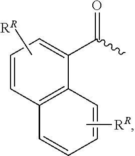

and 2-naphthyl having the following formula

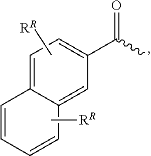

wherein R^{R} are independently selected from the group consisting of hydrogen, deuterium, methyl, tert-butyl, substituted and unsubstituted phenyl, methoxy, CN, CF₃, F, Cl, Br, I, OCF₃, C₁-C₂₀-alkyl ester, C₆-C₄₈-aryl ester, alkyl acyl, aryl acyl, acetyl, benzoyl, NMe₂, SO₂Me, SO₂NH₂.

In a preferred embodiment, C₂-C₄₉-alkyl acyl is preferably C₂-C₈-alkyl acyl, more preferably methyl acyl or ethyl acyl.

In a preferred embodiment, the group of electron withdrawing substituents or electron withdrawing groups contains substituents which are more electron withdrawing in reference to hydrogen (H). The skilled person may refer to: “A survey of Hammett substituent constants and resonance and field parameters”, Chem. Rev. 1991, 91, 2, 165-195, which lists electron withdrawing groups and electron withdrawing substituents.

Preferably, (substituted or unsubstituted) C₁-C₂₀-alkyl ester is methyl ester or ethyl ester.

Preferably, (substituted or unsubstituted) C₆-C₄₈-aryl ester is phenyl ester.

Further preferred embodiments of spiropyran of formula (1) may be stated above in the process for the manufacture of the spiropyran of formula (1).

The following compounds are especially preferred:

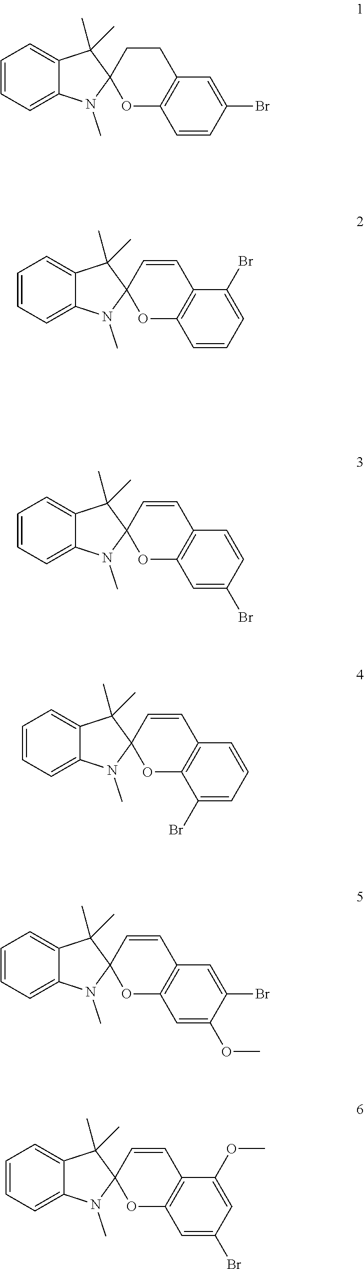 [SMILES: CN1c2ccccc2C(C)(C)C12CCc1cc(Br)ccc1O2 ; CN1c2ccccc2C(C)(C)C12C=Cc1c(Br)cccc1O2 ; CN1c2ccccc2C(C)(C)C12C=Cc1ccc(Br)cc1O2 ; CN1c2ccccc2C(C)(C)C12C=Cc1cccc(Br)c1O2 ; COc1cc2c(cc1Br)C=CC1(O2)N(C)c2ccccc2C1(C)C ; COc1cc(Br)cc2c1C=CC1(O2)N(C)c2ccccc2C1(C)C]

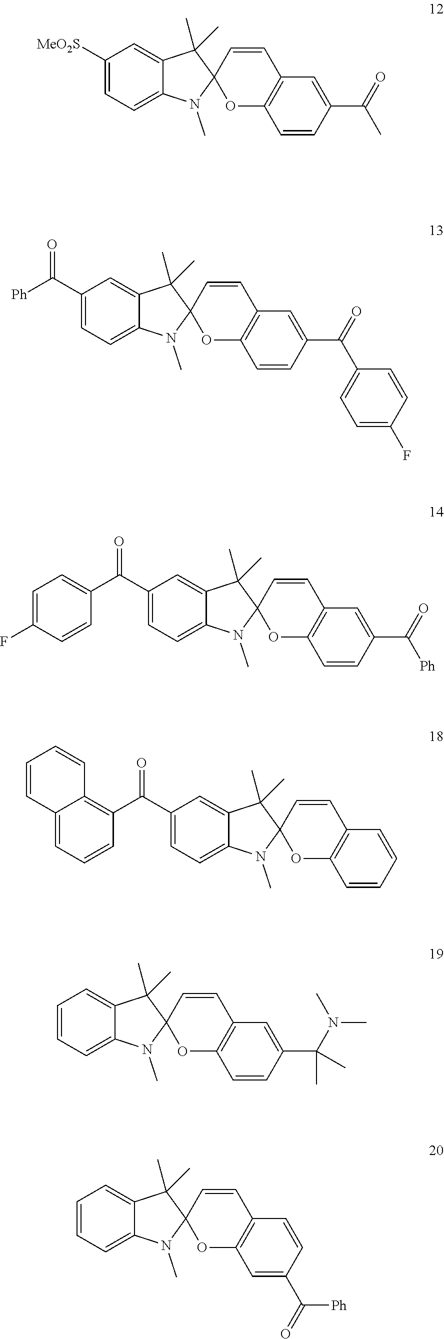 [SMILES: CC(=O)c1ccc2c(c1)C=CC1(O2)N(C)c2ccc(S(C)(=O)=O)cc2C1(C)C ; CN1c2ccc(C(=O)c3ccccc3)cc2C(C)(C)C12C=Cc1cc(C(=O)c3ccc(F)cc3)ccc1O2 ; CN1c2ccc(C(=O)c3ccc(F)cc3)cc2C(C)(C)C12C=Cc1cc(C(=O)c3ccccc3)ccc1O2 ; CN1c2ccc(C(=O)c3cccc4ccccc34)cc2C(C)(C)C12C=Cc1ccccc1O2 ; CN(C)C(C)(C)c1ccc2c(c1)C=CC1(O2)N(C)c2ccccc2C1(C)C ; CN1c2ccccc2C(C)(C)C12C=Cc1ccc(C(=O)c3ccccc3)cc1O2]

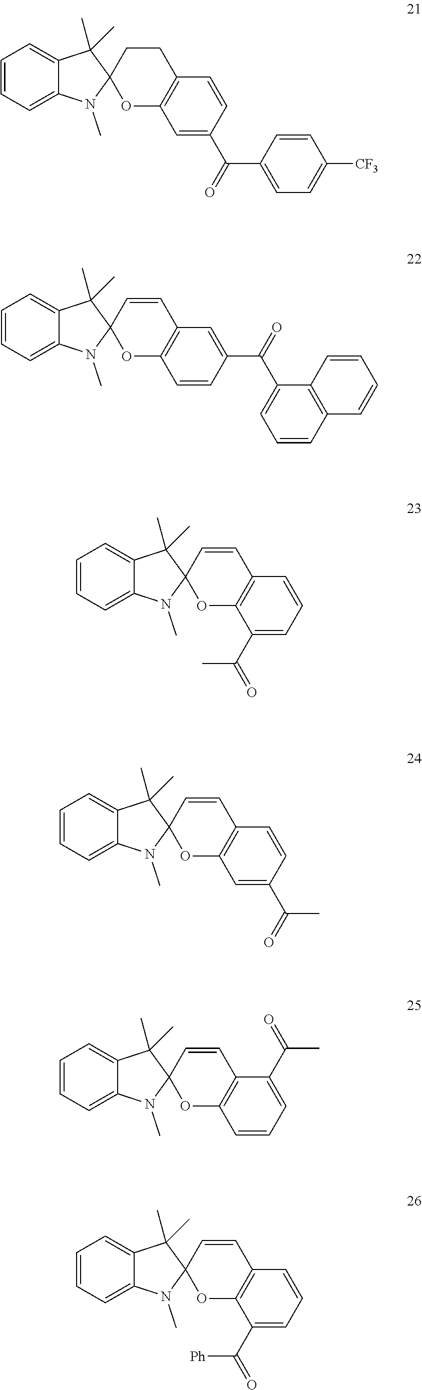 [SMILES: CN1c2ccccc2C(C)(C)C12CCc1ccc(C(=O)c3ccc(C(F)(F)F)cc3)cc1O2 ; CN1c2ccccc2C(C)(C)C12C=Cc1cc(C(=O)c3cccc4ccccc34)ccc1O2 ; CC(=O)c1cccc2c1OC1(C=C2)N(C)c2ccccc2C1(C)C ; CC(=O)c1ccc2c(c1)OC1(C=C2)N(C)c2ccccc2C1(C)C ; CC(=O)c1cccc2c1C=CC1(O2)N(C)c2ccccc2C1(C)C ; CN1c2ccccc2C(C)(C)C12C=Cc1cccc(C(=O)c3ccccc3)c1O2]

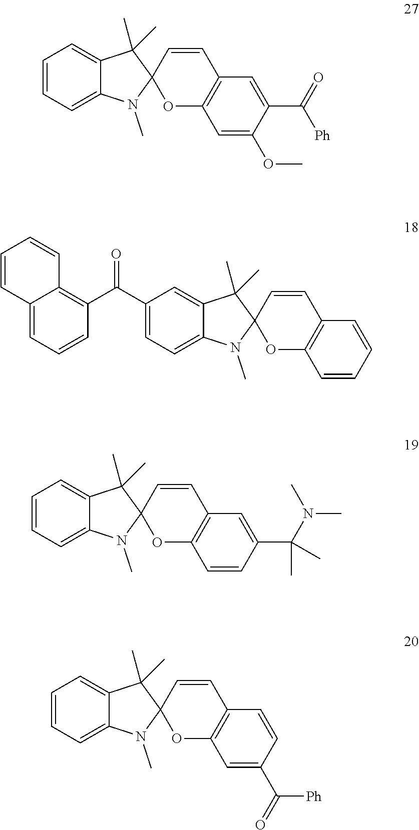 [SMILES: COc1cc2c(cc1C(=O)c1ccccc1)C=CC1(O2)N(C)c2ccccc2C1(C)C ; CN1c2ccc(C(=O)c3cccc4ccccc34)cc2C(C)(C)C12C=Cc1ccccc1O2 ; CN(C)C(C)(C)c1ccc2c(c1)C=CC1(O2)N(C)c2ccccc2C1(C)C ; CN1c2ccccc2C(C)(C)C12C=Cc1ccc(C(=O)c3ccccc3)cc1O2]

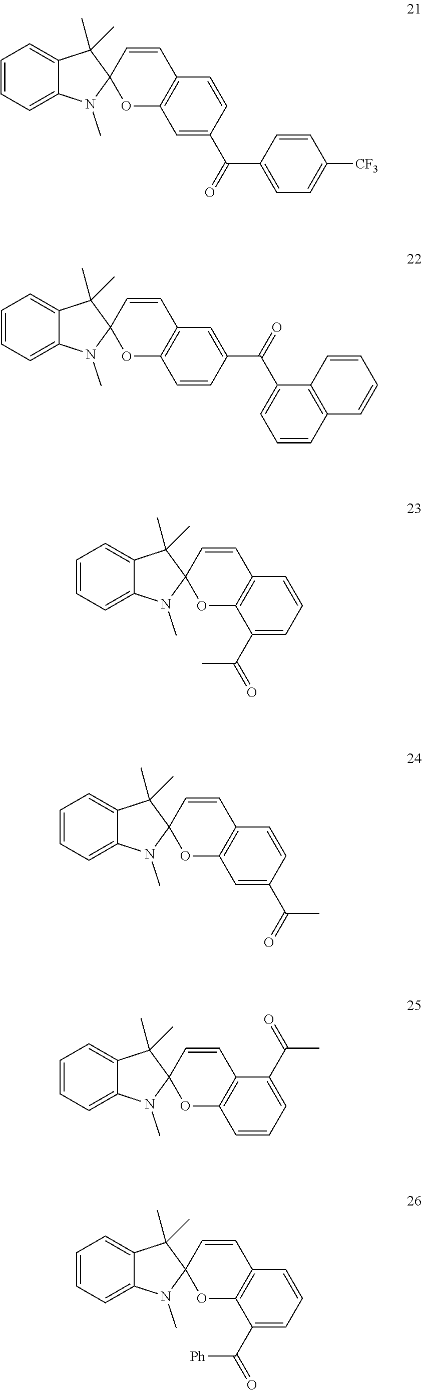 [SMILES: CN1c2ccccc2C(C)(C)C12C=Cc1ccc(C(=O)c3ccc(C(F)(F)F)cc3)cc1O2 ; CN1c2ccccc2C(C)(C)C12C=Cc1cc(C(=O)c3cccc4ccccc34)ccc1O2 ; CC(=O)c1cccc2c1OC1(C=C2)N(C)c2ccccc2C1(C)C ; CC(=O)c1ccc2c(c1)OC1(C=C2)N(C)c2ccccc2C1(C)C ; CC(=O)c1cccc2c1C=CC1(O2)N(C)c2ccccc2C1(C)C ; CN1c2ccccc2C(C)(C)C12C=Cc1cccc(C(=O)c3ccccc3)c1O2]

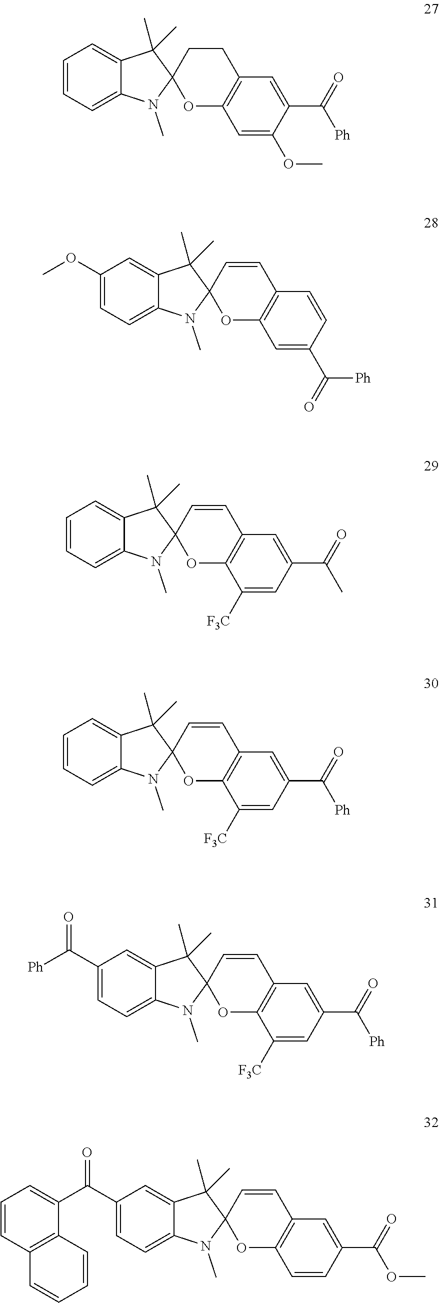 [SMILES: COc1cc2c(cc1C(=O)c1ccccc1)CCC1(O2)N(C)c2ccccc2C1(C)C ; COc1ccc2c(c1)C(C)(C)C1(C=Cc3ccc(C(=O)c4ccccc4)cc3O1)N2C ; CC(=O)c1cc2c(c(C(F)(F)F)c1)OC1(C=C2)N(C)c2ccccc2C1(C)C ; CN1c2ccccc2C(C)(C)C12C=Cc1cc(C(=O)c3ccccc3)cc(C(F)(F)F)c1O2 ; CN1c2ccc(C(=O)c3ccccc3)cc2C(C)(C)C12C=Cc1cc(C(=O)c3ccccc3)cc(C(F)(F)F)c1O2 ; COC(=O)c1ccc2c(c1)C=CC1(O2)N(C)c2ccc(C(=O)c3cccc4ccccc34)cc2C1(C)C]

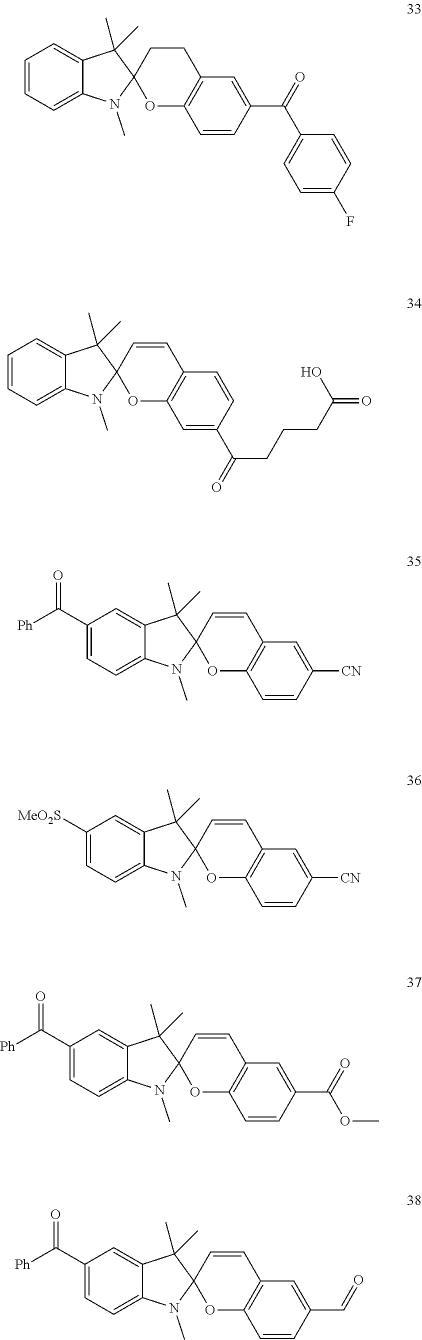 [SMILES: CN1c2ccccc2C(C)(C)C12CCc1cc(C(=O)c3ccc(F)cc3)ccc1O2 ; CN1c2ccccc2C(C)(C)C12C=Cc1ccc(C(=O)CCCC(=O)O)cc1O2 ; CN1c2ccc(C(=O)c3ccccc3)cc2C(C)(C)C12C=Cc1cc(C#N)ccc1O2 ; CN1c2ccc(S(C)(=O)=O)cc2C(C)(C)C12C=Cc1cc(C#N)ccc1O2 ; COC(=O)c1ccc2c(c1)C=CC1(O2)N(C)c2ccc(C(=O)c3ccccc3)cc2C1(C)C ; CN1c2ccc(C(=O)c3ccccc3)cc2C(C)(C)C12C=Cc1cc(C=O)ccc1O2]

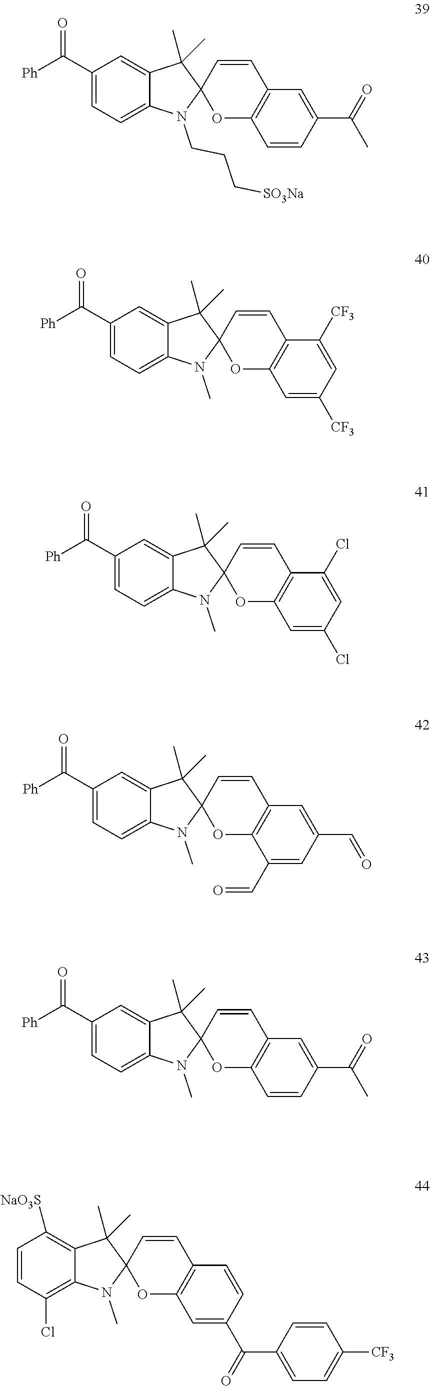

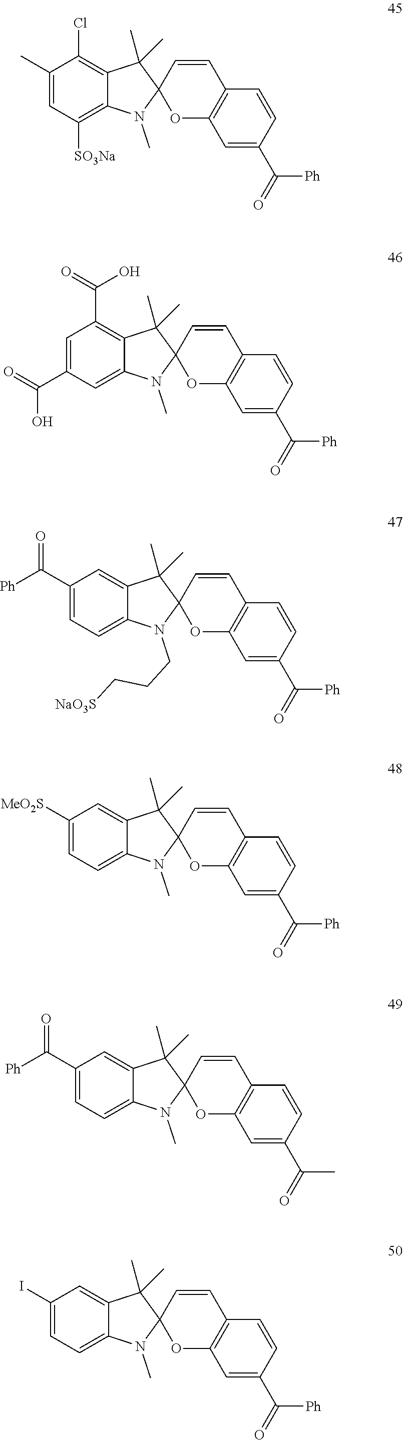

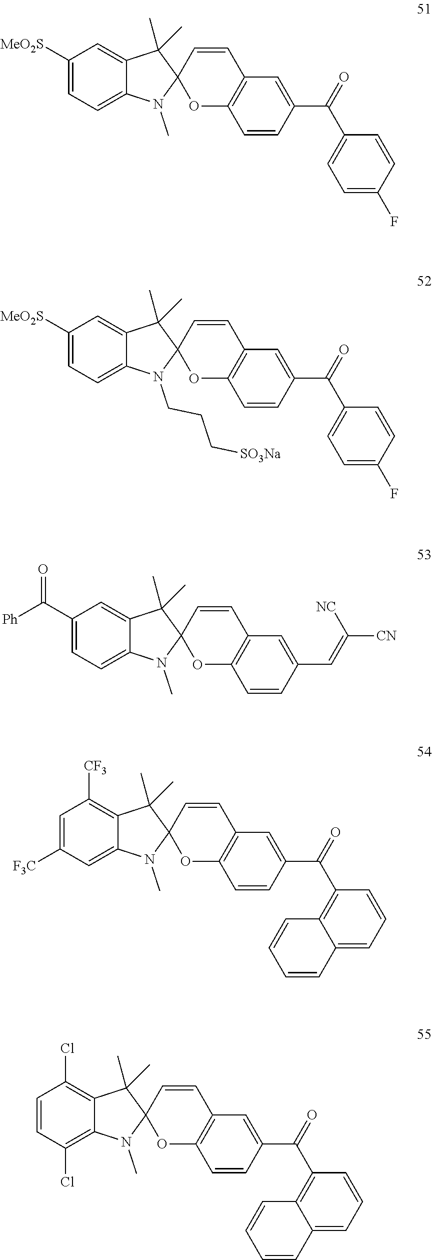

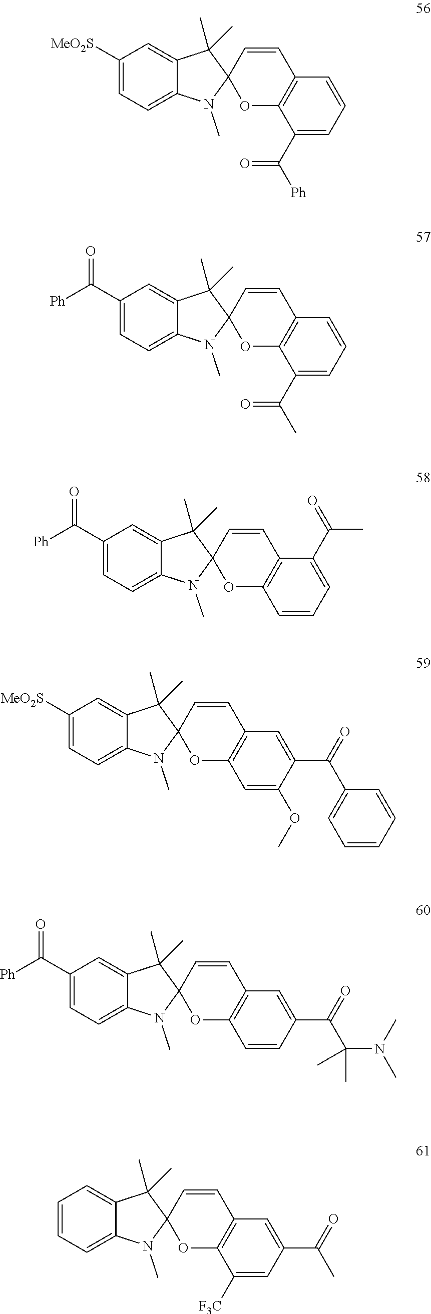 [SMILES: CN1c2ccc(S(C)(=O)=O)cc2C(C)(C)C12C=Cc1cccc(C(=O)c3ccccc3)c1O2 ; CC(=O)c1cccc2c1OC1(C=C2)N(C)c2ccc(C(=O)c3ccccc3)cc2C1(C)C ; CC(=O)c1cccc2c1C=CC1(O2)N(C)c2ccc(C(=O)c3ccccc3)cc2C1(C)C ; COc1cc2c(cc1C(=O)c1ccccc1)C=CC1(O2)N(C)c2ccc(S(C)(=O)=O)cc2C1(C)C ; CN(C)C(C)(C)C(=O)c1ccc2c(c1)C=CC1(O2)N(C)c2ccc(C(=O)c3ccccc3)cc2C1(C)C ; CC(=O)c1cc2c(c(C(F)(F)F)c1)OC1(C=C2)N(C)c2ccccc2C1(C)C]

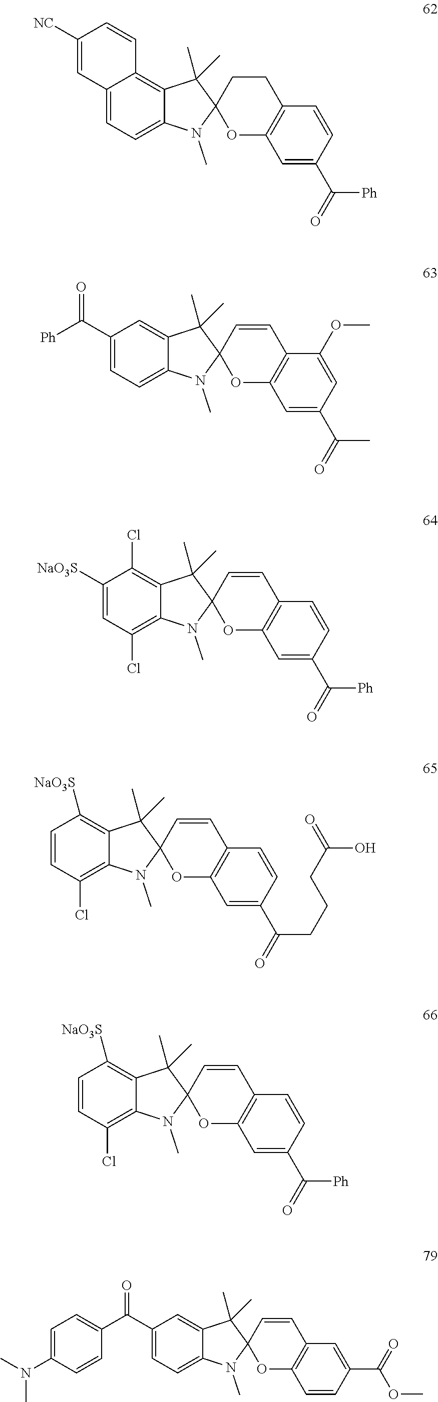

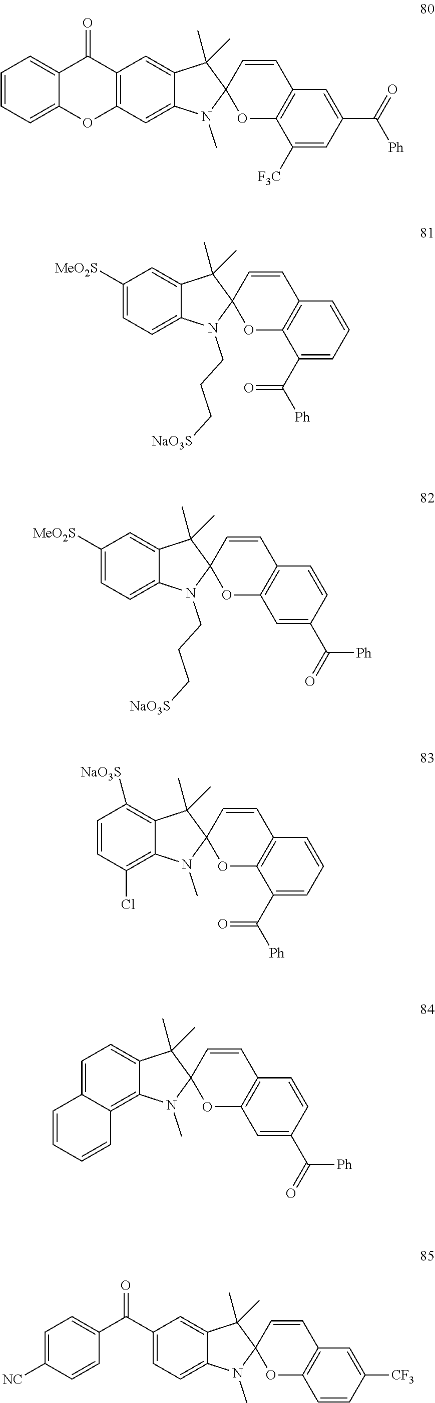

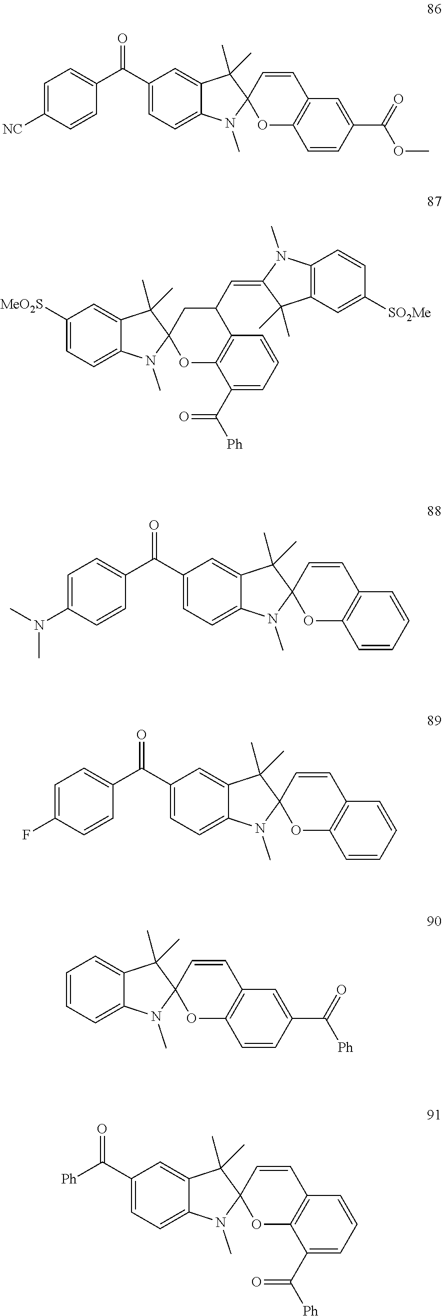

### 3. Dual Color Photoinitiators

In a further aspect of the present invention, the spiropyran of formula (1) may be a photoinitiator, preferably a dual color photoinitiator.

The photoinitiator molecule, and its necessary function can be produced in different ways. One example provides the following:

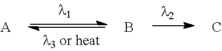

The photoinitiator can exist in three different states, which may be characterized as follows:

### Initial State (A):

Without light irradiation the photoinitiator molecules are present in this state. The spiropyran of formula (1) may be the initial state (A).

### Intermediate State (B):

The B state is an electronic ground state. The corresponding merocyanine form of spiropyran of formula (1) may be the intermediate state B.
The intermediate state is created from the initial state A by absorption of light of wavelength λ₁.
The photoinitiator molecules have a new or more intense absorption band for light of wavelength λ₂.
Alternatively, the absorption band for λ₁ disappears. The photoinitiator molecule returns to the initial state A spontaneously in the absence of light or by absorption of light of wavelength λ₃.

### Reactive State (C):

The reactive state is generated from the intermediate state B by absorption of light of wavelength λ₂.
The reactive state initiates a polymerization reaction in the immediate vicinity of the molecule.
A back reaction to B is not intended.

In another aspect, the present invention discloses a process for locally polymerizing a starting material by using a spiropyran as a photoinitiator and irradiating the spiropyran with light of at least one wavelength, preferably with light of two different wavelengths.

In a special embodiment, the light of at least one wavelength may have a first wavelength provided by the simultaneous absorption of two photons of a second wavelength.

The light of the second wavelength may be provided by a pulsed laser source, which may be of sufficiently high intensity to induce a two-photon-absorption event of the dual color photoinitiator in the initial state. Without being bound by any theory, the photoinitiator may switch to the intermediate state, where it absorbs at least a single photon of the second wavelength and forms the reactive state. The reactive state may initiate a polymerization reaction, which may be used for the manufacture of a shaped body, especially of a shaped body with high resolution. The second wavelength of the pulsed laser source may be in the range of 500-1000 nm, preferably 600-800 nm. Preferably, the first wavelength and the second wavelength may be the same.

The light of the first wavelength may be absorbed by the photoinitiator resulting in the formation of the intermediate state. The intermediate state may have a higher extinction coefficient at the first wavelength. Due to the formation of the intermediate state the absorbance at the first wavelength may increase, thereby limiting the penetration of the light of the first wavelength into the resin. The intermediate state absorbs the light of the first wavelength and forms the reactive state. The reactive state initiates a polymerization. Such an embodiment may be preferably used for 3D printing with stereolithography, digital light processing or similar techniques. Due to the increasing absorption at the first wavelength, the penetration of the light into the resin may be limited. The limited penetration may be advantageous for a special embodiment to reduce the amount of absorber in the resin or to improve the z-resolution of the printed article.

In a preferred embodiment, the process is for locally polymerizing a starting material by using a spiropyran as a photoinitiator and irradiating the spiropyran with light of two different wavelengths.

In one embodiment, the present invention discloses a process for locally polymerizing a starting material by using a spiropyran as a photoinitiator and irradiating the spiropyran with at least one light source of at least one wavelength, preferably with two light sources of different wavelengths, according to the disclosure above.

In yet another aspect, the present invention discloses a process for locally polymerizing a starting material by dual color photopolymerization, comprising:
providing a polymerizable starting material containing photoinitiator molecules wherein the photoinitiator molecule is a spiropyran according to the present invention which can be converted by sequential optical excitation into a reactive state in which the photoinitiator molecules locally trigger polymerization of the starting material; and
photopolymerizing the starting material in a local volume by irradiating light of a first wavelength and light of a second wavelength, different from the first wavelength, into the local volume, whereby in the local volume
the photoinitiator molecules are converted, due to the absorption of the light of the first wavelength, from an initial state in which the photoinitiator molecules substantially do not absorb the light of the second wavelength, into an intermediate state with changed optical properties compared to the initial state, such that the photoinitiator molecules in the intermediate state absorb the light of the second wavelength; and
the photoinitiator molecules are transferred from the intermediate state to the reactive state due to the absorption of the light of the second wavelength, which triggers the polymerization locally, and/or
the photoinitiator molecules may be transferred from the intermediate state to the initial state spontaneously in a thermal reaction.

Dual color photoinitiators, which respond to electromagnetic radiation of a first wavelength by switching from a thermodynamically stable state A to a metastable state B. B is able to absorb electromagnetic radiation of a second wavelength, thereby forming C, that can initiate a polymerization reaction with or without a co-initiator. B can undergo a fast thermal reverse reaction to the form A and may then be deactivated for electromagnetic radiation of the second wavelength. Application of such dual color photoinitiators may allow the curing of a photopolymerizable resin in any volume, where electromagnetic radiation of both wavelengths intersects, for example where an image may be projected on a light sheet.

The disclosed dual color photoinitiators may be characterized in a way that a polymerization may be induced where electromagnetic radiation of two different wavelengths interact with the same volume of polymerizable material in a simultaneous or consecutive fashion. In volumes which interact with electromagnetic irradiation of only one wavelength, no polymerization may be induced.

Due to the absorption of the light of the second wavelength, the photoinitiator molecules can be converted into a reactive state which triggers a radical polymerization in the local volume.

A light beam of light of the first wavelength and a light beam of light of the second wavelength can be irradiated in the local volume at least partially overlapping.

The starting material can be polymerized in several local volumes by means of photo polymerization and thus a three-dimensional shaped body can be produced in the starting material.

Without being bound by any theory, a dual color photoinitiator in the thermodynamically stable form A may absorb a photon from electromagnetic radiation of the first wavelength which induces an isomerization reaction to the metastable form B. The dual color photoinitiator in the metastable form B may absorb a photon from electromagnetic radiation of the second wavelength, which results in an excited state C and may further cause the formation of radicals by hydrogen abstraction from a co-initiator followed by electron transfer or decomposition into radicals, by electron transfer which is followed by hydrogen abstraction or decomposition into radicals, or homolytic bond cleavage which can be preceded or followed by other rearrangement reactions to form radicals. Dual color photoinitiators in the metastable state B which have not absorbed a photon from electromagnetic radiation of the second wavelength, may return spontaneously via a thermal process to the thermodynamically stable state A.

The dual color photoinitiators carry carbonyl functions which are triplet sensitizers for spiropyrans and cause efficient switching to the merocyanine form via the triplet state and therefore may not show curing with UV light alone. Electromagnetic radiation of the first wavelength causes excitation of the initiator in form A and switching to the initiator in form B. The efficient ring opening reaction of the photoswitch motif may prevent the dual color photoinitiators from radical formation by irradiation with the first wavelength alone. The merocyanine type form B can act as an internal triplet sensitizer upon irradiation with the second wavelength, which may cause the carbonyl group to abstract a hydrogen atom from a co-initiator, undergo an electron transfer reaction, or undergo homolytic bond cleavage. Alternatively, the merocyanine type form B can undergo an electron transfer reaction with the co-initiator from the singlet excited state. The substituents may be selected in a way to minimize or extinguish the absorption of the merocyanine type form B at the first wavelength and that form B is thermodynamically destabilized to ensure a fast thermal back reaction from B to A. Substituents are also chosen in a way to adjust the photo redox potential to the respective co-initiator if present. Furthermore, the dual color photoinitiators may benefit from an exceptionally low or neglectable quantum yield for the competing photoreaction from B to A and high extinction coefficients of the B form, where form A does not absorb. The merocyanine form B may typically have a broad absorption in the visible region, which allows for high intensities over a broad range of wavelengths.

Spiropyran of formula (1) that are used as dual color photoinitiators can be functionalized with several strong electron withdrawing groups for R²-R⁵ and R¹⁰-R¹³. In one preferred embodiment, at least one electron withdrawing group is substituted for R¹⁰-R¹³ and at least another electron withdrawing group which may be the same or different may be substituted for R²-R⁵ of spiropyran of formula (1). The electron withdrawing group can be the same or different and include but are not limited to cyano, formyl, keto, nitro, ester, trifluoromethyl, dicyanovinylene, methylsulfone, sulfonamide, fluoro, chloro, bromo, and iodo. In the spiropyran form, the electron withdrawing groups are decoupled. When the spiropyran absorbs UV-light, it opens to the merocyanine, since the ring opening reaction is very efficient. In the merocyanine form, the acceptors are in conjugation, decreasing the HOMO and LUMO of the merocyanine. When the electron deficient merocyanine absorbs visible light, the excited state is formed, which is strongly oxidizing and has a long lifetime. An electron is transferred from a co-initiator to the merocyanine, which is followed by a proton transfer. The hereby formed starting radicals can initiate a polymerization.

In one aspect, the intermediate state may return thermally at the printing temperature in the printing resin to the initial state. Preferably, the intermediate state may return thermally at the printing temperature to the initial state in a mechanism with one or more rate constants with the highest rate constant higher than k=0.01 s⁻¹. Especially preferably, at least one rate constant for the thermal back reaction is higher than 0.02 s⁻¹, more preferably, higher than 0.05 s⁻¹, even more preferably higher than 0.08 s⁻¹, most preferably higher than 0.25 s⁻¹, but optionally not higher than 0.65 s⁻¹. Hence, the rate constant may be in the range of 0.1 s⁻¹ and 0.65 s⁻¹, or any other range which may be formed from the values above.

The spiropyran of formula (1) shown above may be used in connection with the process for locally polymerizing a starting material by dual color photopolymerization. The same applies to the process for 3D-printing of the molded body or the method for volumetric printing of a shaped body. A detailed description in which spiropyran are used in a formulation for dual color photopolymerization in volumetric printing may be found in PCT application [Garmshausen et al., WO2020245456A1], which demonstrates the successful application of spiropyrans as photoinitiators, especially dual color photoinitiators for volumetric printing.

Formulations suitable for volumetric printing may contain the following parts by weight:
1-99.9999 wt %, preferably 5-99.99 wt %, more preferably 20-99.9 wt % of photopolymerizable compound(s), e.g. monomer(s);
0-99 wt %, preferably 1-50 wt %, more preferably 3-20 wt % co-initiator, when the co-initiator contains photopolymerizable groups, e.g. acrylates;
0-50 wt %, preferably 1-40 wt %, more preferably 3-10 wt % of co-initiator, when the co-initiator does not contain photopolymerizable groups;
0.0001-20 wt %, preferably 0.001-10 wt %, more preferably 0.01-5 wt %, most preferably 0.1-1 wt % of a spiropyran of formula (1) which may be used a dual color photoinitiator;
0-20 wt %, preferably 1-10 wt %, more preferably 3-5 wt % of acids or bases;
0-90 wt %, preferably 1-70 wt %, more preferably 5-50 wt %, most preferably 10-30 wt % of other additives, such as organic or inorganic fillers, optical brighteners, inhibitors, chain transfer agents and others;
0-90 wt %, preferably 5-50 wt %, more preferably 10-30 wt % of solvent; and
0-99 wt %, preferably 5-95 wt %, more preferably 20-80 wt %, even more preferably 30-70 wt % of water.

All weight ratios are given with respect to the weight of the total formulation.

In a preferred embodiment the dual color photoinitiator may be lyophilized or freeze dried prior to addition to the formulation.

In another preferred embodiment, prior to addition to the formulation, the dual color photoinitiator may be precipitated as a powder by adding a solution of the dual color photoinitiator to a solvent wherein the dual color photoinitiator has a lower solubility.

Typical curing parameters which are suitable for volumetric printing may be:
any setup which employs light of two different wavelengths;
a temperature of −20° C. to +100° C.; preferably 0° C. to +60° C., more preferably +20° C. to +60° C.
a first wavelength of: 250 nm to 500 nm; preferably, 300 nm to 450 nm and
a second wavelength of: 350 nm to 800 nm.

The co-initiator can be of high molecular weight, such as >1000 g/mol, or bound to a polymer to prevent migration in the cured object. The co-initiator can contain polymerizable groups, such as acrylates or methacrylates which are built into the polymer network during curing to prevent later migration of the co-initiator. A typical example is the following:

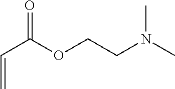

Alternatively, the co-initiator can be a derivative of ethanolamine, preferably a derivative of diethanolamine, more preferably an N-alkyldiethanolamin, alternatively N-methyldiethanolamin, N-phenyldiethanolamin, triethanolamine, 4-(2-hydroxyethyl)morpholine, N-(2-hydroxypropyl)morpholine, N-tert-butyldiethanolamine, N-butyldiethanolamine, N-(3-aminopropyl)diethanolamine, N,N-di(2-hydroxyethyl)glycine, 1-[bis(2-hydroxyethyl)amino]-2-propanol, N-phenyldiethanolamine, m-tolyldiethanolamine, p-tolyldiethanolamine, N-benzyldiethanolamine, bis(2-hydroxyethyl)aminotris(hydroxymethyl)methane, N,N-bis(2-hydroxypropyl)aniline, N,N-bis(2-hydroxyethyl)-2-aminoethanesulfonic Acid, N,N-bis(2-hydroxyethyl)-3-chloroaniline, N,N,N′,N′-tetrakis(2-hydroxyethyl)ethylenediamine, 3-[N,N-bis(2-hydroxyethyl)amino]-2-hydroxypropanesulfonic acid, N-lauryldiethanolamine, N,N,N′,N′-tetrakis(2-hydroxypropyl)ethylenediamine, N,N,N′,N″,N″-pentakis(2-hydroxypropyl)diethylenetriamine, ethyl 4-(dimethylamino)benzoate, isoamyl 4-(dimethylamino)benzoate, 2-butoxyethyl 4-(dimethylamino)benzoate, 2-ethylhexyl 4-(dimethylamino)benzoate.

Any combinations of embodiments, preferred ranges and/or moieties, in particular, preferred 76 moieties of the invention are particularly preferred.

In one aspect, the present invention discloses a process for locally polymerizing a starting material by dual color photopolymerization and forming a shaped body, comprising:
providing a container which is at least partially filled with a polymerizable starting material containing photoinitiator molecules, which can be converted by sequential optical excitation into a reactive state in which the photoinitiator molecules locally trigger polymerization of the starting material; and
photopolymerizing the starting material in a container by irradiating light of a first wavelength and light of a second wavelength, different from the first wavelength, into the container, whereby in the local volume
the photoinitiator molecules are converted, due to the absorption of the light of the first wavelength, from an initial state in which the photoinitiator molecules substantially do not absorb the light of the second wavelength, into an intermediate state with changed optical properties compared to the initial state, such that the photoinitiator molecules in the intermediate state absorb the light of the second wavelength; and
the photoinitiator molecules are transferred from the intermediate state to the reactive state due to the absorption of the light of the second wavelength, which triggers the polymerization locally to form the shaped body; wherein
   the initial state of the photoinitiator in the photopolymerizable material has an extinction coefficient at the first wavelength which is lower than 5000 L mol⁻¹ cm⁻¹, preferably lower than 2500 L mol⁻¹ cm⁻¹, more preferably lower than 1000 L mol⁻¹ cm⁻¹, even more preferably lower than 500 L mol⁻¹ cm⁻¹, most preferably lower than 250 L mol⁻¹ cm⁻¹; and/or
   The initial state of the photoinitiator in the photopolymerizable material may have an extinction coefficient at the first wavelength which is higher than 30 L mol⁻¹ cm⁻¹, preferably higher than 70 L mol⁻¹ cm⁻¹, more preferably higher than 100 L mol⁻¹ cm⁻¹, even more preferably higher than 150 L mol⁻¹ cm⁻¹, most preferably higher than 200 L mol⁻¹ cm⁻¹, like higher than 210 L mol⁻¹ cm⁻¹. Hence, the initial state of the photoinitiator in the photopolymerizable material may have an extinction coefficient at the first wavelength which is in the range of 5000-0 L mol⁻¹ cm⁻¹, preferably 2500-10 L mol⁻¹ cm⁻¹, more preferably 1000-20 L mol⁻¹ cm⁻¹, even more preferably 500-50 L mol⁻¹ cm⁻¹, most preferably 300-100 L mol⁻¹ cm⁻¹, like 300-210 L mol⁻¹ cm⁻¹.

In another aspect, the present invention discloses a process for locally polymerizing a starting material by dual color photopolymerization and forming a shaped body, comprising:
providing a container which is at least partially filled with a polymerizable starting material containing photoinitiator molecules, which can be converted by sequential optical excitation into a reactive state in which the photoinitiator molecules locally trigger polymerization of the starting material; and
photopolymerizing the starting material in the container by irradiating light of a first wavelength and light of a second wavelength, different from the first wavelength, into the container, whereby in the container
the photoinitiator molecules are converted, due to the absorption of the light of the first wavelength, from an initial state in which the photoinitiator molecules substantially do not absorb the light of the second wavelength, into an intermediate state with changed optical properties compared to the initial state, such that the photoinitiator molecules in the intermediate state absorb the light of the second wavelength; and
the photoinitiator molecules are transferred from the intermediate state to the reactive state due to the absorption of the light of the second wavelength, which triggers the polymerization locally to form the shaped body;
   wherein the photopolymerizable material has an absorbance at the first wavelength in a range between 1 and 0.05 absorbance units, preferably in a range between 1 and 0.07 absorbance units, more preferably in a range between 0.90 and 0.10 absorbance units, even more preferably in a range between 0.80 and 0.15 absorbance units when measured in a distance of 1 cm. The absorbance of the photopolymerizable material may be measured by UV/-Vis spectrometry using a Cary60 UV-Vis spectrophotometer supplied by Agilent Technologies. Acetonitrile may be used as a reference to determine the absorbance. The first wavelength may be 250 nm to 500 nm; preferably 300 nm to 450 nm.

In an additional aspect, the present invention discloses a process for locally polymerizing a starting material by dual color photopolymerization and forming a shaped body, comprising:
providing a container which is at least partially filled with a polymerizable starting material containing photoinitiator molecules, which can be converted by sequential optical excitation into a reactive state in which the photoinitiator molecules locally trigger polymerization of the starting material; and
photopolymerizing the starting material in a container by irradiating light of a first wavelength and light of a second wavelength, different from the first wavelength, into the container, whereby in the local volume
the photoinitiator molecules are converted, due to the absorption of the light of the first wavelength, from an initial state in which the photoinitiator molecules substantially do not absorb the light of the second wavelength, into an intermediate state with changed optical properties compared to the initial state, such that the photoinitiator molecules in the intermediate state absorb the light of the second wavelength; and
the photoinitiator molecules are transferred from the intermediate state to the reactive state due to the absorption of the light of the second wavelength, which triggers the polymerization locally to form the shaped body; wherein
   the initial state of the photoinitiator in the photopolymerizable material has an extinction coefficient at the first wavelength which is lower than 5000 L mol⁻¹ cm⁻¹, preferably lower than 2500 L mol⁻¹ cm⁻¹, more preferably lower than 1000 L mol⁻¹ cm⁻¹, even more preferably lower than 500 L mol⁻¹ cm⁻¹, most preferably lower than 250 L mol⁻¹ cm⁻¹; and/or
   The initial state of the photoinitiator in the photopolymerizable material may have an extinction coefficient at the first wavelength which is higher than 30 L mol⁻¹ cm⁻¹, preferably higher than 70 L mol⁻¹ cm⁻¹, more preferably higher than 100 L mol⁻¹ cm⁻¹, even more preferably higher than 150 L mol⁻¹ cm⁻¹, most preferably higher than 200 L mol⁻¹ cm⁻¹. Hence, the initial state of the photoinitiator in the photopolymerizable material may have an extinction coefficient at the first wavelength which is in the range of 5000-0 L mol⁻¹ cm⁻¹, preferably 3000-10 L mol⁻¹ cm⁻¹, more preferably 2000-20 L mol⁻¹ cm⁻¹, even more preferably 1000-50 L mol⁻¹ cm⁻¹, most preferably 500-100 L mol⁻¹ cm⁻¹; and/or
   wherein the photopolymerizable material has an absorbance at the first wavelength in a range between 1 and 0.05 absorbance units, preferably in a range between 0.90 and 0.10 absorbance units, more preferably in a range between 0.80 and 0.15 absorbance units when measured in a distance of 1 cm. The absorbance of the photopolymerizable material may be measured by UV/-Vis spectrometry using a Cary60 UV-Vis spectrophotometer supplied by Agilent Technologies. Acetonitrile may be used as a reference. An extinction coefficient for a specific wavelength may be calculated according to the Beer-Lambert law from the absorbance of a resin with known concentration of photoinitiator, using the same resin without photoinitiator as a reference. The first wavelength may be 250 nm to 500 nm; preferably 300 nm to 450 nm.

Using the specified extinction coefficients and absorbances units may have the surprising effect of an improved dual color effect, so that hardening at the walls of the local volume, preferably container, is minimized, while a high degree of polymerization at the intersection of the first and second wavelength is assured.

Preferably, the photoinitiator molecule is a spiropyran as disclosed in the present invention.

Preferably, a local volume may be container.

Preferably, the container may be at least partially transparent. Hence, the container is configured to at least partially transmit the first wavelength and second wavelength from the source into the container so that a polymerization reaction may be locally triggered. The container may contain a rectangular surface, preferably the container has a cubic shape.

The container may cover a volume of at least 0.5 cm×0.5 cm×0.5 cm, preferably at least 1 cm×1 cm×1 cm, more preferably at least 1.5 cm×1.5 cm×1.5 cm, even more preferably at least 3 cm×3 cm×3 cm, most preferably at least 5 cm×5 cm×5 cm.

Preferably, the light of the first wavelength and the light of the second wavelength are simultaneously irradiated into the local volume.

In an alternative embodiment, the light of the second wavelength may be irradiated into the local volume after the irradiation of the light of the first wavelength in the local volume has ended, while the light of the second wavelength may be irradiated before the end of a decay time of the intermediate state of the photoinitiator molecules.

Furthermore, preferably, the photoinitiator molecules in the intermediate state substantially do not absorb the light of the first wavelength.

Preferably, the photoinitiator molecules in the initial state have an extinction coefficient at the second wavelength which is lower than 2000 L mol⁻¹ cm⁻¹, more preferably lower than 1000 L mol⁻¹ cm⁻¹, even more preferably lower than 500 L mol⁻¹ cm⁻¹, still more preferably lower than 200 L mol⁻¹ cm⁻¹, most preferably lower than 100 L mol⁻¹ cm⁻¹.

Preferably, the photoinitiator molecules may be converted into a reactive state due to the sequential absorption of light of the first and second wavelength which may trigger a polymerization in the local volume.

Preferably, the photoinitiator molecules are converted into a reactive state due to the absorption of the light of the second wavelength, which may trigger a radical polymerization in the local volume.

Preferably, the light of the first wavelength is irradiated as a light beam and/or the light of the second wavelength is irradiated as a light beam. A light beam may have a diameter of 2 cm or less, preferably 1 cm or less, more preferably 0.5 cm or less, most preferably 1 mm or less. The light beam of the light of the first wavelength and the light beam of the light of the second wavelength may at least partially overlap in the local volume, preferably container, when irradiated.

Preferably, the light of the first wavelength is irradiated as a light sheet and the light of the second wavelength is irradiated as a projection of a 2D-image. The light sheet of the light of the first wavelength and the light projection of the light of the second wavelength may at least partially overlap in the local volume, preferably container, when irradiated.

Preferably, the starting material is polymerized in several local volumes by means of photopolymerization and thus a three-dimensional shaped body is produced in the starting material.

A further aspect of the present invention is a process for 3D-printing a shaped body, wherein the shaped body is produced by means of a process as it is disclosed above.

4. Objects with Reduced Color

It is generally difficult to remove chromophores from the inside of a printed three-dimensional object in volumetric 3d-printing processes. As an example, when a photoinitiator fragment or photoinitiator radical initiates a polymerization or reacts with a radical chain end, the resulting chromophore is bound to the polymer structure of the printed three-dimensional object and cannot be removed e.g. by extraction or washing.

Below illustrated is an example of a way of how a photoinitiator radical, PIH⋅, and a co-initiator radical, CI⋅, can be formed from a photoinitiator, PI, and a co-initiator CIH:

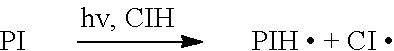

Below illustrated is an example of how a co-initiator fragment, CI, and a photoinitiator, PI, can form part of an exemplary acrylate polymer structure on basis of a radical reaction:

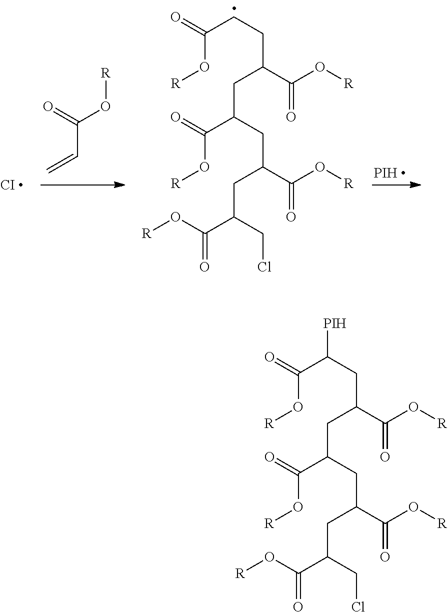

As a consequence, three-dimensional objects manufactured with volumetric 3d-printing processes typically have specific optical properties, namely absorption properties in the visible wavelength range, which result in a color or coloring, respectively. A color or coloring of the three-dimensional objects is not always desired and can indeed even be undesired for certain applications.

As such, there is a need to improve methods for manufacturing three-dimensional objects with respect to the optical properties of the three-dimensional objects which can be manufactured therewith.

According to one aspect of the invention, the process for the formation of a shaped body by dual color photopolymerization comprises further a step of post-processing the shaped body comprising a thermal treatment of the shaped body and/or an optical treatment of the shaped body.

In one embodiment a three-dimensional object may be the shaped body, or a three-dimensional object may be formed from the shaped body. Preferably a three-dimensional object may be formed from a shaped body by removal of the shaped body from the polymerizable starting material.

In one embodiment the post-processing of the three-dimensional object comprises modifying the optical properties of the three-dimensional object resulting in a decrease of the absorption properties of the three-dimensional object for at least one wavelength in a wavelength range between 300 nm and 2000 nm, preferably between 350 nm and 1000 nm, more preferably between 400 nm and 900 nm, most preferably between 450 nm and 800 nm and/or in an increase of the transmissive properties of the three-dimensional object for at least one wavelength in the wavelength range between 300 nm and 2000 nm, preferably between 350 nm and 1000 nm, more preferably between 400 nm and 900 nm, most preferably between 450 nm and 800 nm.

In one embodiment the post-processing of the three-dimensional object comprises modifying the optical properties of the three-dimensional object resulting in that an average transmission or an integral of the transmission between 300 nm and 2000 nm, preferably between 350 nm and 1000 nm, more preferably between 400 nm and 900 nm, most preferably between 450 nm and 800 nm, is increased by at least 1%, preferably at least 2%, more preferably at least 3%, more preferably at least 4%, more preferably at least 5%, more preferably at least 7.5%, more preferably at least 10%, more preferably at least 15%, more preferably at least 20%, more preferably at least 25%, more preferably at least 30%, more preferably at least 35%, more preferably at least 40%, more preferably at least 45%, more preferably at least 50%, preferably relative to a state of the three-dimensional object before it has undergone the at least one method for post-processing of the three-dimensional object; and/or

the post-processing of the three-dimensional object includes modifying the optical properties of the three-dimensional object resulting in that an average absorption or an integral of the absorption between 300 nm and 2000 nm, preferably between 350 nm and 1000 nm, more preferably between 400 nm and 900 nm, most preferably between 450 nm and 800 nm, is decreased by at least 1%, preferably at least 2%, more preferably at least 3%, more preferably at least 4%, more preferably at least 5%, more preferably at least 7.5%, more preferably at least 10%, more preferably at least 15%, more preferably at least 20%, more preferably at least 25%, more preferably at least 30%, more preferably at least 35%, more preferably at least 40%, more preferably at least 45%, more preferably at least 50% preferably relative to a state of the three-dimensional object before it has undergone the at least one method for post-processing.

In one embodiment the post-processing of the three-dimensional object comprises modifying the optical properties of the three-dimensional object resulting in an average or integrated absorption per mm of thickness of the three-dimensional object of less than 0.5, preferably less than 0.3, more preferably less than 0.2, more preferably less than 0.1, in a wavelength range between 300 nm and 2000 nm, preferably between 350 nm and 1000 nm, more preferably between 400 nm and 900 nm, most preferably between 450 nm and 800 nm.

Preferably, the absorption per mm of thickness of the three-dimensional object body or three-dimensional object is less than 0.5, preferably less than 0.3, more preferably less than 0.2, more preferably less than 0.1, for each wavelength in the wavelength range between 300 nm and 2000 nm, preferably between 350 nm and 1000 nm, more preferably between 400 nm and 900 nm, most preferably between 450 nm and 800 nm.

In one embodiment the post-processing comprises a thermal treatment of the three-dimensional object which thermal treatment comprises tempering the three-dimensional object for a specific time at least one specific temperature.

Preferably, the thermal treatment is carried out in specific time ranges between 0.1 min and 24 h, preferably between 0.5 min and 360 min, more preferably between 1 min and 60 min.

Preferably, a the thermal treatment is carried out in specific temperature ranges between 50° C. and 150° C., preferably between 75° C. and 125° C.

In one embodiment the post-processing comprises an optical treatment of the three-dimensional object which optical treatment comprises irradiating the three-dimensional object with light of at least one specific wavelength for a specific time with a specific light intensity.

Preferably, the optical treatment is carried out in a time ranges between 0.1 min and 24 h, preferably between 1 min and 360 min, more preferably between 5 min and 60 min.

Preferably, an optical treatment of the three-dimensional object is carried out with a specific light intensity, wherein the specific light intensity can range between 0.0001-1000 W/cm², preferably between 0.001-100 W/cm², more preferably between 0.1-30 W/cm², most preferably between 1-10 W/cm2.

Preferably, the optical treatment is carried out, wherein the at least one specific wavelength ranges between 350 nm and 1000 nm, preferably between 400 nm and 800 nm, more preferably between 350 nm and 500 nm or between 420 nm and 800 nm.

In one embodiment, the optical treatment is carried out by irradiating light with an intensity and a wavelength by which the three-dimensional object is not removed from the polymerizable material. Preferably, the optical treatment is carried out, wherein the three-dimensional object is irradiated for the specific time and light intensity with the light of the at least one specific wavelength without removing the three-dimensional object from the surrounding polymerizable material.

Preferably, the optical treatment is carried out, wherein the three-dimensional object is subject to tempering after irradiating the three-dimensional object for the specific time with light of the at least one specific wavelength, wherein the tempering preferably comprises heating the three-dimensional object to a temperature ranging between 50° C. and 150° C. for a time ranging between 1 min and 360 min, preferably 5 min and 60 min.

### Claim-Like Clause

Item (33) A process for locally polymerizing a starting material by dual color photopolymerization and forming a shaped body, comprising the following steps:
providing a container which is at least partially filled with a polymerizable starting material containing photoinitiator molecules, which can be converted by sequential optical excitation into a reactive state in which the photoinitiator molecules locally trigger polymerization of the starting material; and
photopolymerizing the starting material in the container by irradiating light of a first wavelength and light of a second wavelength, different from the first wavelength, into the container, whereby in the container
the photoinitiator molecules are converted, due to the absorption of the light of the first wavelength, from an initial state in which the photoinitiator molecules substantially do not absorb the light of the second wavelength, into an intermediate state with changed optical properties compared to the initial state, such that the photoinitiator molecules in the intermediate state absorb the light of the second wavelength; and
the photoinitiator molecules are transferred from the intermediate state to the reactive state due to the absorption of the light of the second wavelength, which triggers the polymerization locally to form the shaped body; and
   wherein the initial state of the photoinitiator molecules in the photopolymerizable material has an extinction coefficient at the first wavelength which is lower than 5000 L mol⁻¹ cm⁻¹.

Item (34) A process for locally polymerizing a starting material by dual color photopolymerization and forming a shaped body, comprising the following steps:
providing a container which is at least partially filled with a polymerizable starting material containing photoinitiator molecules, which can be converted by sequential optical excitation into a reactive state in which the photoinitiator molecules locally trigger polymerization of the starting material; and
photopolymerizing the starting material in a container by irradiating light of a first wavelength and light of a second wavelength, different from the first wavelength, into the container, whereby in the container
the photoinitiator molecules are converted, due to the absorption of the light of the first wavelength, from an initial state in which the photoinitiator molecules substantially do not absorb the light of the second wavelength, into an intermediate state with changed optical properties compared to the initial state, such that the photoinitiator molecules in the intermediate state absorb the light of the second wavelength; and
the photoinitiator molecules are transferred from the intermediate state to the reactive state due to the absorption of the light of the second wavelength, which triggers the polymerization locally to form a shaped body;
   wherein the photopolymerizable material has a absorbance at the first wavelength in a range between 1 and 0.07 absorbance units.

Item (35) A process according to item (33) or item (34), wherein the photoinitiator molecule is the spiropyran as it is defined in the description.

### EXAMPLES

Hereinafter, the action and effect of the invention will be described in detail through specific examples of the invention. However, the examples are provided only to illustrate the present invention, and the scope of the invention is not limited thereto.

### A. Synthesis

### General Synthetic Methods

Method A (Reaction of Indolenium Salt or Respective 2-Methylenindoline with a Salicylaldehyde, Examples 1-12, 35-43, 51, 52, 79, 86)

2-Methyleneindoline (1 mmol), the respective salicylaldehyde derivative (1 mmol), and piperidine (0.1 mmol) are dissolved in 10 mL EtOH and heated to 70° C. until no further formation of the spiropyran derivative is observed, typically 1 h to 12 h. The mixture is cooled to room temperature and worked up as described below.

When an indolenium salt derivative is used instead of 2-methyleneindoline, the reaction is carried out with 1.1 mmol piperidine.

When the product precipitates, the mixture is filtered, and the solid residue is recrystallized from ethanol.

Where the spiropyran is not water soluble, water and ethyl acetate are added to the mixture and the organic phase is washed with water. The organic phase is dried over anhydrous MgSO₄ and evaporated under reduced pressure. The residue is either crystallized or purified by silica-gel column chromatography using petroleum ether-acetone mixtures.

Where the spiropyran is water soluble, the reaction mixture is directly subjected to C-18 functionalized silica-gel and purified by MPLC using methanol-water mixtures. After evaporation of the solvent, the product can be dissolved in acetonitrile or methanol and an ion-exchange resin is added. The mixture is placed on a shaker for 1-3h and filtered. The last step is repeated until the exchange is complete. The solvent is removed under reduced pressure to yield the product spiropyran derivative.

### Method B (Modification of Halogenated Spiropyran, Examples 18-31, 33, 34, 61, 84, 85, 88)

The halogenated spiropyran (1.5 mmol) is dissolved in dry tetrahydrofuran (25 mL) under an argon atmosphere and cooled to −78° C. n-Butyl lithium (1.65 mmol, 2.5 M in hexanes) is added dropwise. After stirring for 15 min at −78° C., a Weinreb amide (2 mmol) is added, and the reaction mixture is allowed to warm to room temperature. The reaction is stirred until no further conversion is observed and 1 M aqueous hydrochloric (10 mL) acid is added. The mixture is stirred for 10 min, 1 M aqueous NaOH solution is added (20 mL) and the mixture is extracted with ethyl acetate. The combined organic layers are dried over anhydrous MgSO₄ and evaporated under reduced pressure. The residue is either crystallized or purified by silica-gel column chromatography using petroleum ether-acetone mixtures.

For derivative 34 glutaric anhydride was used instead of a Weinreb amide and the reaction mixture was neutralized before extraction.

For derivative 19 2-dimethylamino-2-methylpropionitrile was used instead of a Weinreb amide.

For derivative 31 and 91 the reaction was performed with 2.2. eq of n-BuLi and 2.8 eq of Weinrebamide.

### Method C (Reaction of Indolenium Salt and Catalytic Amount of Base, Examples 15-17, 44-50, 54-60, 62-66, 80-83)

An indolenium salt derivative (1.2 mmol), a spiropyran derivative (1 mmol), and piperidine (0.1 mmol) are dissolved in 20 mL ethanol and stirred at 70° C. until no further formation of the product spiropyran derivative is observed, typically 30 min to 12 h. The reaction mixture is cooled to room temperature and worked up as described below.

When the product precipitates, the mixture is filtered, and the solid residue is recrystallized from ethanol.

Where the spiropyran is not water soluble, water and ethyl acetate are added to the mixture and the organic phase is washed with water. The organic phase is dried over anhydrous MgSO₄ and evaporated under reduced pressure. The residue is either crystallized or purified by silica-gel column chromatography using petroleum ether-acetone mixtures.

Where the spiropyran is water soluble, the reaction mixture is directly subjected to C-18 functionalized silica-gel and purified by MPLC using methanol-water mixtures. After evaporation of the solvent, the product can be dissolved in acetonitrile or methanol and an ion-exchange resin is added. The mixture is placed on a shaker for 1-3h and filtered. The last step is repeated until the exchange is complete. The solvent is removed under reduced pressure to yield the product spiropyran derivative.

In all reactions according to method C the respective 2-methyleneindoline is obtained as well. In reactions where the 2-methyleneindoline is the desired product, the reaction may be performed as follows: 1,3,3-Trimethyl-5-nitro-2-methylen-indolin or the respective indolenium salt indolenium salt (1.2 mmol), a spiropyran derivative (1 mmol), piperidine (0.1 mmol), and acetic acid (1 mL) are dissolved in 20 mL ethanol and stirred at 70° C. until no further formation of the product 2-methyleneindoline derivative is observed, typically 30 min to 12 h. The purification is performed as outlined above.

Synthesis of 56: 73 (2.1 mmol), 26 (1 mmol), and piperidine (2 mmol) are dissolved in 20 mL ethanol and stirred at 70° C. for 24 h. The reaction mixture is cooled to room temperature, water and ethyl acetate are added to the mixture and the organic phase is washed with water. The organic phase is dried over anhydrous MgSO₄ and evaporated under reduced pressure. The residue purified by silica-gel column chromatography using petroleum ether-acetone mixtures to yield the intermediate product 87. The intermediate 87 is dissolved in ethanol (10 mL) and acetic acid (1 mL). The mixture is stirred for 12 h. 1 M aqueous NaOH solution and ethyl acetate are added, and the mixture is washed with 1 M aqueous NaOH solution and water. The combined organic phase is dried over anhydrous MgSO₄ and evaporated under reduced pressure. The residue purified by silica-gel column chromatography using petroleum ether-acetone mixtures to yield product 56.

### Method D (Synthesis of Indolenium Salts of the Respective Indoles, Examples 67-78)

The corresponding aniline derivative (50 mmol) is dissolved in a mixture of concentrated aqueous hydrochloric acid (20 mL) and ice water (30 mL). NaNO₂ (100 mmol) in water is added at 0° C. After stirring for 30 min, SnCl₂ (28.4 g) in concentrated aqueous hydrochloric acid (35 mL) is added. The resulting mixture is stirred for 30 min, filtered, and washed with water or 1M aqueous hydrochloric acid to obtain the hydrazine hydrochloride which is used directly in the next step.

The corresponding hydrazine (as hydrochloride salt) (47.5 mmol), 3-methylbutan-2-one and concentrated aqueous sulfuric acid (7 mL) are dissolved in glacial acetic acid (68 mL). The mixture is refluxed until no further formation of the indole is observed. The mixture is cooled to room temperature and worked up as described below.

When the product is not functionalized with a water solubilizing group, the main fraction of the acetic acid is distilled of. The residue is neutralized with saturated aqueous NaHCO₃ solution. The mixture is extracted with ethyl acetate and the combined organic phases are dried over anhydrous MgSO₄, followed by evaporation of the solvent under reduced pressure. Where the indole is not of sufficient purity for the next step, silica gel column chromatography is performed using petroleum ether/acetone mixtures as eluent.

Where the product is functionalized with a water solubilizing group, ethyl acetate (1 L) is added to the mixture. After the precipitation is complete, the mixture is filtered, and the indole is directly used in the next step. Where the indole is not of sufficient purity, it is purified by MPLC using water-methanol solvent mixtures.

The obtained indole derivative (30 mmol) is dissolved in acetonitrile (150 mL) and the corresponding alkyl halide or propane sultone is added (90 mmol). The mixture is refluxed for 24 h. After cooling to room temperature, the product precipitates, is filtered and washed with acetonitrile. Where the product does not precipitate, the solvent is evaporated under reduced pressure. The indole is purified by recrystallization from mixtures of acetonitrile, acetone, and ethyl acetate.

Where the indole is not reactive enough to form the product in the above mentioned procedure, the procedure is modified as follows: The indole derivative (30 mmol) is dissolved in N,N-dimethylformamide (150 mL) and the corresponding alkyl halide or propane sultone is added (90 mmol). The mixture is heated to 120° C. After no further product formation is observed, the reaction is cooled to room temperature and ethyl acetate (1 L) is added. After the precipitation is complete, the product is filtered and washed with ethyl acetate. The product is recrystallized from acetonitrile-ethyl acetate mixtures.

Where the indole is needed, the respective indolenium salt is dissolved in a mixture of ethyl acetate and 1 M aqueous NaOH solution. The mixture is extracted with ethyl acetate. The combined organic layers are dried over anhydrous MgSO₄ and evaporated under reduced pressure to yield the indole derivative.

### Exchange of Two Spiropyrans

43 (1 mmol) and 51 (1 mmol) are dissolved in ethanol (5 mL) with 1% acetic acid and stirred at 70° C. for 7 days. Water and ethyl acetate are added to the mixture and the organic phase is washed with water. The organic phase is dried over anhydrous MgSO₄ and evaporated under reduced pressure. The residue is purified by silica-gel column chromatography using petroleum ether-acetone mixtures to yield 12 and 13.

43 (1 mmol), 51 (1 mmol), 71 (0.1 mmol), and piperidine (0.1 mmol) are dissolved in ethanol (5 mL) with and stirred at 70° C. for 24h. Water and ethyl acetate are added to the mixture and the organic phase is washed with water. The organic phase is dried over anhydrous MgSO₄ and evaporated under reduced pressure. The residue is purified by silica-gel column chromatography using petroleum ether-acetone mixtures to yield 12 and 13.

89 (1 mmol), 90 (1 mmol), and 1,3,3-Trimethyl-2-methylen-indolin (0.1 mmol) are dissolved in ethanol (10 mL) and stirred at 70° C. for 7 days. Water and ethyl acetate are added to the mixture and the organic phase is washed with water. The organic phase is dried over anhydrous MgSO₄ and evaporated under reduced pressure. The residue is purified by silica-gel column chromatography using petroleum ether-acetone mixtures to yield 14 and unsubstituted spiropyran.

Pre-Activation of Spiropyrans with Base and Reaction with Salicylaldehydes

18 (1 mmol) and Methylamine (0.1 mmol) are dissolved in n-butanol (20 mL) and water (2 mL) and heated to 120° C. for 48 h. The mixture is cooled to room temperature and 1M aqueous hydrochloric acid (10 mL) is added. The mixture is stirred for 10 min, 1 M aqueous NaOH solution (20 mL) and ethyl acetate are added. The organic phase is separated and washed once with 1M aqueous NaOH solution and once with water. The organic phase is dried over anhydrous MgSO₄ and evaporated under reduced pressure. The residue can be directly used in the next reaction. For cases, where the separation in the next reaction is difficult, the residue is purified by silica-gel column chromatography using petroleum ether-acetone mixtures. 15 (1 mmol) from the pre-activation is dissolved in ethanol (10 mL) and methyl 3-formyl-4-hydroxybenzoate (1 mmol) and piperidine (0.1 mmol) are added. The reaction is stirred at 70° C. for 24 h. The reaction mixture is cooled to room temperature and water and ethyl acetate are added to the mixture and the organic phase is washed with water. The organic phase is dried over anhydrous MgSO₄ and evaporated under reduced pressure. The residue is purified by silica-gel column chromatography using petroleum ether-acetone mixtures to yield 32.

85 (1 mmol) and Methylamine (0.1 mmol) are dissolved in ethanol (20 mL) and heated to 70° C. for 24 h. The mixture is cooled to room temperature and 1M aqueous hydrochloric acid (10 mL) is added. The mixture is stirred for 10 min, 1 M aqueous NaOH solution (20 mL) and ethyl acetate are added. The organic phase is separated and washed once with 1M aqueous NaOH solution and once with water. The organic phase is dried over anhydrous MgSO₄ and evaporated under reduced pressure. The residue is dissolved in ethanol (10 mL) and methyl 3-formyl-4-hydroxybenzoate (1 mmol) and piperidine (0.1 mmol) are added. The reaction is stirred at 70° C. for 24 h. The reaction mixture is cooled to room temperature and water and ethyl acetate are added to the mixture and the organic phase is washed with water. The organic phase is dried over anhydrous MgSO₄ and evaporated under reduced pressure. The residue is purified by silica-gel column chromatography using petroleum ether-acetone mixtures to yield 86.

### Knoevenagel Condensation on Spiropyrans

38 (1 mmol), malononitrile (1 mmol), and piperidine (0.1 mmol) are dissolved in ethanol (25 ml) and stirred at room temperature for 2h. Water and ethyl acetate are added to the mixture and the organic phase is washed with water. The organic phase is dried over anhydrous MgSO₄ and evaporated under reduced pressure. The residue is purified by silica-gel column chromatography using petroleum ether-acetone mixtures to yield 53.

### Numbering Scheme for Spiropyrans and Indolenium Salts

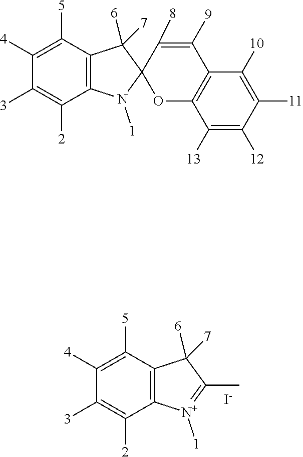

As non-limiting examples, the following spiropyrans and indolenium salts, 2-methylenindoline, have been prepared by following the specified procedures:

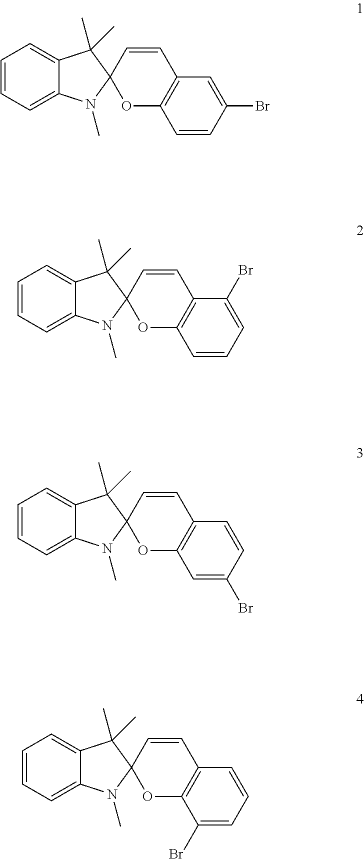 [SMILES: CN1c2ccccc2C(C)(C)C12C=Cc1cc(Br)ccc1O2 ; CN1c2ccccc2C(C)(C)C12C=Cc1c(Br)cccc1O2 ; CN1c2ccccc2C(C)(C)C12C=Cc1ccc(Br)cc1O2 ; CN1c2ccccc2C(C)(C)C12C=Cc1cccc(Br)c1O2]

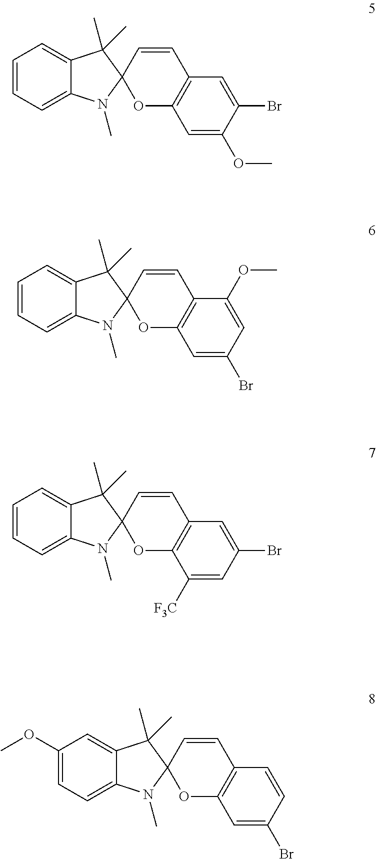

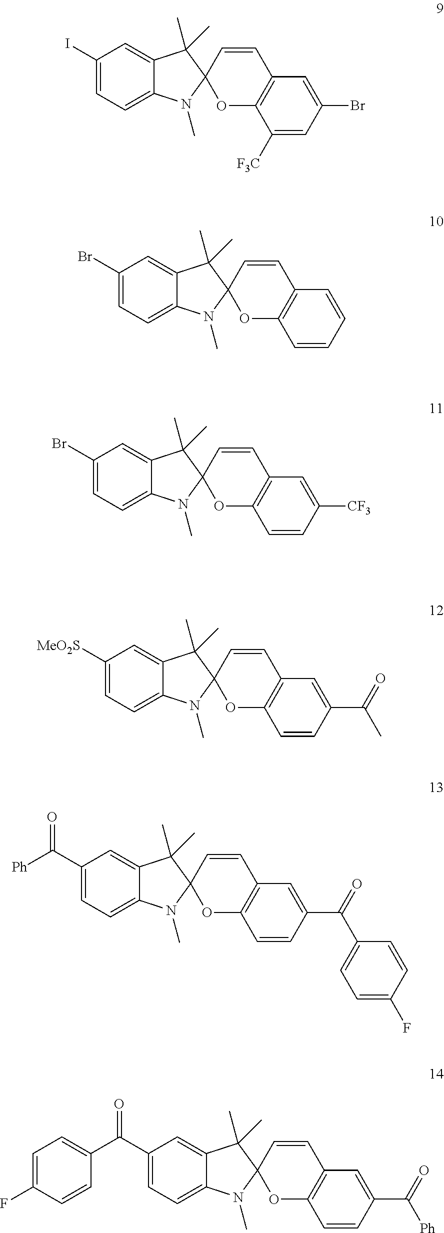 [SMILES: CN1c2ccc(I)cc2C(C)(C)C12C=Cc1cc(Br)cc(C(F)(F)F)c1O2 ; CN1c2ccc(Br)cc2C(C)(C)C12C=Cc1ccccc1O2 ; CN1c2ccc(Br)cc2C(C)(C)C12C=Cc1cc(C(F)(F)F)ccc1O2 ; CC(=O)c1ccc2c(c1)C=CC1(O2)N(C)c2ccc(S(C)(=O)=O)cc2C1(C)C ; CN1c2ccc(C(=O)c3ccccc3)cc2C(C)(C)C12C=Cc1cc(C(=O)c3ccc(F)cc3)ccc1O2 ; CN1c2ccc(C(=O)c3ccc(F)cc3)cc2C(C)(C)C12C=Cc1cc(C(=O)c3ccccc3)ccc1O2]

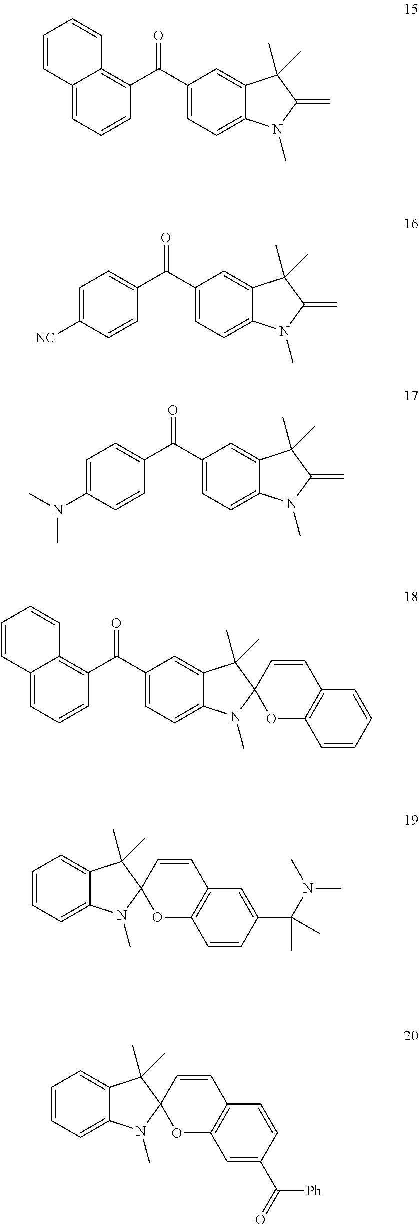

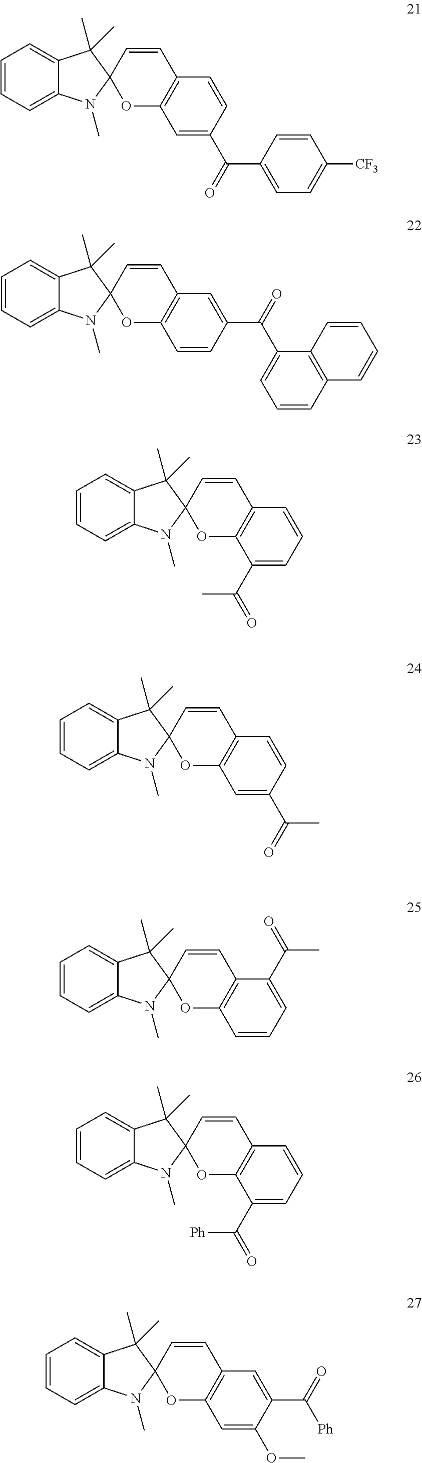

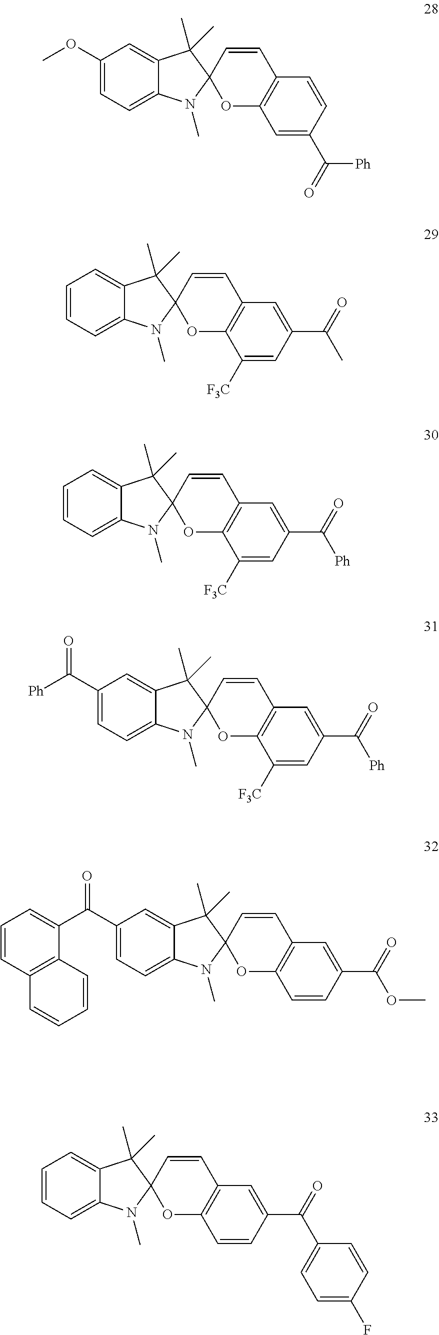 [SMILES: COc1ccc2c(c1)C(C)(C)C1(C=Cc3ccc(C(=O)c4ccccc4)cc3O1)N2C ; CC(=O)c1cc2c(c(C(F)(F)F)c1)OC1(C=C2)N(C)c2ccccc2C1(C)C ; CN1c2ccccc2C(C)(C)C12C=Cc1cc(C(=O)c3ccccc3)cc(C(F)(F)F)c1O2 ; CN1c2ccc(C(=O)c3ccccc3)cc2C(C)(C)C12C=Cc1cc(C(=O)c3ccccc3)cc(C(F)(F)F)c1O2 ; COC(=O)c1ccc2c(c1)C=CC1(O2)N(C)c2ccc(C(=O)c3cccc4ccccc34)cc2C1(C)C ; CN1c2ccccc2C(C)(C)C12C=Cc1cc(C(=O)c3ccc(F)cc3)ccc1O2]

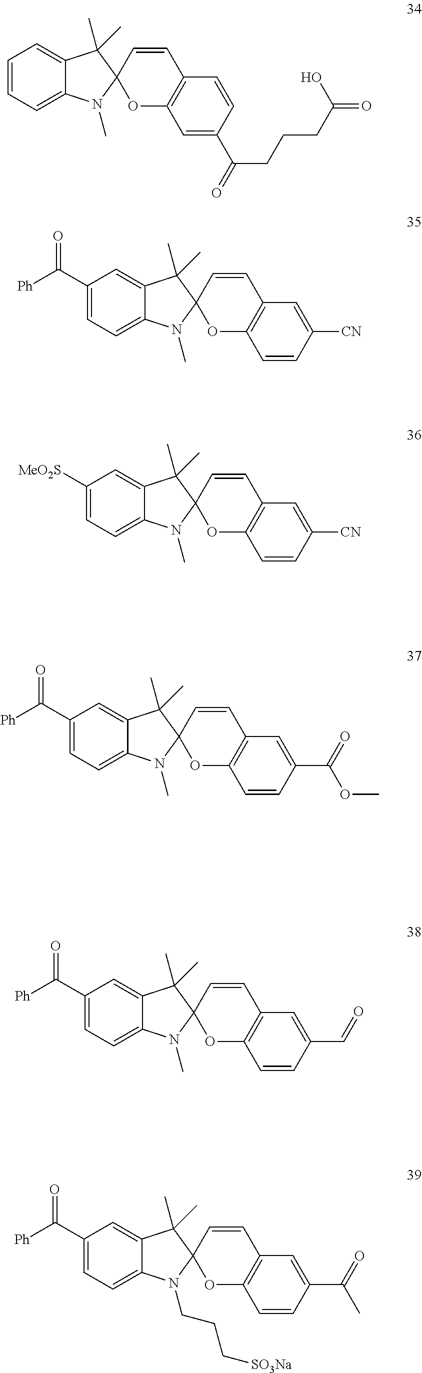

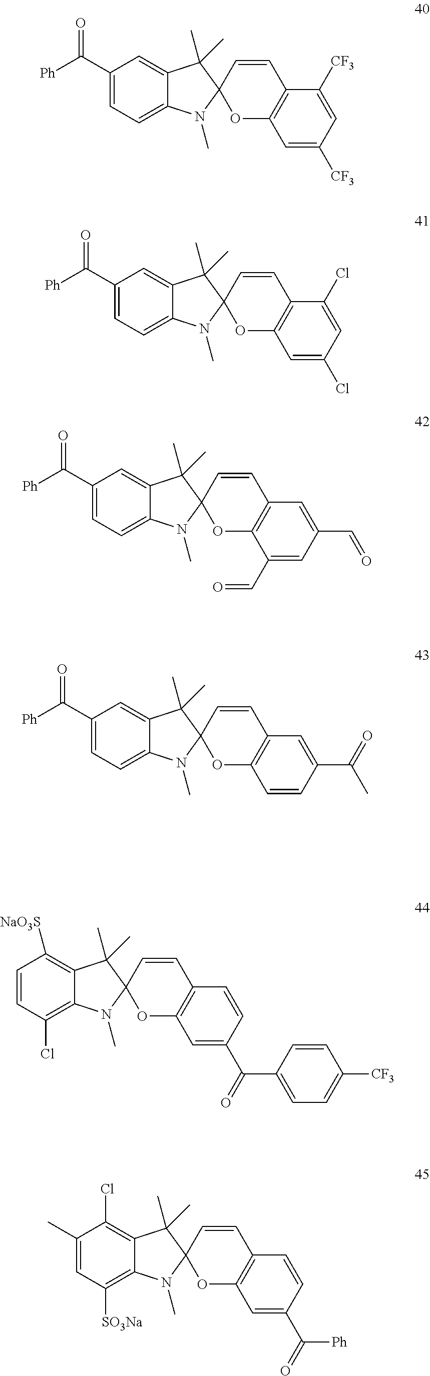

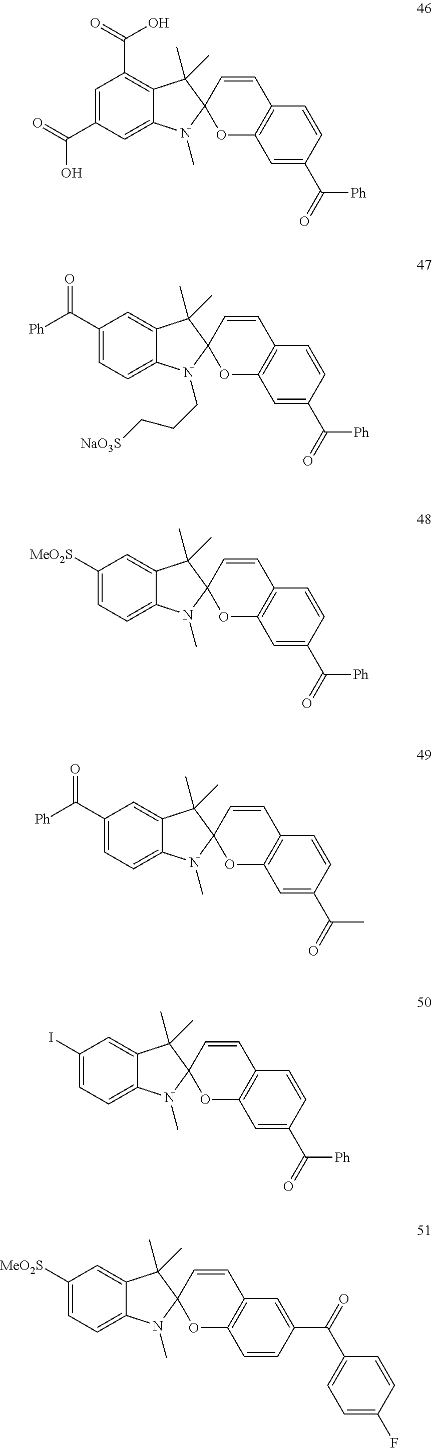

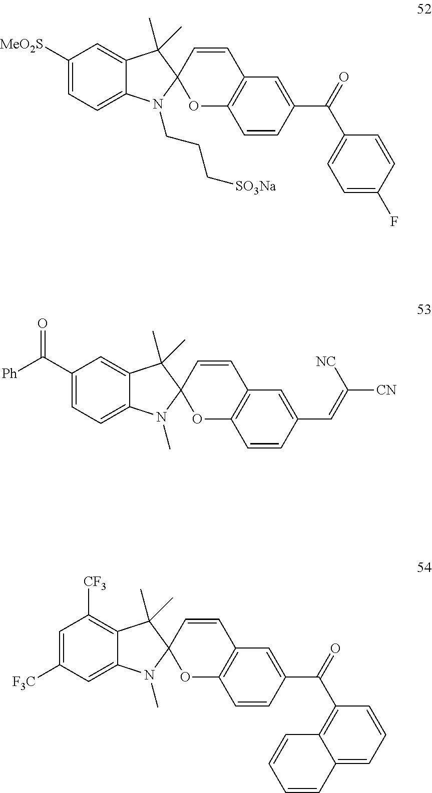

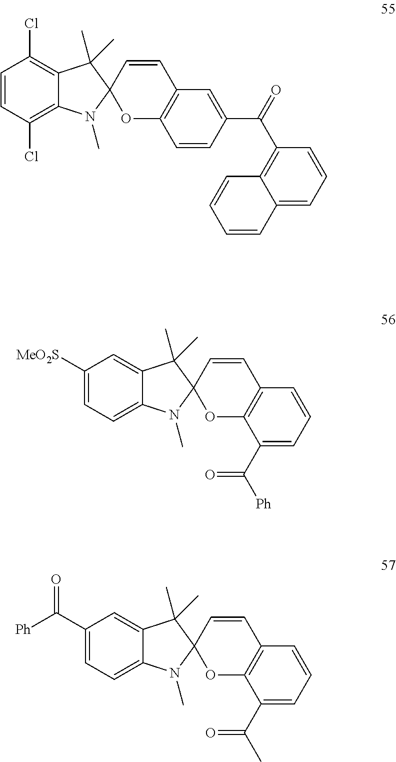 [SMILES: CN1c2c(Cl)ccc(Cl)c2C(C)(C)C12C=Cc1cc(C(=O)c3cccc4ccccc34)ccc1O2 ; CN1c2ccc(S(C)(=O)=O)cc2C(C)(C)C12C=Cc1cccc(C(=O)c3ccccc3)c1O2 ; CC(=O)c1cccc2c1OC1(C=C2)N(C)c2ccc(C(=O)c3ccccc3)cc2C1(C)C]

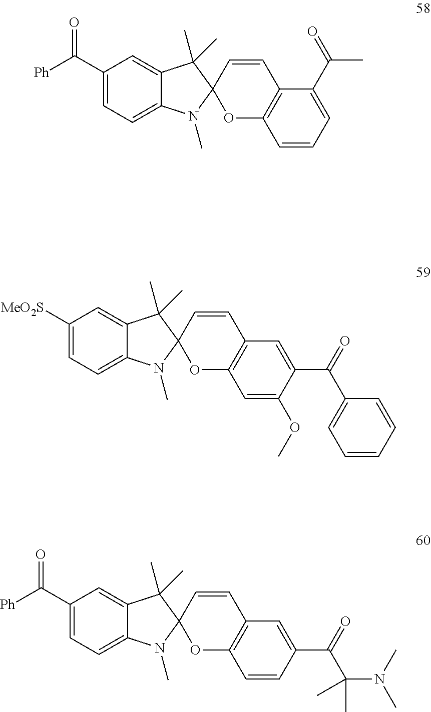 [SMILES: CC(=O)c1cccc2c1C=CC1(O2)N(C)c2ccc(C(=O)c3ccccc3)cc2C1(C)C ; COc1cc2c(cc1C(=O)c1ccccc1)C=CC1(O2)N(C)c2ccc(S(C)(=O)=O)cc2C1(C)C ; CN(C)C(C)(C)C(=O)c1ccc2c(c1)C=CC1(O2)N(C)c2ccc(C(=O)c3ccccc3)cc2C1(C)C]

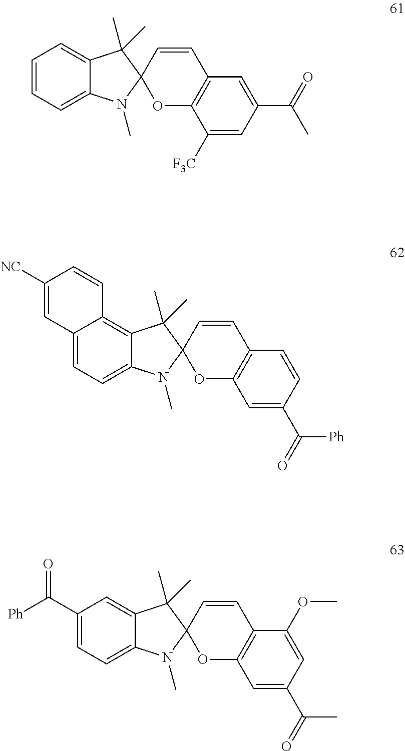 [SMILES: CC(=O)c1cc2c(c(C(F)(F)F)c1)OC1(C=C2)N(C)c2ccccc2C1(C)C ; CN1c2ccc3cc(C#N)ccc3c2C(C)(C)C12C=Cc1ccc(C(=O)c3ccccc3)cc1O2 ; COc1cc(C(C)=O)cc2c1C=CC1(O2)N(C)c2ccc(C(=O)c3ccccc3)cc2C1(C)C]

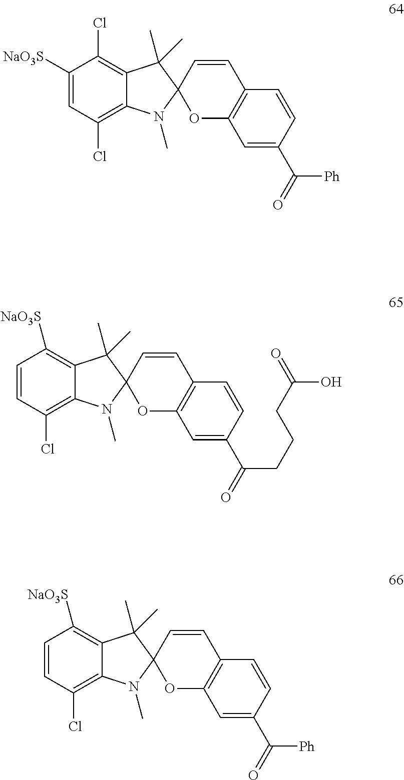

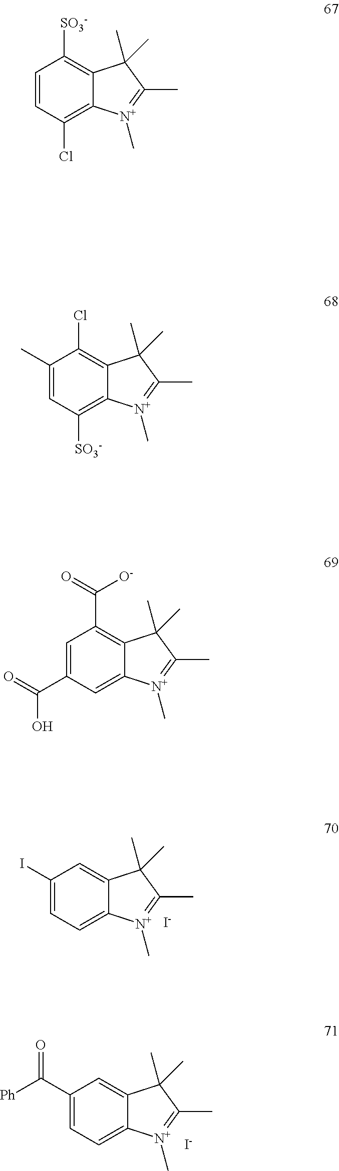

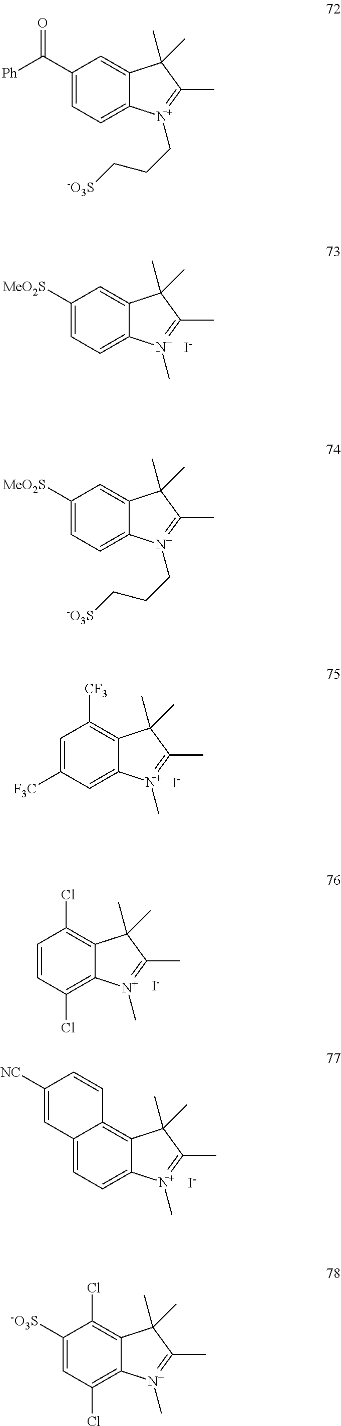

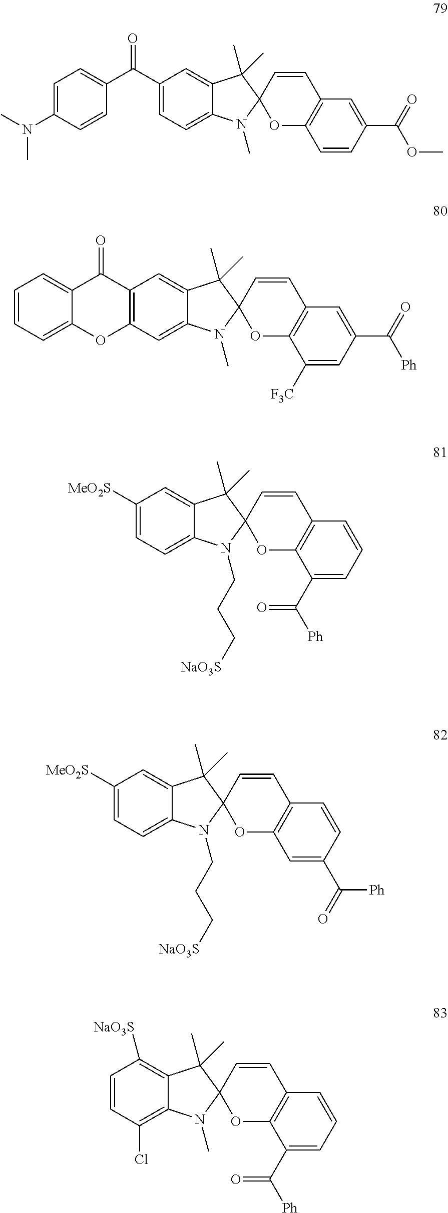

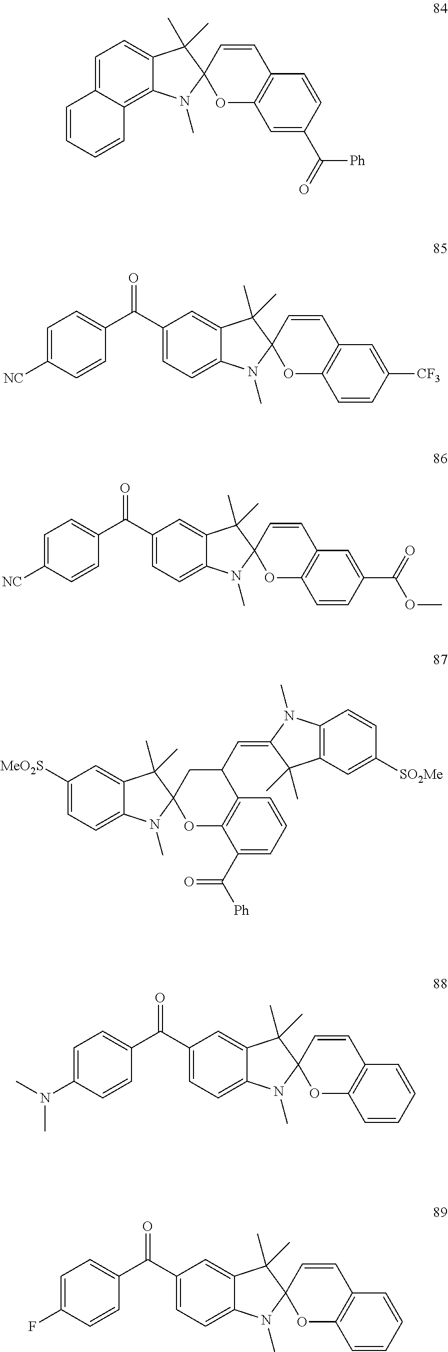

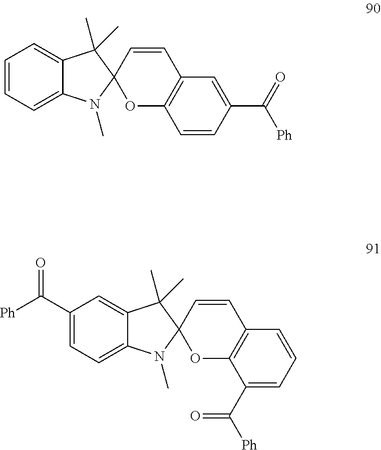 [SMILES: CN1c2ccccc2C(C)(C)C12C=Cc1cc(C(=O)c3ccccc3)ccc1O2 ; CN1c2ccc(C(=O)c3ccccc3)cc2C(C)(C)C12C=Cc1cccc(C(=O)c3ccccc3)c1O2]

### ¹H-NMR Data

(The methylene group of the free Fischer Base and indolenium salts exchanges in the deuterated methanol and is not visible.)

1 ¹H NMR (300 MHz, Methylene Chloride-d₂) δ 7.23-7.10 (m, 3H), 7.06 (m, 1H), 6.88-6.77 (m, 2H), 6.60 (d, J=8.1 Hz, 1H), 6.52 (dd, J=7.8, 0.8 Hz, 1H), 5.76 (d, J=10.3 Hz, 1H), 2.71 (s, 3H), 1.28 (s, 3H), 1.15 (s, 3H).

4 ¹H NMR (300 MHz, Methylene Chloride-d₂) δ 7.32 (dd, J=8.0, 1.5 Hz, 1H), 7.17 (td, J=7.6, 1.3 Hz, 1H), 7.12-7.01 (m, 2H), 6.90-6.80 (m, 2H), 6.73 (dd, J=8.0, 7.5 Hz, 1H), 6.60-6.49 (m, 1H), 5.74 (d, J=10.2 Hz, 1H), 2.72 (s, 3H), 1.30 (s, 3H), 1.16 (s, 3H).

5 ¹H NMR (300 MHz, Methylene Chloride-d₂) δ 7.22 (s, 1H), 7.20-7.11 (m, 1H), 7.11-7.04 (m, 1H), 6.90-6.74 (m, 2H), 6.52 (dt, J=7.7, 0.7 Hz, 1H), 6.38 (d, J=0.6 Hz, 1H), 5.61 (d, J=10.2 Hz, 1H), 3.76 (s, 3H), 2.72 (s, 3H), 1.29 (s, 3H), 1.15 (s, 3H).

9 ¹H NMR (300 MHz, Methylene Chloride-d₂) δ 7.52-7.43 (m, 2H), 7.40 (d, J=2.5 Hz, 1H), 7.33 (d, J=1.8 Hz, 1H), 6.88 (d, J=10.4 Hz, 1H), 6.33 (d, J=8.1 Hz, 1H), 5.84 (d, J=10.4 Hz, 1H), 2.65 (s, 3H), 1.27 (s, 3H), 1.17 (s, 3H). 11 ¹H NMR (500 MHz, Chloroform-d) δ 7.35 (ddd, J=8.5, 2.3, 0.8 Hz, 1H), 7.31 (d, J=2.2 Hz, 1H), 7.27 (dd, J=8.2, 2.0 Hz, 1H), 7.15 (d, J=2.0 Hz, 1H), 6.89 (dd, J=10.4, 0.7 Hz, 1H), 6.77 (d, J=8.5 Hz, 1H), 6.41 (d, J=8.2 Hz, 1H), 5.75 (d, J=10.3 Hz, 1H), 2.70 (s, 3H), 1.27 (s, 3H), 1.17 (s, 3H).

12 ¹H NMR (500 MHz, Methylene Chloride-d₂) δ 7.78-7.73 (m, 3H), 7.56 (d, J=1.9 Hz, 1H), 7.00 (dd, J=10.4, 0.7 Hz, 1H), 6.74 (dt, J=8.2, 0.7 Hz, 1H), 6.62 (d, J=8.3 Hz, 1H), 5.77 (d, J=10.3 Hz, 1H), 3.03 (s, 3H), 2.82 (s, 3H), 2.51 (s, 3H), 1.32 (s, 3H), 1.20 (s, 3H).

13 ¹H NMR (500 MHz, Methylene Chloride-d₂) δ 7.72 (dd, J=8.4, 3.1, 4H), 7.70 (d, J=8.0 Hz, 2H), 7.68-7.52 (m, 3H), 7.50-7.42 (m, 4H), 6.96 (dd, J=10.4, 0.7 Hz, 1H), 6.79 (dd, J=8.2, 1.0 Hz, 1H), 6.60 (dd, J=8.0, 0.8 Hz, 1H), 5.80 (d, J=10.1 Hz, 1H), 2.86 (s, 3H), 1.36 (s, 3H), 1.23 (s, 3H).

14 ¹H NMR (500 MHz, Methylene Chloride-d₂) δ 7.80-7.72 (m, 4H), 7.71 (d, J=7.8 Hz, 2H), 7.68-7.60 (m, 2H), 7.57-7.51 (m, 2H), 7.49 (d, J=7.5 Hz, 4H), 6.98 (d, J=10.4 Hz, 1H), 6.79 (d, J=8.2, 1H), 6.58 (dd, J=8.1, 0.9 Hz, 1H), 5.76 (d, J=10.2 Hz, 1H), 2.87 (s, 3H), 1.37 (s, 3H), 1.22 (s, 3H).

18 ¹H NMR (500 MHz, Methylene Chloride-d₂) δ 8.01-7.96 (m, 2H), 7.96-7.92 (m, 1H), 7.79 (d, J=1.8 Hz, 1H), 7.58 (dd, J=8.2, 1.8 Hz, 1H), 7.56-7.46 (m, 4H), 7.13 (ddd, J=8.1, 7.3, 1.7 Hz, 1H), 7.09 (dd, J=7.5, 1.7 Hz, 1H), 6.92 (dd, J=10.2, 0.7 Hz, 1H), 6.86 (td, J=7.4, 1.1 Hz, 1H), 6.72 (dt, J=8.1, 0.9 Hz, 1H), 6.44 (d, J=8.2 Hz, 1H), 5.69 (d, J=10.2 Hz, 1H), 2.81 (s, 3H), 1.32 (s, 3H), 1.19 (s, 3H).

19 ¹H NMR (300 MHz, Methylene Chloride-d₂) δ 7.24-7.01 (m, 3H), 7.00-6.76 (m, 2H), 6.75-6.62 (m, 1H), 6.62-6.47 (m, 2H), 5.82-5.67 (m, 1H), 2.74 (s, 3H), 2.23 (s, 6H), 1.29 (s, 3H), 1.25 (s, 6H), 1.17 (s, 3H).

20 ¹H NMR (300 MHz, Methylene Chloride-d₂) δ 7.80-7.68 (m, 2H), 7.61-7.52 (m, 1H), 7.51-7.42 (m, 2H), 7.30 (dd, J=7.8, 1.6 Hz, 1H), 7.25-7.10 (m, 3H), 7.10-7.04 (m, 1H), 6.97 (dd, J=10.3, 0.8 Hz, 1H), 6.82 (td, J=7.4, 1.0 Hz, 1H), 6.57-6.50 (m, 1H), 5.87 (d, J=10.3 Hz, 1H), 2.75 (s, 3H), 1.32 (s, 3H), 1.18 (s, 3H).

21 ¹H NMR (300 MHz, Methylene Chloride-d₂) δ 7.87-7.79 (m, 2H), 7.76-7.69 (m, 2H), 7.32 (dd, J=7.8, 1.6 Hz, 1H), 7.22 (d, J=7.8 Hz, 1H), 7.15 (td, J=7.6, 1.3 Hz, 1H), 7.11-7.04 (m, 2H), 6.98 (dd, J=10.3, 0.8 Hz, 1H), 6.82 (td, J=7.4, 1.0 Hz, 1H), 6.52 (dd, J=7.8, 0.8 Hz, 1H), 5.89 (d, J=10.3 Hz, 1H), 2.74 (s, 3H), 1.30 (s, 3H), 1.18 (s, 3H).

22 ¹H NMR (300 MHz, Methylene Chloride-d₂) δ 8.07-7.92 (m, 3H), 7.71-7.44 (m, 6H), 7.16 (td, J=7.7, 1.3 Hz, 1H), 7.12-7.05 (m, 1H), 6.95-6.79 (m, 2H), 6.78-6.71 (m, 1H), 6.62-6.49 (m, 1H), 5.78 (d, J=10.3 Hz, 1H), 2.74 (s, 3H), 1.30 (s, 3H), 1.17 (s, 3H).

23 ¹H NMR (300 MHz, Methylene Chloride-d₂) δ 7.61 (ddd, J=7.9, 1.7, 0.3 Hz, 1H), 7.25 (dd, J=7.3, 1.8 Hz, 1H), 7.16 (td, J=7.6, 1.3 Hz, 1H), 7.09 (ddd, J=7.3, 1.3, 0.6 Hz, 1H), 6.97 (d, J=10.4 Hz, 1H), 6.92-6.80 (m, 2H), 6.54 (d, J=7.7 Hz, 1H), 5.85 (d, J=10.4 Hz, 1H), 2.72 (s, 3H), 2.00 (s, 3H), 1.31 (s, 3H), 1.21 (s, 3H).

24 ¹H NMR (300 MHz, Methylene Chloride-d₂) δ 7.44 (dd, J=7.8, 1.7 Hz, 1H), 7.33-7.27 (m, 1H), 7.23-7.14 (m, 2H), 7.09 (ddd, J=7.3, 1.3, 0.6 Hz, 1H), 6.95 (dd, J=10.3, 0.8 Hz, 1H), 6.85 (td, J=7.4, 1.0 Hz, 1H), 6.58-6.51 (m, 1H), 5.86 (d, J=10.2 Hz, 1H), 2.73 (s, 3H), 2.48 (s, 3H), 1.31 (s, 3H), 1.18 (s, 3H).

25 ¹H NMR (300 MHz, Methylene Chloride-d₂) δ 7.66 (dd, J=10.7, 0.8 Hz, 1H), 7.27 (dd, J=7.7, 1.2 Hz, 1H), 7.23-7.11 (m, 2H), 7.11-7.03 (m, 1H), 6.92-6.79 (m, 2H), 6.55-6.47 (m, 1H), 5.81 (d, J=10.7 Hz, 1H), 2.72 (s, 3H), 2.58 (s, 3H), 1.28 (s, 3H), 1.16 (s, 3H).

26 ¹H NMR (300 MHz, Methylene Chloride-d₂) δ 7.48-7.36 (m, 3H), 7.28-7.15 (m, 4H), 7.01 (td, J=7.6, 1.3 Hz, 1H), 6.96-6.88 (m, 2H), 6.88-6.80 (m, 1H), 6.70 (td, J=7.4, 1.0 Hz, 1H), 6.24 (dd, J=7.7, 0.8 Hz, 1H), 5.70 (d, J=10.3 Hz, 1H), 2.55 (s, 3H), 1.07 (s, 3H), 1.05 (s, 3H).

27 ¹H NMR (300 MHz, Methylene Chloride-d₂) δ 7.79-7.70 (m, 2H), 7.60-7.51 (m, 1H), 7.50-7.39 (m, 2H), 7.24-7.06 (m, 3H), 6.91-6.79 (m, 2H), 6.59-6.53 (m, 1H), 6.40 (d, J=0.6 Hz, 1H), 5.64 (d, J=10.2 Hz, 1H), 3.58 (s, 3H), 2.77 (s, 3H), 1.34 (s, 3H), 1.18 (s, 3H).

28 ¹H NMR (300 MHz, Methylene Chloride-d₂) δ 7.76-7.65 (m, 2H), 7.60-7.51 (m, 1H), 7.49-7.40 (m, 2H), 7.29 (dd, J=7.8, 1.6 Hz, 1H), 7.19 (d, J=7.8 Hz, 1H), 7.13-7.07 (m, 1H), 6.95 (dd, J=10.3, 0.8 Hz, 1H), 6.73-6.61 (m, 2H), 6.48-6.38 (m, 1H), 5.86 (d, J=10.2 Hz, 1H), 3.75 (s, 3H), 2.69 (s, 3H), 1.29 (s, 3H), 1.18 (s, 3H).

29 ¹H NMR (500 MHz, Methylene Chloride-d₂) δ 8.01-7.97 (m, 1H), 7.89 (d, J=2.1 Hz, 1H), 7.18 (td, J=7.7, 1.3 Hz, 1H), 7.09 (ddd, J=7.3, 1.3, 0.5 Hz, 1H), 7.01 (d, J=10.4 Hz, 1H), 6.88 (td, J=7.4, 1.0 Hz, 1H), 6.55 (dt, J=7.8, 0.7 Hz, 1H), 5.90 (d, J=10.4 Hz, 1H), 2.69 (s, 3H), 2.54 (s, 3H), 1.31 (s, 3H), 1.20 (s, 3H).

30 ¹H NMR (500 MHz, Methylene Chloride-d₂) δ 7.85-7.80 (m, 2H), 7.74-7.70 (m, 2H), 7.32 (dd, J=7.8, 1.6 Hz, 1H), 7.22 (d, J=7.8 Hz, 1H), 7.15 (td, J=7.7, 1.3 Hz, 1H), 7.10 (dd, J=1.7, 0.7 Hz, 1H), 7.06 (ddd, J=7.2, 1.3, 0.6 Hz, 1H), 6.98 (dd, J=10.2, 0.7 Hz, 1H), 6.82 (td, J=7.4, 1.0 Hz, 1H), 6.52 (dt, J=7.8, 0.7 Hz, 1H), 5.89 (d, J=10.2 Hz, 1H), 2.74 (s, 3H), 1.31 (s, 3H), 1.18 (s, 3H).

31 ¹H NMR (300 MHz, Methylene Chloride-d₂) δ 7.96-7.89 (m, 1H), 7.81 (d, J=2.2 Hz, 1H), 7.79-7.68 (m, 6H), 7.67-7.59 (m, 1H), 7.59-7.37 (m, 5H), 7.04 (d, J=10.5 Hz, 1H), 6.58 (d, J=8.6 Hz, 1H), 5.92 (d, J=10.4 Hz, 1H), 2.82 (s, 3H), 1.39 (s, 3H), 1.26 (s, 3H).

32 ¹H NMR (500 MHz, Methylene Chloride-d₂) δ 8.06-7.91 (m, 2H), 7.88 (d, J=1.7 Hz, 1H), 7.83-7.78 (m, 1H), 7.69 (dd, J=8.2, 1.8 Hz, 1H), 7.59 (dd, J=8.2, 1.8 Hz, 1H), 7.57-7.46 (m, 3H), 7.40 (t, J=2.2 Hz, 1H), 7.16 (dd, J=8.6, 2.5 Hz, 1H), 6.97 (dd, J=10.3, 0.7 Hz, 1H), 6.85 (d, J=8.2 Hz, 1H), 6.78-6.74 (m, 1H), 6.46 (d, J=8.2 Hz, 1H), 5.77 (d, J=10.3 Hz, 1H), 3.85 (s, 3H), 2.81 (s, 3H), 1.35 (s, 3H), 1.26 (s, 3H).

33 ¹H NMR (500 MHz, Methylene Chloride-d₂) δ 7.81-7.75 (m, 2H), 7.61-7.54 (m, 2H), 7.23-7.13 (m, 3H), 7.08 (ddd, J=7.3, 1.4, 0.5 Hz, 1H), 6.94 (dd, J=10.3, 0.7 Hz, 1H), 6.85 (td, J=7.4, 1.0 Hz, 1H), 6.77 (dt, J=8.3, 0.7 Hz, 1H), 6.55 (dt, J=7.8, 0.7 Hz, 1H), 5.81 (d, J=10.3 Hz, 1H), 2.75 (s, 3H), 1.31 (s, 3H), 1.18 (s, 3H).

34 ¹H NMR (500 MHz, Methanol-d₄) δ 7.46 (dd, J=7.9, 1.7 Hz, 1H), 7.24 (d, J=1.6 Hz, 1H), 7.22 (d, J=7.9 Hz, 1H), 7.12 (td, J=7.7, 1.3 Hz, 1H), 7.05 (dd, J=7.3, 1.2 Hz, 1H), 7.01 (dd, J=10.3, 0.7 Hz, 1H), 6.80 (td, J=7.4, 1.0 Hz, 1H), 6.53 (d, J=7.7 Hz, 1H), 5.92 (d, J=10.2 Hz, 1H), 2.98-2.94 (m, 2H), 2.70 (s, 3H), 2.56-2.22 (m, 2H), 1.95-1.78 (m, 2H), 1.28 (s, 3H), 1.15 (s, 3H).

35 ¹H NMR (300 MHz, Methylene Chloride-d₂) δ 7.82-7.66 (m, 4H), 7.62-7.39 (m, 5H), 6.93 (dd, J=10.4, 0.7 Hz, 1H), 6.87-6.77 (m, 1H), 6.64-6.54 (m, 1H), 5.84 (d, J=10.3 Hz, 1H), 2.83 (s, 3H), 1.32 (s, 3H), 1.21 (s, 3H).

36 ¹H NMR (300 MHz, Methylene Chloride-d₂) δ 7.76 (dd, J=8.3, 1.9 Hz, 1H), 7.56 (d, J=1.9 Hz, 1H), 7.46-7.36 (m, 2H), 6.97-6.91 (m, 1H), 6.80-6.74 (m, 1H), 6.62 (d, J=8.3 Hz, 1H), 5.82 (d, J=10.3 Hz, 1H), 3.02 (s, 3H), 2.81 (s, 3H), 1.32 (s, 3H), 1.19 (s, 3H).

37 ¹H NMR (300 MHz, Methylene Chloride-d₂) δ 7.81 (m, 2H), 7.77-7.65 (m, 4H), 7.60-7.53 (m, 1H), 7.52-7.44 (m, 2H), 6.98 (dd, J=10.4, 0.7 Hz, 1H), 6.75 (dd, J=9.1, 0.7 Hz, 1H), 6.60-6.50 (m, 1H), 5.78 (d, J=10.3 Hz, 1H), 3.85 (s, 3H), 2.83 (s, 3H), 1.33 (s, 3H), 1.20 (s, 3H).

38 ¹H NMR (300 MHz, Methylene Chloride-d₂) δ 9.84 (s, 1H), 7.81-7.63 (m, 6H), 7.62-7.53 (m, 1H), 7.53-7.45 (m, 2H), 7.02 (dd, J=10.3, 0.7 Hz, 1H), 6.89-6.81 (m, 1H), 6.57 (dd, J=7.8, 0.9 Hz, 1H), 5.83 (d, J=10.3 Hz, 1H), 2.84 (s, 3H), 1.33 (s, 3H), 1.22 (s, 3H).

39 ¹H NMR (300 MHz, Methanol-d₄) δ 7.88-7.80 (m, 2H), 7.75-7.57 (m, 5H), 7.57-7.47 (m, 2H), 7.10 (dd, J=10.4, 0.7 Hz, 1H), 6.87-6.72 (m, 2H), 5.96 (d, J=10.3 Hz, 1H), 3.58-3.37 (m, 2H), 2.83 (ddd, J=8.0, 6.8, 3.1 Hz, 2H), 2.55 (s, 3H), 2.21-2.02 (m, 2H), 1.32 (s, 3H), 1.22 (s, 3H).

40 ¹H NMR (500 MHz, Methylene Chloride-d₂) δ 7.75-7.73 (m, 1H), 7.73 (d, J=1.5 Hz, 1H), 7.71 (dd, J=8.1, 1.8 Hz, 1H), 7.68 (dd, J=1.8, 0.5 Hz, 1H), 7.60-7.55 (m, 1H), 7.52-7.47 (m, 2H), 7.44 (dt, J=1.5, 0.8 Hz, 1H), 7.27 (ddt, J=10.7, 2.9, 1.9 Hz, 1H), 7.24-7.21 (m, 1H), 6.58 (d, J=8.1 Hz, 1H), 6.04 (d, J=10.7 Hz, 1H), 2.84 (s, 3H), 1.35 (s, 3H), 1.23 (s, 3H).

41 ¹H NMR (500 MHz, Methylene Chloride-d₂) δ 7.73 (t, J=1.4 Hz, 1H), 7.72 (d, J=1.5 Hz, 1H), 7.69 (dd, J=8.2, 1.8 Hz, 1H), 7.67-7.66 (m, 1H), 7.60-7.53 (m, 1H), 7.53-7.44 (m, 2H), 7.25 (dd, J=10.5, 0.8 Hz, 1H), 6.95 (d, J=2.0 Hz, 1H), 6.69 (dd, J=2.0, 0.8 Hz, 1H), 6.55 (d, J=8.1 Hz, 1H), 5.84 (d, J=10.5 Hz, 1H), 2.82 (s, 3H), 1.32 (s, 3H), 1.20 (s, 3H).

42 ¹H NMR (500 MHz, Methylene Chloride-d₂) δ 10.16 (s, 1H), 9.90 (s, 1H), 8.14 (d, J=2.1 Hz, 1H), 7.88 (d, J=2.1 Hz, 1H), 7.77-7.72 (m, 2H), 7.72-7.69 (m, 2H), 7.59-7.55 (m, 1H), 7.53-7.45 (m, 2H), 7.08 (d, J=10.5 Hz, 1H), 6.60 (d, J=8.7 Hz, 1H), 5.95 (d, J=10.5 Hz, 1H), 2.88 (s, 3H), 1.35 (s, 3H), 1.29 (s, 3H).

43 1H NMR (500 MHz, Methylene Chloride-d₂) δ 7.78-7.72 (m, 4H), 7.71-7.67 (m, 2H), 7.59-7.55 (m, 1H), 7.51-7.47 (m, 2H), 6.99 (dd, J=10.3, 0.7 Hz, 1H), 6.77 (dt, J=8.3, 0.7 Hz, 1H), 6.58-6.53 (m, 1H), 5.79 (d, J=10.3 Hz, 1H), 2.84 (s, 3H), 2.51 (s, 3H), 1.33 (s, 3H), 1.21 (s, 3H).

44 ¹H NMR (300 MHz, Methanol-d₄) δ 7.88 (d, J=8.5 Hz, 2H), 7.82 (d, J=8.0 Hz, 2H), 7.42 (d, J=8.6 Hz, 1H), 7.32-7.22 (m, 2H), 7.17-7.05 (m, 3H), 5.96 (d, J=10.4 Hz, 1H), 3.18 (s, 3H), 1.63 (s, 3H), 1.36 (s, 3H).

45 ¹H NMR (300 MHz, Methanol-d₄) δ 8.28 (d, J=1.9 Hz, 1H), 8.00 (d, J=8.9 Hz, 1H), 7.85 (d, J=8.5 Hz, 1H), 7.80 (dd, J=9.0, 1.9 Hz, 1H), 7.75-7.69 (m, 1H), 7.62-7.55 (m, 1H), 7.48 (t, J=7.4 Hz, 1H), 7.30 (d, J=7.7 Hz, 1H), 7.25 (dd, J=7.8, 1.6 Hz, 1H), 7.17-7.04 (m, 1H), 6.04 (d, J=10.3 Hz, 1H), 3.11 (s, 3H), 2.89 (s, 3H), 1.66 (s, 3H), 1.36 (s, 3H).

47 ¹H NMR (300 MHz, Methanol-d₄) δ 7.76-7.54 (m, 8H), 7.54-7.43 (m, 4H), 7.34-7.21 (m, 2H), 7.15-7.02 (m, 2H), 6.78 (d, J=8.8 Hz, 1H), 6.03 (d, J=10.2 Hz, 1H), 3.62-3.34 (m, 2H), 2.94-2.75 (m, 2H), 2.25-2.01 (m, 2H), 1.33 (s, 3H), 1.22 (s, 3H).

49 ¹H NMR (300 MHz, Methylene Chloride-d₂) δ 7.80-7.66 (m, 4H), 7.63-7.54 (m, 1H), 7.53-7.43 (m, 3H), 7.35-7.25 (m, 1H), 7.20 (d, J=7.9 Hz, 1H), 6.98 (dd, J=10.3, 0.8 Hz, 1H), 6.55 (dd, J=7.8, 0.8 Hz, 1H), 5.86 (d, J=10.2 Hz, 1H), 2.83 (s, 3H), 2.49 (s, 3H), 1.35 (s, 3H), 1.21 (s, 3H).

50 ¹H NMR (300 MHz, Methylene Chloride-d₂) δ 7.84-7.66 (m, 2H), 7.56 (m, 1H), 7.51-7.39 (m, 2H), 7.36-7.24 (m, 2H), 7.23-7.15 (m, 1H), 7.16-7.08 (m, 1H), 6.97 (dd, J=10.3, 0.8 Hz, 1H), 6.33 (d, J=8.3 Hz, 1H), 5.83 (d, J=10.2 Hz, 1H), 2.72 (s, 3H), 1.28 (s, 3H), 1.17 (s, 3H).

51 ¹H NMR (500 MHz, Methylene Chloride-d₂) δ 7.83-7.73 (m, 3H), 7.63-7.54 (m, 3H), 7.24-7.15 (m, 2H), 6.99 (dd, J=10.4, 0.7 Hz, 1H), 6.77 (dt, J=8.2, 0.8 Hz, 1H), 6.63 (d, J=8.3 Hz, 1H), 5.79 (d, J=10.3 Hz, 1H), 3.03 (s, 3H), 2.85 (s, 3H), 1.36 (s, 3H), 1.21 (s, 3H).

52 ¹H NMR (500 MHz, Methanol-d₄) δ 7.89-7.81 (m, 2H), 7.79 (dd, J=8.4, 2.0 Hz, 1H), 7.70-7.60 (m, 3H), 7.35-7.24 (m, 2H), 7.11 (d, J=10.3 Hz, 1H), 6.94 (d, J=8.3 Hz, 1H), 6.82 (d, J=8.4 Hz, 1H), 5.98 (d, J=10.3 Hz, 1H), 3.61-3.42 (m, 2H), 3.12 (s, 3H), 2.93-2.78 (m, 2H), 2.23-2.03 (m, 2H), 1.38 (s, 3H), 1.25 (s, 3H).

53 ¹H NMR (300 MHz, Methylene Chloride-d₂) δ 7.79-7.63 (m, 7H), 7.62-7.54 (m, 1H), 7.53-7.45 (m, 2H), 6.99 (d, J=10.4 Hz, 1H), 6.85 (d, J=8.5 Hz, 1H), 6.62-6.54 (m, 1H), 5.87 (d, J=10.3 Hz, 1H), 2.85 (s, 3H), 1.33 (s, 3H), 1.22 (s, 3H).

55 ¹H NMR (300 MHz, Methylene Chloride-d₂) δ 8.05-7.92 (m, 2H), 7.68-7.60 (m, 2H), 7.58-7.43 (m, 3H), 7.14 (d, J=8.8 Hz, 1H), 7.03 (d, J=8.6 Hz, 1H), 6.96-6.89 (m, 2H), 6.80-6.74 (m, 1H), 6.70 (d, J=8.6 Hz, 1H), 5.69 (d, J=10.3 Hz, 1H), 3.15 (s, 3H), 1.46 (s, 3H), 1.22 (s, 3H).

56 ¹H NMR (300 MHz, Methylene Chloride-d₂) δ 7.59 (dd, J=8.3, 1.9 Hz, 1H), 7.51-7.15 (m, 8H), 7.06-6.91 (m, 2H), 6.29 (d, J=8.2 Hz, 1H), 5.68 (d, J=10.3 Hz, 1H), 2.99 (s, 3H), 2.64 (s, 3H), 1.08 (s, 6H).

57 ¹H NMR (300 MHz, Methylene Chloride-d₂) δ 7.82-7.41 (m, 8H), 7.28 (dd, J=7.4, 1.8 Hz, 1H), 7.01 (d, J=10.3 Hz, 1H), 6.92 (dd, J=7.9, 7.3 Hz, 1H), 6.56 (d, J=8.6 Hz, 1H), 5.85 (d, J=10.3 Hz, 1H), 2.83 (s, 3H), 2.09 (s, 3H), 1.36 (s, 3H), 1.25 (s, 3H).

58 ¹H NMR (300 MHz, Methylene Chloride-d₂) δ 7.78-7.64 (m, 5H), 7.61-7.53 (m, 1H), 7.52-7.44 (m, 2H), 7.31 (dd, J=7.8, 1.2 Hz, 1H), 7.20 (t, J=7.9 Hz, 1H), 6.89 (ddd, J=8.1, 1.2, 0.8 Hz, 1H), 6.53 (dd, J=7.9, 0.8 Hz, 1H), 5.82 (d, J=10.7 Hz, 1H), 2.83 (s, 3H), 2.59 (s, 3H), 1.32 (s, 3H), 1.21 (s, 3H).

59 ¹H NMR (300 MHz, Methylene Chloride-d₂) δ 7.83-7.66 (m, 3H), 7.63-7.49 (m, 2H), 7.50-7.36 (m, 2H), 7.21 (s, 1H), 6.91 (d, J=10.3 Hz, 1H), 6.63 (d, J=8.3 Hz, 1H), 6.38 (s, 1H), 5.62 (d, J=10.2 Hz, 1H), 3.59 (s, 3H), 3.03 (s, 3H), 2.86 (s, 3H), 1.39 (s, 3H), 1.21 (s, 3H).

60 ¹H NMR (300 MHz, Methylene Chloride-d₂) δ 7.79-7.65 (m, 6H), 7.62-7.53 (m, 1H), 7.49 (m, 2H), 7.22-7.14 (m, 1H), 6.79-6.66 (m, 1H), 6.63-6.50 (m, 1H), 5.74 (d, J=10.2 Hz, 1H), 2.84 (s, 3H), 2.23 (s, 6H), 1.29 (s, 3H), 1.24 (s, 6H), 1.17 (s, 3H).

61 ¹H NMR (300 MHz, Methylene Chloride-d₂) δ 8.02 (dd, J=2.2, 0.7 Hz, 1H), 7.92 (d, J=2.2 Hz, 1H), 7.79-7.68 (m, 4H), 7.62-7.53 (m, 1H), 7.53-7.45 (m, 2H), 7.05 (d, J=10.5 Hz, 1H), 6.61-6.54 (m, 1H), 5.91 (d, J=10.4 Hz, 1H), 2.79 (s, 3H), 2.55 (s, 3H), 1.36 (s, 3H), 1.25 (s, 3H).

62 ¹H NMR (300 MHz, Methylene Chloride-d₂) δ 8.16 (d, J=1.8 Hz, 1H), 8.00-7.92 (m, 1H), 7.85-7.78 (m, 1H), 7.75-7.67 (m, 2H), 7.59-7.38 (m, 4H), 7.34-7.18 (m, 2H), 7.13-6.98 (m, 3H), 5.93 (d, J=10.3 Hz, 1H), 2.90 (s, 3H), 1.64 (s, 3H), 1.34 (s, 3H).

63 ¹H NMR (500 MHz, Methylene Chloride-d₂) δ 7.77-7.65 (m, 4H), 7.59-7.54 (m, 1H), 7.53-7.46 (m, 2H), 7.33-7.25 (m, 1H), 7.03 (d, J=1.4 Hz, 1H), 6.97 (s, 1H), 6.54 (d, J=8.2 Hz, 1H), 5.79 (d, J=10.5 Hz, 1H), 3.92 (s, 3H), 2.83 (s, 3H), 2.47 (s, 3H), 1.34 (s, 3H), 1.21 (s, 3H).

64 ¹H NMR (300 MHz, Methanol-d₄) δ 7.83 (s, 1H), 7.77-7.68 (m, 2H), 7.67-7.58 (m, 1H), 7.50 (tt, J=7.5, 0.8 Hz, 2H), 7.36-7.21 (m, 2H), 7.19-7.06 (m, 2H), 5.91 (d, J=10.3 Hz, 1H), 3.20 (s, 3H), 1.51 (s, 3H), 1.28 (s, 3H).

65 ¹H NMR (300 MHz, Methanol-d₄) δ 7.49 (dd, J=7.8, 1.7 Hz, 1H), 7.43 (d, J=8.6 Hz, 1H), 7.29 (d, J=1.4 Hz, 1H), 7.22 (d, J=7.9 Hz, 1H), 7.13 (d, J=8.6 Hz, 1H), 7.10-7.04 (m, 1H), 5.91 (d, J=10.3 Hz, 1H), 3.15 (s, 3H), 2.95 (q, J=5.9, 4.7 Hz, 2H), 2.27-2.16 (m, 2H), 1.98-1.87 (m, 2H), 1.61 (s, 3H), 1.35 (s, 3H).

66 ¹H NMR (300 MHz, Methanol-d₄) δ 7.77-7.68 (m, 2H), 7.65-7.57 (m, 1H), 7.54-7.46 (m, 2H), 7.42 (d, J=8.6 Hz, 1H), 7.32-7.21 (m, 2H), 7.18-7.02 (m, 3H), 5.93 (d, J=10.3 Hz, 1H), 3.18 (s, 3H), 1.64 (s, 3H), 1.36 (s, 3H).

67 ¹H NMR (500 MHz, Methanol-d₄) δ 8.11 (d, J=8.6 Hz, 1H), 7.71 (d, J=8.6 Hz, 1H), 4.32 (s, 3H), 1.89 (s, 6H).

68 ¹H NMR (300 MHz, Methanol-d₄) δ 8.16 (s, 1H), 4.57 (s, 3H), 2.51 (s, 3H), 1.76 (s, 6H).

69 ¹H NMR (500 MHz, Methanol-d₄) δ 8.88 (d, J=1.4 Hz, 1H), 8.60 (d, J=1.4 Hz, 1H), 4.12 (s, 3H), 1.82 (s, 6H).

70 ¹H NMR (300 MHz, Methanol-d₄) δ 8.19 (dd, J=1.7, 0.5 Hz, 1H), 8.02 (dd, J=8.4, 1.6 Hz, 1H), 7.65-7.60 (m, 1H), 4.02 (s, 3H), 1.60 (s, 6H).

71 ¹H NMR (300 MHz, Methanol-d₄) δ 8.16 (t, J=1.1 Hz, 1H), 8.00 (d, J=1.1 Hz, 2H), 7.85-7.74 (m, 2H), 7.74-7.66 (m, 1H), 7.62-7.51 (m, 2H), 4.13 (s, 3H), 1.67 (s, 6H).

72 ¹H NMR (300 MHz, Methanol-d₄) δ 8.20-8.05 (m, 1H), 8.03-7.84 (m, 1H), 7.84-7.60 (m, 2H), 7.60-7.39 (m, 2H), 7.36-7.00 (m, 2H), 4.85-4.21 (m, 2H), 3.12-2.75 (m, 2H), 2.45-1.92 (m, 2H), 1.71-1.45 (m, 6H).

73 ¹H NMR (500 MHz, Methanol-d₄) δ 8.43 (s, 1H), 8.31 (d, J=8.3 Hz, 1H), 8.17 (s, 1H), 4.19 (s, 3H), 3.33 (s, 3H), 1.75 (s, 6H).

74 ¹H NMR (500 MHz, Deuterium Oxide) δ 8.38 (s, 1H), 8.26 (d, J=8.5 Hz, 1H), 8.13 (d, J=8.7 Hz, 1H), 3.34 (s, 3H), 3.14 (t, J=6.9 Hz, 2H), 2.42 (p, J=7.3 Hz, 2H), 2.20-1.99 (m, 2H), 1.66 (s, 6H).

75 ¹H NMR (300 MHz, Methanol-d₄) δ 8.64 (s, 1H), 8.27 (s, 1H), 4.18 (s, 3H), 1.75 (s, 6H).

76 ¹H NMR (300 MHz, Methanol-d₄) δ 8.00 (s, 1H), 7.66 (s, 1H), 4.31 (s, 3H), 1.76 (s, 6H).

77 ¹H NMR (300 MHz, Methanol-d₄) δ 8.69 (s, 1H), 8.51 (dd, J=8.8, 0.9 Hz, 1H), 8.45-8.32 (m, 1H), 8.16 (d, J=9.0 Hz, 1H), 7.98 (dd, J=8.8, 1.7 Hz, 1H), 4.19 (s, 3H), 1.85 (s, 6H).

79 ¹H NMR (500 MHz, Methylene Chloride-d₂) δ 7.82-7.78 (m, 2H), 7.77-7.72 (m, 2H), 7.66 (dd, J=8.1, 1.7 Hz, 1H), 7.57 (d, J=1.7 Hz, 1H), 6.97 (dd, J=10.4, 0.7 Hz, 1H), 6.78-6.74 (m, 1H), 6.74-6.69 (m, 2H), 6.55 (d, J=8.1 Hz, 1H), 5.78 (d, J=10.3 Hz, 1H), 3.85 (s, 3H), 3.06 (s, 6H), 2.82 (s, 3H), 1.32 (s, 3H), 1.20 (s, 3H).

80 ¹H NMR (500 MHz, Methylene Chloride-d₂) δ 8.27 (dd, J=8.0, 1.8 Hz, 1H), 8.22 (d, J=8.4 Hz, 1H), 7.91 (d, J=2.1 Hz, 1H), 7.83 (d, J=2.1 Hz, 1H), 7.76 (dd, J=8.2, 1.4 Hz, 2H), 7.70 (ddd, J=8.7, 7.1, 1.8 Hz, 1H), 7.66-7.61 (m, 1H), 7.53 (t, J=7.7 Hz, 2H), 7.46 (dd, J=8.4, 1.0 Hz, 1H), 7.36 (ddd, J=8.1, 7.1, 1.1 Hz, 1H), 7.07 (d, J=10.4 Hz, 1H), 6.69 (d, J=8.4 Hz, 1H), 5.94 (d, J=10.4 Hz, 1H), 2.87 (s, 3H), 1.69 (s, 3H), 1.42 (s, 3H).

81 ¹H NMR (500 MHz, Methanol-d₄) δ 7.62-7.59 (m, 1H), 7.45-7.42 (m, 3H), 7.40 (d, J=7.6 Hz, 1H), 7.36-7.34 (m, 2H), 7.33-7.27 (m, 2H), 7.11 (d, J=10.3 Hz, 1H), 7.05 (t, J=7.6 Hz, 1H), 6.58 (d, J=8.4 Hz, 1H), 5.90 (d, J=10.3 Hz, 1H), 3.58-3.39 (m, 2H), 3.09 (s, 3H), 2.86-2.76 (m, 2H), 2.09-1.97 (m, 2H), 1.34 (s, 3H), 1.13 (s, 3H).

82 ¹H NMR (500 MHz, Methanol-d₄) δ 7.77 (ddd, J=11.0, 8.4, 1.7 Hz, 3H), 7.68-7.59 (m, 2H), 7.58-7.50 (m, 2H), 7.35 (d, J=7.8 Hz, 1H), 7.30 (dd, J=7.8, 1.6 Hz, 1H), 7.15 (d, J=10.3 Hz, 1H), 7.07 (d, J=1.4 Hz, 1H), 6.93 (d, J=8.4 Hz, 1H), 6.06 (d, J=10.3 Hz, 1H), 3.59-3.43 (m, 2H), 3.12 (s, 3H), 2.91-2.80 (m, 2H), 2.23-2.06 (m, 2H), 1.39 (s, 3H), 1.26 (s, 3H).

83 ¹H NMR (500 MHz, Methanol-d₄) δ 7.43-7.33 (m, 5H), 7.30 (d, J=8.6 Hz, 1H), 7.24 (dd, J=8.7, 6.9 Hz, 2H), 7.05 (d, J=10.4 Hz, 1H), 7.02-6.95 (m, 2H), 5.72 (d, J=10.4 Hz, 1H), 2.97 (s, 3H), 1.44 (s, 3H), 1.24 (s, 3H).

84 ¹H NMR (300 MHz, Methylene Chloride-d₂) δ 8.45-8.31 (m, 1H), 7.90-7.77 (m, 1H), 7.77-7.68 (m, 2H), 7.60-7.49 (m, 1H), 7.49-7.28 (m, 7H), 7.23 (t, J=8.3 Hz, 1H), 7.14-6.98 (m, 2H), 5.96 (d, J=10.3 Hz, 1H), 3.37 (s, 3H), 1.39 (s, 3H), 1.19 (s, 3H).

87 ¹H NMR (500 MHz, Methylene Chloride-d₂) δ 7.72 (dd, J=8.4, 1.9 Hz, 1H), 7.61 (dd, J=8.3, 1.9 Hz, 1H), 7.59 (d, J=1.9 Hz, 1H), 7.50-7.47 (m, 2H), 7.45 (ddd, J=7.5, 1.6, 0.8 Hz, 1H), 7.42-7.37 (m, 1H), 7.35 (d, J=1.9 Hz, 1H), 7.28-7.21 (m, 3H), 6.96 (t, J=7.6 Hz, 1H), 6.64 (d, J=8.4 Hz, 1H), 6.36 (d, J=8.3 Hz, 1H), 4.42 (d, J=10.2 Hz, 1H), 3.13 (s, 3H), 3.02 (s, 3H), 2.99 (s, 3H), 2.54 (s, 3H), 2.37 (dd, J=14.2, 4.9 Hz, 1H), 2.20-2.10 (m, 1H), 1.67 (s, 3H), 1.65 (s, 3H), 1.31 (s, 3H), 1.25 (s, 3H), 1.22-1.18 (m, 1H).

91 1H NMR (500 MHz, Methylene Chloride-d₂) δ 7.72-7.68 (m, 2H), 7.58-7.54 (m, 2H), 7.53-7.47 (m, 4H), 7.41 (d, J=1.8 Hz, 1H), 7.40-7.37 (m, 1H), 7.31 (dd, J=7.6, 1.7 Hz, 1H), 7.27 (dd, J=7.5, 1.7 Hz, 1H), 7.26-7.22 (m, 2H), 6.97 (dd, J=8.9, 1.4 Hz, 2H), 6.25 (d, J=8.2 Hz, 1H), 5.70 (d, J=10.3 Hz, 1H), 2.66 (s, 3H), 1.09 (s, 3H), 1.08 (s, 3H).

### B. Formulation

In several cases it may be advantageous, when the dual color photoinitiator is lyophilized before formulating.

### Example Formulation 1

Initiator 66 (1 mg) was dissolved in a mixture of methyldiethanolamine (1 g), PEG-diacrylate (MW575) (1 g) and gelatine methacrylate (8 g). The mixture was shaken at 50° C. until the components were well mixed and allowed to cool to room temperature before printing.

### Example Formulation 2

Initiator 43 (2 mg) was dissolved in methyldiethanolamine (0.4 g) and diurethane dimethacrylate, mixture of isomers, CAS 72869-86-4 (10 g). The mixture was stirred until homogenization and could be directly used for printing.

### Example Formulation 3

Initiator 55 (2 mg) was dissolved in methyldiethanolamine (0.4 g) and diurethane dimethacrylate, mixture of isomers, CAS 72869-86-4 (10 g). The mixture was stirred until homogenization and could be directly used for printing.

### Example Formulation 4

Initiator 56 (10 mg) was dissolved in acryloyl morpholine (0.4 g), methyldiethanolamine (0.4 g) and diurethane dimethacrylate, mixture of isomers, CAS 72869-86-4 (10 g). The mixture was stirred until homogenization and could be directly used for printing.

### C. Printing

The above described formulations are used for volumetric printing.

The setup for volumetric printing is as follows: a cuvette with four transparent windows is irradiated in one direction with a light sheet of wavelength 1, while an image is projected onto the light sheet from a different angle with wavelength 2. The image is changed to produce a movie, while the light sheet is moved through the cuvette. The prints result in solidification only in volumes where the light of both wavelengths intersects. The residual uncured resin is removed to obtain the shaped body, which is further washed with solvent and post-processed.

It has been found that Formulation 1, Formulation 2, Formulation 3 and Formulation 4 were successful for volumetric printing under the use of light of two different wavelengths. Exemplarily the following wavelengths can be used:
Formulation 1, 3, 4:375 nm and 500-700 nm
Formulation 2:405 nm and 500-700 nm

The post-processing is exemplarily described for the shaped body produced from Formulation 4:

Post-processing example 1: After washing with ethanol, the three-dimensional object is subjected to irradiation with light of a wavelength of 420 nm for 30 min (Thorlabs M420L3, 750 mW) which results in that the absorption of the object in the visible wavelength range is significantly reduced.

Post-processing example 2: After washing with ethanol, the three-dimensional object is placed in a heatable chamber in which the three-dimensional object is tempered at 120° C. for 30 min which results in that the absorption in the visible wavelength range is significantly reduced.

### Dual Color Photoinitiation Properties Depending on the Spiropyran Motif

In an example it has been found that, when R⁴ is H and R¹¹ is benzoyl the resulting spiropyran is not suitable as dual color photoinitiator due to very inefficient switching reflected in a low quantum yield and a very slow thermal back reaction, which does not allow for prints with z-resolution.

When R⁴ and R¹¹ are benzoyl, the resulting spiropyran is suitable as dual color photoinitiator, with a sufficiently fast thermal back reaction, a sufficiently high switching efficiency and a sufficiently high reactivity to allow volumetric printing in the xolography process.

When R⁴ and R¹³ (structure 91) are benzoyl which is accessible with the method described here in, a similar switching efficiency but surprisingly also an enhanced thermal back reaction rate and a higher reactivity are observed.

Furthermore, it has been found that, when R⁴ is benzoyl the resulting motif surprisingly results in an extinction coefficient at 405 nm which is sufficiently high to use 405 nm as the first wavelength necessary for switching to the intermediate state.

In contrast, when R¹¹ is benzoyl and R⁴ is H, the extinction coefficient at 405 nm is not sufficiently high, so that the first wavelength needs to be shifted to 375 nm. An irradiation with the first wavelength being 405 nm may be advantageous compared to 375 nm, since laser diodes are available with higher power at lower costs. Furthermore, 405 nm is less harmful to cells, which may be of high relevance when prints shall be performed in the presence of cells. When R¹¹ is acyl the extinction coefficient at 405 nm is not sufficient for printing but the reactivity of the intermediate state with visible light and a co-initiator is surprisingly high, compared to respective structures where R¹¹ is benzoyl, ester, cyano or CF₃. The combination of R⁴ being benzoyl and R¹¹ being acyl (43) results in a surprisingly well performing dual color photoinitiator which has superior properties compared to dual color photoinitiators with just one carbonyl group or two identical carbonyl substituents.

In another example it has been found that, when R⁴ is SO₂Me and R¹¹ is 4-F-benzoyl (51), the switching is surprisingly efficient, with a quantum yield near unity and the thermal back reaction is accelerated to the few seconds thermal half-life regime, allowing for decent print performance. When R⁴ is SO₂Me and R¹³ is benzoyl (instead of R¹¹) (56), the switching is as efficient with a quantum yield near unity, but surprisingly the thermal back reaction rate as well as the initiation efficiency are increased even further, so that enhanced print performance over 51 regarding resolution, print speed and hardness of the green state is achieved.

From large sets of examples design rules for specific properties can be derived, exemplarily:
The back reaction rate as well as the switching efficiency may be accelerated with stronger acceptors at R⁴.
A benzoyl substituent may cause a bathochromic shift of the spiropyran absorption spectrum from strongest to weakest: R⁴>R¹²>R¹¹>R¹³>R¹⁰
A benzoyl substituent on the pyran ring may cause a suitable thermal back reaction rate in the order from high to low: R¹⁰>R¹²>R¹³>R¹¹

The features disclosed in the description and claims may be relevant to the realization of the various designs either individually or in any combination.

### General Findings for the Printing Process

The photoinitiators 43, 48, 51 and 56 have been investigated in terms of printing at different concentrations of dual color photoinitiators and with variations of the first wavelength. The initiators each bear one aroyl substituent in a different position, which causes shifts in the absorption spectra of the initial state. Volumetric printing is performed in a setup, where a cuvette with four transparent windows is irradiated in one direction with a light sheet of wavelength 1, while an image is projected onto the light sheet from a perpendicular direction with wavelength 2. The image is changed to produce a movie, while the cuvette is moved through the light sheet. The prints may result in solidification only in volumes where the light of both wavelengths intersects. The residual uncured resin is removed to obtain the shaped body, which is further washed with solvent and post-processed.

TABLE 1
Extinction coefficients in L mol⁻¹ cm⁻¹ of dual color
photoinitiators in the tail region of their absorption spectrum
of the initial state and thermal half-life at room temperature.
Measurements have been performed in a typical resin suited
for volumetric printing: urethane dimethacrylate containing
3.5% N-methyldiethanolamine and 4.5% acryloyl morpholine.

| Dual color | | | | | Thermal | |
|---|---|---|---|---|---|---|
| photoinitiator | 405 nm | 395 nm | 385 nm | 375 nm | half-life | |
| 43 | 260 | 640 | 1700 | 4100 | 15 | s |
| 48 | 90 | 170 | 490 | 1500 | 2.8 | s |
| 51 | <10 | 60 | 150 | 350 | 10 | s |
| 56 | <10 | <10 | 40 | 160 | 3.4 | s |

While photochemistry is ideally performed at a wavelength, where the absorbing species has its absorption maximum, surprisingly the tests did not result in successful prints when the light sheet is generated at a wavelength around the absorption maximum of the initial state. Instead, only curing at the walls of the cuvette is observed. This may be explained by Beer's law, since at the wavelength around the maximum, the photons are absorbed in close proximity to the cuvette wall, causing also the polymerization in close proximity to the cuvette wall, while only a minor fraction of the light reaches the middle of the cuvette.

To circumvent this problem, the concentration has been lowered, so that the absorption at the absorption maximum/first wavelength has been reduced to 1 or less.

Surprisingly, the penetration depth issue has been solved, but no curing was observed at all. Presumably, the concentration of the dual color photoinitiator has been too low in these experiments to allow the formation of a sufficient number of radicals.

Surprisingly, prints in the middle of the cuvette have been successful without curing at the walls, when the first wavelength was chosen to irradiate in the red tail of the absorption spectrum, where the initial state has a lower extinction coefficient. This is counter-intuitive, as one would expect less radicals to be formed, when the initiator absorbs less photons, but surprisingly more radicals are formed in the middle of the cuvette. However, when the first wavelength is shifted further, where the initial state has an even lower extinction coefficient, no curing is observed anymore or the intensity is largely increased, resulting in only curing at the cuvette walls. When the concentration is increased to an extent, that curing can be observed, the curing is caused mainly at the walls again. This may be explained by little impurities, with a relatively higher extinction coefficient than the initial state or the relatively higher intensity of the first wavelength necessary to cause the switching. By comparing prints of different concentrations and first wavelengths, the following conditions have been found to result in improved print results compared to the state of the art. When the first wavelength is adjusted accordingly to the extinction coefficient, the concentration of the initiator can be sufficiently increased to achieve a dual color effect. Surprisingly, these conditions are independent of the structure of the initiator itself, as they rather seem to be related to the surprising combination of restrictions given by Beer's law and the dual color effect:
1) The extinction coefficient of the dual color photoinitiator at the first wavelength should be in the range of 5000-0 L mol⁻¹ cm⁻¹, preferably 2500-10 L mol⁻¹ cm⁻¹, more preferably 1000-20 L mol⁻¹ cm⁻¹, even more preferably 500-50 L mol⁻¹ cm⁻¹, most preferably 300-100 L mol⁻¹ cm⁻¹.
2) The absorption of the resin at the first wavelength over a distance of 1 cm, using a non-absorbing solvent as reference, e.g. acetonitrile should be in the range of 1-0.05, preferably 0.9-0.1, more preferably 0.8-0.15.

For the Initiators given in Table 1, the ideal first wavelengths are: 43: 405 nm, 48:395 nm, 51:385 nm, and 56:375 nm.

Surprisingly, the preferred properties of the initiator and the resin are independent of the object which shall be printed but rely on the size of the container and the path length the light of the first wavelength has to travel. Since Beer's law involves the path length of the light, the problem of penetration depth is of lower importance for smaller build rooms, e.g. 1×1×1 mm³, while for build rooms >5×5×5 mm³, preferably >10×10×10 mm³, the above criteria ensure a highly improved print performance. To give a non-limiting example, when formulations containing 51 are dual color polymerized, no curing is observed using 405 nm as first wavelength, while 395 nm and 375 nm give green states of acceptable quality, the highest degree of polymerization was achieved using 385 nm. Furthermore, the adaption of the extinction coefficient allowed to increase the build room and still maintain sufficiently polymerized green states.

### Part B

In addition to the aspects and embodiments described above in PART A, the invention further relates, as described in the following in the PART B to further aspects and embodiments.

According to one aspect, a process for local polymerization of a starting material by means of dual color photopolymerization is provided. The process comprises the steps:
providing a polymerizable starting material containing photoinitiator molecules which can be converted by sequential optical excitation into a reactive state in which the photoinitiator molecules locally trigger polymerization of the starting material; and
photopolymerizing the starting material in a local volume by irradiating light of a first wavelength and light of a second wavelength, different from the first wavelength, into the local volume, whereby in the local volume
the photoinitiator molecules are converted, due to the absorption of the light of the first wavelength, from an initial state, into an intermediate state with changed optical properties compared to the initial state, such that the photoinitiator molecules in the intermediate state absorb the light of the second wavelength; and
the photoinitiator molecules are transferred from the intermediate state to the reactive state due to the absorption of the light of the second wavelength, which triggers the polymerization locally.

The terms “photoinitiator molecules” and “photoinitiator” are synonymous.

The terms “photopolymerizable composition”, “photopolymerizable material”, “photopolymerizable starting material”, “formulation”, and “resin” are synonymous.

It may be provided that the initial state does not substantially absorb the light of the second wavelength. The expression “does not substantially absorb” may be understood in that the absorption of the starting material which is caused by the photoinitiator molecules in the initial state at a path length of 10 mm is not larger than 1, less than 0.5, less than 0.1, less than 0.05, less than 0.01, ideally zero, at the emission maximum of the second wavelength in the range between 300 and 1000 nm.

In this context, it is further to be understood that the initial state corresponds to the closed (=spiropyran) form of the photoinitiator molecule such as the photoinitiator molecule comprising the structural motif (BI) as described below, whereas the intermediate state may represent one or more of the respective open merocyanine structures. Although the formation of the intermediate state is predominantly induced by irradiation, it may, in some cases, also occur thermally without exposure to light. Even in such thermally generated intermediate states, or in mixtures where the initial and intermediate forms are present simultaneously in the absence of irradiation, the absorption at the second wavelength remains within the same limits as defined above, i.e. the absorption at a path length of 10 mm at the emission maximum of the second wavelength between 300 and 1000 nm does not exceed the specified values.

Photopolymerizable starting materials suitable for use in the process according to the invention, such as for volumetric printing may contain the following parts by weight:
1-99.9999 wt %, preferably 5-99 wt %, more preferably 20-98 wt %, most preferably 50-95 wt % of photopolymerizable compound(s), e.g. monomer(s);
0-99 wt %, preferably 1-50 wt %, more preferably 2-20 wt % co-initiator, most preferably 3-10 wt % co-initiator
0.0001-20 wt %, preferably 0.001-10 wt %, more preferably 0.01-5 wt %, most preferably 0.1-1 wt % of a dual color photoinitiator;
0-20 wt %, preferably 1-10 wt %, more preferably 3-5 wt % of acids or bases;
0-90 wt %, preferably 1-80 wt %, more preferably 5-70 wt %, even more preferably 10-60 wt %, most preferably 20-50 wt % of fillers, such as organic or inorganic fillers, particularly inorganic fillers, such as fumed silica or nanoparticles, preferably functionalized or non-functionalized nanoparticles;
0-20 wt %, preferably 0.1-15 wt %, more preferably 0.2-10 wt %, most preferably 0.5-5 wt % of at least one additive, preferably at least one additive chosen from the group of optical brighteners, inhibitors, chain transfer agents, rheology modifiers, UV-blockers, more preferably an organic rheology modifier;
0-20 wt %, preferably 0.1-15 wt %, more preferably 0.2-10 wt %, most preferably 0.5-5 wt % of salts, preferably organo borate salts or iodonium salts, more preferably salts comprising a diphenyliodonium cation or a butyl-triphenylborate anion;
0-90 wt %, preferably 0.1-10 wt %, more preferably 0.2-2 wt % of solvent; and
0-99 wt %, preferably 5-95 wt %, more preferably 20-80 wt %, even more preferably 30-70 wt % of water.

The co-initiator can be a derivative of ethanolamine, preferably a derivative of diethanolamine, more preferably an N-alkyldiethanolamin, alternatively N-methyldiethanolamin,

N-phenyldiethanolamin, triethanolamine, 4-(2-hydroxyethyl)morpholine, N-(2-hydroxypropyl)morpholine, N-tert-butyldiethanolamine, N-butyldiethanolamine, N-(3-aminopropyl) diethanolamine, N,N-di(2-hydroxyethyl)glycine, 1-[bis(2-hydroxyethyl)amino]-2-propanol, N-phenyldiethanolamine, m-tolyldiethanolamine, p-tolyldiethanolamine, N-benzyldiethanolamine, bis(2-hydroxyethyl)aminotris(hydroxymethyl) methane, N,N-bis(2-hydroxypropyl) aniline, N,N-bis(2-hydroxyethyl)-2-aminoethanesulfonic Acid, N,N-bis(2-hydroxyethyl)-3-chloroaniline, N,N,N′,N′-tetrakis(2-hydroxyethyl)ethylenediamine, 3-[N,N-bis(2-hydroxyethyl)amino]-2-hydroxypropanesulfonic acid, N-lauryldiethanolamine, N,N,N′,N′-tetrakis(2-hydroxypropyl)ethylenediamine, N,N,N′,N″,N″-pentakis (2-hydroxypropyl) diethylenetriamine, ethyl 4-(dimethylamino)benzoate, isoamyl 4-(dimethylamino)benzoate, 2-butoxyethyl 4-(dimethylamino)benzoate, 2-ethylhexyl 4-(dimethylamino)benzoate.

Alternatively or in addition, a corresponding yield strength of the starting material can be set by mixing in oligomers and polymers having non-Newtonian flow properties and/or mixing in additives (rheology modifiers). Examples for oligomers having non-Newtonian flow properties are, in a non-exclusive listing: 1) linear polymers having average and/or high molar masses, e.g. PMMA, PS, PC, PIM, 2) celluloses and cellulose esters, 3) polyacrylic acids and polyacrylic acid/polyacrylic acid ester copolymers, 4) polyacrylamides, 5) polyethylene oxide, and 6) polyurethanes. Examples for rheology modifiers are, in a non-exclusive listing: 1) inorganic (nano) particles, e.g. pyrogenic silicic acid, natural and synthetic clay minerals, sheet silicates, glass, 2) organically modified inorganic (nano) particles, 3) organic molecules, oligomers and polymers (urea derivatives, polysaccharides, polyacrylic acids, polyacrylates, polyamides, polyethers, polyurethanes, polyurea derivatives), and 4) urea-modified polyacrylates, polyethers, polyamides and polyurethanes. Examples for commercial organic rheology modifiers which can be used are, in a non-exclusive listing: RHEOBYK 410, RHEOBYK 420, RHEOBYK 430, RHEOBYK 440, BYK-LP R21675, RHEOBYK 7410CA, RHEOBYK 7420CA, RHEOBYK 7420ET (all available from BYK-Chemie GmbH, 46486 Wesel (Germany)), JL-106 (available from Bomar Chem, 51 Greenwoods Rd, Torrington, CT 06790).

For any rheology modifier, it is preferably the case that this is selected and/or used, e.g. in view of its chemical and/or physical properties, concentration, etc., in such a way that it does not or barely leads to turbidity of the starting material and/or of the cured material after optical processing.

A UV blocker may be any compound which absorbs light at wavelength below 400 nm. A UV blocker can also be deemed a stabilizer, since it reduces damage to the manufactured object which may be caused by UV light. Typically, a UV blocker is characterized by a steep absorption edge at 400 nm. Where the photopolymerizable starting material comprises a UV blocker, it is preferred when the light of the first wavelength has a wavelength >400 nm, preferably 405 nm and/or the dual color photoinitiator can be switched from the initial state to the intermediate state by irradiation with light of the first wavelength being >400 nm, preferably 405 nm.

In one embodiment, the polymerizable starting material is photopolymerized in a local volume by irradiating light of a first wavelength and light of a second wavelength, wherein the light of the first wavelength and the light of the second wavelength can be intersected in the local volume.

In this embodiment, the process for local polymerization of a starting material by means of dual color photopolymerization according to the invention is a process for volumetric printing of a shaped body from a starting material by means of dual color photopolymerization. Here a polymerizable starting material is provided which contains a photoinitiator (=photoinitiator molecules) which can be converted by means of sequential optical excitation with several wavelengths into a reactive state in which the photoinitiator molecules locally trigger a polymerization of the starting material, in particular a local curing or hardening of the starting material due to the polymerization. The starting material is photopolymerized in a local volume by irradiating light of a first wavelength simultaneously with or followed by light of a second wavelength different from the first wavelength into the local volume.

In the local volume is provided that, by absorption of a photon of the first wavelength, the photoinitiator molecules are converted from an initial state, in which the photoinitiator molecules do not substantially absorb the light of the second wavelength, to an intermediate state with optical properties different from those of the initial state, in such a way that the photoinitiator molecules in the intermediate state absorb the light of the second wavelength and enter the reactive state. In the local volume, due to the gradual absorption of the light of the first wavelength and the light of the second wavelength, the photoinitiator molecules are transferred from the initial state via the intermediate state into the reactive state, which locally triggers polymerization. Outside of the local volume the photoinitiator molecules which have been in the intermediate state return to the initial state.

According to another aspect, an apparatus for local polymerization of a starting material by means of dual color photopolymerization is created. The apparatus comprises the following: a container for a polymerizable starting material, light generating means arranged to provide light of a first wavelength and light of a second wavelength, the second wavelength being different from the first wavelength, and light guide means arranged to irradiate the light of the first wavelength and the light of the second wavelength into a local volume. The apparatus is arranged to perform the following processes: taking up the polymerizable starting material by the container, the starting material containing photoinitiator molecules which can be converted by means of sequential optical excitation into a reactive state in which the photoinitiator molecules locally trigger polymerization of the starting material, and thus result in photopolymerization of the starting material in a local volume by means of irradiation of the light of the first wavelength and the light of the second wavelength in a defined local volume. The following is provided in the local volume: the photoinitiator molecules are converted from an initial state, in which the photoinitiator molecules essentially do not absorb the light of the second wavelength, into an intermediate metastable ground state with changed optical properties compared to the initial state due to absorption of a photon of the first wavelength, in such a way that the photoinitiator molecules in the intermediate state absorb the light of the second wavelength. In the local volume, due to the gradual absorption of light of the first wavelength and light of the second wavelength, the photoinitiator molecules are converted from the initial state via the intermediate state into the reactive state, which locally triggers polymerization. Outside of the local volume the photoinitiator molecules which have been in the intermediate state return to the initial state.

According to one aspect, the apparatus for local polymerization of a starting material by means of dual color photopolymerization can comprise a container which can be at least partially filled with the photopolymerizable starting material.

According to another aspect, the container can comprise a working volume, wherein the working volume comprises the photopolymerizable starting material, especially the volume of the photopolymerizable starting material, which is irradiated with light of at least one of light of a first wavelength and light of a second wavelength.

According to one aspect, the light of the first wavelength is provided as a light sheet and the light of the second wavelength is provided as an image, wherein the position of the light sheet with respect to the working volume can be changed, preferably in a translational motion, and the image can be changed depending on the position of light sheet. To give an example, a container containing the photopolymerizable starting material is moved relative to a light sheet, while a movie is projected onto the light sheet from an orthogonal direction. It may be preferably provided in this regard that the light of the first wavelength is irradiated from at least two opposite sides, creating an overlapping light section or light sheet of the light of the first wavelength, more preferably the light of the first wavelength is irradiated from at least four directions, e.g. through the four side walls of a rectangular container, in which case the irradiation of the light of the second wavelength can be made through the bottom or lid, preferably the bottom, of the container.

In the process for volumetric printing of a shaped body, the local photopolymerisation leads to hardening or curing of the starting material and thus to production of the shaped body.

The absorptive capacity of the photoinitiator molecules in the intermediate state with respect to light of the second wavelength can be based on a change in the optical properties of the photoinitiator molecules, by which the absorptive capacity for light of the second wavelength is initially formed, in particular in that a spectral absorption band is formed in the region of the second wavelength due to the transition from the initial state to the intermediate state. Alternatively, it can be provided that an absorption band for light of the second wavelength already existing in the initial state of the photoinitiator molecules is amplified or enlarged in the intermediate state.

In the intermediate state, the photoinitiator molecules in the range of the first wavelength may be still absorbing or no longer absorbing. Preferably, the photoinitiator molecules in the intermediate state may only sparsely absorb the light of the first wavelength. Further preferred, the photoinitiator molecules in the intermediate state may absorb the light of the first wavelength as little as possible. More preferred, the photoinitiator molecules in the intermediate state may substantially not absorb the light of the first wavelength. Most preferred, the photoinitiator molecules in the intermediate state may not absorb the light of the first wavelength.

In one aspect, the intermediate state may return thermally at the printing temperature in the printing resin to the initial state. Preferably, the intermediate state may return thermally at the printing temperature to the initial state in a mechanism with one or more rate constants with the highest rate constant higher than k=0.01 s⁻¹. Especially preferably, at least one rate constant for the thermal back reaction is higher than 0.02 s⁻¹, more preferably, higher than 0.1 s⁻¹, even more preferably higher than 0.25 s⁻¹, most preferably higher than 0.65 s⁻¹, but optionally not higher than 10 s⁻¹. Hence, at least one rate constant may be in the range of 0.01 s⁻¹ and 1 s⁻¹, or any other range which may be formed from the values above. In the various embodiments, local photopolymerization in the local area can lead to hardening or curing of the starting material.

Light of the first and second wavelength can be irradiated simultaneously into the local volume.

Light of the second wavelength can be irradiated into the local volume after the irradiation of the light of the first wavelength into the local volume has ended, the light of the second wavelength being irradiated before the end of a decay time of the intermediate state of the photoinitiator molecules.

Due to the absorption of light of the second wavelength, the photoinitiator molecules can be converted into a reactive state, which triggers polymerization in the local volume. It may be intended that this reactive state is produced in analogy to a Norrish type I reaction or a Norrish type II reaction which is known to the skilled person. Alternatively, it may be intended that this reactive state undergoes an electron transfer reaction with a co-initiator.

Dual color photoinitiators can be used, which respond to electromagnetic radiation of a first wavelength by switching from a thermodynamically stable state A to a metastable state B. B is able to absorb electromagnetic radiation of a second wavelength, thereby forming C, that can initiate a polymerization reaction. B can undergo a fast thermal reverse reaction to form A and is then deactivated for electromagnetic radiation of the second wavelength. Application of such dual color photoinitiators allows the curing of a photopolymerizable resin in any volume, where electromagnetic radiation of both wavelengths intersects, for example where an image is projected on a light sheet. Certain substitution patterns ensure a fast thermal back reaction and no curing with the first wavelength alone, preventing the curing in unwanted areas. Application of the disclosed dual color photoinitiators in a polymerizable mixture allows fast volumetric printing with high resolution. No support structures are required, which saves material and allows for the fabrication of soft and fragile products. A broad range of possible resin viscosities can be covered, and high reactivity is achieved, due to minimized quenching of the polymerization by oxygen and water. The utilization of the disclosed dual color photoinitiators allows faster printing than common photopolymerization based additive manufacturing techniques due to fewer mechanical operations.

The dual color photoinitiators may be characterized in a way that a polymerization is induced where electromagnetic radiation of two different wavelengths interacts with the same volume of polymerizable material in a simultaneous or consecutive fashion. In volumes which interact with electromagnetic irradiation of only one wavelength, less or no polymerization is induced.

A dual color photoinitiator in the thermodynamically stable form A may absorb a photon from electromagnetic radiation of the first wavelength which induces an isomerization reaction to the metastable form B. The dual color photoinitiator in the metastable form B absorbs a photon from electromagnetic radiation of the second wavelength, which results in an excited state C and further causes the formation of radicals by hydrogen abstraction from a co-initiator followed by electron transfer or decomposition into radicals, by electron transfer which is followed by hydrogen abstraction or decomposition into radicals, or homolytic bond cleavage which can be preceded or followed by other rearrangement reactions to form radicals. Dual color photoinitiators in the metastable state B which have not absorbed a photon from electromagnetic radiation of the second wavelength, return spontaneously via a thermal process to the thermodynamically stable state A.

The dual color photoinitiators may carry carbonyl functions which are weak triplet sensitizers for spiropyrans and related structures and therefore show no curing with UV light alone. Electromagnetic radiation of the first wavelength causes excitation of the initiator in form A and switching to the initiator in form B mainly via a singlet state. The efficient ring opening reaction of the photoswitch motif prevents the dual color initiators from radical formation by irradiation with the first wavelength alone. The merocyanine type form B can act as an internal triplet sensitizer upon irradiation with the second wavelength, which causes the carbonyl group to abstract a hydrogen atom from a co-initiator, undergo an electron transfer reaction, or undergo homolytic bond cleavage. The substituents are chosen in a way to minimize or extinguish the absorption of the merocyanine type form B at the first wavelength and that form B is thermodynamically destabilized to ensure a fast thermal back reaction from B to A. Furthermore, the disclosed dual color initiators benefit from an exceptionally low or neglectable quantum yield for the competing photoreaction from B to A and high extinction coefficients of the B form, where form A does not absorb, preferably at the second wavelength. The merocyanine form B typically has a broad absorption in the visible region, which allows for high intensities over a broad range of wavelengths.

According to one aspect the dual color Photoinitiator may be free of halogen atoms.

According to one aspect the intermediate state B can be an excited triplet state and/or the reactive state C can be a higher excited state, preferably a higher excited triplet state.

Due to the absorption of the light of the second wavelength, the photoinitiator molecules can be converted into a reactive state which triggers a radical polymerization in the local volume.

A light beam of light of the first wavelength and a light beam of light of the second wavelength can be irradiated in the local volume at least partially overlapping.

A light sheet of light of the first wavelength and an image projection of light of the second wavelength can be irradiated in the local volume at least partially overlapping.

The starting material can be polymerized in several local volumes by means of photo polymerization and thus a three-dimensional shaped body can be produced in the starting material.

The dual color photoinitiators are not limited to a specific setup, but to give an illustrative example which shows the necessity of the thermal back reaction the following setup can be used: Volumetric printing can be conducted in a setup, which consists of a light sheet generator, a projector, a container that has four transparent sides and contains the resin including at least one of the disclosed dual color photoinitiators and a moveable stage to move either the container or the light sheet generator. Such a setup ensures a fast curing process over the whole volume, compared to the point by point setups described by Swainson [Swainson, U.S. Pat. Nos. 4,041,476, 4,078,229, 4,238,840, 4,466,080, 4,471,470, 4,333,165]. One layer of a volume is irradiated with electromagnetic radiation of a first wavelength to switch the dual color photoinitiators from A to B. An image is projected on that layer from a different direction, using electromagnetic radiation of the second wavelength, which induces a photopolymerization, where image and layer overlay. After a given amount of time, the electromagnetic radiation of the first wavelength is shifted to a neighboring layer and the next image is projected on this layer with electromagnetic radiation of the second wavelength. In the previous layer no further polymerization occurs due to the fast thermal back reaction to the A form, which causes a deactivation for electromagnetic radiation of the second wavelength. After a number of iteration steps, the 3-dimensional object is cured in the volume and can be taken out, or the residual resin can be washed away. The remaining object can be subjected to a post-processing.

The post-processing may comprise at least one of the following steps:
removal of the object from the container;
removal of residual or uncured polymerizable starting materials and/or constituents of the polymerizable starting materials and/or residual or uncured resin from the object;
washing the object with at least one solvent, preferably a solvent which does not alter the object, e.g. by swelling;
immersing the object in a solvent containing a thermal initiator or a photoinitiator;
drying of the object, preferably by a gas stream or heat;
post curing by irradiation or heating, preferably under a protective gas atmosphere, preferably an atmosphere of nitrogen, argon, or carbondioxide;
post-irradiation after post-curing, preferably using visible light; polishing of the object;
coating of the object.

Any atmosphere inside of the apparatus or around the object can be filtered, preferably to remove dust particles, preferably during the manufacturing process of the object. Preferably the manufacture can be performed in a clean room environment.

The product of the process, such as the 3-dimensional object, may be an optical element, wherein the term “optical element” refers to an object having a transparency of 90% or more.

The optical element can be refractive or diffractive optical element, preferably a lens, such as a convex or concave lens, more preferably an aspheric lens, most preferably a freeform lens.

In one embodiment,
the light of the first wavelength is irradiated into the local volume from a first direction and from a second direction different from the first direction;
the light of the first wavelength irradiated into the local volume from the first direction has a first intensity or a first intensity distribution;
the light of the first wavelength irradiated into the local volume from the second direction has a second intensity or a second intensity distribution

The first intensity or first intensity distribution can be different from the second intensity or second intensity distribution. The first intensity can be different from the second intensity. The first intensity distribution can be different from the second intensity distribution

Irradiating the photopolymerizable starting material from at least two different directions (including the first direction and the second direction) can be achieved by at least one of:
motion of the local volume relative to at least one (static) radiation device emitting light of a first wavelength;
motion of at least one radiation device emitting the light of the first wavelength relative to the (static) local volume
motion of the local volume and motion of at least one irradiation device emitting the light of a first wavelength.

As such, the local volume and/or the radiation device can be moveably supported in at least one degree of freedom of motion which can be or comprise a translatory and/or rotatory degree of freedom of motion.

In either case, irradiating the photopolymerizable starting material can be effected simultaneously or sequentially with moving the local volume relative to the at least one radiation device. Typically, the resulting intensity distribution within the local volume depends on the intensity of the light, the irradiation direction, the irradiation location, the absorption properties of the photopolymerizable staring material and the absorption properties of the photopolymerized material, respectively.

The irradiating of the photopolymerizable starting material with light of the first wavelength into the local volume can be performed wherein the working volume is not moved relative to a radiation device and the radiation device is shaped in a way that light emitted from the radiation device travels different distances from the radiation device to the local volume, for example is shaped as conical or pyramidal.

One or more radiation device(s) can be used emitting the light of the first wavelength at a specific angle or a range of angles with respect to the surface of the photopolymerizable starting material, which includes other angles than 90°, preferably at least two angles different from 90°.

The light of the first wavelength can be irradiated into the local volume from more than two different directions (first direction, second direction, third direction etc.), preferably from five or more different directions.

The light of the first wavelength can be irradiated into the local volume in the form of an image, wherein the same image or different images can be irradiated from the different directions (first direction, second direction, third direction etc.). The light of the first wavelength may be irradiated into the local volume in parallel or diverging.

The intensity of the light of the first wavelength irradiated into the local volume from a first direction and the intensity of the light of the first wavelength irradiated into the local volume from a second direction (and optionally that of any further direction) may be selected to maximize the absorbed light (absorbed dose) in the local volume (to be polymerized) and to minimize the absorbed light (absorbed dose) in the remaining volume (not to be polymerized). It may be provided that the local volume (to be polymerized) is a volume in which a threshold of absorbed light is exceeded.

According to one aspect the polymerizable starting material has an optical density referring to an optical path length of 10 mm according to the Beer-Lambert law of <0.1 at at least one wavelength between 400 nm and 2000 nm, preferably at least one wavelength between 450 nm and 1500 nm. Alternatively, the polymerizable starting material is characterized by an optical density referring to an optical path length of 10 mm according to the Beer-Lambert law of <0.1 at at least one wavelength between 400 nm and 1000 nm, preferably at least one wavelength between 450 nm and 900 nm, more preferably at least one wavelength between 500 nm and 800 nm. The optical density can be measured using a Cary 60 UV-Vis Spectrophotometer from Agilent Technologies.

According to one aspect the printed object has an optical density referring to an optical path length of 10 mm according to the Beer-Lambert law of <0.1 at at least one wavelength between 400 nm and 2000 nm, preferably at least one wavelength between 450 nm and 1500 nm. Alternatively, the printed object is characterized by an optical density referring to an optical path length of 10 mm according to the Beer-Lambert law of <0.1 at at least one wavelength between 400 nm and 1000 nm, preferably at least one wavelength between 450 nm and 900 nm, more preferably at least one wavelength between 500 nm and 800 nm. The optical density can be measured using a Cary 60 UV-Vis Spectrophotometer from Agilent Technologies.

The first wavelength and/or the second wavelength can comprise a range of wavelengths, respectively.

According to one aspect, the light of the first wavelength and the light of the second wavelength each comprises light of at least one wavelength between 300 nm and 1000 nm, wherein the highest intensity or peak emission of the light of the first wavelength is at a lower wavelength than the wavelength of the highest intensity or peak emission of the light of the second wavelength.

Typical polymerization parameters which are suitable for volumetric printing may be:
any setup which employs light as described above;
a temperature of −20° C. to +100° C.; preferably 0° C. to +60° C., more preferably +20° C. to +60° C.
a first wavelength of: 300 nm to 500 nm; preferably, 325 nm to 450 nm, more preferably 350 nm to 425 nm, most preferably 375 nm or 405 nm and
if used, a second wavelength of: 350 nm to 1000 nm, preferably 450 nm to 900 nm, more preferably 500 nm to 800 nm.

To prevent migration and release of remaining photoinitiator and/or their reaction products from the cured object the following measures are disclosed:
a) Substitution in a way, that the molecular weight exceeds 1000 g/mol
b) Binding of the photoinitiator to a structure of higher molecular weight, such as an oligomer or a polymer
c) Functionalization of the initiator with polymerizable groups, which are build in the polymer network during curing, these include but are not limited to: acrylates, acrylamides, methacrylates, thiol+ene, epoxides, oxiranes, oxetanes, vinylethers.

The initial state of the photoinitiator in the photopolymerizable material has an extinction coefficient at the first wavelength which is lower than 5000 L mol⁻¹ cm⁻¹, preferably lower than 2500 L mol⁻¹ cm⁻¹, more preferably lower than 1000 L mol⁻¹ cm⁻¹, even more preferably lower than 500 L mol⁻¹ cm⁻¹, most preferably lower than 250 L mol⁻¹ cm⁻¹; and/or

The initial state of the photoinitiator in the photopolymerizable material may have an extinction coefficient at the first wavelength which is higher than 30 L mol⁻¹ cm⁻¹, preferably higher than 70 L mol⁻¹ cm⁻¹, more preferably higher than 100 L mol⁻¹ cm⁻¹, even more preferably higher than 150 L mol⁻¹ cm⁻¹, most preferably higher than 200 L mol⁻¹ cm⁻¹, like higher than 210 L mol⁻¹ cm⁻¹. Hence, the initial state of the photoinitiator in the photopolymerizable material may have an extinction coefficient at the first wavelength which is in the range of 5000-0 L mol⁻¹ cm⁻¹, preferably 2500-10 L mol⁻¹ cm⁻¹, more preferably 1000-20 L mol⁻¹ cm⁻¹, even more preferably 500-50 L mol⁻¹ cm⁻¹, most preferably 300-100 L mol⁻¹ cm⁻¹, like 300-210 L mol⁻¹ cm⁻¹.

The photopolymerizable material has an absorbance at the first wavelength in a range between 1 and 0.01 absorbance units, preferably in a range between 1 and 0.05 absorbance units, more preferably in a range between 0.8 and 0.05 absorbance units, even more preferably in a range between 0.7 and 0.1 absorbance units corresponding to a path length of 1 cm. The absorbance of the photopolymerizable material may be measured by UV/-Vis spectrometry using a Cary60 UV-Vis spectrophotometer supplied by Agilent Technologies. Acetonitrile may be used as a reference (baseline) to determine the absorbance. The first wavelength may be 250 nm to 500 nm; preferably 300 nm to 450 nm, more preferably 360 nm to 420 nm, most preferably 375 nm or 405 nm.

The photopolymerizable material has an absorbance at the first wavelength in a range between 1 and 0.01 absorbance units, preferably in a range between 1 and 0.05 absorbance units, more preferably in a range between 0.8 and 0.05 absorbance units, even more preferably in a range between 0.7 and 0.1 absorbance units which may correspond to a path length which equals the path length the light of the first wavelength has to travel through the container used for volumetric printing. The absorbance of the photopolymerizable material may be measured by UV/-Vis spectrometry using a Cary60 UV-Vis spectrophotometer supplied by Agilent Technologies. Acetonitrile may be used as a reference (baseline) to determine the absorbance. The first wavelength may be 250 nm to 500 nm; preferably 300 nm to 450 nm, more preferably 360 nm to 420 nm, most preferably 375 nm or 405 nm.

Furthermore, preferably, the photoinitiator molecules in the intermediate state substantially do not absorb the light of the first wavelength.

Preferably, the photoinitiator molecules in the initial state have an extinction coefficient at the second wavelength which is lower than 2000 L mol⁻¹ cm⁻¹, more preferably lower than 1000 L mol⁻¹ cm⁻¹, even more preferably lower than 500 L mol⁻¹ cm⁻¹, still more preferably lower than 200 L mol⁻¹ cm⁻¹, most preferably lower than 100 L mol⁻¹ cm⁻¹.

Preferred spectral properties of dual color photoinitiators can be investigated in a volumetric printing setup, where a cuvette with four transparent windows is irradiated in one direction with a light sheet of light of the first wavelength, while an image is projected onto the light sheet from a perpendicular direction with light of the second wavelength. The image is changed to produce a movie, while the cuvette is moved through the light sheet. The prints may result in solidification only in volumes where the light of both wavelengths intersects. The residual uncured resin is removed to obtain the shaped body, which is further washed with solvent and post-processed.

While photochemistry is typically performed at a wavelength, where the absorbing species has its absorption maximum, such attempts do not result in successful prints when the light sheet is generated at a wavelength around the absorption maximum of the initial state. Instead, only curing at the walls of the cuvette is observed. This may be explained by Beer's law, since at the wavelength around the maximum, the photons are absorbed in close proximity to the cuvette wall, causing also the polymerization in close proximity to the cuvette wall, while only a minor fraction of the light reaches the middle of the cuvette.

To circumvent this problem, the concentration can been lowered, so that the absorption at the absorption maximum/first wavelength can be reduced to 1 or less.

When the penetration depth issue is solved by reducing the concentration, no curing is observed at all, since the concentration of the dual color photoinitiator is too low to allow the formation of a sufficient number of radicals.

Surprisingly, prints in the middle of the cuvette are successful without curing at the walls, when the first wavelength was chosen to irradiate in the red tail of the absorption spectrum, where the initial state has a low extinction coefficient. This is counter-intuitive, as one would expect less radicals to be formed, when the initiator absorbs less photons, but surprisingly more radicals are formed in the middle of the cuvette. However, when the first wavelength is shifted further, where the initial state has an even lower extinction coefficient, no curing is observed anymore or the intensity is largely increased, resulting in only curing at the cuvette walls. When the concentration is increased to an extent, that curing can be observed, the curing is caused mainly at the walls again. This may be explained by little impurities, with a relatively higher extinction coefficient than the initial state or the relatively higher intensity of the first wavelength necessary to cause the switching. By comparing prints of different concentrations and first wavelengths, the following conditions have been found to result in improved print results compared to the state of the art. When the first wavelength is adjusted accordingly to the extinction coefficient, the concentration of the initiator can be sufficiently increased to achieve a dual color effect. Surprisingly, these conditions are independent of the structure of the initiator itself, as they rather seem to be related to the surprising combination of restrictions given by Beer's law and the dual color effect:
3) The extinction coefficient of the dual color photoinitiator in the photopolymerizable starting material at the first wavelength should be in the range of 5000-0 L mol⁻¹ cm⁻¹, preferably 2500-10 L mol⁻¹ cm⁻¹, more preferably 1000-20 L mol⁻¹ cm⁻¹, even more preferably 500-50 L mol⁻¹ cm⁻¹, most preferably 300-100 L mol⁻¹ cm⁻¹.
4) The absorption of the resin at the first wavelength over a distance of 1 cm, using a non-absorbing solvent as reference, e.g. acetonitrile, should be in the range of 1-0.05, preferably 0.8-0.5, more preferably 0.7-0.10, or
5) The absorption of the resin at the first wavelength over the path length the light of the first wavelength has to travel through the container, using a non-absorbing solvent as reference, e.g. acetonitrile, should be in the range of 1-0.05, preferably 0.8-0.5, more preferably 0.7-0.10.

Surprisingly, the preferred properties of the photoinitiator and the resin are independent of the object which shall be printed but rely on the size of the container and the path length the light of the first wavelength has to travel. Since Beer's law involves the path length of the light, the problem of penetration depth is especially important for larger build rooms, exceeding 1×1×1 mm³.

The photoinitiator molecule comprises the following structural motif (BI)

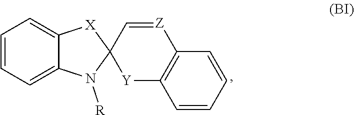

wherein
X is selected from the group consisting of S, CR^{S}₂, and NR^{S};
Y is selected from the group consisting of O, S, and NR^{S};
Z is selected from the group consisting of N and CR^{S′}; and
   R^{S} are independently selected.

In the structural motif (BI), the different R and R^{S} are independently arbitrary groups known in the art as substituents in organic compounds.

The different R and R^{S} in the structural motif (BI) may be independently selected from the group consisting of H, D, halogen, NO₂, CN, OH, SH, substituted or unsubstituted C₁-C₂₀-alkyl, substituted or unsubstituted C₃-C₂₀-cycloalkyl, substituted or unsubstituted C₅-C₄₈-aryl, substituted or unsubstituted C₁-C₂₀-alkyl-C₅-C₄₈-aryl, preferably C₁-C₂₀-alkyl-electron withdrawing-C₅-C₄₈-aryl; substituted or unsubstituted C₃-C₄₂-heteroaryl, substituted or unsubstituted C₁-C₂₀-alkyl-C₃-C₄₂-heteroaryl, preferably C₁-C₂₀-alkyl-electron withdrawing-C₃-C₄₂-heteroaryl; substituted or unsubstituted C₂-C₄₉-alkyl acyl, substituted or unsubstituted C₂-C₄₉-aryl acyl, substituted or unsubstituted C₁-C₂₀-alkoxy, substituted or unsubstituted C₅-C₄₈-aryloxy, C₁-C₂₀-alkyl-C₅-C₄₈-aryloxy, C₅-C₄₈-aryl-C₁-C₂₀-alkoxy, NH₂, substituted or unsubstituted C₁-C₂₀-alkyl ester, substituted or unsubstituted C₅-C₄₈-aryl ester, substituted or unsubstituted C₁-C₂₀ alkyl amide, substituted or unsubstituted C₅-C₄₈-aryl amide, NR′₂, SiR′₃, —O—SiR′₃, substituted or unsubstituted carboxylic acids and salts thereof, substituted or unsubstituted sulfonic acids and salts thereof, substituted or unsubstituted sulfonic esters, substituted or unsubstituted sulfonic amides, formyl, ether, thioether, carbonate, carbonate ester, sulfates, boronic acids, boronic esters, phosphonic acids, phosphonic esters, phosphines, phosphates, peroxycarbonic acids, thiocarbonic acids, sulfinic acids, sulfinic esters, sulfonates, thiolesters, sulfoxides, sulfones, hydrazides, thioaldehydes, ketones, thioketones, oximes, hydrazines, nitroso, azo, diazo, diazonium; isocyanides, cyanate, isocyanate, thiocyanate, isothiocyanate, hydroperoxide, peroxide, acetals, ketal, orthoester, orthocarbonate esters, ammonium, imines, imides, azide, nitrate, isonitrile, nitrosoxy, substituted or unsubstituted carbamates, substituted or unsubstituted ethers, substituted or unsubstituted polyether carbamates, substituted or unsubstituted arylazo, substituted or unsubstituted C₂-C₂₀-alkynyl, substituted or unsubstituted C₂-C₂₀-alkenyl, or any group from Table 1 in “A survey of Hammett substituent constants and resonance and field parameters”, Chem. Rev. 1991, 91, 2, 165-195.

In terms of the present disclosure, n in Cn designates the number of carbon atoms in the moiety following the Cn. For example, C₁-C₂₀-alkyl-C₅-C₄₈-aryl designates an alkyl group having 1 to 20 carbon atoms which is substituted with an aryl group having 6 to 48 carbon atoms. In another example, C₁ to C₂₀-alkyl acyl refers to an alkyl acyl group having 2 to 21 carbon atoms in total. In terms of the present disclosure, the groups (substituents) referred to herein may be positively or negatively charged, e.g. C₅-aryl may be cyclopentadienyl (Cp⁻).

Two R^{s} attached to the same atom, e.g. if X is CR^{s}₂, may be linked to each other to form a ring.

R′ may be independently selected from the group consisting of substituted or unsubstituted C₁-C₂₀-alkyl and substituted or unsubstituted C₅-C₄₈-aryl, and two R′ may form a ring structure.

If one or more of R and R^{S} and/or a ring formed by two or more thereof is substituted, the respective substituent(s) can be selected from the group consisting of D, halogen, NO₂, CN, OH, SH, substituted or unsubstituted C₁-C₂₀-alkyl, substituted or unsubstituted C₃-C₂₀-cycloalkyl, substituted or unsubstituted C₅-C₄₈-aryl, substituted or unsubstituted C₁-C₂₀-alkyl-C₅-C₄₈-aryl, preferably C₁-C₂₀-alkyl-electron withdrawing-C₅-C₄₈-aryl; substituted or unsubstituted C₃-C₄₂-heteroaryl, substituted or unsubstituted C₁-C₂₀-alkyl-C₃-C₄₂-heteroaryl, preferably C₁-C₂₀-alkyl-electron withdrawing-C₃-C₄₂-heteroaryl; substituted or unsubstituted C₂-C₄₉-alkyl acyl, substituted or unsubstituted C₂-C₄₉-aryl acyl, substituted or unsubstituted C₁-C₂₀-alkoxy, substituted or unsubstituted C₅-C₄₈-aryloxy, C₁-C₂₀-alkyl-C₅-C₄₈-aryloxy, C₅-C₄₈-aryl-C₁-C₂₀-alkoxy, NH₂, substituted or unsubstituted C₁-C₂₀-alkyl ester, substituted or unsubstituted C₅-C₄₈-aryl ester, substituted or unsubstituted C₁-C₂₀ alkyl amide, substituted or unsubstituted C₅-C₄₈-aryl amide, NR′₂, SiR′₃, —O—SiR′₃, substituted or unsubstituted carboxylic acids and salts thereof, substituted or unsubstituted sulfonic acids and salts thereof, substituted or unsubstituted sulfonic esters, substituted or unsubstituted sulfonic amides, formyl, ether, thioether, carbonate, carbonate ester, sulfates, boronic acids, boronic esters, phosphonic acids, phosphonic esters, phosphines, phosphates, peroxycarbonic acids, thiocarbonic acids, sulfinic acids, sulfinic esters, sulfonates, thiolesters, sulfoxides, sulfones, hydrazides, thioaldehydes, ketones, thioketones, oximes, hydrazines, nitroso, azo, diazo, diazonium; isocyanides, cyanate, isocyanate, thiocyanate, isothiocyanate, hydroperoxide, peroxide, acetals, ketal, orthoester, orthocarbonate esters, ammonium, imines, imides, azide, nitrate, isonitrile, nitrosoxy, substituted or unsubstituted carbamates, substituted or unsubstituted ethers, substituted or unsubstituted polyether carbamates, substituted or unsubstituted arylazo, substituted or unsubstituted C₂-C₂₀-alkynyl, substituted or unsubstituted C₂-C₂₀-alkenyl. R′ may be independently selected from the group consisting of substituted or unsubstituted C₁-C₂₀-alkyl and substituted or unsubstituted C₅-C₄₈-aryl, and two R′ may form a ring structure, wherein the respective substituents can, in turn be preferably be substituted with D or C₁ to C₆ alky.

It may be provided that if one or more of R and R^{S} and/or a ring formed by two or more thereof is substituted, the respective substituent(s) may be independently selected from the group consisting of D, methyl, tert-butyl, cyclohexyl, phenyl, CF₃, F, Cl, Br, I, CN, nitro, formyl, ethylester, methylester, acetyl, methoxy, OH, dimethylamino, NH₂, amide (CO—NH₂), carboxylic acid or salts thereof, sulfonic acid or salt thereof, methylsulfone, and sulfonamide.

Preferably X is selected from S or CR^{S}₂. More preferably X is CR^{S}₂ and R^{s} is independently selected from H, D, C₅-C₄₈-aryl, C₆-C₁₀-aryl, C₁-C₂₀-alkyl, C₁-C₁₀-alkyl, C₁-C₆-alkyl, and C₁-C₄-alkyl or the two R^{S} form together with the C (the atom X in the structural motif (BI)) a substituted or unsubstituted C₁ to C₂₀ cycloalkyl ring. More preferably, X is CR^{S}₂ and the two R^{S} are methyl or the two R^{S} form together with the C (the atom X in the structural motif (BI)) a C₁ to C₆ cycloalkyl ring, preferably a cyclohexyl ring.

Preferably Z is CR^{S}. R^{S} may be independently selected from H, D, CN, C₁-C₁₂-alkyl, fluorinated C₁-C₁₂-alkyl, or C₆-C₁₄-aryl. More preferably, Z is CR^{S} and R^{S} is H.

Preferably, Y is S or O. More preferably Y is O.

The structural motif (BI) may be the structural motif (BII)

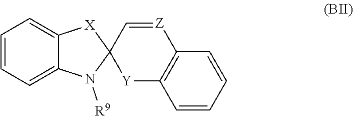

That is, the photoinitiator molecule may comprise the structural motif (BII).

In the structural motif (BII),
X is selected from the group consisting of S, CR^{S}₂, and NR^{S};
Y is selected from the group consisting of O, S, and NR^{S};
Z is selected from the group consisting of N and CR^{S};
R^{S} are independently selected; and
R⁹ is alkyl electron withdrawing aryl or alkyl electron withdrawing heteroaryl.
   R^{S} is as defined above.

While Hammet constants are typically used to describe the electron withdrawing or electron donating nature of a substituent, such a description has limits, since Hammet constants are neither described for substituents in the ortho position nor are they suitable where several substituents are in place or the electron withdrawing/donating nature of a whole group is at quest. A simple way to describe the electron withdrawing or donating nature of a whole group is to use a reference compound with a “marker”, where the “marker” resembles the substituent group and the rest of the molecule. The chemical shift of an NMR signal is related to the electron density and therefore the NMR signal corresponding to the marker group can be used to define the electron withdrawing or electron donating nature of the functional group. To give an example a benzyl substituent links a phenyl ring via a CH₂ group to a molecule. Replacing that molecule by “H”, yields the compound toluene, where the hydrogen of the methyl group experiences the same electron withdrawing effect/donating effect as the replaced molecule experiences when substituted with benzyl and hence the chemical shift of the methyl group can be used as a measure for the nature of the benzyl substituent.

The term “alkyl electron withdrawing aryl” as used herein refers to an alkyl group (which can be referred to as alkylene), preferably a C1-alkylene group (=methylene; —CH₂—), substituted with an electron withdrawing aryl group.

The term “electron withdrawing aryl group” as used herein refers to a substituted aryl group, wherein the one or more substituent(s) of the substituted aryl group is/are independently selected in a way that the ¹H-NMR (CDCl₃) signal (chemical shift) of the bold printed hydrogen in a compound of the formula

(substituted aryl group)-CH₂—H
is larger than the ¹H-NMR (CDCl₃) signal of the bold printed hydrogen in a compound of the formula

### Phenyl-CH₂—H.

The term “alkyl electron withdrawing heteroaryl” as used herein refers to an alkyl group (which can be referred to as alkylene), preferably a C1-alkylene group (=methylene; —CH₂—), substituted with an electron withdrawing heteroaryl group. The term “heteroaryl” in this regard encompasses a polycyclic planar fragment, wherein at least one of the atoms of the two or more (poly) cycles is a heteroatom, alternatively or in particular encompasses a derivative of coumarin, xanthone or thioxanthone.

The term “electron withdrawing heteroaryl group” as used herein refers to an unsubstituted or substituted heteroaryl group, wherein the one or more substituent(s) of the substituted heteroaryl group, if present, or the heteroaryl itself is/are selected in a way that the ¹H-NMR (CDCl₃) signal (chemical shift) of the bold printed hydrogen in a compound of the formula

(substituted heteroaryl group)-CH₂—H
is larger than the ¹H-NMR (CDCl₃) signal of the bold printed hydrogen in a compound of the formula

### Phenyl-CH₂—H.

Exemplary compounds (substituted heteroaryl group)-CH₂—H, (substituted aryl group)-CH₂—H and Phenyl-CH₂—H are listed in the following Table 1.

TABLE 1

| | ¹H-NMR (CDCl₃) |
|---|---|
| Structure | [ppm] |
| 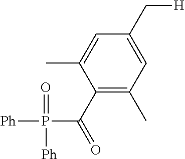 [SMILES: Cc1cc(C)c(C(=O)P(=O)(c2ccccc2)c2ccccc2)c(C)c1] | 2.253 |
| 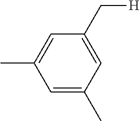 | 2.26 |
| 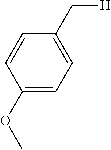 | 2.26 |
| 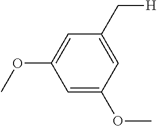 | 2.28 |
| 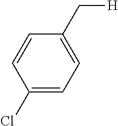 | 2.29 |
| 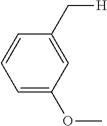 | 2.30 |
| 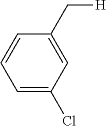 | 2.31 |
| 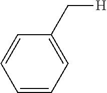 | 2.34 |
| 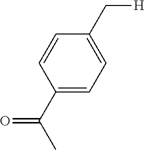 | 2.38 |
| 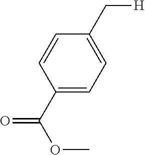 | 2.39 |
| 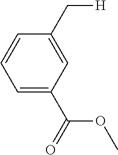 | 2.39 |
| 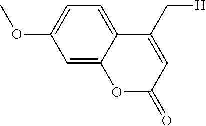 | 2.39 |
| 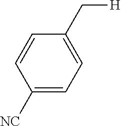 | 2.41 |
| 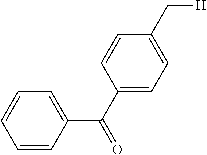 [SMILES: Cc1ccc(C(=O)c2ccccc2)cc1] | 2.42 |
| 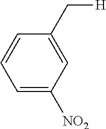 | 2.46 |
| 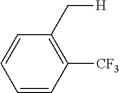 [SMILES: Cc1ccccc1C(F)(F)F] | 2.47 |
| 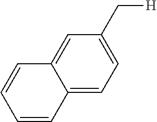 [SMILES: Cc1ccc2ccccc2c1] | 2.50 |
| 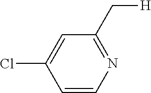 | 2.55 |
| 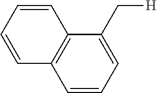 [SMILES: Cc1cccc2ccccc12] | 2.65 |
| 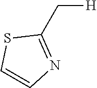 | 2.68 |
| 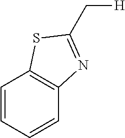 | 2.79 |

Source: https://sdbs.db.aist.go.jp/Disclaimer.aspx (National Institute of Advanced Industrial Science and Technology/AIST), Japan at the priority date

¹H-NMR shifts of a large number of compounds are known in the art and described in the literature, e.g. The spectral database for Organic Compounds freely available and organized by the National Institute of Advanced Industrial Science and Technology/AIST), Japan.

Preferably X is selected from S or CR^{S}₂. More preferably X is CR^{S}₂ and R^{S} is independently selected from H, D, C₅-C₄₈-aryl, C₆-C₁₀-aryl, C₁-C₂₀-alkyl, C₁-C₁₀-alkyl, C₁-C₆-alkyl, and C₁-C₄-alkyl or the two R^{S} form together with the C (the atom X in the structural motif (BII)) a substituted or unsubstituted C₁ to C₂₀ cycloalkyl ring. More preferably, X is CR^{S}₂ and the two R^{S} are methyl or the two R^{S} form together with the C (the atom X in the structural motif (BII)) a C₁ to C₆ cycloalkyl ring, preferably a cyclohexyl ring.

Preferably Z is CR^{S}. R^{S} may be independently selected from H, D, CN, C₁-C₁₂-alkyl, fluorinated C₁-C₁₂-alkyl, or C₆-C₁₄-aryl. More preferably, Z is CR^{S} and R^{S} is H.

Preferably, Y is S or O. More preferably Y is O.

Dual color photoinitiators according to structure (BII), wherein R⁹ is an alkyl electron withdrawing aryl group, e.g. an electron withdrawing benzylic group, show unexpected advantages with respect to dual color photoinitiators which have been used in the state of the art. Although the electron withdrawing group is not in conjugation with the spiropyran nitrogen atom, it has a surprisingly large influence on the kinetics of the thermal back reaction. For the reasons of good initiation reactivity, ideal spectral properties, high quantum yields for switching to the merocyanine, and low quantum yields for a potential (unwanted) switching back from merocyanine to spiropyran, spiropyrans are the superior class among known dual color photoinitiators. However, when the acceptor strength on the pyran half (former salicylaldehyde) increases, the thermal back reaction is slowed down. A slow thermal back reaction reduces the resolution along the print direction in dual color volumetric printing or xolography and therefore leads to reduced applicability. A higher acceptor strength on the pyran half can be achieved by using stronger acceptors or more than one acceptor. To illustrate the effect of the R⁹ substituent, the following compounds can be compared with respect to their thermal half-life in UDMA at 23° C., where the acceptor substituted benzylic group reduces the thermal half-life from barely usable 127 s for I-23 to a usable value of 9 s for B31, which is even faster than the reference I-22 without the CF₃ group:

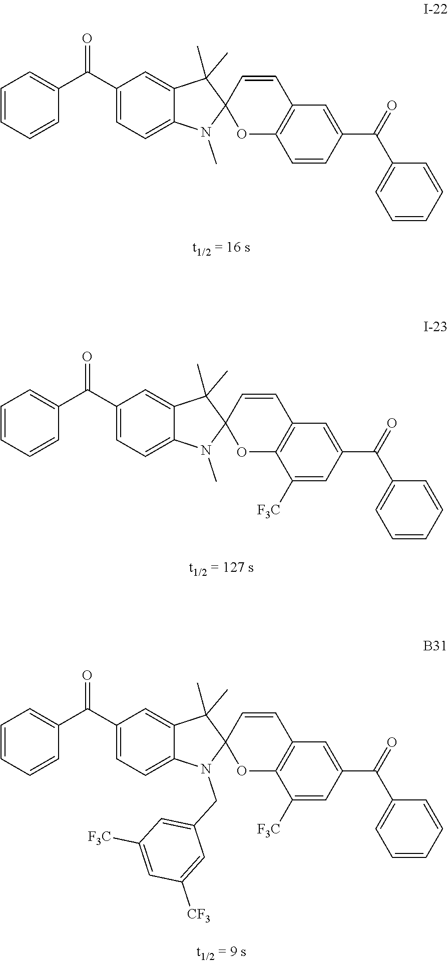 [SMILES: CN1c2ccc(C(=O)c3ccccc3)cc2C(C)(C)C12C=Cc1cc(C(=O)c3ccccc3)ccc1O2 ; CN1c2ccc(C(=O)c3ccccc3)cc2C(C)(C)C12C=Cc1cc(C(=O)c3ccccc3)cc(C(F)(F)F)c1O2 ; CC1(C)c2cc(C(=O)c3ccccc3)ccc2N(Cc2cc(C(F)(F)F)cc(C(F)(F)F)c2)C12C=Cc1cc(C(=O)c3ccccc3)cc(C(F)(F)F)c1O2]

The same applies to examples, where a strong acceptor, such as a nitro group is used, which is of particular relevance, since nitro groups are the most explored in spiropyran chemistry, comparing the thermal half-lives at 23° C. of formulations F4 (15 s) and F7 (30 s) from Table 2. The finding is general and not limited to specific acceptor substituted spiropyrans.

Another relevant situation is given for examples, where the indole part comprises a naphthyl moity. The naphthyl derivatives are typically not electron deficient enough and suffer from slow thermal back reactions. Furthermore, derivatives with electron accepting groups on the naphthyl moiety are synthetically difficult to obtain. The substitution in the R⁹ position with an alkyl electron withdrawing group provides a simple solution to the problem, since it accelerates the back reaction and is easy to introduce, comparing formulations F36 (44 s) and F37 (132 s) from Table 2.

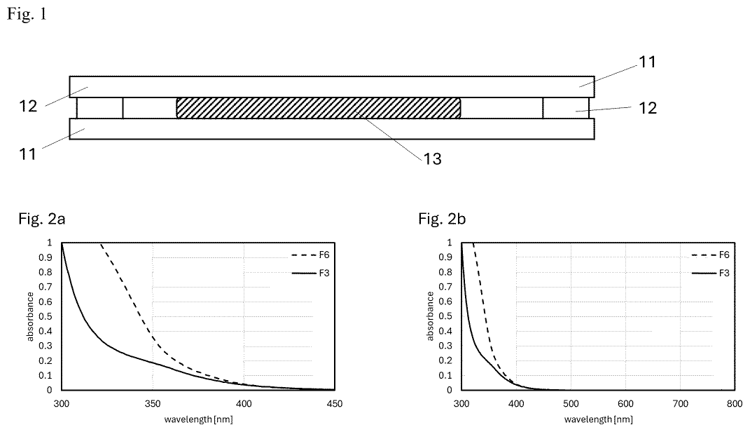

Another surprising finding is an enhanced transparency of cured objects with respect to the UV region (FIGS. 1, 2a and 2b, formulations F3 (t_{1/2} (25° C.)=3.8 s) and F6 (t_{1/2} (25° C.)=2.3 s) from Table 2). For measuring the transparency property of a resin, the photopolymerizable starting material can be placed between two glass slides with space holders of defined thickness. In the example according to FIG. 1, the space holders have a thickness of 1 mm. The arrangement according to FIG. 1 is placed in the CURE automatic exposure system (available from Rapid Shape GmbH) and cured for 20 min under full power irradiation. The resulting sample plate of the cured material has a thickness of 1 mm and absorption spectra of plates can be measured and compared (FIGS. 2a and 2b). As outlined above an acceptor is generally required on the indole part of the spiropyran to adjust the thermal half-life. However, compared to derivatives with an acceptor on the aromatic ring, placing the acceptor at R⁹ in the form of an alkyl electron withdrawing aryl generates advanced optical properties and still retains usable thermal back reaction kinetics. Without being bound by any theory, during photopolymerization the merocyanine forms a yellow species, which is covalently linked to the polymer network. By post-irradiation this yellow species may undergo a follow-up reaction to form a bleached species, which has its absorption maximum in the UV region. However, the absorption spectrum of the bleached species tails to the blue region of the spectrum, leaving a little residual color after bleaching. The structural motifs described above result in a shift of the absorption spectrum of the bleached species to even shorter wavelength, thus increasing transparency in the near UV and blue region.

It is generally difficult to remove chromophores from the inside of a printed three-dimensional object in volumetric 3d-printing processes. As an example, when a photoinitiator fragment or photoinitiator radical initiates a polymerization or reacts with a radical chain end, the resulting chromophore is bound to the polymer structure of the printed three-dimensional object and cannot be removed e.g. by extraction or washing.

Below illustrated is an example of a way of how a photoinitiator radical, PIH⋅, and a co-initiator radical, CI⋅, can be formed from a photoinitiator, PI, and a co-initiator CIH:

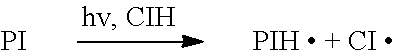

Below illustrated is an example of how a co-initiator fragment, CI, and a photoinitiator, PI, can form part of an exemplary acrylate polymer structure on basis of a radical reaction:

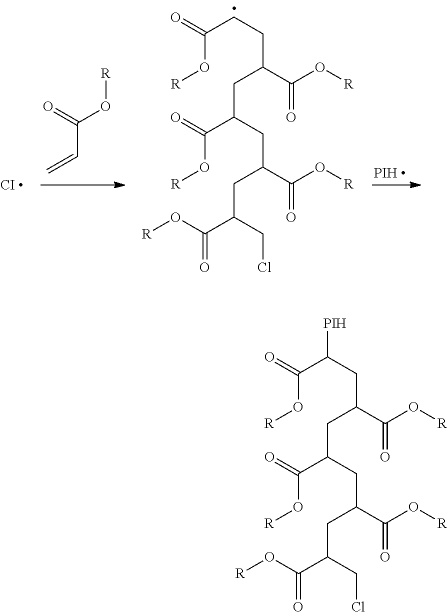

As a consequence, three-dimensional objects manufactured with volumetric 3d-printing processes typically have specific optical properties, namely absorption properties in the visible wavelength range, which result in a color or coloring, respectively. A color or coloring of the three-dimensional objects is not always desired and can indeed even be undesired for certain applications.

As such, there is a need to improve methods for manufacturing three-dimensional objects with respect to the optical properties of the three-dimensional objects which can be manufactured therewith.

In view of this need, according to one aspect of the invention, the process for locally polymerizing a starting material by dual color photopolymerization is a process for the formation of a shaped body by dual color photopolymerization comprising a further a step of post-processing the shaped body comprising a thermal treatment of the shaped body and/or an optical treatment of the shaped body.

In one embodiment a three-dimensional object may be the shaped body, or a three-dimensional object may be formed from the shaped body. Preferably a three-dimensional object may be formed from a shaped body by removal of the shaped body from the polymerizable starting material.

In one embodiment the post-processing of the three-dimensional object comprises modifying the optical properties of the three-dimensional object resulting in a decrease of the absorption properties of the three-dimensional object for at least one wavelength in a wavelength range between 300 nm and 2000 nm, preferably between 350 nm and 1000 nm, more preferably between 400 nm and 900 nm, most preferably between 450 nm and 800 nm and/or in an increase of the transmissive properties of the three-dimensional object for at least one wavelength in the wavelength range between 300 nm and 2000 nm, preferably between 350 nm and 1000 nm, more preferably between 400 nm and 900 nm, most preferably between 450 nm and 800 nm.

In one embodiment the post-processing of the three-dimensional object comprises modifying the optical properties of the three-dimensional object resulting in that an average transmission or an integral of the transmission between 300 nm and 2000 nm, preferably between 350 nm and 1000 nm, more preferably between 400 nm and 900 nm, most preferably between 450 nm and 800 nm, is increased by at least 1%, preferably at least 2%, more preferably at least 3%, more preferably at least 4%, more preferably at least 5%, more preferably at least 7.5%, more preferably at least 10%, more preferably at least 15%, more preferably at least 20%, more preferably at least 25%, more preferably at least 30%, more preferably at least 35%, more preferably at least 40%, more preferably at least 45%, more preferably at least 50%, preferably relative to a state of the three-dimensional object before it has undergone the at least one method for post-processing of the three-dimensional object; and/or the post-processing of the three-dimensional object includes modifying the optical properties of the three-dimensional object resulting in that an average absorption or an integral of the absorption between 300 nm and 2000 nm, preferably between 350 nm and 1000 nm, more preferably between 400 nm and 900 nm, most preferably between 450 nm and 800 nm, is decreased by at least 1%, preferably at least 2%, more preferably at least 3%, more preferably at least 4%, more preferably at least 5%, more preferably at least 7.5%, more preferably at least 10%, more preferably at least 15%, more preferably at least 20%, more preferably at least 25%, more preferably at least 30%, more preferably at least 35%, more preferably at least 40%, more preferably at least 45%, more preferably at least 50% preferably relative to a state of the three-dimensional object before it has undergone the at least one method for post-processing.

In one embodiment the post-processing of the three-dimensional object comprises modifying the optical properties of the three-dimensional object resulting in an average or integrated absorption per mm of thickness of the three-dimensional object of less than 0.5, preferably less than 0.3, more preferably less than 0.2, more preferably less than 0.1, in a wavelength range between 300 nm and 2000 nm, preferably between 350 nm and 1000 nm, more preferably between 400 nm and 900 nm, most preferably between 450 nm and 800 nm.

Preferably, the absorption per mm of thickness of the three-dimensional object body or three-dimensional object is less than 0.5, preferably less than 0.3, more preferably less than 0.2, more preferably less than 0.1, for each wavelength in the wavelength range between 300 nm and 2000 nm, preferably between 350 nm and 1000 nm, more preferably between 400 nm and 900 nm, most preferably between 450 nm and 800 nm.

In one embodiment the post-processing comprises a thermal treatment of the three-dimensional object which thermal treatment comprises tempering the three-dimensional object for a specific time at least one specific temperature.

Preferably, the thermal treatment is carried out in specific time ranges between 0.1 min and 24 h, preferably between 0.5 min and 360 min, more preferably between 1 min and 60 min.

Preferably, the thermal treatment is carried out in specific temperature ranges between 50° C. and 150° C., preferably between 75° C. and 125° C.

In one embodiment the post-processing comprises an optical treatment of the three-dimensional object which optical treatment comprises irradiating the three-dimensional object with light of at least one specific wavelength for a specific time with a specific light intensity.

Preferably, the optical treatment is carried out in a time ranges between 0.1 min and 24 h, preferably between 1 min and 360 min, more preferably between 5 min and 60 min.

Preferably, an optical treatment of the three-dimensional object is carried out with a specific light intensity, wherein the specific light intensity can range between 0.0001-1000 W/cm², preferably between 0.001-100 W/cm², more preferably between 0.1-30 W/cm², most preferably between 1-10 W/cm2.

Preferably, the optical treatment is carried out, wherein the at least one specific wavelength ranges between 350 nm and 1000 nm, preferably between 400 nm and 800 nm, more preferably between 350 nm and 500 nm or between 420 nm and 800 nm.

In one embodiment, the optical treatment is carried out by irradiating light with an intensity and a wavelength by which the three-dimensional object is not removed from the polymerizable material. Preferably, the optical treatment is carried out, wherein the three-dimensional object is irradiated for the specific time and light intensity with the light of the at least one specific wavelength without removing the three-dimensional object from the surrounding polymerizable material.

Preferably, the optical treatment is carried out, wherein the three-dimensional object is subject to tempering after irradiating the three-dimensional object for the specific time with light of the at least one specific wavelength, wherein the tempering preferably comprises heating the three-dimensional object to a temperature ranging between 50° C. and 150° C. for a time ranging between 1 min and 360 min, preferably 5 min and 60 min.

The structural motif (BII) may be the structural motif (BIIa)

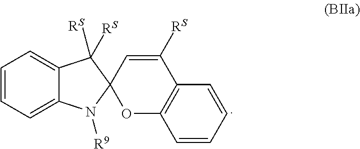

That is, the photoinitiator molecule may comprise the structural motif (BIIa).

In the structural motifs (BII) and (BIIa), R⁹ may be C₁-C₂₀-alkyl-electron withdrawing-C₅-C₄₈-aryl or C₁-C₂₀-alkyl-electron withdrawing-C₃-C₄₂-heteroaryl. R⁹ may be C₁-C₁₀-alkyl-electron withdrawing-C₅-C₄₈-aryl or C₁-C₁₀-alkyl-electron withdrawing-C₅-C₄₂-heteroaryl. R⁹ may be C₁-C₆-alkyl-electron withdrawing-C₅-C₄₈-aryl or C₁-C₆-alkyl-electron withdrawing-C₃-C₄₂-heteroaryl. R⁹ may be C₁-C₄-alkyl-electron withdrawing-C₅-C₄₈-aryl or C₁-C₄-alkyl-electron withdrawing-C₃-C₄₂-heteroaryl. R⁹ may be C₁-C₂-alkyl-electron withdrawing-C₅-C₄₈-aryl or C₁-C₂-alkyl-electron withdrawing-C₃-C₄₂-heteroaryl. R⁹ may be —CH₂-electron withdrawing-C₅-C₄₈-aryl or —CH₂-electron withdrawing-C₃-C₄₂-heteroaryl.

In the structural motifs (BII) and (BIIa), R⁹ may be C₁-C₂₀-alkyl-electron withdrawing-C₆-C₁₈-aryl or C₁-C₂₀-alkyl-electron withdrawing-C₃-C₁₇-heteroaryl. R⁹ may be C₁-C₁₀-alkyl-electron withdrawing-C₆-C₁₈-aryl or C₁-C₁₀-alkyl-electron withdrawing-C₃-C₁₇-heteroaryl. R⁹ may be C₁-C₆-alkyl-electron withdrawing-C₆-C₁₈-aryl or C₁-C₆-alkyl-electron withdrawing-C₃-C₁₇-heteroaryl. R⁹ may be C₁-C₄-alkyl-electron withdrawing-C₆-C₁₈-aryl or C₁-C₄-alkyl-electron withdrawing-C₃-C₁₇-heteroaryl. R⁹ may be C₁-C₂-alkyl-electron withdrawing-C₆-C₁₈-aryl or C₁-C₂-alkyl-electron withdrawing-C₃-C₁₇-heteroaryl. R⁹ may be —CH₂-electron withdrawing-C₆-C₁₈-aryl or —CH₂-electron withdrawing-C₃-C₁₇-heteroaryl.

In the structural motifs (BII) and (BIIa), R⁹ may be C₁-C₂₀-alkyl-electron withdrawing-C₅-C₄₈-aryl. R⁹ may be C₁-C₁₀-alkyl-electron withdrawing-C₅-C₄₈-aryl. R⁹ may be C₁-C₆-alkyl-electron withdrawing-C₅-C₄₈-aryl. R⁹ may be C₁-C₄-alkyl-electron withdrawing-C₅-C₄₈-aryl. R⁹ may be C₁-C₂-alkyl-electron withdrawing-C₅-C₄₈-aryl. R⁹ may be —CH₂-electron withdrawing-C₅-C₄₈-aryl.

In the structural motifs (BII) and (BIIa), R⁹ may be C₁-C₂₀-alkyl-electron withdrawing-C₆-C₁₈-aryl. R⁹ may be C₁-C₁₀-alkyl-electron withdrawing-C₆-C₁₈-aryl. R⁹ may be C₁-C₆-alkyl-electron withdrawing-C₆-C₁₈-aryl. R⁹ may be C₁-C₄-alkyl-electron withdrawing-C₆-C₁₈-aryl. R⁹ may be C₁-C₂-alkyl-electron withdrawing-C₆-C₁₈-aryl. R⁹ may be —CH₂-electron withdrawing-C₆-C₁₈-aryl.

In the structural motifs (BII) and (BIIa), R⁹ may be —CH₂-electron withdrawing-C₅-C₄₈-aryl. R⁹ may be —CH₂-electron withdrawing-C₅-C₄₂-aryl. R⁹ may be —CH₂-electron withdrawing-C₆-C₃₆-aryl. R⁹ may be —CH₂-electron withdrawing-C₆-C₃₀-aryl. R⁹ may be —CH₂-electron withdrawing-C₆-C₂₄-aryl. R⁹ may be —CH₂-electron withdrawing-C₆-C₁₈-aryl. R⁹ may be —CH₂-electron withdrawing-C₆-C₁₂-aryl. R⁹ may be —CH₂-electron withdrawing-C₆-C₁₀-aryl.

In the structural motifs (BII) and (BIIa), R⁹ may be —CH₂-(electron withdrawing phenyl). “—CH₂-(electron withdrawing phenyl)” is benzyl substituted with one or more electron withdrawing groups in a way that the ¹H-NMR (CDCl₃) signal (chemical shift) of the bold printed hydrogen in a compound of the formula

(substituted phenyl group)-CH₂—H
is large than the ¹H-NMR (CDCl₃) signal of the bold printed carbon in a compound of the formula Phenyl-CH₂—H.

In the structural motifs (BII) and (BIIa), the electron withdrawing aryl group in any of the foregoing alkyl electron withdrawing aryl may be aryl which is substituted with one or more electron withdrawing group(s). The electron withdrawing heteroaryl group in any of the foregoing alkyl electron withdrawing heteroaryl may be heteroaryl which is substituted with one or more electron withdrawing group(s) or which is, due to the one or more heteroatom(s) comprised in the electron withdrawing heteroaryl group, electron withdrawing itself. Examples include 4-chloro-2-methylpyridin and 2-methylthiazole.

An “electron withdrawing group” in terms of the present disclosure may be a group which is, when being comprised as a substituent in a molecule, more electron withdrawing in reference to hydrogen (H). The skilled person may refer to: “A survey of Hammett substituent constants and resonance and field parameters”, Chem. Rev. 1991, 91, 2, 165-195, which lists electron withdrawing groups and electron withdrawing substituents. Hence an electron-withdrawing group can be any group which has sₚ>0 and/or sₘ>0, preferably only the criterion sₚ>0 is considered if no reference is made to the position. Respective values are preferably taken from Table 1 in Chem. Rev. 1991, 91, 2, 165-195, where also a detailed description and definition can be found. For substituents which are not listed in Table 1 in Chem. Rev. 1991, 91, 2, 165-195, the respective value for sₚ and/or sₘ can be measured or calculated according to one of the methods which is applicable to the respective substituent as described in Chem. Rev. 1991, 91, 2, 165-195 or the references cited therein.

In other words, an “electron withdrawing group” in terms of the present disclosure may be an atom or group of atoms that pulls electron density away from other parts of a molecule through inductive or resonance effects. This makes nearby atoms or bonds more electrophilic and/or less nucleophilic.

In the structural motif (BII) and (BIIa), the electron withdrawing group may be selected from the group consisting of F, Cl, Br, I, NO₂, CN, carbonyl, carbonate, carbonate ester, partially fluorinated C₁-C₂₀ alkyl, partially fluorinated C₅-C₄₈ aryl, perfluorinated C₁ to C₂₀ alkyl, perfluorinated C₅-C₄₈ aryl, C₂-C₂₀-alkyl acyl, C₅-C₄₈-aryl acyl, substituted C₂-C₂₀-alkyl acyl, substituted C₅-C₄₈-aryl acyl, substituted or unsubstituted C₂ to C₂₀-alkoxycarbonyl, substituted or unsubstituted (meth)acrylate, C₁-C₂₀ alkylsulfone, C₅-C₄₈-aryl sulfone, sulfone, SO₂Me, SO₂NH₂, methoxy, C₅-C₄₈-aryloxy carbonyl, formyl, tosyl, mesyl, C₂-C₂₀ alkyl amide, and C₅-C₄₈-aryl amide.

If C₂ to C₂₀-alkoxycarbonyl (alkyl ester substituent) or C₅-C₄₈-aryloxy carbonyl (aryl ester substituent) is chosen as the electron withdrawing group, the binding position may be via the carbonyl-C or via the oxygen atom, preferably via the carbonyl-C.

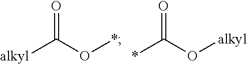

*=binding position

The same applies mutatis mutandis for the other electron withdrawing groups bearing a carbonyl group, such as amide etc.

The partially fluorinated C₁-C₂₀ alkyl may be partially fluorinated C₁-C₁₀ alkyl, partially fluorinated C₁-C₆ alkyl, partially fluorinated C₁-C₄ alkyl, partially fluorinated C₁-C₂ alkyl, CF₂H or CFH₂.

The perfluorinated C₁-C₂₀ alkyl may be perfluorinated C₁-C₁₀ alkyl, perfluorinated C₁-C₆ alkyl, perfluorinated C₁-C₄ alkyl, perfluorinated C₁-C₂ alkyl, CF₃.

The partially fluorinated C₅-C₄₈ aryl may be partially fluorinated C₆-C₄₂ aryl, partially fluorinated C₆-C₃₆ aryl, partially fluorinated C₆-C₃₀ aryl, partially fluorinated C₆-C₂₄ aryl, partially fluorinated C₆-C₁₈ aryl, partially fluorinated C₆-C₁₂ aryl, partially fluorinated C₆-C₁₀ aryl, or partially fluorinated phenyl.

The perfluorinated C₅-C₄₈ aryl may be perfluorinated C₆-C₄₂ aryl, perfluorinated C₆-C₃₆ aryl, perfluorinated C₆-C₃₀ aryl, perfluorinated C₆-C₂₄ aryl, perfluorinated C₆-C₁₈ aryl, perfluorinated C₆-C₁₂ aryl, perfluorinated C₆-C₁₀ aryl, or perfluorinated phenyl.

The C₂-C₂₀-alkyl acyl may be C₂-C₁₀-alkyl acyl, C₂-C₆-alkyl acyl, C₂-C₄-alkyl acyl, or C₂-alkyl acyl (methylacyl).

The C₅-C₄₈-aryl acyl may be C₆-C₄₂-aryl acyl, C₆-C₃₆-aryl acyl, C₆-C₃₀-aryl acyl, C₆-C₂₄-aryl acyl, C₆-C₁₈-aryl acyl, C₆-C₁₂-aryl acyl, or phenylacyl or benzoyl.

The C₂ to C₂₀-alkoxycarbonyl may be C₂ to C₁₀-alkoxycarbonyl, C₂ to C₆-alkoxycarbonyl, C₂ to C₄-alkoxycarbonyl, C₂ to C₂-alkoxycarbonyl, or methoxycarbonyl.

The C₅-C₄₈-aryloxy carbonyl may be C₆-C₄₂-aryloxy carbonyl, C₆-C₃₆-aryloxy carbonyl, C₆-C₃₀-aryloxy carbonyl, C₆-C₂₄-aryloxy carbonyl, C₆-C₁₈-aryloxy carbonyl, C₆-C₁₂-aryloxy carbonyl, or phenoxycarbonyl.

The C₅-C₄₈-aryl sulfone may be C₆-C₄₂-aryl sulfone, C₆-C₃₆-aryl sulfone, C₆-C₃₀-aryl sulfone, C₆-C₂₄-aryl sulfone, C₆-C₁₈-aryl sulfone, C₆-C₁₂-aryl sulfone, or phenylsulfone.

The C₁-C₂₀ alkylsulfone may be C₁-C₁₀ alkylsulfone, C₁-C₆ alkylsulfone, C₁-C₄ alkylsulfone, C₁-C₂ alkylsulfone, or methylsulfone.

The C₂-C₂₀ alkyl amide may be C₂-C₁₀ alkyl amide, C₂-C₆ alkyl amide, C₂-C₄ alkyl amide, C₁-C₃ alkyl amide, or methylamide.

The C₅-C₄₈-aryl amide may be C₆-C₄₂-aryl amide, C₆-C₃₆-aryl amide, C₆-C₃₀-aryl amide, C₆-C₂₄-aryl amide, C₆-C₁₈-aryl amide, C₆-C₁₂-aryl amide, or phenylamide.

The respective electron withdrawing groups may be unsubstituted or substituted, preferably with one or more substituents independently selected from the group consisting of group consisting of D, methyl, tert-butyl, cyclohexyl, phenyl, CF₃, F, Cl, Br, I, CN, nitro, formyl, ethylester, methylester, acetyl, methoxy, OH, dimethylamino, NH₂, amide (CO—NH₂), carboxylic acid or salt thereof, sulfonic acid or salt thereof, methylsulfone, and sulfonamide.

In the structural motifs (BII) and (BIIa), the electron withdrawing group may be selected from the group consisting of NO₂, CN, perfluorinated C₁ to C₂₀ alkyl, C₂-C₂₀-alkyl acyl, C₅-C₄₈-aryl acyl, and substituted or unsubstituted C₂ to C₂₀ alkoxycarbonyl. In the structural motifs (BII) and (BIIa), the electron withdrawing group may be selected from the group consisting of NO₂, CN, perfluorinated C₁ to C₁₀ alkyl, C₂-C₁₀-alkyl acyl, C₆-C₃₀-aryl acyl, and substituted or unsubstituted C₂ to C₁₀ alkoxycarbonyl. In the structural motifs (BII) and (BIIa), the electron withdrawing group may be selected from the group consisting of NO₂, CN, perfluorinated C₁ to C₆ alkyl, C₂-C₆-alkyl acyl, C₆-C₁₈-aryl acyl, and substituted or unsubstituted C₂ to C₆ alkoxycarbonyl. In the structural motifs (BII) and (BIIa), the electron withdrawing group may be selected from the group consisting of NO₂, CN, perfluorinated C₁ to C₄ alkyl, C₂-C₄-alkyl acyl, C₆-C₁₂-aryl acyl, and substituted or unsubstituted C₂ to C₄ alkoxycarbonyl. In the structural motifs (BII) and (BIIa), the electron withdrawing group may be selected from the group consisting of NO₂, CN, CF₃, methylacyl, phenylacyl, and methoxycarbonyl.

In the structural motifs (BII) and (BIIa), R⁹ may be selected from the following groups,

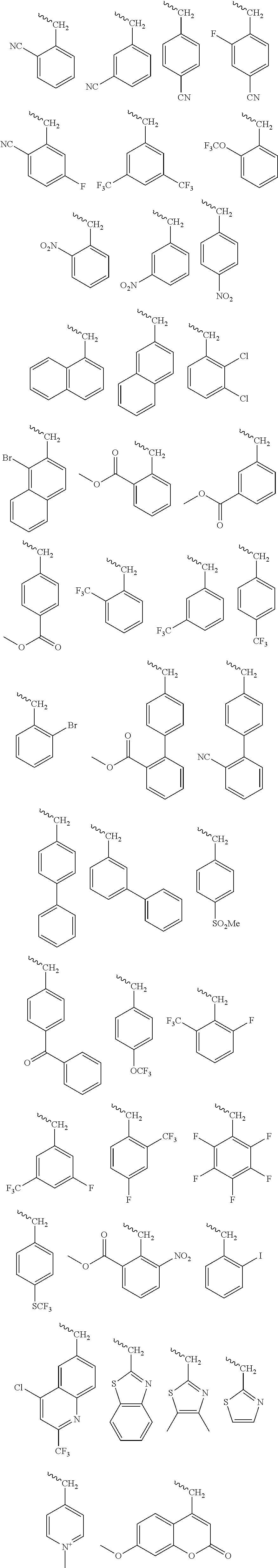

wherein “ [symbol] ” represents the binding position.

The structural motifs (BI) or (BII) may be the structural motif (BIII)

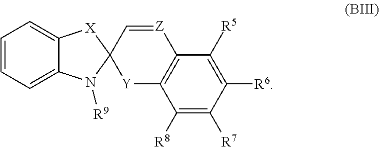

In other words, the photoinitiator molecule may comprise the structural motif (BIII).

In the structural motif (BIII), at least one of R⁵ to R⁸ is an electron withdrawing group.

Preferably X is selected from S or CR^{S}₂. More preferably X is CR^{S}₂ and R^{S} is independently selected from H, D, C₅-C₄₈-aryl, C₆-C₁₀-aryl, C₁-C₂₀-alkyl, C₁-C₁₀-alkyl, C₁-C₆-alkyl, and C₁-C₄-alkyl or the two R^{S} form together with the C (the atom X in the structural motif (BIII)) a substituted or unsubstituted C₁ to C₂₀ cycloalkyl ring. More preferably, X is CR^{S}₂ and the two R^{S} are methyl or the two R^{S} form together with the C (the atom X in the structural motif (BIII)) a C₁ to C₆ cycloalkyl ring, preferably a cyclohexyl ring.

Preferably Z is CR^{S}. R^{S} may be independently selected from H, D, CN, C₁-C₁₂-alkyl, fluorinated C₁-C₁₂-alkyl, or C₆-C₁₄-aryl. More preferably, Z is CR^{S} and R^{S} is H.

Preferably, Y is S or O. More preferably Y is O.

The structural motif (BIII) may be the structural motif (BIIIa)

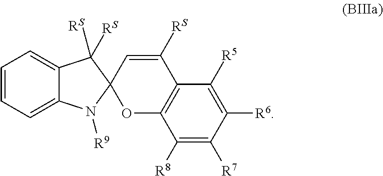

That is, the photoinitiator molecule may comprise the structural motif (BIIIa).

In the structural motifs (BIII) and (BIIIa), R⁹ is C₁ to C₂₀-alkyl electron withdrawing C₅ to C₄₈-aryl or C₁ to C₂₀-alkyl electron withdrawing C₃ to C₄₂ heteroaryl and may be as described above for the structural motifs (BII) and (BIIa).

In the structural motifs (BIII) and (BIIIa), at least one of R⁵ to R⁸ is an electron withdrawing group.

In the structural motifs (BIII) and (BIIIa), the electron withdrawing group which is part of the structural motifs (BIII) and (BIIIa) as at least one of R⁵ to R⁸ may be selected from the group consisting of F, Cl, Br, I, NO₂, CN, SF₅, carbonyl, carbonate, carbonate ester, partially fluorinated C₁-C₂₀ alkyl, partially fluorinated C₁-C₂₀ alkylether, partially fluorinated C₅-C₄₈ aryl, perfluorinated C₁ to C₂₀ alkyl, perfluorinated C₁ to C₂₀ alkylether, perfluorinated C₅-C₄₈ aryl, C₂-C₂₀-alkyl acyl, C₅-C₄₈-aryl acyl, substituted or unsubstituted C₂-C₄₉-alkyl acyl, substituted or unsubstituted C₅-C₄₈-aryl acyl, substituted or unsubstituted C₃-C₄₈-heteroaryl acyl, substituted or unsubstituted C₂ to C₂₀-alkoxycarbonyl, substituted or unsubstituted (meth)acrylate, C₁-C₂₀ alkylsulfone, C₅-C₄₈-aryl sulfone, sulfone, SO₂Me, SO₂NH₂, methoxy, C₅-C₄₈-aryloxy carbonyl, formyl, tosyl, mesyl, C₂-C₂₀ alkyl amide, and C₅-C₄₈-aryl amide; substituted C₅-C₄₈ aryl, particularly C₅-C₄₈ aryl which is substituted with an electron withdrawing group; alpha-diketones.

The electron withdrawing group may be selected from the following groups EW1 to EW3, preferably EW1

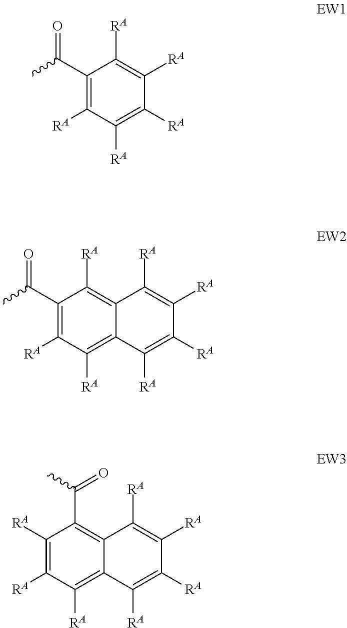

wherein
The different RA in the structural motifs EW1, EW2, or EW3 may be independently selected from the group consisting of H, D, halogen, NO₂, CN, OH, SH, substituted or unsubstituted C₁-C₂₀-alkyl, substituted or unsubstituted C₃-C₂₀-cycloalkyl, substituted or unsubstituted C₅-C₄₈-aryl, substituted or unsubstituted C₁-C₂₀-alkyl-C₅-C₄₈-aryl, preferably C₁-C₂₀-alkyl-electron withdrawing-C₅-C₄₈-aryl; substituted or unsubstituted C₃-C₄₂-heteroaryl, substituted or unsubstituted C₁-C₂₀-alkyl-C₃-C₄₂-heteroaryl, preferably C₁-C₂₀-alkyl-electron withdrawing-C₃-C₄₂-heteroaryl; substituted or unsubstituted C₂-C₄₉-alkyl acyl, substituted or unsubstituted C₂-C₄₉-aryl acyl, substituted or unsubstituted C₁-C₂₀-alkoxy, substituted or unsubstituted C₅-C₄₈-aryloxy, C₁-C₂₀-alkyl-C₅-C₄₈-aryloxy, C₅-C₄₈-aryl-C₁-C₂₀-alkoxy, NH₂, substituted or unsubstituted C₁-C₂₀-alkyl ester, substituted or unsubstituted C₅-C₄₈-aryl ester, substituted or unsubstituted C₁-C₂₀ alkyl amide, substituted or unsubstituted C₅-C₄₈-aryl amide, NR′₂, SiR′₃, —O—SiR′₃, substituted or unsubstituted carboxylic acids and salts thereof, substituted or unsubstituted sulfonic acids and salts thereof, substituted or unsubstituted sulfonic esters, substituted or unsubstituted sulfonic amides, formyl, ether, thioether, carbonate, carbonate ester, sulfates, boronic acids, boronic esters, phosphonic acids, phosphonic esters, phosphines, phosphates, peroxycarbonic acids, thiocarbonic acids, sulfinic acids, sulfinic esters, sulfonates, thiolesters, sulfoxides, sulfones, hydrazides, thioaldehydes, ketones, thioketones, oximes, hydrazines, nitroso, azo, diazo, diazonium; isocyanides, cyanate, isocyanate, thiocyanate, isothiocyanate, hydroperoxide, peroxide, acetals, ketal, orthoester, orthocarbonate esters, ammonium, imines, imides, azide, nitrate, isonitrile, nitrosoxy, substituted or unsubstituted carbamates, substituted or unsubstituted ethers, substituted or unsubstituted polyether carbamates, substituted or unsubstituted arylazo, substituted or unsubstituted C₂-C₂₀-alkynyl, substituted or unsubstituted C₂-C₂₀-alkenyl, or any group from Table 1 in “A survey of Hammett substituent constants and resonance and field parameters”, Chem. Rev. 1991, 91, 2, 165-195.

R′ may be independently selected from the group consisting of substituted or unsubstituted C₁-C₂₀-alkyl and substituted or unsubstituted C₅-C₄₈-aryl, and two R′ may form a ring structure.

If one or more of RA and/or a ring formed by two or more thereof is substituted, the respective substituent(s) can be selected from the group consisting of D, halogen, NO₂, CN, OH, SH, substituted or unsubstituted C₁-C₂₀-alkyl, substituted or unsubstituted C₃-C₂₀-cycloalkyl, substituted or unsubstituted C₅-C₄₈-aryl, substituted or unsubstituted C₁-C₂₀-alkyl-C₅-C₄₈-aryl, preferably C₁-C₂₀-alkyl-electron withdrawing-C₅-C₄₈-aryl; substituted or unsubstituted C₃-C₄₂-heteroaryl, substituted or unsubstituted C₁-C₂₀-alkyl-C₃-C₄₂-heteroaryl, preferably C₁-C₂₀-alkyl-electron withdrawing-C₃-C₄₂-heteroaryl; substituted or unsubstituted C₂-C₄₉-alkyl acyl, substituted or unsubstituted C₂-C₄₉-aryl acyl, substituted or unsubstituted C₁-C₂₀-alkoxy, substituted or unsubstituted C₅-C₄₈-aryloxy, C₁-C₂₀-alkyl-C₅-C₄₈-aryloxy, C₅-C₄₈-aryl-C₁-C₂₀-alkoxy, NH₂, substituted or unsubstituted C₁-C₂₀-alkyl ester, substituted or unsubstituted C₅-C₄₈-aryl ester, substituted or unsubstituted C₁-C₂₀ alkyl amide, substituted or unsubstituted C₅-C₄₈-aryl amide, NR′₂, SiR′₃, —O—SiR′₃, substituted or unsubstituted carboxylic acids and salts thereof, substituted or unsubstituted sulfonic acids and salts thereof, substituted or unsubstituted sulfonic esters, substituted or unsubstituted sulfonic amides, formyl, ether, thioether, carbonate, carbonate ester, sulfates, boronic acids, boronic esters, phosphonic acids, phosphonic esters, phosphines, phosphates, peroxycarbonic acids, thiocarbonic acids, sulfinic acids, sulfinic esters, sulfonates, thiolesters, sulfoxides, sulfones, hydrazides, thioaldehydes, ketones, thioketones, oximes, hydrazines, nitroso, azo, diazo, diazonium; isocyanides, cyanate, isocyanate, thiocyanate, isothiocyanate, hydroperoxide, peroxide, acetals, ketal, orthoester, orthocarbonate esters, ammonium, imines, imides, azide, nitrate, isonitrile, nitrosoxy, substituted or unsubstituted carbamates, substituted or unsubstituted ethers, substituted or unsubstituted polyether carbamates, substituted or unsubstituted arylazo, substituted or unsubstituted C₂-C₂₀-alkynyl, substituted or unsubstituted C₂-C₂₀-alkenyl. R′ may be independently selected from the group consisting of substituted or unsubstituted C₁-C₂₀-alkyl and substituted or unsubstituted C₅-C₄₈-aryl, and two R′ may form a ring structure, wherein the respective substituents can, in turn be preferably substituted with D or C₁ to C₆ alky.

R^{A} may be independently selected from H, D, halogen, preferably F or Cl, CN, C₁-C₁₂-alkyl, C₁-C₁₂ alkoxy, fluorinated C₁-C₁₂-alkyl, preferably perfluorinated C₁ to C₁₂ alkyl, such as CF₃, C₆-C₁₄-aryl or the group of electron withdrawing groups.

In one embodiment, at least of R^{A} may be an electron withdrawing group, preferably F, Cl, Br, I, NO₂, CN, SF₅, carbonyl, carbonate, carbonate ester, partially fluorinated C₁-C₂₀ alkyl, partially fluorinated C₁-C₂₀ alkylether, partially fluorinated C₅-C₄₈ aryl, perfluorinated C₁ to C₂₀ alkyl, perfluorinated C₁ to C₂₀ alkylether, perfluorinated C₅-C₄₈ aryl, C₂-C₂₀-alkyl acyl, C₅-C₄₈-aryl acyl, substituted or unsubstituted C₂-C₄₉-alkyl acyl, substituted or unsubstituted C₅-C₄₈-aryl acyl, substituted or unsubstituted C₃-C₄₈-heteroaryl acyl, substituted or unsubstituted C₂ to C₂₀-alkoxycarbonyl, substituted or unsubstituted (meth)acrylate, C₁-C₂₀ alkylsulfone, C₅-C₄₈-aryl sulfone, sulfone, SO₂Me, SO₂NH₂, methoxy, C₅-C₄₈-aryloxy carbonyl, formyl, tosyl, mesyl, C₂-C₂₀ alkyl amide, and C₅-C₄₈-aryl amide; substituted C₅-C₄₈ aryl, particularly C₅-C₄₈ aryl which is substituted with an electron withdrawing group; alpha-diketones.

In another embodiment, R^{A} may be independently selected from the group consisting of H, D, electron withdrawing groups, F, Cl, CN, CF₃ or phenyl.

In one embodiment, R^{A} may be independently selected from the group consisting of H, D F, Cl, CN, CF₃ and phenyl.

If C₂ to C₂₀-alkoxycarbonyl (alkyl ester substituent) or C₅-C₄₈-aryloxy carbonyl (aryl ester substituent) is chosen as the electron withdrawing group, the binding position may be via the carbonyl-C or the oxygen atom.

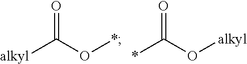

*=binding position

The same applies mutatis mutandis for the other electron withdrawing groups bearing a carbonyl group, such as amide etc.

The partially fluorinated C₁-C₂₀ alkyl may be partially fluorinated C₁-C₁₀ alkyl, partially fluorinated C₁-C₆ alkyl, partially fluorinated C₁-C₄ alkyl, partially fluorinated C₁-C₂ alkyl, CF₂H or CFH₂.

The perfluorinated C₁-C₂₀ alkyl may be perfluorinated C₁-C₁₀ alkyl, perfluorinated C₁-C₆ alkyl, perfluorinated C₁-C₄ alkyl, perfluorinated C₁-C₂ alkyl, CF₃.

The partially fluorinated C₅-C₄₈ aryl may be partially fluorinated C₆-C₄₂ aryl, partially fluorinated C₆-C₃₆ aryl, partially fluorinated C₆-C₃₀ aryl, partially fluorinated C₆-C₂₄ aryl, partially fluorinated C₆-C₁₈ aryl, partially fluorinated C₆-C₁₂ aryl, partially fluorinated C₆-C₁₀ aryl, or partially fluorinated phenyl.

The perfluorinated C₅-C₄₈ aryl may be perfluorinated C₆-C₄₂ aryl, perfluorinated C₆-C₃₆ aryl, perfluorinated C₆-C₃₀ aryl, perfluorinated C₆-C₂₄ aryl, perfluorinated C₆-C₁₈ aryl, perfluorinated C₆-C₁₂ aryl, perfluorinated C₆-C₁₀ aryl, or perfluorinated phenyl.

The C₂-C₂₀-alkyl acyl may be C₂-C₁₀-alkyl acyl, C₂-C₆-alkyl acyl, C₂-C₄-alkyl acyl, or C₂-alkyl acyl (methylacyl).

The C₅-C₄₈-aryl acyl may be C₆-C₄₂-aryl acyl, C₆-C₃₆-aryl acyl, C₆-C₃₀-aryl acyl, C₆-C₂₄-aryl acyl, C₆-C₁₈-aryl acyl, C₆-C₁₂-aryl acyl, or phenylacyl.

The C₂ to C₂₀-alkoxycarbonyl may be C₂ to C₁₀-alkoxycarbonyl, C₂ to C₆-alkoxycarbonyl, C₂ to C₄-alkoxycarbonyl, C₂ to C₃-alkoxycarbonyl, or methoxycarbonyl.

The C₅-C₄₈-aryloxy carbonyl may be C₆-C₄₂-aryloxy carbonyl, C₆-C₃₆-aryloxy carbonyl, C₆-C₃₀-aryloxy carbonyl, C₆-C₂₄-aryloxy carbonyl, C₆-C₁₈-aryloxy carbonyl, C₆-C₁₂-aryloxy carbonyl, or phenoxycarbonyl.

The C₅-C₄₈-aryl sulfone may be C₆-C₄₂-aryl sulfone, C₆-C₃₆-aryl sulfone, C₆-C₃₀-aryl sulfone, C₆-C₂₄-aryl sulfone, C₆-C₁₈-aryl sulfone, C₆-C₁₂-aryl sulfone, or phenylsulfone.

The C₁-C₂₀ alkylsulfone may be C₁-C₁₀ alkylsulfone, C₁-C₆ alkylsulfone, C₁-C₄ alkylsulfone, C₁-C₂ alkylsulfone, or methylsulfone.

The C₂-C₂₀ alkyl amide may be C₂-C₁₀ alkyl amide, C₂-C₆ alkyl amide, C₂-C₄ alkyl amide, C₂-C₃ alkyl amide, or methylamide.

The C₅-C₄₈-aryl amide may be C₆-C₄₂-aryl amide, C₆-C₃₆-aryl amide, C₆-C₃₀-aryl amide, C₆-C₂₄-aryl amide, C₆-C₁₈-aryl amide, C₆-C₁₂-aryl amide, or phenylamide.

In the structural motifs (BIII) and (BIIIa), the electron withdrawing group may be selected from the group consisting of Cl, NO₂, CN, perfluorinated C₁ to C₂₀ alkyl, C₂-C₂₀-alkyl acyl, C₃-C₄₈-heteroaryl acyl, C₅-C₄₈-aryl acyl, and substituted or unsubstituted C₂ to C₂₀ alkoxycarbonyl. In the structural motifs (BIII) and (BIIIa), the electron withdrawing group may be selected from the group consisting of Cl, NO₂, CN, perfluorinated C₁ to C₁₀ alkyl, C₂-C₁₀-alkyl acyl, C₆-C₃₀-aryl acyl, C₃-C₃₀-heteroaryl acyl, and substituted or unsubstituted C₂ to C₁₀ alkoxycarbonyl. In the structural motifs (BIII) and (BIIIa), the electron withdrawing group may be selected from the group consisting of C₁, NO₂, CN, perfluorinated C₁ to C₆ alkyl, C₂-C₆-alkyl acyl, C₆-C₁₈-aryl acyl, C₃-C₁₈-heteroaryl acyl, and substituted or unsubstituted C₂ to C₆ alkoxycarbonyl. In the structural motifs (BIII) and (BIIIa), the electron withdrawing group may be selected from the group consisting of C₁, NO₂, CN, perfluorinated C₁ to C₄ alkyl, C₂-C₄-alkyl acyl, C₆-C₁₂-aryl acyl, C₃-C₁₂-heteroaryl acyl, and substituted or unsubstituted C₂ to C₄ alkoxycarbonyl. In the structural motif (BIII) and (BIIIa), the electron withdrawing group may be selected from the group consisting of C₁, NO₂, CN, CF₃, methylacyl, phenylacyl, and methoxycarbonyl.

The structural motifs (BI), (BII) or (BIII) may be the structural motif (BIV)

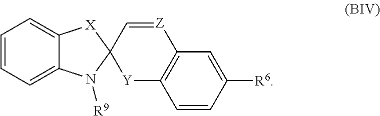

In other words, the photoinitiator molecule may comprise the structural motif (BIV).

In the structural motif (BIV), R⁶ is an electron withdrawing group.

Preferably X is selected from S or CR^{S}₂. More preferably X is CR^{S}₂ and R^{S} is independently selected from H, D, C₅-C₄₈-aryl, C₆-C₁₀-aryl, C₁-C₂₀-alkyl, C₁-C₁₀-alkyl, C₁-C₆-alkyl, and C₁-C₄-alkyl or the two R^{S} form together with the C (the atom X in the structural motif (BIV)) a substituted or unsubstituted C₁ to C₂₀ cycloalkyl ring. More preferably, X is CR^{S}₂ and the two R^{S} are methyl or the two R^{S} form together with the C (the atom X in the structural motif (BIV)) a C₁ to C₆ cycloalkyl ring, preferably a cyclohexyl ring.

Preferably Z is CR^{S}. R^{S} may be independently selected from H, D, CN, C₁-C₁₂-alkyl, fluorinated C₁-C₁₂-alkyl, or C₆-C₁₄-aryl. More preferably, Z is CR^{S} and R^{S} is H.

Preferably, Y is S or O. More preferably Y is O.

The structural motif (BIV) may be the structural motif (BIVa)

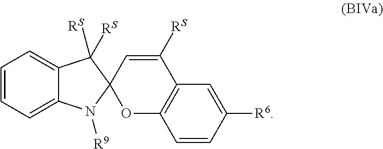

That is, the photoinitiator molecule may comprise the structural motif (BIVa).

In the structural motifs (BIV) and (BIVa), R⁹ is C₁ to C₂₀-alkyl electron withdrawing C₅ to C₄₈-aryl or C₁ to C₂₀-alkyl electron withdrawing C₃ to C₄₂ heteroaryl and may be as described above for the structural motifs (BII) and (BIIa).

In the structural motifs (BIV) and (BIVa), R⁶ is an electron withdrawing group and is as described above for the structural motifs (BIII) and (BIIIa) for the at least one of R⁵ to R⁸.

The structural motif (BI), (BII) or (BIII) may be the structural motif (BV)

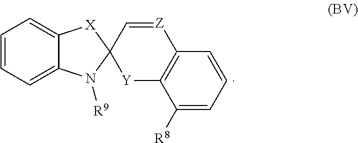

In other words, the photoinitiator molecule may comprise the structural motif (BV).

In the structural motif (BV), R⁸ is an electron withdrawing group.

Preferably X is selected from S or CR^{S}₂. More preferably X is CR^{S}₂ and R^{S} is independently selected from H, D, C₅-C₄₈-aryl, C₆-C₁₀-aryl, C₁-C₂₀-alkyl, C₁-C₁₀-alkyl, C₁-C₆-alkyl, and C₁-C₄-alkyl or the two R^{S} form together with the C (the atom X in the structural motif (BV)) a substituted or unsubstituted C₁ to C₂₀ cycloalkyl ring. More preferably, X is CR^{S}₂ and the two R^{S} are methyl or the two R^{S} form together with the C (the atom X in the structural motif (BV)) a C₁ to C₆ cycloalkyl ring, preferably a cyclohexyl ring.

Preferably Z is CR^{S}. R^{S} may be independently selected from H, D, CN, C₁-C₁₂-alkyl, fluorinated C₁-C₁₂-alkyl, or C₆-C₁₄-aryl. More preferably, Z is CR^{S} and R^{S} is H.

Preferably, Y is S or O. More preferably Y is O.

The structural motif (BV) may be the structural motif (BVa)

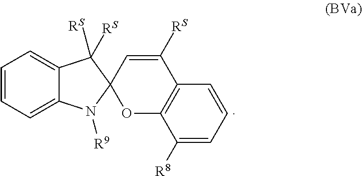

That is, the photoinitiator molecule may comprise the structural motif (BVa).

In the structural motifs (BV) and (BVa), R⁹ is C₁ to C₂₀-alkyl electron withdrawing C₅ to C₄₈-aryl or C₁ to C₂₀-alkyl electron withdrawing C₃ to C₄₂ heteroaryl and may be as described above for the structural motifs (BII) and (BIIa).

In the structural motifs (BV) and (BVa), R⁸ is an electron withdrawing group and is as described above for the structural motifs (BIII) and (BIIIa) for the at least one of R⁵ to R⁸.

The structural motif (BI), (BII) or (BIII) may be the structural motif (BVI)

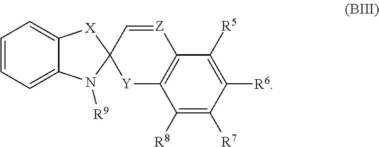

In other words, the photoinitiator molecule may comprise the structural motif (BVI).

In the structural motif (BVI) at least one of R⁵ to R⁸ is an electron donating group.

Preferably X is selected from S or CR^{S}₂. More preferably X is CR^{S}₂ and R^{S} is independently selected from H, D, C₅-C₄₈-aryl, C₆-C₁₀-aryl, C₁-C₂₀-alkyl, C₁-C₁₀-alkyl, C₁-C₆-alkyl, and C₁-C₄-alkyl or the two R^{S} form together with the C (the atom X in the structural motif (BVI)) a substituted or unsubstituted C₁ to C₂₀ cycloalkyl ring. More preferably, X is CR^{S}₂ and the two R^{S} are methyl or the two R^{S} form together with the C (the atom X in the structural motif (BVI)) a C₁ to C₆ cycloalkyl ring, preferably a cyclohexyl ring.

Preferably Z is CR^{S}. R^{S} may be independently selected from H, D, CN, C₁-C₁₂-alkyl, fluorinated C₁-C₁₂-alkyl, or C₆-C₁₄-aryl. More preferably, Z is CR^{S} and R^{S} is H.

Preferably, Y is S or O. More preferably Y is O.

The structural motif (BVI) may be the structural motif (BVIa)

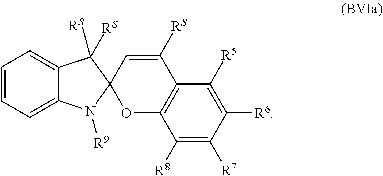

That is, the photoinitiator molecule may comprise the structural motif (BVIa).

In the structural motifs (BVI) and (BVIa), R⁹ is C₁ to C₂₀-alkyl electron withdrawing C₅ to C₄₈-aryl or C₁ to C₂₀-alkyl electron withdrawing C₃ to C₄₂ heteroaryl and may be as described above for the structural motifs (BII) and (BIIa).

In the structural motifs (BVI) and (BVIa) at least one of R⁵ to R⁸ is an electron donating group. The one or more electron donating group(s) from which at least one of R⁵ to R⁸ is selected is/are independently selected from the group consisting of substituted or unsubstituted C₁-C₂₀-alkoxy, C₅-C₄₈-aryloxy, and NR″₂, wherein R″ is independently selected from the group consisting of H, D, substituted or unsubstituted C₁-C₂₀-alkyl and substituted or unsubstituted C₅-C₄₈-aryl, and two R″ may form a ring structure.

The C₁-C₂₀-alkoxy may be C₁-C₁₀-alkoxy, C₁-C₆-alkoxy, C₁-C₄-alkoxy, C₁-C₂-alkoxy, or methoxy.

The C₅-C₄₈-aryloxy may be C₆-C₄₂-aryloxy, C₆-C₃₆-aryloxy, C₆-C₃₀-aryloxy, C₆-C₂₄-aryloxy, C₆-C₁₈-aryloxy, C₆-C₁₂-aryloxy, or phenyloxy.

The C₁-C₂₀-alkyl which can be R″ in NR″₂ may be C₁-C₁₀-alkyl, C₁-C₆-alkyl, C₁-C₄-alkyl, C₁-C₂-alkyl, or methyl. If the two R″ are alkyl and form together a ring, the ring may be a C₅ ring, a C₆ ring or a C₇ ring, preferably forming the structure of piperidine, morpholine, or pyrrolidine.

The C₅-C₄₈-aryl which can be R″ in NR″₂ may be C₆-C₄₂-aryl, C₆-C₃₆-aryl, C₆-C₃₀-aryl, C₆-C₂₄-aryl, C₆-C₁₈-aryl, C₆-C₁₂-aryl, C₆-C₁₀-aryl, or phenyl. If the two R″ are aryl and form together a ring, preferably the ring may form together with the N-atom of NR″₂ a carbazolyl functional group.

If the respective electron donating group is substituted, the one or more substituents may be independently selected from the group consisting of H, D, halogen, NO₂, CN, OH, SH, substituted or unsubstituted C₁-C₂₀-alkyl, substituted or unsubstituted C₃-C₂₀-cycloalkyl, substituted or unsubstituted C₅-C₄₈-aryl, substituted or unsubstituted C₁-C₂₀-alkyl-C₅-C₄₈-aryl, substituted or unsubstituted C₃-C₄₂-heteroaryl, substituted or unsubstituted C₁-C₂₀-alkyl-C₃-C₄₂-heteroaryl, substituted or unsubstituted C₂-C₄₉-alkyl acyl, substituted or unsubstituted C₇-C₄₉-aryl acyl, substituted or unsubstituted C₁-C₂₀-alkoxy, substituted or unsubstituted C₅-C₄₈-aryloxy, C₁-C₂₀-alkyl-C₅-C₄₈-aryloxy, C₅-C₄₈-aryl-C₁-C₂₀-alkoxy, NH₂, substituted or unsubstituted C₁-C₂₀-alkyl ester, substituted or unsubstituted C₅-C₄₈-aryl ester, substituted or unsubstituted C₁-C₂₀ alkyl amide, substituted or unsubstituted C₅-C₄₈-aryl amide, NR′₂, SiR′₃, —O—SiR′₃, substituted or unsubstituted carboxylic acids and salts thereof, substituted or unsubstituted sulfonic acids and salts thereof, substituted or unsubstituted sulfonic esters, substituted or unsubstituted sulfonic amides, formyl, ether, thioether, carbonate, carbonate ester, sulfates, boronic acids, boronic esters, phosphonic acids, phosphonic esters, phosphines, phosphates, peroxycarbonic acids, thiocarbonic acids, sulfinic acids, sulfinic esters, sulfonates, thiolesters, sulfoxides, sulfones, hydrazides, thioaldehydes, ketones, thioketones, oximes, hydrazines, nitroso, azo, diazo, diazonium; isocyanides, cyanate, isocyanate, thiocyanate, isothiocyanate, hydroperoxide, peroxide, acetals, ketal, orthoester, orthocarbonate esters, ammonium, imines, imides, azide, nitrate, isonitrile, nitrosoxy, substituted or unsubstituted carbamates, substituted or unsubstituted ethers, substituted or unsubstituted polyether carbamates, substituted or unsubstituted arylazo, substituted or unsubstituted C₂-C₂₀-alkynyl, substituted or unsubstituted C₂-C₂₀-alkenyl, preferably substituted or unsubstituted C₅-C₄₈-aryl. Substituents on the respective substituents may be selected as set forth above for R^{S}.

Preferably, electron donating group is selected from the following groups EDI to ED 3

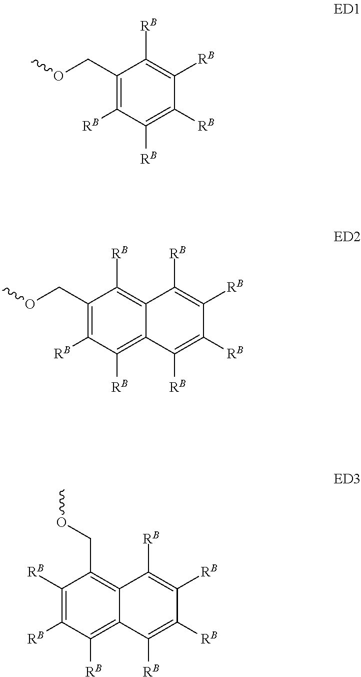

wherein

R^{B} is independently selected from the group consisting of H, D, halogen, NO₂, CN, OH, SH, substituted or unsubstituted C₁-C₂₀-alkyl, substituted or unsubstituted C₃-C₂₀-cycloalkyl, substituted or unsubstituted C₅-C₄₈-aryl, substituted or unsubstituted C₁-C₂₀-alkyl-C₅-C₄₈-aryl, substituted or unsubstituted C₃-C₄₂-heteroaryl, substituted or unsubstituted C₁-C₂₀-alkyl-C₃-C₄₂-heteroaryl, substituted or unsubstituted C₂-C₄₉-alkyl acyl, substituted or unsubstituted C₇-C₄₉-aryl acyl, substituted or unsubstituted C₁-C₂₀-alkoxy, substituted or unsubstituted C₅-C₄₈-aryloxy, C₁-C₂₀-alkyl-C₅-C₄₈-aryloxy, C₅-C₄₈-aryl-C₁-C₂₀-alkoxy, NH₂, substituted or unsubstituted C₁-C₂₀-alkyl ester, substituted or unsubstituted C₅-C₄₈-aryl ester, substituted or unsubstituted C₁-C₂₀ alkyl amide, substituted or unsubstituted C₅-C₄₈-aryl amide, NR′₂, SiR′₃, —O—SiR′₃, substituted or unsubstituted carboxylic acids and salts thereof, substituted or unsubstituted sulfonic acids and salts thereof, substituted or unsubstituted sulfonic esters, substituted or unsubstituted sulfonic amides, formyl, ether, thioether, carbonate, carbonate ester, sulfates, boronic acids, boronic esters, phosphonic acids, phosphonic esters, phosphines, phosphates, peroxycarbonic acids, thiocarbonic acids, sulfinic acids, sulfinic esters, sulfonates, thiolesters, sulfoxides, sulfones, hydrazides, thioaldehydes, ketones, thioketones, oximes, hydrazines, nitroso, azo, diazo, diazonium; isocyanides, cyanate, isocyanate, thiocyanate, isothiocyanate, hydroperoxide, peroxide, acetals, ketal, orthoester, orthocarbonate esters, ammonium, imines, imides, azide, nitrate, isonitrile, nitrosoxy, substituted or unsubstituted carbamates, substituted or unsubstituted ethers, substituted or unsubstituted polyether carbamates, substituted or unsubstituted arylazo, substituted or unsubstituted C₂-C₂₀-alkynyl, substituted or unsubstituted C₂-C₂₀-alkenyl, preferably substituted or unsubstituted C₅-C₄₈-aryl. Substituents on the respective substituents may be selected as set forth above for R^{S}.

Preferably, R^{B} is independently selected from the group consisting of H, D, C₅-C₄₈-aryl, C₆-C₁₄-aryl, C₆-C₁₀-aryl, C₅-C₄₈-aryloxy, C₆-C₁₄-aryloxy, C₆-C₁₀-aryloxy, C₁-C₂₀-alkyl, C₁-C₁₀-alkyl, C₁-C₆-alkyl, and C₁-C₄-alkyl, C₁-C₂₀-alkoxy, C₁-C₁₀-alkoxy, C₁-C₆-alkoxy, and C₁-C₄-alkoxy, C₁-C₂₀-alkyl-C₅-C₄₈-aryloxy, C₁-C₁₀-alkyl-C₆-C₁₄-aryloxy, C₁-C₄-alkyl-C₆-C₁₀-aryloxy, preferably independently selected from the group consisting of H, methoxy, benzylether and methyl.

In the structural motifs (BVI) and (BVIa), at least one of R⁵ and R⁷, preferably R⁵ and R⁷, may be an electron donating group as described above for at least one of R⁵ to R⁸, preferably the electron donating group being selected from substituted or unsubstituted C₁-C₂₀-alkoxy, preferably methoxy or benzyloxy, C₅-C₄₈-aryloxy, preferably phenyloxy, and NR″₂, wherein R″ is independently selected from the group consisting of substituted or unsubstituted C₁-C₂₀-alkyl and substituted or unsubstituted C₅-C₄₈-aryl, and two R″ may form a ring structure, preferably forming the structure of piperidine, morpholine, or pyrrolidine.

The structural motif (BI), (BII) or (BIII) may be the structural motif (BVII)

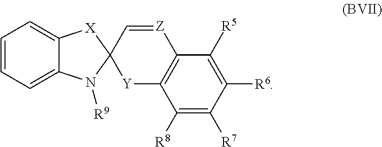

In other words, the photoinitiator molecule may comprise the structural motif (BVII).

Preferably X is selected from S or CR^{S}₂. More preferably X is CR^{S}₂ and R^{S} is independently selected from H, D, C₅-C₄₈-aryl, C₅-C₁₀-aryl, C₁-C₂₀-alkyl, C₁-C₁₀-alkyl, C₁-C₆-alkyl, and C₁-C₄-alkyl or the two R^{S} form together with the C (the atom X in the structural motif (BVII)) a substituted or unsubstituted C₁ to C₂₀ cycloalkyl ring. More preferably, X is CR^{S}₂ and the two R^{S} are methyl or the two R^{S} form together with the C (the atom X in the structural motif (BVII)) a C₁ to C₆ cycloalkyl ring, preferably a cyclohexyl ring.

Preferably Z is CR^{S}. R^{S} may be independently selected from H, D, CN, C₁-C₁₂-alkyl, fluorinated C₁-C₁₂-alkyl, or C₆-C₁₄-aryl. More preferably, Z is CR^{S} and R^{S} is H.

Preferably, Y is S or O. More preferably Y is O.

The structural motif (BVII) may be the structural motif (BVIIa)

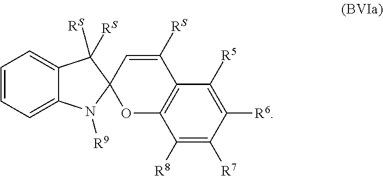

In other words, the photoinitiator molecule may comprise the structural motif (BVIIa).

In the structural motifs (BVII) and (BVIIa), at least one of R⁵ and R⁷ is independently an electron donating group. In the structural motifs (BVII) and (BVIIa), the electron donating group may be independently selected from the group consisting of substituted or unsubstituted C₁-C₂₀-alkoxy, C₅-C₄₈-aryloxy, and NR″₂, wherein R″ is independently selected from the group consisting of substituted or unsubstituted C₁-C₂₀-alkyl and substituted or unsubstituted C₅-C₄₈-aryl, and two R″ may form a ring structure. The electron donating group(s) may be as described above for the structural motifs (BVI) and (BVIa).

In the structural motifs (BVII) and (BVIIa), at least one of R⁶ and R⁸, preferably R⁸, is independently an electron withdrawing group. In the structural motifs (BVII) and (BVIIa), the electron withdrawing group which is part of the structural motifs (BVII) and (BVIIa) as at least one of R⁵ to R⁸ may be selected from the group consisting of F, Cl, Br, I, NO₂, CN, carbonyl, carbonate, carbonate ester, partially fluorinated C₁-C₂₀ alkyl, partially fluorinated C₅-C₄₈ aryl, perfluorinated C₁ to C₂₀ alkyl, perfluorinated C₅-C₄₈ aryl, C₂-C₂₀-alkyl acyl, C₅-C₄₈-aryl acyl, substituted C₂-C₄₉-alkyl acyl, substituted C₅-C₄₈-aryl acyl, C₃-C₄₈-heteroaryl acyl, substituted C₃-C₄₈-heteroaryl acyl substituted or unsubstituted C₁ to C₂₀-alkoxycarbonyl, substituted or unsubstituted (meth)acrylate, C₁-C₂₀ alkylsulfone, C₅-C₄₈-aryl sulfone, sulfone, SO₂Me, SO₂NH₂, methoxy, C₅-C₄₈-aryloxy carbonyl, formyl, tosyl, mesyl, C₃-C₄₈-heteroaryl acyl, C₂-C₂₀ alkyl amide, and C₅-C₄₈-aryl amide, preferably C₂-C₂₀-alkyl acyl, C₅-C₄₈-aryl acyl, substituted C₂-C₄₉-alkyl acyl, substituted C₅-C₄₈-aryl acyl, C₃-C₄₈-heteroaryl acyl, substituted C₃-C₄₈-heteroaryl acyl, C₁ to C₁₀-alkoxycarbonyl, such as ethoxycarbonyl or methoxycarbonyl, NO₂, CF₃, C₁, and CN, more preferred substituted or unsubstituted C₅-C₄₈-aryl acyl, even more preferred substituted or unsubstituted C₆-C₃₀-aryl acyl, most preferred substituted or unsubstituted (with unsubstituted being even more preferred) C₆ to C₂₀-aryl acyl.

If C₂ to C₂₀-alkoxycarbonyl (alkyl ester substituent) or C₅-C₄₈-aryloxy carbonyl (aryl ester substituent) is chosen as the electron withdrawing group, the binding position may be via the carbonyl-C or the oxygen atom.

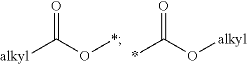

*=binding position

The same applies mutatis mutandis for the other electron withdrawing groups bearing a carbonyl group, such as amide etc.

The partially fluorinated C₁-C₂₀ alkyl may be partially fluorinated C₁-C₁₀ alkyl, partially fluorinated C₁-C₆ alkyl, partially fluorinated C₁-C₄ alkyl, partially fluorinated C₁-C₂ alkyl, CF₂H or CFH₂.

The perfluorinated C₁-C₂₀ alkyl may be perfluorinated C₁-C₁₀ alkyl, perfluorinated C₁-C₆ alkyl, perfluorinated C₁-C₄ alkyl, perfluorinated C₁-C₂ alkyl, CF₃.

The partially fluorinated C₅-C₄₈ aryl may be partially fluorinated C₆-C₄₂ aryl, partially fluorinated C₆-C₃₆ aryl, partially fluorinated C₆-C₃₀ aryl, partially fluorinated C₆-C₂₄ aryl, partially fluorinated C₆-C₁₈ aryl, partially fluorinated C₆-C₁₂ aryl, partially fluorinated C₆-C₁₀ aryl, or partially fluorinated phenyl.

The perfluorinated C₅-C₄₈ aryl may be perfluorinated C₆-C₄₂ aryl, perfluorinated C₆-C₃₆ aryl, perfluorinated C₆-C₃₀ aryl, perfluorinated C₆-C₂₄ aryl, perfluorinated C₆-C₁₈ aryl, perfluorinated C₆-C₁₂ aryl, perfluorinated C₆-C₁₀ aryl, or perfluorinated phenyl.

The C₂-C₂₀-alkyl acyl may be C₂-C₁₀-alkyl acyl, C₂-C₆-alkyl acyl, C₂-C₄-alkyl acyl, C₂-C₃-alkyl acyl, or methylacyl.

The C₅-C₄₈-aryl acyl may be C₆-C₄₂-aryl acyl, C₆-C₃₆-aryl acyl, C₆-C₃₀-aryl acyl, C₆-C₂₄-aryl acyl, C₆-C₁₈-aryl acyl, C₆-C₁₂-aryl acyl, or phenylacyl.

The C₂ to C₂₀-alkoxycarbonyl may be C₂ to C₁₀-alkoxycarbonyl, C₂ to C₆-alkoxycarbonyl, C₂ to C₄-alkoxycarbonyl, C₂ to C₃-alkoxycarbonyl, or methoxycarbonyl.

The C₅-C₄₈-aryloxy carbonyl may be C₆-C₄₂-aryloxy carbonyl, C₆-C₃₆-aryloxy carbonyl, C₆-C₃₀-aryloxy carbonyl, C₆-C₂₄-aryloxy carbonyl, C₆-C₁₈-aryloxy carbonyl, C₆-C₁₂-aryloxy carbonyl, or phenoxycarbonyl.

The C₅-C₄₈-aryl sulfone may be C₆-C₄₂-aryl sulfone, C₆-C₃₆-aryl sulfone, C₆-C₃₀-aryl sulfone, C₆-C₂₄-aryl sulfone, C₆-C₁₈-aryl sulfone, C₆-C₁₂-aryl sulfone, or phenylsulfone.

The C₁-C₂₀ alkylsulfone may be C₁-C₁₀ alkylsulfone, C₁-C₆ alkylsulfone, C₁-C₄ alkylsulfone, C₁-C₂ alkylsulfone, or methylsulfone.

The C₂-C₂₀ alkyl amide may be C₂-C₁₀ alkyl amide, C₂-C₆ alkyl amide, C₂-C₄ alkyl amide, C₂-C₃ alkyl amide, or methylamide.

The C₅-C₄₈-aryl amide may be C₆-C₄₂-aryl amide, C₆-C₃₆-aryl amide, C₆-C₃₀-aryl amide, C₆-C₂₄-aryl amide, C₆-C₁₈-aryl amide, C₆-C₁₂-aryl amide, or phenylamide.

In the structural motifs (BVII) and (BVIIa), the electron withdrawing group may be selected from the group consisting of NO₂, CN, perfluorinated C₁ to C₂₀ alkyl, C₂-C₂₀-alkyl acyl, C₅-C₄₈-aryl acyl, and substituted or unsubstituted C₂ to C₂₀ alkoxycarbonyl. In the structural motifs (BVII) and (BVIIa), the electron withdrawing group may be selected from the group consisting of NO₂. CN, perfluorinated C₁ to C₁₀ alkyl, C₂-C₁₀-alkyl acyl, C₆-C₃₀-aryl acyl, and substituted or unsubstituted C₂ to C₁₀ alkoxycarbonyl. In the structural motifs (BVII) and (BVIIa), the electron withdrawing group may be selected from the group consisting of NO₂, CN, perfluorinated C₁ to C₆ alkyl, C₂-C₆-alkyl acyl, C₆-C₁₈-aryl acyl, and substituted or unsubstituted C₂ to C₆ alkoxycarbonyl. In the structural motifs (BVII) and (BVIIa), the electron withdrawing group may be selected from the group consisting of NO₂, CN, perfluorinated C₁ to C₄ alkyl, C₂-C₄-alkyl acyl, C₆-C₁₂-aryl acyl, and substituted or unsubstituted C₂ to C₄ alkoxycarbonyl. In the structural motifs (BVII) and (BVIIa), the electron withdrawing group may be selected from the group consisting of NO₂, CN, CF₃, methylacyl, phenylacyl, and methoxycarbonyl.

In the structural motifs (BVII) and (BVIIa), at least one of R⁵ and R⁷ is an electron donating group. The one or more electron donating group(s) from which at least one of R⁵ to R⁸ may be independently selected may be independently selected from the group consisting of substituted or unsubstituted C₁-C₂₀-alkoxy, C₅-C₄₈-aryloxy, and NR″₂, wherein R″ is independently selected from the group consisting of substituted or unsubstituted C₁-C₂₀-alkyl and substituted or unsubstituted C₅-C₄₈-aryl, and two R″ may form a ring structure.

The C₁-C₂₀-alkoxy may be C₁-C₁₀-alkoxy, C₁-C₆-alkoxy, C₁-C₄-alkoxy, C₁-C₂-alkoxy, or methoxy.

The C₅-C₄₈-aryloxy may be C₆-C₄₂-aryloxy, C₆-C₃₆-aryloxy, C₆-C₃₀-aryloxy, C₆-C₂₄-aryloxy, C₆-C₁₈-aryloxy, C₆-C₁₂-aryloxy, or phenyloxy.

The C₁-C₂₀-alkyl which can be R″ in NR″₂ may be C₁-C₁₀-alkyl, C₁-C₆-alkyl, C₁-C₄-alkyl, C₁-C₂-alkyl, or methyl. If the two R″ are alkyl and form together a ring, the ring may be a C₅ ring, a C₆ ring or a C₇ ring.

The C₅-C₄₈-aryl which can be R″ in NR″₂ may be C₆-C₄₂-aryl, C₆-C₃₆-aryl, C₆-C₃₀-aryl, C₆-C₂₄-aryl, C₆-C₁₈-aryl, C₆-C₁₂-aryl, C₆-C₁₀-aryl, or phenyl. If the two R″ are alkyl and form together a ring, the ring may form together with the N-atom of NR″₂ a carbazolyl functional group.

If the respective electron donating group is substituted, the one or more substituents may be independently selected from the group consisting of H, D, halogen, NO₂, CN, OH, SH, substituted or unsubstituted C₁-C₂₀-alkyl, substituted or unsubstituted C₃-C₂₀-cycloalkyl, substituted or unsubstituted C₅-C₄₈-aryl, substituted or unsubstituted C₁-C₂₀-alkyl-C₅-C₄₈-aryl, substituted or unsubstituted C₃-C₄₂-heteroaryl, substituted or unsubstituted C₁-C₂₀-alkyl-C₃-C₄₂-heteroaryl, substituted or unsubstituted C₂-C₄₉-alkyl acyl, substituted or unsubstituted C₂-C₄₉-aryl acyl, substituted or unsubstituted C₁-C₂₀-alkoxy, substituted or unsubstituted C₅-C₄₈-aryloxy, C₁-C₂₀-alkyl-C₅-C₄₈-aryloxy, C₅-C₄₈-aryl-C₁-C₂₀-alkoxy, NH₂, substituted or unsubstituted C₁-C₂₀-alkyl ester, substituted or unsubstituted C₅-C₄₈-aryl ester, substituted or unsubstituted C₁-C₂₀ alkyl amide, substituted or unsubstituted C₅-C₄₈-aryl amide, NR′₂, SiR′₃, —O—SiR′₃, substituted or unsubstituted carboxylic acids and salts thereof, substituted or unsubstituted sulfonic acids and salts thereof, substituted or unsubstituted sulfonic esters, substituted or unsubstituted sulfonic amides, formyl, ether, thioether, carbonate, carbonate ester, sulfates, boronic acids, boronic esters, phosphonic acids, phosphonic esters, phosphines, phosphates, peroxycarbonic acids, thiocarbonic acids, sulfinic acids, sulfinic esters, sulfonates, thiolesters, sulfoxides, sulfones, hydrazides, thioaldehydes, ketones, thioketones, oximes, hydrazines, nitroso, azo, diazo, diazonium; isocyanides, cyanate, isocyanate, thiocyanate, isothiocyanate, hydroperoxide, peroxide, acetals, ketal, orthoester, orthocarbonate esters, ammonium, imines, imides, azide, nitrate, isonitrile, nitrosoxy, substituted or unsubstituted carbamates, substituted or unsubstituted ethers, substituted or unsubstituted polyether carbamates, substituted or unsubstituted arylazo, substituted or unsubstituted C₂-C₂₀-alkynyl, substituted or unsubstituted C₂-C₂₀-alkenyl, preferably substituted or unsubstituted C₅-C₄₈-aryl. Substituents on the respective substituent may be selected as set forth above for R^{S}

Preferably, electron donating group is selected from the following groups EDI to ED 3

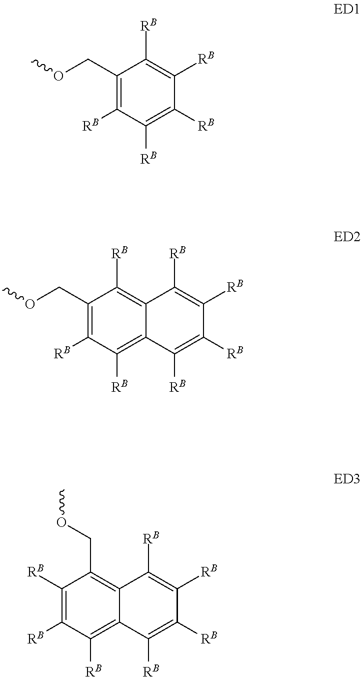

wherein
R^{B} is independently selected from the group consisting of H, D, halogen, NO₂, CN, OH, SH, substituted or unsubstituted C₁-C₂₀-alkyl, substituted or unsubstituted C₃-C₂₀-cycloalkyl, substituted or unsubstituted C₅-C₄₈-aryl, substituted or unsubstituted C₁-C₂₀-alkyl-C₅-C₄₈-aryl, substituted or unsubstituted C₃-C₄₂-heteroaryl, substituted or unsubstituted C₁-C₂₀-alkyl-C₃-C₄₂-heteroaryl, substituted or unsubstituted C₂-C₄₉-alkyl acyl, substituted or unsubstituted C₇-C₄₉-aryl acyl, substituted or unsubstituted C₁-C₂₀-alkoxy, substituted or unsubstituted C₅-C₄₈-aryloxy, C₁-C₂₀-alkyl-C₅-C₄₈-aryloxy, C₅-C₄₈-aryl-C₁-C₂₀-alkoxy, NH₂, substituted or unsubstituted C₁-C₂₀-alkyl ester, substituted or unsubstituted C₅-C₄₈-aryl ester, substituted or unsubstituted C₁-C₂₀ alkyl amide, substituted or unsubstituted C₅-C₄₈-aryl amide, NR′₂, SiR′₃, —O—SiR′₃, substituted or unsubstituted carboxylic acids and salts thereof, substituted or unsubstituted sulfonic acids and salts thereof, substituted or unsubstituted sulfonic esters, substituted or unsubstituted sulfonic amides, formyl, ether, thioether, carbonate, carbonate ester, sulfates, boronic acids, boronic esters, phosphonic acids, phosphonic esters, phosphines, phosphates, peroxycarbonic acids, thiocarbonic acids, sulfinic acids, sulfinic esters, sulfonates, thiolesters, sulfoxides, sulfones, hydrazides, thioaldehydes, ketones, thioketones, oximes, hydrazines, nitroso, azo, diazo, diazonium; isocyanides, cyanate, isocyanate, thiocyanate, isothiocyanate, hydroperoxide, peroxide, acetals, ketal, orthoester, orthocarbonate esters, ammonium, imines, imides, azide, nitrate, isonitrile, nitrosoxy, substituted or unsubstituted carbamates, substituted or unsubstituted ethers, substituted or unsubstituted polyether carbamates, substituted or unsubstituted arylazo, substituted or unsubstituted C₂-C₂₀-alkynyl, substituted or unsubstituted C₂-C₂₀-alkenyl, preferably substituted or unsubstituted C₅-C₄₈-aryl. Substituents on the respective substituents may be selected as set forth above for R^{S}.

Preferably, R^{B} is independently selected from the group consisting of H, D, C₅-C₄₈-aryl, C₆-C₁₄-aryl, C₆-C₁₀-aryl, C₅-C₄₈-aryloxy, C₆-C₁₄-aryloxy, C₆-C₁₀-aryloxy, C₁-C₂₀-alkyl, C₁-C₁₀-alkyl, C₁-C₆-alkyl, and C₁-C₄-alkyl, C₁-C₂₀-alkoxy, C₁-C₁₀-alkoxy, C₁-C₆-alkoxy, and C₁-C₄-alkoxy, C₁-C₂₀-alkyl-C₅-C₄₈-aryloxy, C₁-C₁₀-alkyl-C₆-C₁₄-aryloxy, C₁-C₄-alkyl-C₆-C₁₀-aryloxy, preferably independently selected from the group consisting of H, methoxy, benzylether and methyl.

The structural motif (BI) may be the structural motif (BVIII)

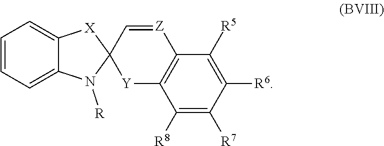

In other words, the photoinitiator molecule may comprise the structural motif (BVIII).

In the structural motif (BVIII) R may be R⁹ as defined above with respect to the structural motif (BII).

Preferably X is selected from S or CR^{S}₂. More preferably X is CR^{S}₂ and R^{S} is independently selected from H, D, C₅-C₄₈-aryl, C₆-C₁₀-aryl, C₁-C₂₀-alkyl, C₁-C₁₀-alkyl, C₁-C₆-alkyl, and C₁-C₄-alkyl or the two R^{S} form together with the C (the atom X in the structural motif (BVIII)) a substituted or unsubstituted C₁ to C₂₀ cycloalkyl ring. More preferably, X is CR^{S}₂ and the two R^{S} are methyl or the two R^{S} form together with the C (the atom X in the structural motif (BVIII)) a C₁ to C₆ cycloalkyl ring, preferably a cyclohexyl ring.

Preferably Z is CR^{S}. R^{S} may be independently selected from H, D, CN, C₁-C₁₂-alkyl, fluorinated C₁-C₁₂-alkyl, or C₆-C₁₄-aryl. More preferably, Z is CR^{S} and R^{S} is H.

Preferably, Y is S or O. More preferably Y is O.

The structural motif (BVIII) may be the structural motif (BVIIIa)

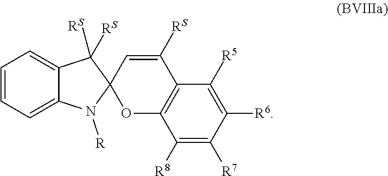

That is, the photoinitiator molecule may comprise the structural motif (BVIIIa).

In the structural motifs (BVIII) and (BVIIIa), R and R^{S} are independently selected as defined above with respect to the structural motif (BI).

In a preferred embodiment in the structural motifs (BVIII) and (BVIIIa), R is substituted or unsubstituted C₁-C₂₀-alkyl, preferably substituted or unsubstituted C₁-C₆-alkyl, more preferably substituted or unsubstituted C₁-C₄-alkyl, most preferably methyl.

In the structural motifs (BVIII) and (BVIIIa) where R is alkyl or aryl improved optical properties of the printed objects are observed.

At least one of R⁵ to R⁸ is an electron donating group. The one or more electron donating group(s) from which at least one of R⁵ to R⁸ are independently selected, are independently selected from the group consisting of substituted or unsubstituted C₁-C₂₀-alkoxy, C₅-C₄₈-aryloxy, and NR″₂, wherein R″ is independently selected from the group consisting of substituted or unsubstituted C₁-C₂₀-alkyl and substituted or unsubstituted C₅-C₄₈-aryl, and two R″ may form a ring structure.

The C₁-C₂₀-alkoxy may be C₁-C₁₀-alkoxy, C₁-C₆-alkoxy, C₁-C₄-alkoxy, C₁-C₂-alkoxy, or methoxy.

The C₅-C₄₈-aryloxy may be C₆-C₄₂-aryloxy, C₆-C₃₆-aryloxy, C₆-C₃₀-aryloxy, C₆-C₂₄-aryloxy, C₆-C₁₈-aryloxy, C₆-C₁₂-aryloxy, or phenyloxy.

The C₁-C₂₀-alkyl which can be R″ in NR″₂ may be C₁-C₁₀-alkyl, C₁-C₆-alkyl, C₁-C₄-alkyl, C₁-C₂-alkyl, or methyl. If the two R″ are alkyl and form together a ring, the ring may be a C₅ ring, a C₆ ring or a C₇ ring.

The C₅-C₄₈-aryl which can be R″ in NR″₂ may be C₆-C₄₂-aryl, C₆-C₃₆-aryl, C₆-C₃₀-aryl, C₆-C₂₄-aryl, C₆-C₁₈-aryl, C₆-C₁₂-aryl, C₆-C₁₀-aryl, or phenyl. If the two R″ are alkyl and form together a ring, the ring may form together with the N-atom of NR″₂ a carbazolyl functional group.

If the respective electron donating group is substituted, the one or more substituents may be independently selected from the group consisting of H, D, halogen, NO₂, CN, OH, SH, substituted or unsubstituted C₁-C₂₀-alkyl, substituted or unsubstituted C₃-C₂₀-cycloalkyl, substituted or unsubstituted C₅-C₄₈-aryl, substituted or unsubstituted C₁-C₂₀-alkyl-C₅-C₄₈-aryl, substituted or unsubstituted C₃-C₄₂-heteroaryl, substituted or unsubstituted C₁-C₂₀-alkyl-C₃-C₄₂-heteroaryl; substituted or unsubstituted C₂-C₄₉-alkyl acyl, substituted or unsubstituted C₂-C₄₉-aryl acyl, substituted or unsubstituted C₁-C₂₀-alkoxy, substituted or unsubstituted C₅-C₄₈-aryloxy, C₁-C₂₀-alkyl-C₅-C₄₈-aryloxy, C₅-C₄₈-aryl-C₁-C₂₀-alkoxy, NH₂, substituted or unsubstituted C₁-C₂₀-alkyl ester, substituted or unsubstituted C₅-C₄₈-aryl ester, substituted or unsubstituted C₁-C₂₀ alkyl amide, substituted or unsubstituted C₅-C₄₈-aryl amide, NR′₂, SiR′₃, —O—SiR′₃, substituted or unsubstituted carboxylic acids and salts thereof, substituted or unsubstituted sulfonic acids and salts thereof, substituted or unsubstituted sulfonic esters, substituted or unsubstituted sulfonic amides, formyl, ether, thioether, carbonate, carbonate ester, sulfates, boronic acids, boronic esters, phosphonic acids, phosphonic esters, phosphines, phosphates, peroxycarbonic acids, thiocarbonic acids, sulfinic acids, sulfinic esters, sulfonates, thiolesters, sulfoxides, sulfones, hydrazides, thioaldehydes, ketones, thioketones, oximes, hydrazines, nitroso, azo, diazo, diazonium; isocyanides, cyanate, isocyanate, thiocyanate, isothiocyanate, hydroperoxide, peroxide, acetals, ketal, orthoester, orthocarbonate esters, ammonium, imines, imides, azide, nitrate, isonitrile, nitrosoxy, substituted or unsubstituted carbamates, substituted or unsubstituted ethers, substituted or unsubstituted polyether carbamates, substituted or unsubstituted arylazo, substituted or unsubstituted C₂-C₂₀-alkynyl, substituted or unsubstituted C₂-C₂₀-alkenyl. Substituents on the respective substituent may be selected as set forth above for R^{S}.

In the structural motifs (BVIII) and (BVIIIa), at least one of R⁵ and R⁷ may be the electron donating group, preferably selected from substituted or unsubstituted C₁-C₂₀-alkoxy, preferably methoxy, C₅-C₄₈-aryloxy, and NR″₂, wherein R″ is independently selected from the group consisting of substituted or unsubstituted C₁-C₂₀-alkyl and substituted or unsubstituted C₅-C₄₈-aryl, and two R″ may form a ring structure.

In the structural motifs (BVIII) and (BVIIIa), at least one of R⁵-R⁸, preferably R⁶ and R⁸, most preferably R⁸, is independently an electron withdrawing group. In the structural motifs (BVIII) and (BVIIIa), the electron withdrawing group which is part of the structural motifs (BVIII) and (BVIIIa) as at least one of R⁵ to R⁸ may be selected from the group consisting of F, Cl, Br, I, NO₂, CN, carbonyl, carbonate, carbonate ester, partially fluorinated C₁-C₂₀ alkyl, partially fluorinated C₅-C₄₈ aryl, perfluorinated C₁ to C₂₀ alkyl, perfluorinated C₅-C₄₈ aryl, C₂-C₂₀-alkyl acyl, C₅-C₄₈-aryl acyl, substituted C₂-C₄₉-alkyl acyl, substituted C₅-C₄₈-aryl acyl, C₃-C₄₈-heteroaryl acyl, substituted C₃-C₄₈-heteroaryl acyl substituted or unsubstituted C₂ to C₂₀-alkoxycarbonyl, substituted or unsubstituted (meth)acrylate, C₁-C₂₀ alkylsulfone, C₅-C₄₈-aryl sulfone, sulfone, SO₂Me, SO₂NH₂, methoxy, C₅-C₄₈-aryloxy carbonyl, formyl, tosyl, mesyl, C₃-C₄₈-heteroaryl acyl, C₂-C₂₀ alkyl amide, and C₅-C₄₈-aryl amide, preferably C₂-C₂₀-alkyl acyl, C₅-C₄₈-aryl acyl, substituted C₂-C₄₉-alkyl acyl, substituted C₅-C₄₈-aryl acyl, C₃-C₄₈-heteroaryl acyl, substituted C₃-C₄₈-heteroaryl acyl NO₂, CF₃, C₁, and CN, more preferred substituted or unsubstituted C₅-C₄₈-aryl acyl, even more preferred substituted or unsubstituted C₆-C₃₀-aryl acyl, most preferred substituted or unsubstituted (with unsubstituted being even more preferred) C₆ to C₂₀-aryl acyl.

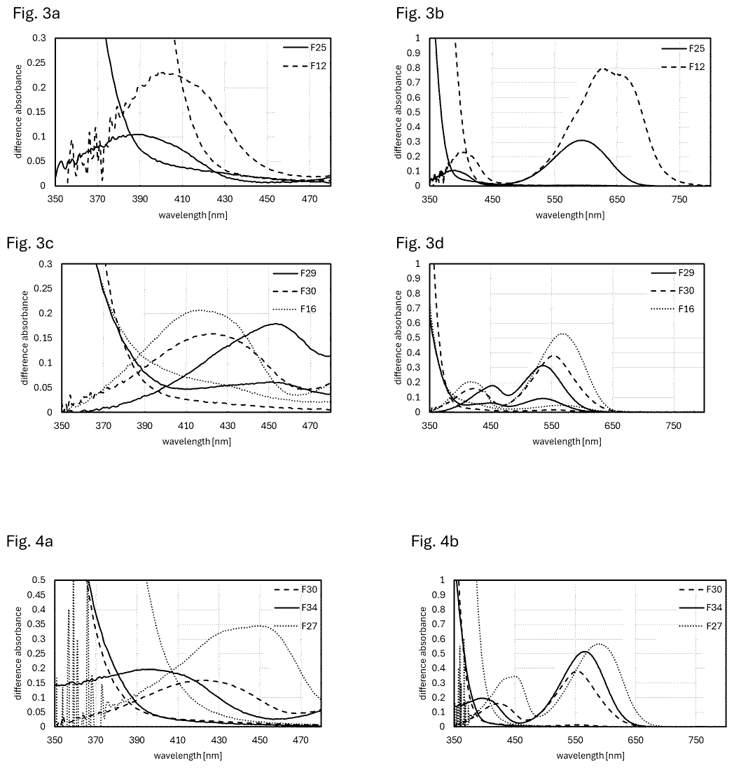

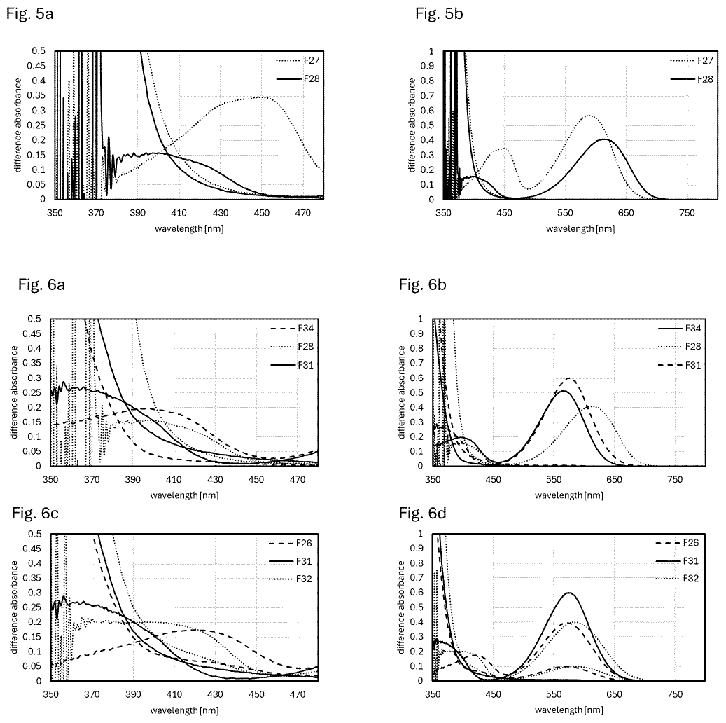

The structural motifs (BVIII) and (BVIIIa) show advantageous absorption properties in dual color photopolymerization processes. Typical dual color photoinitiators based on spiropyrans show a larger absorption band in the visible region around 550-650 nm, which is used for irradiation with the second wavelength. However, typical dual color photoinitiators show an additional prominent second absorption band around 400 nm, which is broad enough to cover part of the near UV and blue region. This absorption band usually overlays with the first wavelength of the dual color photopolymerization, meaning that during irradiation with the first wavelength, the absorption at the first wavelength increases. As a consequence, the penetration of the light of the first wavelength into the volume is reduced during irradiation limiting the achievable print object size and causing inhomogeneous curing or reduced structure fidelity. Surprisingly, dual color photoinitiators according to the structural motifs (BVIII) and (BVIIIa), where at least one of R⁵ to R⁷ is an electron donating group, are characterized in a shifted second absorption band to longer wavelengths. This leads to strongly reduced extinction coefficients of the intermediate state in the near UV and deep blue region (350-420 nm). This is also reflected in lower difference ratios of photopolymerizable starting materials which use dual color photoinitiators according to the structural motifs (BVIII) and (BVIIIa). Such a property is particularly relevant in structures, where R⁸ is an electron withdrawing group, such as an arylacyl group, which allows the production of colorless objects. The common procedure to manufacture colorless objects by dual color photopolymerization requires an additional bleaching step after the standard post-processing to remove a yellow species which has been formed during the polymerization process. In dual color photoinitiators according to the structural motifs (BVIII) and (BVIIIa) with the substituents as outlined above this bleaching process is efficient enough to be complete after post-curing omitting the necessity of an extra process step. Hence dual color photoinitiators of the structural motifs (BVIII) and (BVIIIa) are of particular relevance for applications, that require high precision and colorless print objects such as e.g. printing of optical elements. Surprisingly, the positive effect of electron donating groups in the R⁵ to R⁷ position on the absorption spectra is much stronger when an electron withdrawing group is placed at R⁸ compared to an electron withdrawing group at R⁶ or R⁷ (difference ratio of Formulations F27, F30, F34 and respective FIGS. 4a and 4b). The dual color photoinitiators employed in F27, F30, and F34 are substituted with an electron donating methoxy group in R⁵ and differ only in the position of a benzoyl substituent. The advantageous shift of the second absorption band of the merocyanine is small for F34, where the benzoyl substituent is in position R⁶ of the dual color photoinitiator. When the benzoyl substituent is placed in R⁷ or R⁸ a stronger shift is observed. However, placing the benzoyl in R⁷ also leads to a shift of the spiropyran absorption band and an intensity increase of the second absorption band, which has a shift of the usable first wavelength as a consequence, leading again to a reduction in the difference ratio (FIG. 5a). Regarding preferred positions and the advantageous effect of the electron donating substituents formulations F12, F16, F25, F29, and F30 can be compared together with the respective FIGS. 3a-3d, where all employed dual color photoinitiators have a benzoyl substituent in the R⁸ position. Placing a donor in the R⁶ position leads to a shift of the second absorption band (comparing F12 and F25, FIG. 3a), however without substantially altering the difference ratio, since the donor in R⁶ forces a longer first wavelength due to a shift of the spiropyran absorption band. Surprisingly, placing an electron donating group in position R⁵ or R⁷ or both also leads to the advantageous shift of the second merocyanine band, but without substantially altering the spiropyran absorption at the first wavelength (FIG. 3c). Consequently, the difference ratio for F16, F29, and F30 is significantly reduced compared to F12 and F25. A similar comparison of dual color photoinitiators substituted with benzoyl in R⁶ shows that also placing the electron donating groups in other positions or using more than one electron donating group does not lead to substantial improvements regarding the absorption spectra or difference ratio (formulations F26, F28, F31, F32, F34 and FIGS. 6a-6d).

Dual color photoinitators with an acyl group in the R⁸ position such as I-28 usually suffer from low reactivity with respect to the visible light irradiation. Consequently, for successful printing a high intensity of the light of the second wavelength is required. Surprisingly, this problem is solved by introducing at least one electron donating group in the R⁵ to R⁷ position. This effect is reflected in the minimum energy of the light of the first wavelength which is required to cause curing under dual color irradiation in dependency of the energy of the light of the second wavelength. While at low intensities of the light of the second wavelength dual color photoinitiators such as I-28 are not reactive or require a high energy of the light of the first wavelength, dual color photoinitiators comprising the electron donating group in at least one of R⁵ to R⁷ and an electron withdrawing group in the R⁸ position still show normal reactivity and can be used for volumetric printing. This is particularly relevant for larger build rooms, since the light of the second wavelength is usually applied from a projector and increasing the build room also increases the projection size and thus reduces the available light intensity of the light of the second wavelength.

Dual color photoinitators according to the structural motifs (BVIII) and (BVIIIa) with an acyl group in the R⁸ position can be synthesized directly from respective salicylaldehyde derivatives. The common synthetic way to spiropyran derivatives with an acyl group in the R⁸ position involves the preparation of a halogenated spiropyran (step 1), followed by halogen-metal exchange and reaction with a Weinreb amide (step 2), and exchange reaction to introduce the desired indole derivative (step 3). This approach is limited to electron withdrawing substituents on the indole side of the spiropyran since otherwise the exchange reaction is low yielding or not working at all. For salicylaldehyde derivatives with an electron donating group in one of the positions meta to the hydroxy group (later positions R⁵ or R⁷ in the spiropyran) such as 2-Hydroxy-6-methoxybenzaldehyde, a Friedel Crafts reaction is directed to the position ortho to the hydroxy group (later position R⁸ in the spiropyran). Therefore, dual color photoinitators according to the structural motifs (BVIII) and (BVIIIa) can profit from a shorter synthetic route and more flexibility regarding the substituents on the indole part of the molecule. A similar “direct” synthetic approach without the electron donating group leads to the common spiropyrans with an acyl group in the R⁶ position:

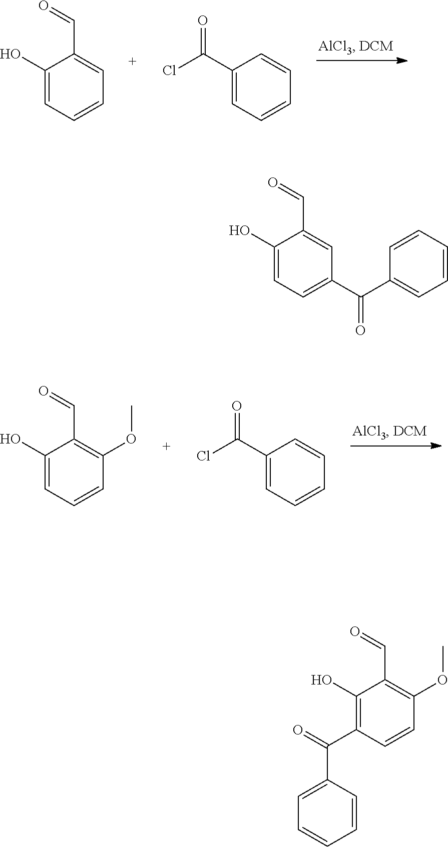 [SMILES: O=Cc1ccccc1O ; O=C(Cl)c1ccccc1 ; O=Cc1cc(C(=O)c2ccccc2)ccc1O ; COc1cccc(O)c1C=O ; O=C(Cl)c1ccccc1 ; COc1ccc(C(=O)c2ccccc2)c(O)c1C=O]

The photoinitiator molecule comprises one or more, preferably one of the above structural motifs (BI) to (BVIII), including the structural motifs (BIIa) to (BVIIIa). This means that the photoinitiator molecule either consists of one of said structural motifs or comprises the respective structural motif and at least one arbitrary group, preferably at least one arbitrary organic group attached thereto.

The photoinitiator molecule comprising the above structural motifs (BI) to (BVIII) may be represented by the following formula (BIX).

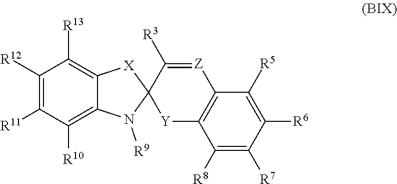

wherein
X is selected from the group consisting of S, CR¹R², and NR¹;
Y is selected from the group consisting of O, S, and NR^{c}; when Y is NR^{c}, R^{c} contains the atoms necessary to complete a cyclic structure with R⁸, wherein the cyclic structure is selected from the group consisting of benzimidazole, indoline, indole, dihydroquinoline, and tetrahydroquinoline;
Z is selected from the group consisting of N and CR⁴.

Any one of R¹ to R¹³ defined above with respect to one of the structural motifs (BI) to (BVIII), including the structural motifs (BIIa) to (BVIIIa), is then defined the same in the compound of formula (BIX) (for example, if the photoinitiator molecule has the formula (BIX) and comprises the structural motif (BII), the R⁹ is as defined above with respect to the structural motif (BII)) and the remaining of R¹ to R¹³ not defined above with respect to the respective structural motif are independently selected from the group consisting of:

H, D, halogen, NO₂, CN, OH, SH, substituted or unsubstituted C₁-C₂₀-alkyl, substituted or unsubstituted C₃-C₂₀-cycloalkyl, substituted or unsubstituted C₅-C₄₈-aryl, substituted or unsubstituted C₁-C₂₀-alkyl-C₅-C₄₈-aryl, preferably C₁-C₂₀-alkyl-electron withdrawing-C₅-C₄₈-aryl; substituted or unsubstituted C₃-C₄₂-heteroaryl, substituted or unsubstituted C₁-C₂₀-alkyl-C₃-C₄₂-heteroaryl, preferably C₁-C₂₀-alkyl-electron withdrawing-C₃-C₄₂-heteroaryl; substituted or unsubstituted C₂-C₄₉-alkyl acyl, substituted or unsubstituted C₂-C₄₉-aryl acyl, substituted or unsubstituted C₁-C₂₀-alkoxy, substituted or unsubstituted C₅-C₄₈-aryloxy, C₁-C₂₀-alkyl-C₅-C₄₈-aryloxy, C₅-C₄₈-aryl-C₁-C₂₀-alkoxy, NH₂, substituted or unsubstituted C₁-C₂₀-alkyl ester, substituted or unsubstituted C₅-C₄₈-aryl ester, substituted or unsubstituted C₁-C₂₀ alkyl amide, substituted or unsubstituted C₅-C₄₈-aryl amide, NR′₂, SiR′₃, —O—SiR′₃, substituted or unsubstituted carboxylic acids and salts thereof, substituted or unsubstituted sulfonic acids and salts thereof, substituted or unsubstituted sulfonic esters, substituted or unsubstituted sulfonic amides, formyl, ether, thioether, carbonate, carbonate ester, sulfates, boronic acids, boronic esters, phosphonic acids, phosphonic esters, phosphines, phosphates, peroxycarbonic acids, thiocarbonic acids, sulfinic acids, sulfinic esters, sulfonates, thiolesters, sulfoxides, sulfones, hydrazides, thioaldehydes, ketones, thioketones, oximes, hydrazines, nitroso, azo, diazo, diazonium; isocyanides, cyanate, isocyanate, thiocyanate, isothiocyanate, hydroperoxide, peroxide, acetals, ketal, orthoester, orthocarbonate esters, ammonium, imines, imides, azide, nitrate, isonitrile, nitrosoxy, substituted or unsubstituted carbamates, substituted or unsubstituted ethers, substituted or unsubstituted polyether carbamates, substituted or unsubstituted arylazo, substituted or unsubstituted C₂-C₂₀-alkynyl, substituted or unsubstituted C₂-C₂₀-alkenyl. R′ may be independently selected from the group consisting of substituted or unsubstituted C₁-C₂₀-alkyl and substituted or unsubstituted C₅-C₄₈-aryl, and two R′ may form a ring structure.

Two R″ attached to the same atom, such as R¹ and R², or attached to adjacent carbon atoms, such as R¹² and R¹³, may be linked to each other to form a ring. Especially, two R″ attached to the same atom, such as R¹ and R² may form together with the carbon atom to which they are attached an aliphatic ring, such as a C₆ ring. Especially, two R″ attached to adjacent carbon atoms, such as R¹² and R¹³, may be linked to each other to form a fused aromatic ring or a fused aromatic ring system. The formed ring may be unsubstituted or substituted

If one or more of R¹ to R¹³ and/or a ring formed by two or more thereof is substituted, the respective substituents can be selected from the group consisting of: H, D, halogen, NO₂, CN, OH, SH, substituted or unsubstituted C₁-C₂₀-alkyl, substituted or unsubstituted C₃-C₂₀-cycloalkyl, substituted or unsubstituted C₅-C₄₈-aryl, substituted or unsubstituted C₁-C₂₀-alkyl-C₅-C₄₈-aryl, preferably C₁-C₂₀-alkyl-electron withdrawing-C₅-C₄₈-aryl; substituted or unsubstituted C₃-C₄₂-heteroaryl, substituted or unsubstituted C₁-C₂₀-alkyl-C₃-C₄₂-heteroaryl, preferably C₁-C₂₀-alkyl-electron withdrawing-C₃-C₄₂-heteroaryl; substituted or unsubstituted C₂-C₄₉-alkyl acyl, substituted or unsubstituted C₂-C₄₉-aryl acyl, substituted or unsubstituted C₁-C₂₀-alkoxy, substituted or unsubstituted C₅-C₄₈-aryloxy, C₁-C₂₀-alkyl-C₅-C₄₈-aryloxy, C₅-C₄₈-aryl-C₁-C₂₀-alkoxy, NH₂, substituted or unsubstituted C₁-C₂₀-alkyl ester, substituted or unsubstituted C₅-C₄₈-aryl ester, substituted or unsubstituted C₁-C₂₀ alkyl amide, substituted or unsubstituted C₅-C₄₈-aryl amide, NR′₂, SiR′₃, —O—SiR′₃, substituted or unsubstituted carboxylic acids and salts thereof, substituted or unsubstituted sulfonic acids and salts thereof, substituted or unsubstituted sulfonic esters, substituted or unsubstituted sulfonic amides, formyl, ether, thioether, carbonate, carbonate ester, sulfates, boronic acids, boronic esters, phosphonic acids, phosphonic esters, phosphines, phosphates, peroxycarbonic acids, thiocarbonic acids, sulfinic acids, sulfinic esters, sulfonates, thiolesters, sulfoxides, sulfones, hydrazides, thioaldehydes, ketones, thioketones, oximes, hydrazines, nitroso, azo, diazo, diazonium; isocyanides, cyanate, isocyanate, thiocyanate, isothiocyanate, hydroperoxide, peroxide, acetals, ketal, orthoester, orthocarbonate esters, ammonium, imines, imides, azide, nitrate, isonitrile, nitrosoxy, substituted or unsubstituted carbamates, substituted or unsubstituted ethers, substituted or unsubstituted polyether carbamates, substituted or unsubstituted arylazo, substituted or unsubstituted C₂-C₂₀-alkynyl, substituted or unsubstituted C₂-C₂₀-alkenyl. R′ may be independently selected from the group consisting of substituted or unsubstituted C₁-C₂₀-alkyl and substituted or unsubstituted C₅-C₄₈-aryl, and two R′ may form a ring structure, wherein the respective substituents can, in turn be preferably substituted with D or C₁ to C₆ alky.

R¹ to R¹³ not defined above with respect to the respective structural motif may be independently selected from the group consisting of:

H, D, halogen, NO₂, CN, OH, SH, substituted or unsubstituted C₁-C₂₀-alkyl, substituted or unsubstituted C₃-C₂₀-cycloalkyl, substituted or unsubstituted C₅-C₄₈-aryl, substituted or unsubstituted C₁-C₂₀-alkyl-C₅-C₄₈-aryl, substituted or unsubstituted C₃-C₄₂-heteroaryl, substituted or unsubstituted C₁-C₂₀-alkyl-C₃-C₄₂-heteroaryl, substituted or unsubstituted C₂-C₄₉-alkyl acyl, substituted or unsubstituted C₂-C₄₉-aryl acyl, substituted or unsubstituted C₁-C₂₀-alkoxy, substituted or unsubstituted C₅-C₄₈-aryloxy, C₁-C₂₀-alkyl-C₅-C₄₈-aryloxy, C₆-C₄₈-aryl-C₁-C₂₀-alkoxy, NH₂, substituted or unsubstituted C₁-C₂₀-alkyl ester, substituted or unsubstituted C₅-C₄₈-aryl ester, substituted or unsubstituted C₁-C₂₀ alkyl amide, substituted or unsubstituted C₅-C₄₈-aryl amide, NR′₂, SiR′₃, —O—SiR′₃, substituted or unsubstituted carboxylic acids and salts thereof, substituted or unsubstituted sulfonic acids and salts thereof, substituted or unsubstituted sulfonic esters, substituted or unsubstituted sulfonic amides, formyl, ether, thioether, carbonate, carbonate ester, sulfates, boronic acids, boronic esters, phosphonic acids, phosphonic esters, phosphines, phosphates, peroxycarbonic acids, thiocarbonic acids, sulfinic acids, sulfinic esters, sulfonates, thiolesters, sulfoxides, sulfones, hydrazides, thioaldehydes, ketones, thioketones, oximes, hydrazines, nitroso, azo, diazo, diazonium; isocyanides, cyanate, isocyanate, thiocyanate, isothiocyanate, hydroperoxide, peroxide, acetals, ketal, orthoester, orthocarbonate esters, ammonium, imines, imides, azide, nitrate, isonitrile, nitrosoxy, substituted or unsubstituted carbamates, substituted or unsubstituted ethers, substituted or unsubstituted polyether carbamates, substituted or unsubstituted arylazo, substituted or unsubstituted C₂-C₂₀-alkynyl, substituted or unsubstituted C₂-C₂₀-alkenyl. R′ may be independently selected from the group consisting of substituted or unsubstituted C₁-C₂₀-alkyl and substituted or unsubstituted C₅-C₄₈-aryl, and two R′ may form a ring structure.

If one or more of R¹ to R¹³ and/or a ring formed by two or more thereof is substituted, the respective substituents can be selected from the group consisting of: H, D, halogen, NO₂, CN, OH, SH, substituted or unsubstituted C₁-C₂₀-alkyl, substituted or unsubstituted C₃-C₂₀-cycloalkyl, substituted or unsubstituted C₅-C₄₈-aryl, substituted or unsubstituted C₁-C₂₀-alkyl-C₅-C₄₈-aryl, preferably C₁-C₂₀-alkyl-electron withdrawing-C₅-C₄₈-aryl; substituted or unsubstituted C₃-C₄₂-heteroaryl, substituted or unsubstituted C₁-C₂₀-alkyl-C₃-C₄₂-heteroaryl, preferably C₁-C₂₀-alkyl-electron withdrawing-C₃-C₄₂-heteroaryl; substituted or unsubstituted C₂-C₄₉-alkyl acyl, substituted or unsubstituted C₂-C₄₉-aryl acyl, substituted or unsubstituted C₁-C₂₀-alkoxy, substituted or unsubstituted C₅-C₄₈-aryloxy, C₁-C₂₀-alkyl-C₅-C₄₈-aryloxy, C₅-C₄₈-aryl-C₁-C₂₀-alkoxy, NH₂, substituted or unsubstituted C₁-C₂₀-alkyl ester, substituted or unsubstituted C₅-C₄₈-aryl ester, substituted or unsubstituted C₁-C₂₀ alkyl amide, substituted or unsubstituted C₅-C₄₈-aryl amide, NR′₂, SiR′₃, —O—SiR′₃, substituted or unsubstituted carboxylic acids and salts thereof, substituted or unsubstituted sulfonic acids and salts thereof, substituted or unsubstituted sulfonic esters, substituted or unsubstituted sulfonic amides, formyl, ether, thioether, carbonate, carbonate ester, sulfates, boronic acids, boronic esters, phosphonic acids, phosphonic esters, phosphines, phosphates, peroxycarbonic acids, thiocarbonic acids, sulfinic acids, sulfinic esters, sulfonates, thiolesters, sulfoxides, sulfones, hydrazides, thioaldehydes, ketones, thioketones, oximes, hydrazines, nitroso, azo, diazo, diazonium; isocyanides, cyanate, isocyanate, thiocyanate, isothiocyanate, hydroperoxide, peroxide, acetals, ketal, orthoester, orthocarbonate esters, ammonium, imines, imides, azide, nitrate, isonitrile, nitrosoxy, substituted or unsubstituted carbamates, substituted or unsubstituted ethers, substituted or unsubstituted polyether carbamates, substituted or unsubstituted arylazo, substituted or unsubstituted C₂-C₂₀-alkynyl, substituted or unsubstituted C₂-C₂₀-alkenyl. R′ may be independently selected from the group consisting of substituted or unsubstituted C₁-C₂₀-alkyl and substituted or unsubstituted C₅-C₄₈-aryl, and two R′ may form a ring structure, wherein the respective substituents can, in turn be preferably substituted with D or C₁ to C₆ alky.

R¹ to R¹³ not defined above with respect to the respective structural motif may be independently selected from the group consisting of:

H, D, F, Cl, Br, I, NO₂, CN, OH, CHO, SH, CF₃, substituted or unsubstituted C₁-C₂₀-alkyl, substituted or unsubstituted C₃-C₂₀-cycloalkyl, substituted or unsubstituted C₅-C₄₈-aryl, substituted or unsubstituted C₂-C₄₉-alkyl acyl, substituted or unsubstituted C₂-C₄₉-aryl acyl, substituted or unsubstituted C₁-C₂₀-alkoxy, NH₂, substituted or unsubstituted C₁-C₂₀-alkyl ester, NR′₂, carboxylic acids and salts thereof, sulfonic acids and salts thereof, C₁-C₂₀-alkyl sulfone, SO₂NH₂, R′ may be independently selected from the group consisting of substituted or unsubstituted C₁-C₂₀-alkyl, and two R′ may form a ring structure.

R¹ to R¹³ not defined above with respect to the respective structural motif may be independently selected from the group consisting of:

H, D, F, Cl, Br, I, NO₂, CF₃, CN, OH, OMe, COOH, CONH₂, CHO, COPh, COMe, methyl, SO₃⁻ substituted or unsubstituted C₂-C₄₉-alkyl acyl, substituted or unsubstituted C₂-C₄₉-aryl acyl, NH₂, substituted or unsubstituted C₁-C₂₀-alkyl ester, NMe₂, NEt₂, SO₂Me, SO₂NH₂.

In one embodiment, the photoinitiator molecule is represented by the following formula (BX)

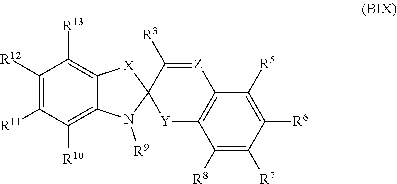

wherein
R⁹ is selected from the group consisting of H, D, halogen, NO₂, CN, OH, SH, substituted or unsubstituted C₁-C₂₀-alkyl, substituted or unsubstituted C₃-C₂₀-cycloalkyl, substituted or unsubstituted C₅-C₄₈-aryl, substituted or unsubstituted C₁-C₂₀-alkyl-C₅-C₄₈-aryl, preferably C₁-C₂₀-alkyl-electron withdrawing-C₅-C₄₈-aryl; substituted or unsubstituted C₃-C₄₂-heteroaryl, substituted or unsubstituted C₁-C₂₀-alkyl-C₃-C₄₂-heteroaryl, preferably C₁-C₂₀-alkyl-electron withdrawing-C₃-C₄₂-heteroaryl; substituted or unsubstituted C₂-C₄₉-alkyl acyl, substituted or unsubstituted C₂-C₄₉-aryl acyl, substituted or unsubstituted C₁-C₂₀-alkoxy, substituted or unsubstituted C₅-C₄₈-aryloxy, C₁-C₂₀-alkyl-C₅-C₄₈-aryloxy, C₅-C₄₈-aryl-C₁-C₂₀-alkoxy, NH₂, substituted or unsubstituted C₁-C₂₀-alkyl ester, substituted or unsubstituted C₅-C₄₈-aryl ester, substituted or unsubstituted C₁-C₂₀ alkyl amide, substituted or unsubstituted C₅-C₄₈-aryl amide, NR′₂, SiR′₃, —O—SiR′₃, substituted or unsubstituted carboxylic acids and salts thereof, substituted or unsubstituted sulfonic acids and salts thereof, substituted or unsubstituted sulfonic esters, substituted or unsubstituted sulfonic amides, formyl, ether, thioether, carbonate, carbonate ester, sulfates, boronic acids, boronic esters, phosphonic acids, phosphonic esters, phosphines, phosphates, peroxycarbonic acids, thiocarbonic acids, sulfinic acids, sulfinic esters, sulfonates, thiolesters, sulfoxides, sulfones, hydrazides, thioaldehydes, ketones, thioketones, oximes, hydrazines, nitroso, azo, diazo, diazonium; isocyanides, cyanate, isocyanate, thiocyanate, isothiocyanate, hydroperoxide, peroxide, acetals, ketal, orthoester, orthocarbonate esters, ammonium, imines, imides, azide, nitrate, isonitrile, nitrosoxy, substituted or unsubstituted carbamates, substituted or unsubstituted ethers, substituted or unsubstituted polyether carbamates, substituted or unsubstituted arylazo, substituted or unsubstituted C₂-C₂₀-alkynyl, substituted or unsubstituted C₂-C₂₀-alkenyl. R′ may be independently selected from the group consisting of substituted or unsubstituted C₁-C₂₀-alkyl and substituted or unsubstituted C₅-C₄₈-aryl, and two R′ may form a ring structure;
R¹ to R⁸ and R¹⁰ to R¹³ are independently selected from the group consisting of H, D, halogen, NO₂. CN, OH, SH, substituted or unsubstituted C₁-C₂₀-alkyl, substituted or unsubstituted C₃-C₂₀-cycloalkyl, substituted or unsubstituted C₅-C₄₈-aryl, substituted or unsubstituted C₁-C₂₀-alkyl-C₅-C₄₈-aryl, substituted or unsubstituted C₃-C₄₂-heteroaryl, substituted or unsubstituted C₁-C₂₀-alkyl-C₃-C₄₂-heteroaryl, substituted or unsubstituted C₂-C₄₉-alkyl acyl, substituted or unsubstituted C₂-C₄₉-aryl acyl, substituted or unsubstituted C₁-C₂₀-alkoxy, substituted or unsubstituted C₅-C₄₈-aryloxy, C₁-C₂₀-alkyl-C₅-C₄₈-aryloxy, C₅-C₄₈-aryl-C₁-C₂₀-alkoxy, NH₂, substituted or unsubstituted C₁-C₂₀-alkyl ester, substituted or unsubstituted C₅-C₄₈-aryl ester, substituted or unsubstituted C₁-C₂₀ alkyl amide, substituted or unsubstituted C₅-C₄₈-aryl amide, NR′₂, SiR′₃, —O—SiR′₃, substituted or unsubstituted carboxylic acids and salts thereof, substituted or unsubstituted sulfonic acids and salts thereof, substituted or unsubstituted sulfonic esters, substituted or unsubstituted sulfonic amides, formyl, ether, thioether, carbonate, carbonate ester, sulfates, boronic acids, boronic esters, phosphonic acids, phosphonic esters, phosphines, phosphates, peroxycarbonic acids, thiocarbonic acids, sulfinic acids, sulfinic esters, sulfonates, thiolesters, sulfoxides, sulfones, hydrazides, thioaldehydes, ketones, thioketones, oximes, hydrazines, nitroso, azo, diazo, diazonium; isocyanides, cyanate, isocyanate, thiocyanate, isothiocyanate, hydroperoxide, peroxide, acetals, ketal, orthoester, orthocarbonate esters, ammonium, imines, imides, azide, nitrate, isonitrile, nitrosoxy, substituted or unsubstituted carbamates, substituted or unsubstituted ethers, substituted or unsubstituted polyether carbamates, substituted or unsubstituted arylazo, substituted or unsubstituted C₂-C₂₀-alkynyl, substituted or unsubstituted C₂-C₂₀-alkenyl. R′ may be independently selected from the group consisting of substituted or unsubstituted C₁-C₂₀-alkyl and substituted or unsubstituted C₅-C₄₈-aryl, and two R′ may form a ring structure;
two R″ attached to the same atom, such as R¹ and R², or attached to adjacent carbon atoms, such as R¹² and R¹³, may be linked to each other to form a ring, especially, two R″ attached to the same atom, such as R¹ and R² may form together with the carbon atom to which they are attached an aliphatic ring, such as a C₆ ring, or especially, two R″ attached to adjacent carbon atoms, such as R¹² and R¹³, may be linked to each other to form a fused aromatic ring or a fused aromatic ring system. The formed ring may be unsubstituted or substituted; and if one or more of R¹ to R¹³ and/or a ring formed by two or more thereof is substituted, the respective substituents can be selected H, D, halogen, NO₂, CN, OH, SH, substituted or unsubstituted C₁-C₂₀-alkyl, substituted or unsubstituted C₃-C₂₀-cycloalkyl, substituted or unsubstituted C₅-C₄₈-aryl, substituted or unsubstituted C₁-C₂₀-alkyl-C₅-C₄₈-aryl, preferably C₁-C₂₀-alkyl-electron withdrawing-C₅-C₄₈-aryl; substituted or unsubstituted C₃-C₄₂-heteroaryl, substituted or unsubstituted C₁-C₂₀-alkyl-C₃-C₄₂-heteroaryl, preferably C₁-C₂₀-alkyl-electron withdrawing-C₃-C₄₂-heteroaryl; substituted or unsubstituted C₂-C₄₉-alkyl acyl, substituted or unsubstituted C₂-C₄₉-aryl acyl, substituted or unsubstituted C₁-C₂₀-alkoxy, substituted or unsubstituted C₅-C₄₈-aryloxy, C₁-C₂₀-alkyl-C₅-C₄₈-aryloxy, C₅-C₄₈-aryl-C₁-C₂₀-alkoxy, NH₂, substituted or unsubstituted C₁-C₂₀-alkyl ester, substituted or unsubstituted C₅-C₄₈-aryl ester, substituted or unsubstituted C₁-C₂₀ alkyl amide, substituted or unsubstituted C₅-C₄₈-aryl amide, NR′₂, SiR′₃, —O—SiR′₃, substituted or unsubstituted carboxylic acids and salts thereof, substituted or unsubstituted sulfonic acids and salts thereof, substituted or unsubstituted sulfonic esters, substituted or unsubstituted sulfonic amides, formyl, ether, thioether, carbonate, carbonate ester, sulfates, boronic acids, boronic esters, phosphonic acids, phosphonic esters, phosphines, phosphates, peroxycarbonic acids, thiocarbonic acids, sulfinic acids, sulfinic esters, sulfonates, thiolesters, sulfoxides, sulfones, hydrazides, thioaldehydes, ketones, thioketones, oximes, hydrazines, nitroso, azo, diazo, diazonium; isocyanides, cyanate, isocyanate, thiocyanate, isothiocyanate, hydroperoxide, peroxide, acetals, ketal, orthoester, orthocarbonate esters, ammonium, imines, imides, azide, nitrate, isonitrile, nitrosoxy, substituted or unsubstituted carbamates, substituted or unsubstituted ethers, substituted or unsubstituted polyether carbamates, substituted or unsubstituted arylazo, substituted or unsubstituted C₂-C₂₀-alkynyl, substituted or unsubstituted C₂-C₂₀-alkenyl. R′ may be independently selected from the group consisting of substituted or unsubstituted C₁-C₂₀-alkyl and substituted or unsubstituted C₅-C₄₈-aryl, and two R′ may form a ring structure, wherein the respective substituents can, in turn be preferably substituted with D or C₁ to C₆ alky.

It may be provided that if one or more of R¹ to R¹³ and/or a ring formed by two or more thereof is substituted, the respective substituents may be independently selected from the group consisting of D, methyl, tert-butyl, cyclohexyl, phenyl, CF₃, F, Cl, Br, I, CN, nitro, formyl, ethylester, methylester, acetyl, methoxy, OH, dimethylamino, NH₂, amide (CO—NH₂), carboxylic acid, sulfonic Acid, methylsulfone, and sulfonamide.

In a preferred embodiment two of the substituents R⁵-R⁸ may contain the atoms necessary to complete a coumarin structure, preferably two of R⁵-R⁸ form together the following structural motif

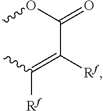

wherein the wavy lines represent the bonds connecting the structural motif formed by two of R⁵-R⁸ with the spiropyran and Rf is defined as R¹-R¹³ in formula (BX), preferably Rf is selected from H or methoxy.

Embodiments described above for any of the structural motifs (BI) to (BVIII) including the structural motifs (BIIa) to (BVIIIa) are also embodiments for the compound of formula (BX). For structures (BVIII) and subsequent structures in particular, it is advantageous to shift the first wavelength (especially its maximum) into the UV range, i.e. in a preferred variant <410 nm, better <400 nm, <390 nm, <380 nm, <370 nm, <360 nm, or <350 nm.

Surprisingly, it has been found that the performance of a xolography process is not just dependent on the extinction coefficient at the first wavelength or the extinction coefficient at the second wavelength but rather depends on the ability of the photopolymerizable starting material to generate as much absorption as possible at the second wavelength when the photopolymerizable starting material is irradiated with the first wavelength, while at the same time the absorption at the first wavelength should decrease as much as possible or at least increase just as little as possible. The relative change of simultaneous absorption changes can be described via the absorption ratio (AR) or the difference ratio (DR), which relate to how much absorption at the first wavelength is generated per absorption which is generated at the second wavelength during irradiation with light of the first wavelength. A high positive AR or DR resembles a photopolymerizable starting material where the absorption at the first wavelength increases during irradiation, thereby absorbing more light of the first wavelength and thus limiting the penetration of the light of the first wavelength into the volume. A low AR or DR is desired as it enhances penetration of the light of the first wavelength into the volume and as such allows for a more homogeneous intensity distribution of the light of the first wavelength and a more homogeneous degree of polymerization. Consequently, a photopolymerizable starting material with a low AR or DR can lead to increased structure fidelity, improved mechanical properties and especially enable also prints in larger build rooms along the irradiation direction of the light of the first wavelength. Photopolymerizable starting materials described in the state of the art suffer from high AR and DR, limiting build volumes and reducing structure fidelity, which is overcome by the present invention.

The Absorption Ratio (AR) of a photopolymerizable starting material depends on the nature of the dual color Photoinitiator and the composition of the other components of the photopolymerizable starting material, since extinction coefficients usually depend on the environment. The first wavelength can be shorter than the second wavelength and second wavelength can refer to the visible region (400 nm to 800 nm). The AR is defined as

$$AR = \frac{{Abs}_{UV , IS}}{{Abs}_{vis , IS}}$$

wherein Abs_{vis,IS} describes the absorption at the absorption maximum of only the intermediate state at the second wavelength in the photopolymerizable starting material and Abs_{UV,IS} describes the absorption of only the intermediate state at the first wavelength in the photopolymerizable starting material, where the intermediate state can be photogenerated and Abs_{vis,IS} and Abs_{UV,IS} refer to the same concentration of intermediate state. As such AR is equivalent to the ratio of extinction coefficient of the intermediate state at the first wavelength and the extinction coefficient of the intermediate state at the absorption maximum at the second wavelength.

Measuring the AR or extinction coefficient of the intermediate state in the photopolymerizable starting material is a complex and difficult process, since the obtained spectra usually contain a mixture of initial state and intermediate state. Despite this analysis problem the AR neglects the effect which the initial concentration of the dual color photoinitiator has on relative absorption changes. For a more practical and meaningful performance description of a photopolymerizable starting material difference spectra can be analyzed alternatively. For such an analysis the photopolymerizable starting material is filled in a cuvette with 10 mm path length. An initial spectrum before irradiation is recorded. The sample is irradiated using a light source of the first wavelength to obtain irradiation spectra. By subtraction of the initial spectrum from the irradiation spectra, difference spectra are obtained. It should be noted that prolonged irradiation can lead to photopolymerization with the first wavelength. To minimize this effect, the irradiation can be performed with high intensity of the light of the first wavelength for a short time until the absorption at the absorption maximum at the second wavelength reaches a defined value, such as 0.5. Where this value cannot be reached the intensity of the first wavelength can be the same or similar to the intensity used for the dual color photopolymerization process at quest and the irradiation time kept as short as possible. Analysis of the difference spectra yields the Difference Ratio (DR):

$$DR = \frac{{Abs}_{dif , UV , irr}}{{Abs}_{dif , vis , irr}}$$

Wherein Abs_{dif,vis,irr} describes the absorption at the absorption maximum of the difference spectrum at the second wavelength and Abs_{dif,UV,irr} describes the absorption of the difference spectrum at the first wavelength, where Abs_{dif,vis,irr} and Abs_{dif,UV,irr} refer to difference spectra where the concentration of the intermediate state is the same or similar or if not applicable Abs_{dif,vis,irr} and Abs_{dif,UV,irr} refer to the same difference spectrum. Compared to AR, DR additionally depends on the initial concentration of the dual color photoinitiator and is therefore a material parameter. AR and DR can refer to a single first wavelength, which refers to the wavelength of highest emission of the light of the first wavelength. AR and DR can refer to a second wavelength which can be longer than the first wavelength and where the second wavelength can refer to an absorption maximum within the wavelength range of the second wavelength.

For a typical scenario, where the initial state and the intermediate state absorb at the first wavelength, since the first wavelength is a shorter wavelength than the second wavelength, the absorption in the difference spectrum at the first wavelength is composed of the absorption of the intermediate state and the residual absorption of the initial state which has not been switched to the intermediate state subtracted by the absorption of the initial state before irradiation. As a consequence, the DR value is lower than the AR value or in other words, AR cannot be smaller than DR. While AR is specific for only a dual color photoinitiator, DR is specific for a photopolymerizable starting material including the initial concentration of the dual color photoinitiator. Hence DR is a well-defined and easy to measure parameter for a photopolymerizable starting material containing a dual color Photoinitiator. It has to be further noticed that although the exact concentration of the intermediate state remains unknown, DR is independent of the amount of intermediate state which has been formed during the irradiation process.

In one embodiment a photopolymerizable starting material is provided, wherein the difference ratio is DR <0.2, preferably DR <0.1, more preferably DR <=0, even more preferably DR <−0.1, still preferably <−0.3, most preferably <−0.5.

According to another aspect, the DR can refer to the first wavelength and the absorption difference maximum in the range of the second wavelength, preferably wherein the first wavelength is a wavelength between 360 nm-420 nm and/or the second wavelength covers the range from 450 nm-800 nm.

### Preferred Compounds:

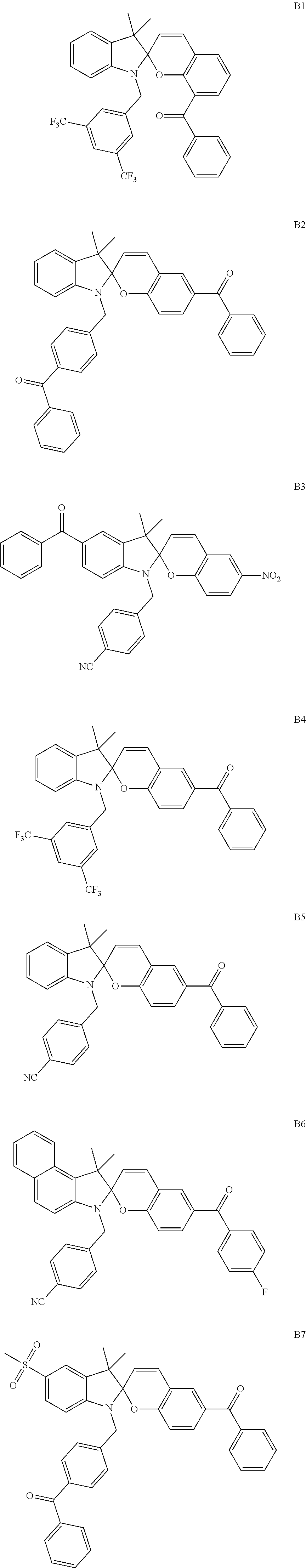

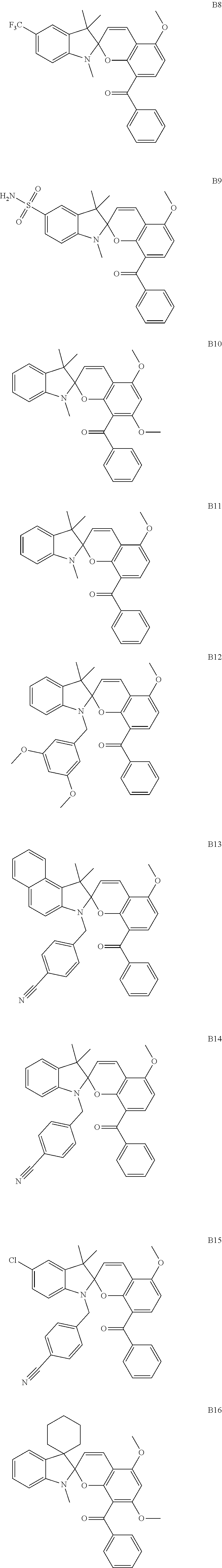

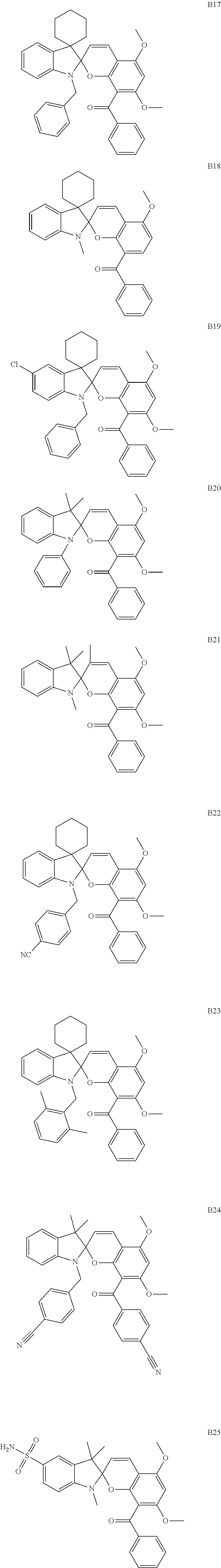

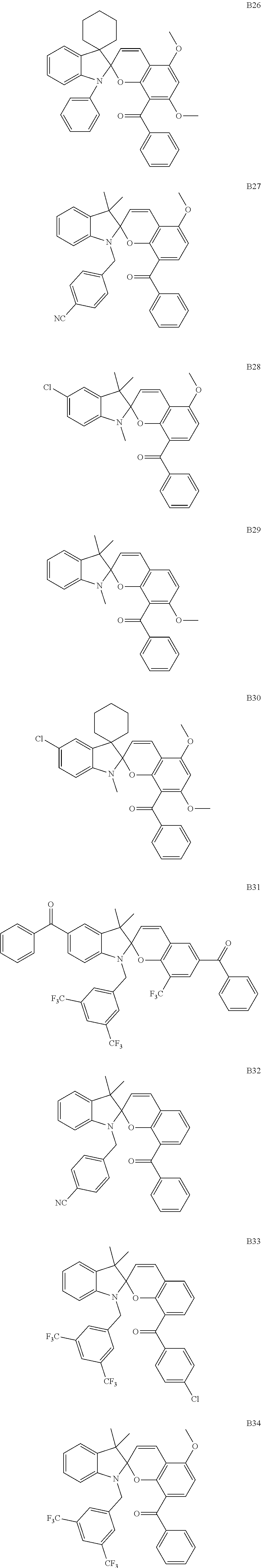

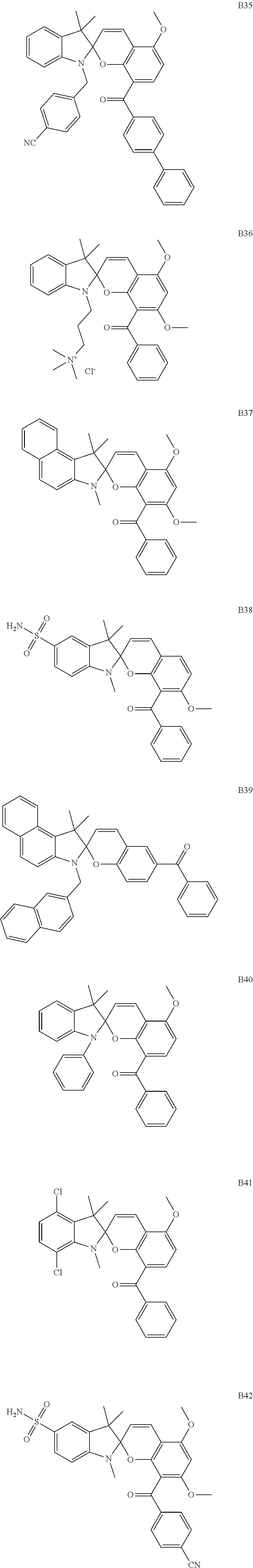

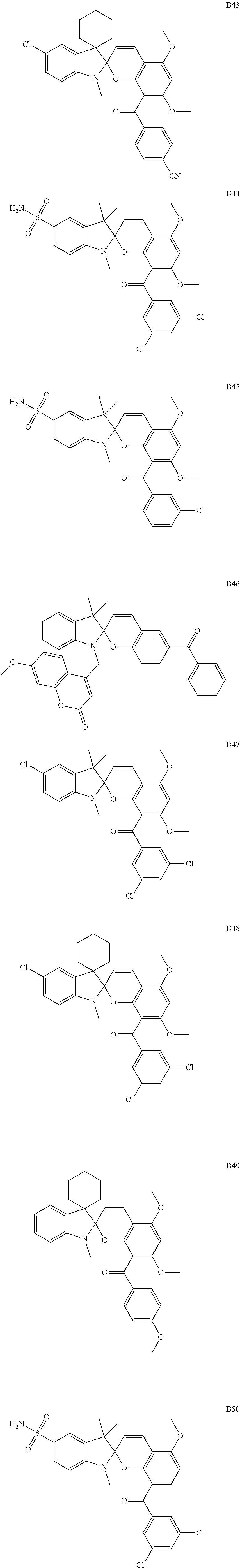

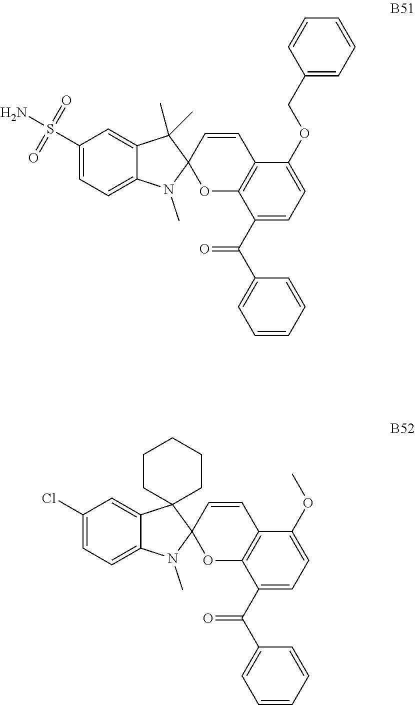 [SMILES: CN1c2ccc(S(N)(=O)=O)cc2C(C)(C)C12C=Cc1c(OCc3ccccc3)ccc(C(=O)c3ccccc3)c1O2 ; COc1ccc(C(=O)c2ccccc2)c2c1C=CC1(O2)N(C)c2ccc(Cl)cc2C12CCCCC2]

FIG. 1 shows a sample preparation or arrangement used for producing a cured disc of defined thickness. FIGS. 2a and 2b show absorption spectra of cured polymer discs obtained from formulations F3 and F6.

FIGS. 3a to 3d show adsorption spectra and difference spectra of formulations F12, F16, F25, F29, and F30.

FIGS. 4a and 4b show adsorption spectra and difference spectra of formulations F27, F30, and F34.

FIGS. 5a and 5b show adsorption spectra and difference spectra of formulations F27 and F28.

FIGS. 6a to 6d show adsorption spectra and difference spectra of formulations F26, F28, F31, F32, and F34.

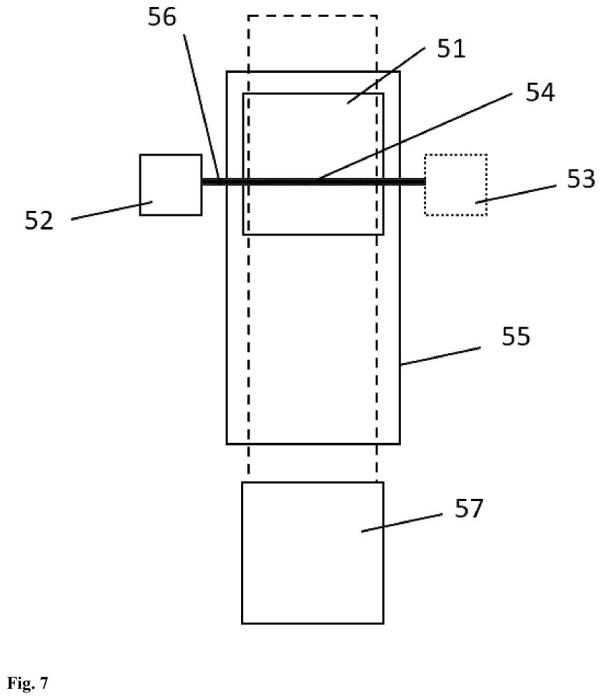

FIG. 7 is a schematic view of a device for processing an optically reactive material from above.

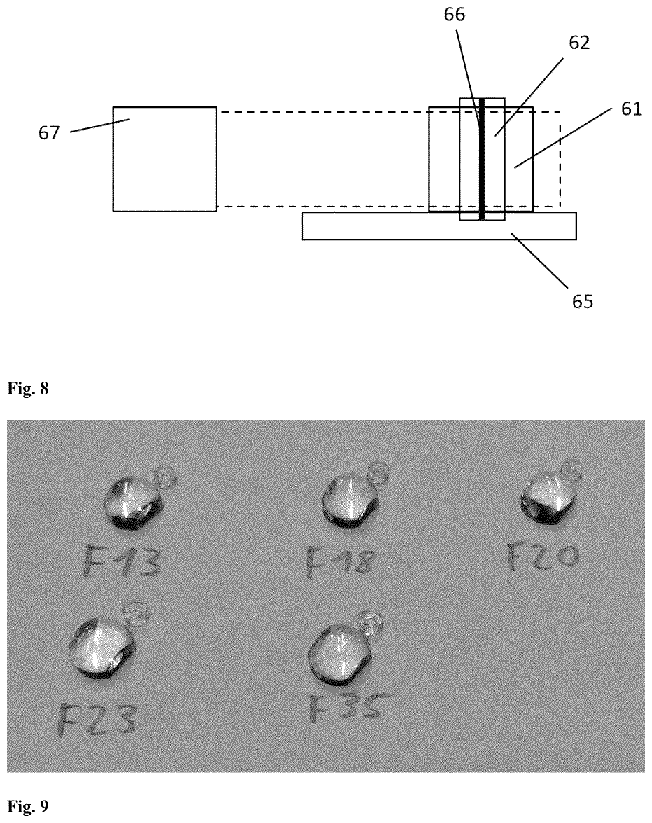

FIG. 8 is a schematic view of the device of FIG. 5 from the side.

FIG. 9 shows a photograph of optical lenses.

FIG. 1 shows the sample preparation or arrangement used for producing a cured disc of a formulation. The photopolymerizable starting material 13 is placed between two glass plates 11. The glass plates 11 are separated by spacer elements 12. As such the photopolymerizable starting material between the glass plates has a defined thickness. A typical thickness used in the experiments described herein is 1 mm. The arrangement is placed in a Rapidshape RS PRO cure and polymerized to obtain a photopolymerized disc of defined thickness as described in the experimental section.

FIGS. 7 and 8 show a schematic representation of an arrangement for a device for optical processing of an optically reactive material from above and from the side. A working volume is provided in a receiving container 51, 61, which is at least partially filled by a starting material. The starting material can comprise one or more substances, which can be solid, liquid or pasty. For processing, the starting material is irradiated with light of a first wavelength from a first light generator 52, 62 and optionally light of a first wavelength from a second light generator 53, which are irradiated in an overlapping manner in the working volume to trigger an optically activated reaction in a layer subvolume 54. The receiving container 51, 61, which is at least partially filled by a starting material may be moved along an axis for cuvette moving 55, 65 to shift the position of the subvolume 54 in the receiving container 51, 61 and in the starting material filled therein.

Alternatively, the first light generator 52, 62 and optionally the second light generator 53 may be moved along an axis to shift the position of the subvolume 54 in the receiving container 51, 61 and in the starting material filled therein (not shown in FIGS. 7 and 8).

The first light generator 52, 62 and optionally the second light generator 53 may be a light sheet generator.

Depending on the current position of the irradiation area 56, 66 (light sheet), the light of the second wavelength is projected with the aid of a projector 57, 67 into the layered part volume currently irradiated with the light of the first wavelength (projector or projection image). This means that the projection plane or projection volume of the projector 57, 67 is located in the layer subvolume that is currently being irradiated with the light of the first wavelength. The light of the first wavelength (light section) and the light of the second wavelength (light projection) thus at least partially overlap spatially or in a projection plane of the projector 57, 67 in a macroscopic partial layer volume of the starting material, which as a partial volume of the working volume which is just being irradiated by means of the light section.

In this way, at least one material property of the starting material is changed in the currently irradiated layered partial volume, for example to the effect that the original starting material hardens. In this case, polymerization can be triggered in the starting material due to the coincidence of the light of the two wavelengths. In this way, it is possible, for example, to continuously produce a three-dimensionally shaped body layer by layer in the working volume. The three-dimensional shaping of the body is influenced and determined with the aid of the projection of the projector 57, 67 formed in the respective layer volume. A concrete example for the apparatus described in FIGS. 7 and 8 is the Xube2 device, available from xolo GmbH.

FIG. 9 shows a photograph of optical lenses manufactured using a Xube2 device available from xolo GmbH according to the processes described herein using formulations F13, F18, F20, F23, and F35.

### EXAMPLES

Hereinafter, the action and effect of the invention will be described in detail through specific examples of the invention. However, the examples are provided only to illustrate the present invention, and the scope of the invention is not limited thereto.

### A. Synthesis

### General Synthetic Methods

Method A (Reaction of Indolenium Salt or Respective 2-Methylenindoline Derivate with a Salicylaldehyde Derivative, examples B2-B28, B30, B34-B37, B39-B52, I-16, I-17, I-18, I-22, I-27, I-30, I-34, I-39)

2-Methyleneindoline derivative (1 mmol), the respective salicylaldehyde derivative (1 mmol), and piperidine (0.1 mmol) are dissolved in 10 mL EtOH and heated to 70° C. until no further formation of the spiropyran derivative is observed, typically 1 h to 12 h. The mixture is cooled to room temperature and worked up as described below.

When an indolenium salt derivative is used instead of 2-methyleneindoline derivative, the reaction is carried out with 1.1 mmol piperidine.

When the product precipitates, the mixture is filtered, and the solid residue is recrystallized from ethanol.

Where the spiropyran does not precipitate or forms only little precipitate, water and ethyl acetate are added to the mixture and the organic phase is washed with water. The organic phase is dried over anhydrous MgSO₄ and evaporated under reduced pressure. The residue is either crystallized from ethanol or methyl-tert-butyl ether or purified by silica-gel column chromatography using petroleum ether-acetone mixtures.

For spiropyran derivative B36 water and ethyl acetate are added to the mixture and the organic phase is washed with water. The organic phase is washed with aqueous sodium hydroxide solution (1M), and saturated sodium chloride solution (5×). The organic phase is dried over anhydrous MgSO₄ and evaporated under reduced pressure. The crude product is purified by crystallization from ethanol and further crystallization from ethyl acetate.

### Method B (Modification of Halogenated Spiropyran, Examples B29, I-19, I-24, I-31, I-32, I-33, I-35, I-36, I-37, I-38)

The halogenated spiropyran (1.5 mmol) is dissolved in dry tetrahydrofuran (25 mL) under an argon atmosphere and cooled to −78° C. n-Butyl lithium (1.65 mmol, 2.5 M in hexanes) is added dropwise. After stirring for 15 min at −78° C., a Weinreb amide (2 mmol) is added, and the reaction mixture is allowed to warm to room temperature. The reaction is stirred until no further conversion is observed and 1 M aqueous hydrochloric (10 mL) acid is added. The mixture is stirred for 10 min, 1 M aqueous NaOH solution is added (20 mL) and the mixture is extracted with ethyl acetate. The combined organic layers are dried over anhydrous MgSO₄ and evaporated under reduced pressure. The residue is either crystallized or purified by silica-gel column chromatography using petroleum ether-acetone mixtures.

### Method C (Reaction of Indolenium Salt Derivative and Spiropyran Derivate, Examples B1, B31-B33, B38, I-23, I-28)

An indolenium salt derivative (1.2 mmol), a spiropyran derivative (1 mmol), and piperidine (0.1 mmol) are dissolved in 20 mL ethanol and stirred at 70° C. until no further formation of the product spiropyran derivative is observed, typically 30 min to 12 h. The reaction mixture is cooled to room temperature and worked up as described below.

When the product precipitates, the mixture is filtered, and the solid residue is recrystallized from ethanol.

Where the spiropyran does not precipitate or forms only little precipitate, water and ethyl acetate are added to the mixture and the organic phase is washed with water. The organic phase is dried over anhydrous MgSO₄ and evaporated under reduced pressure. The residue is either crystallized from ethanol or methyl-tert-butyl ether or purified by silica-gel column chromatography using petroleum ether-acetone mixtures.

### Method D (Synthesis of Indolenium Salts of the Respective Indoles, Examples I-1-I-12, I-25, I-40, I-43)

The corresponding aniline derivative (50 mmol) is dissolved in a mixture of concentrated aqueous hydrochloric acid (20 mL) and ice water (30 mL). NaNO₂ (100 mmol) in water is added at 0° C. After stirring for 30 min, SnCl₂ (28.4 g) in concentrated aqueous hydrochloric acid (35 mL) is added. The resulting mixture is stirred for 30 min, filtered, and washed with water or 1M aqueous hydrochloric acid to obtain the hydrazine hydrochloride which is used directly in the next step.

The corresponding hydrazine (as hydrochloride salt) (47.5 mmol), 3-methylbutan-2-one or cyclohexyl methyl ketone or and concentrated aqueous sulfuric acid (7 mL) are dissolved in glacial acetic acid (68 mL). The mixture is refluxed until no further formation of the indole is observed. The mixture is cooled to room temperature and worked up as described below.

The main fraction of the acetic acid is distilled of. The residue is neutralized with saturated aqueous NaHCO₃ solution. The mixture is extracted with ethyl acetate and the combined organic phases are dried over anhydrous MgSO₄, followed by evaporation of the solvent under reduced pressure. Where the indole is not of sufficient purity for the next step, silica gel column chromatography is performed using petroleum ether/acetone mixtures as eluent.

The obtained indole derivative (30 mmol) is dissolved in acetonitrile (150 mL) and the corresponding alkyl halide or benzylbromide derivative is added (90 mmol). The mixture is refluxed for 24 h. After cooling to room temperature, the product precipitates, is filtered and washed with acetonitrile. Where the product does not precipitate, the solvent is evaporated under reduced pressure. The indole is purified by recrystallization from mixtures of acetonitrile, acetone, and ethyl acetate.

### Method E (Friedl-Crafts Acylation of Salicylaldehyde Derivatives, Examples I-13, I-14, 1-20, I-21, I-26, I-41, I-42, I-44, I-45, I-46)

The acid chloride derivative (0.21 mol) and AlCl₃ (0.25 mol) are mixed with dichloromethane (125 mL) and cooled to 0° C. The respective salicylaldehyde derivative (1.6 mol) is added and the mixture is stirred overnight. The mixture is poured on a mixture of ice (60 g) and aqueous hydrochloric acid (1M, 200 mL). The precipitate is filtered and crystallized from 2-butanone or purified by silica-gel column chromatography using petroleum ether-acetone mixtures. The aqueous phase is extracted with dichloromethane and the combined organic phase is washed with saturated sodium chloride solution. The organic phase is dried over anhydrous MgSO₄, followed by evaporation of the solvent under reduced pressure. The residue is either crystallized or purified by silica-gel column chromatography using petroleum ether-acetone mixtures.

### Preparation of I-15

3,4-Dimethoxysalicylaldehyde (28 mmol) is dissolved in dichloromethane (150 mL) and cooled to −78° C. N-Bromosuccinimide (28 mmol) is added and the mixture is allowed to warm to room temperature and stirred for 10 h. An aqueous solution of sodium thiosulfate (1 M) is added and the aqueous phase is extracted with ethyl acetate. The combined organic phase is washed with water, dried over anhydrous MgSO₄ and the solvent is evaporated under reduced pressure. The crude residue is purified by silica-gel column chromatography using petroleum ether-ethyl acetate (10/1) to yield the product as a white solid.

### Benzylation of I-45

I-45 (21 mmol) and benzyl bromide (21 mmol) are dissolved in DMF. K₂CO₃ (45 mmol) is added and the solution is stirred at room temperature for 18 h. Ethyl acetate is added to the solution and the organic phase is washed 5× with water, dried over anhydrous Na2SO4 and the solvent is evaporated under reduced pressure. The crude product is purified by silica gel column chromatography (heptane/acetone 5/1) to yield a mixture of two products (2.4 g, 1:1). The mixture contains the product, where both hydroxy groups have been benzylated and the product where a benzyl ether has been formed on the oxygen para to the benzoyl substituent. The mixture is used for the preparation of B51, where only the salicylaldehyde forms a spiropyran product.

As non-limiting examples, the following spiropyrans have been prepared by following the specified procedures or procedures known from the state of the art as dual color photoinitiators, intermediates, and reference compounds:

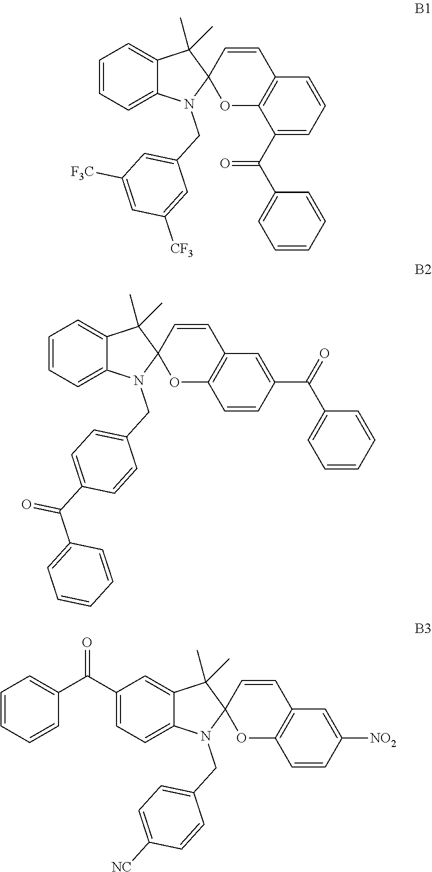

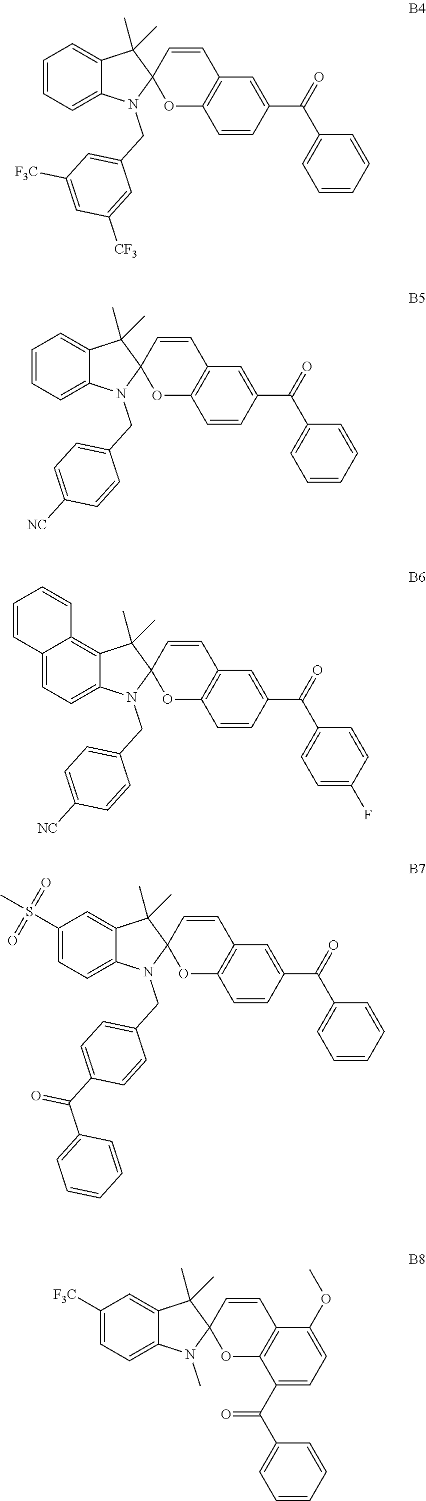 [SMILES: CC1(C)c2ccccc2N(Cc2cc(C(F)(F)F)cc(C(F)(F)F)c2)C12C=Cc1cc(C(=O)c3ccccc3)ccc1O2 ; CC1(C)c2ccccc2N(Cc2ccc(C#N)cc2)C12C=Cc1cc(C(=O)c3ccccc3)ccc1O2 ; CC1(C)c2c(ccc3ccccc23)N(Cc2ccc(C#N)cc2)C12C=Cc1cc(C(=O)c3ccc(F)cc3)ccc1O2 ; CC1(C)c2cc(S(C)(=O)=O)ccc2N(Cc2ccc(C(=O)c3ccccc3)cc2)C12C=Cc1cc(C(=O)c3ccccc3)ccc1O2 ; COc1ccc(C(=O)c2ccccc2)c2c1C=CC1(O2)N(C)c2ccc(C(F)(F)F)cc2C1(C)C]

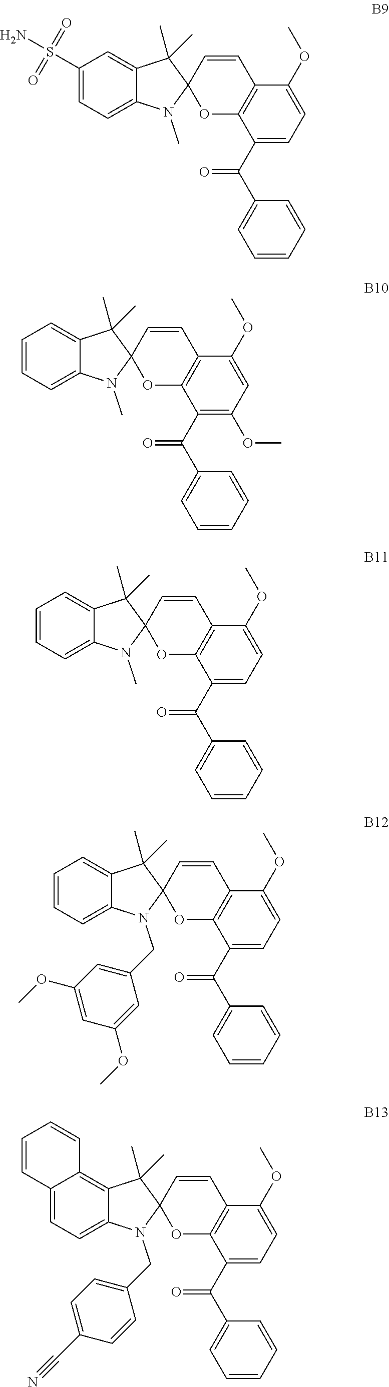

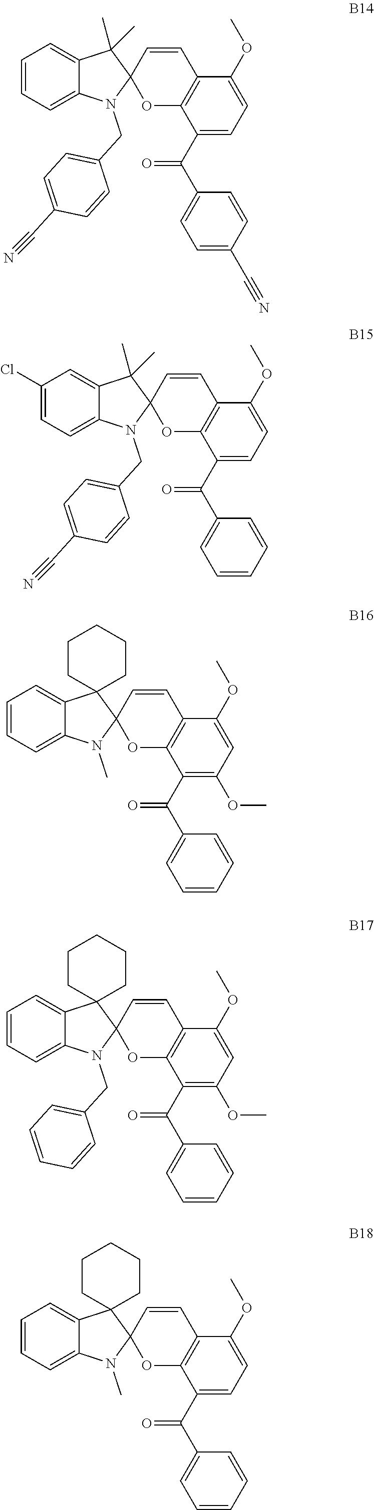

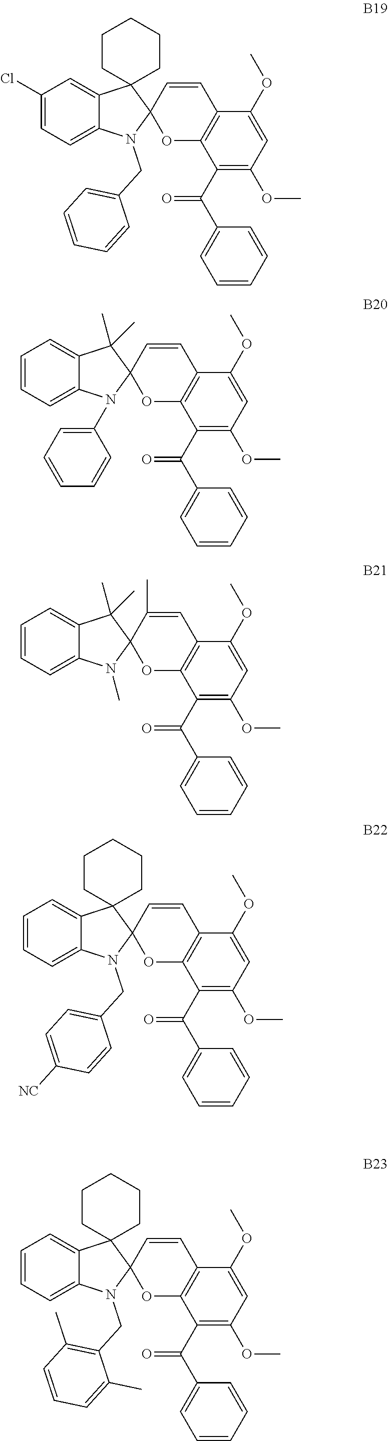

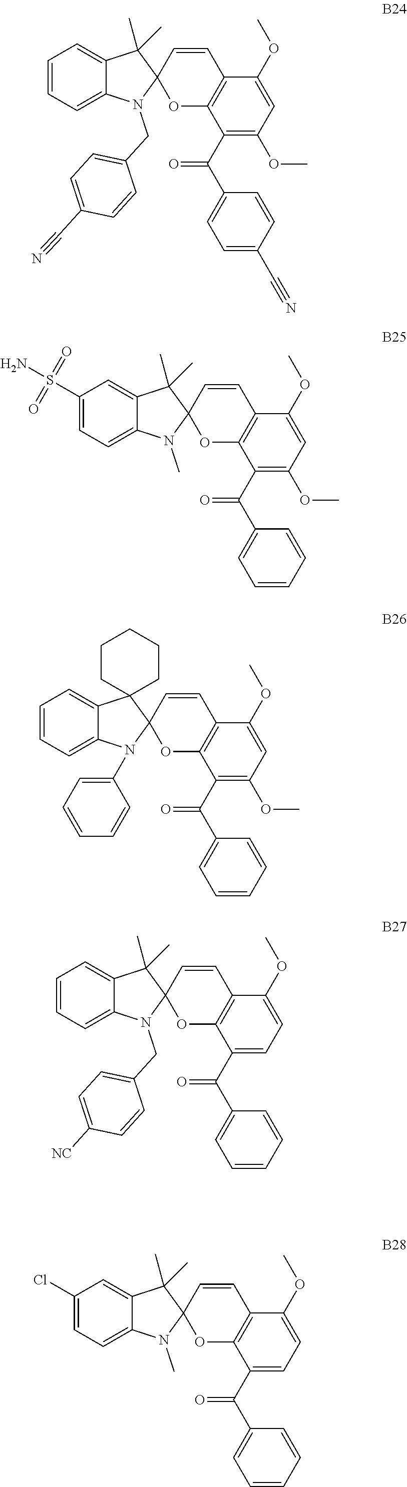

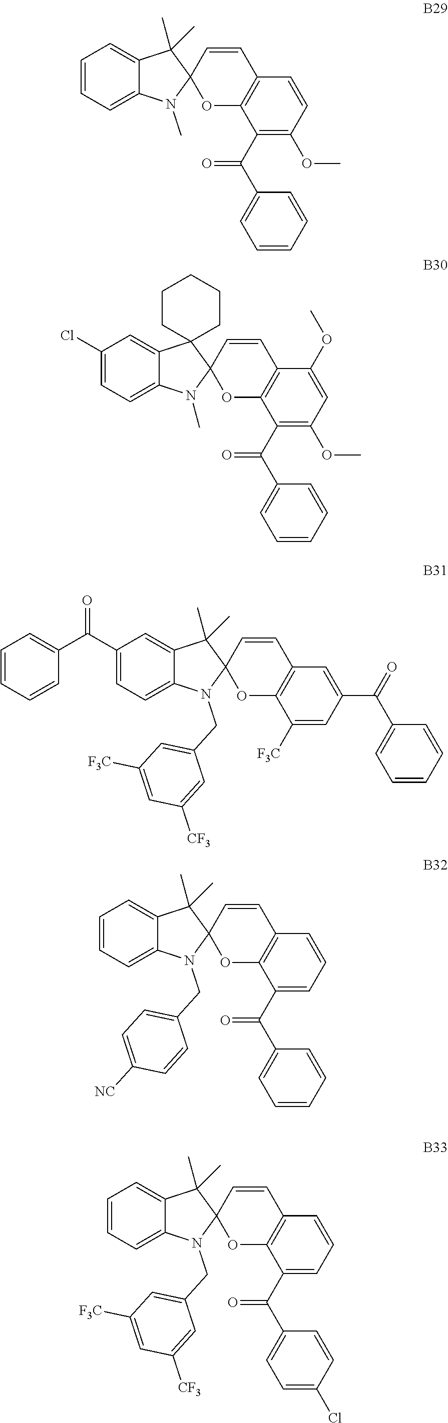

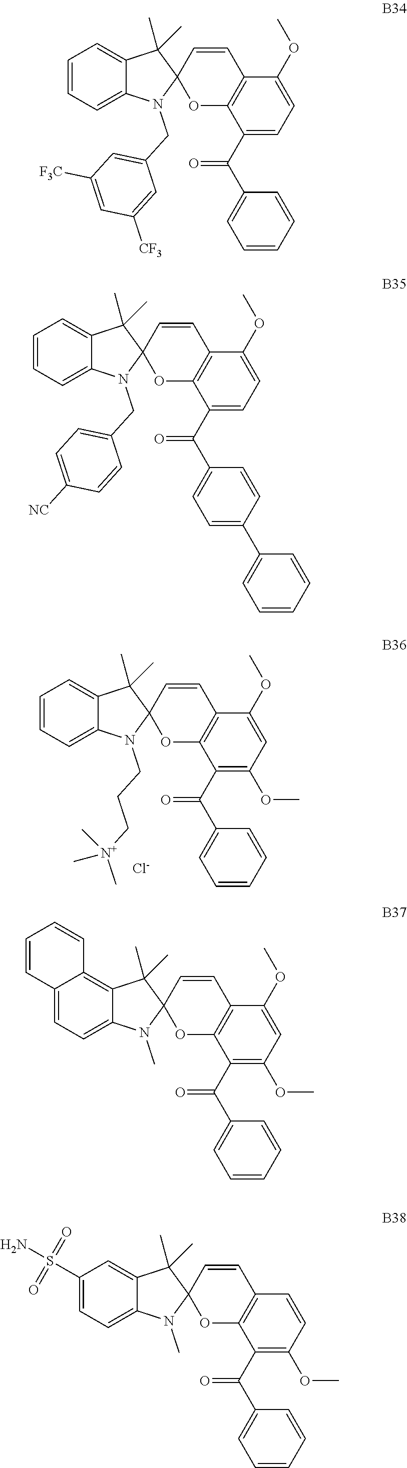

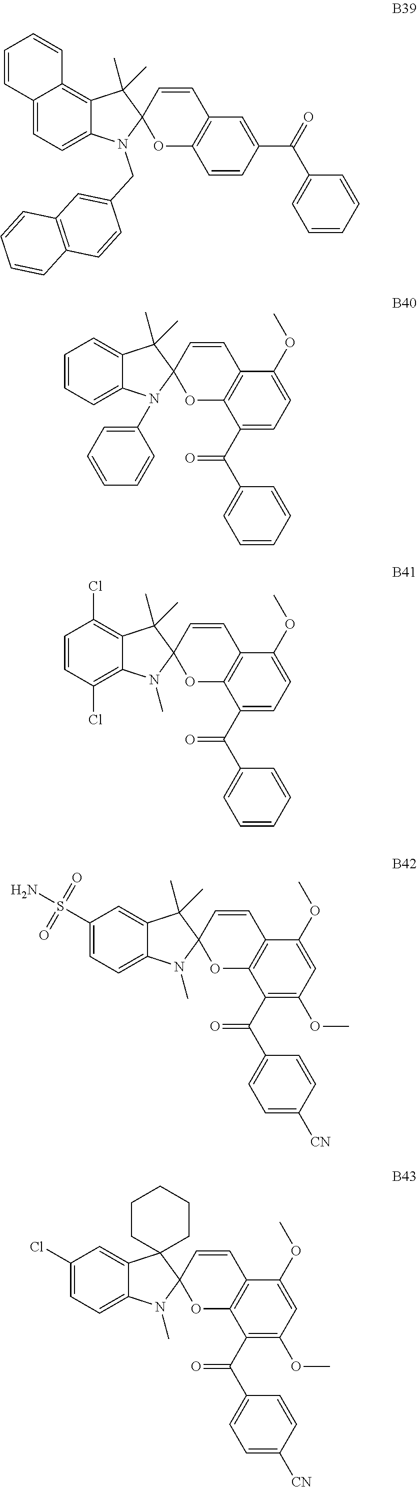 [SMILES: CC1(C)c2c(ccc3ccccc23)N(Cc2ccc3ccccc3c2)C12C=Cc1cc(C(=O)c3ccccc3)ccc1O2 ; COc1ccc(C(=O)c2ccccc2)c2c1C=CC1(O2)N(c2ccccc2)c2ccccc2C1(C)C ; COc1ccc(C(=O)c2ccccc2)c2c1C=CC1(O2)N(C)c2c(Cl)ccc(Cl)c2C1(C)C ; COc1cc(OC)c(C(=O)c2ccc(C#N)cc2)c2c1C=CC1(O2)N(C)c2ccc(S(N)(=O)=O)cc2C1(C)C ; COc1cc(OC)c(C(=O)c2ccc(C#N)cc2)c2c1C=CC1(O2)N(C)c2ccc(Cl)cc2C12CCCCC2]

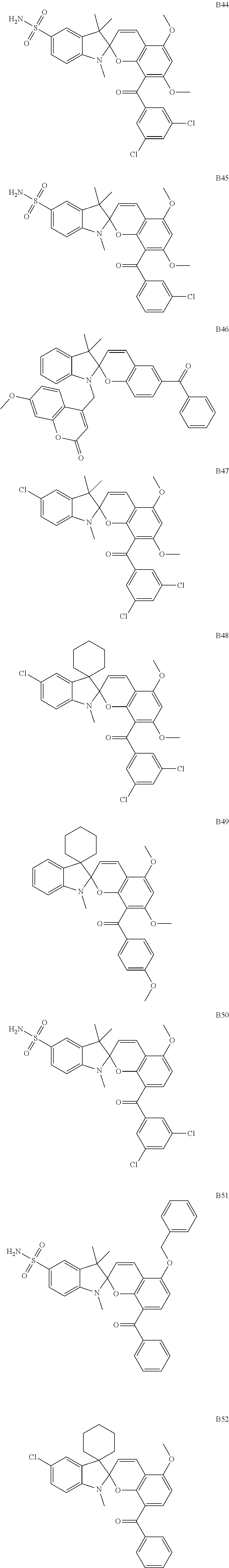

As non-limiting examples, the following precursors and reference compounds have been prepared by following the specified procedures:

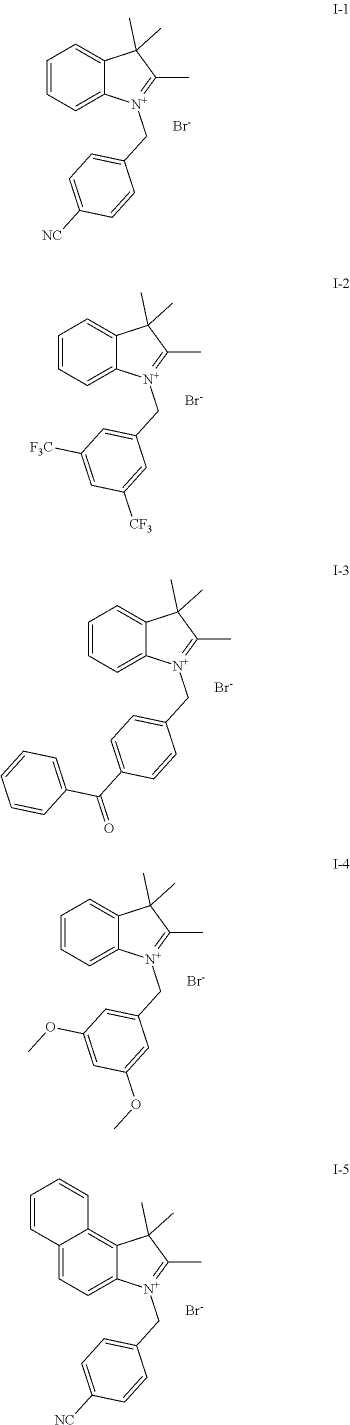

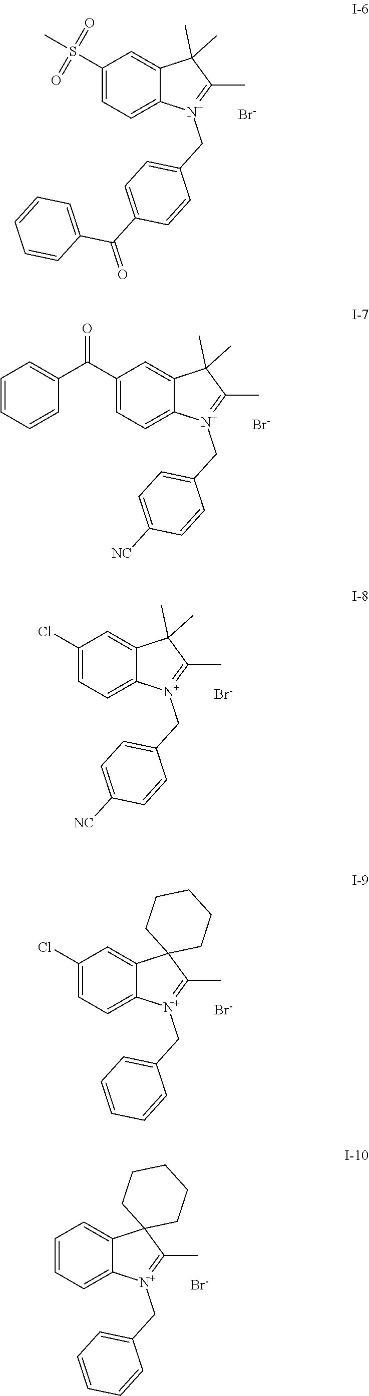

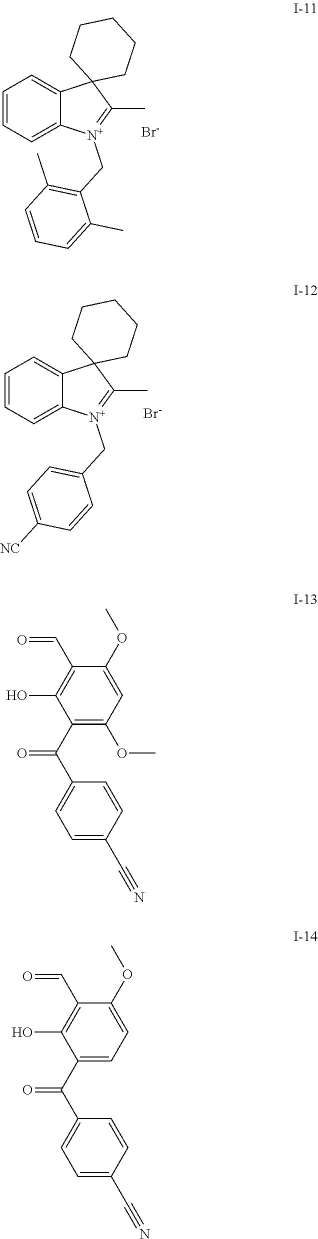 [SMILES: CC1=[N+](Cc2c(C)cccc2C)c2ccccc2C12CCCCC2 ; [Br-] ; CC1=[N+](Cc2ccc(C#N)cc2)c2ccccc2C12CCCCC2 ; [Br-] ; COc1cc(OC)c(C(=O)c2ccc(C#N)cc2)c(O)c1C=O ; COc1ccc(C(=O)c2ccc(C#N)cc2)c(O)c1C=O]

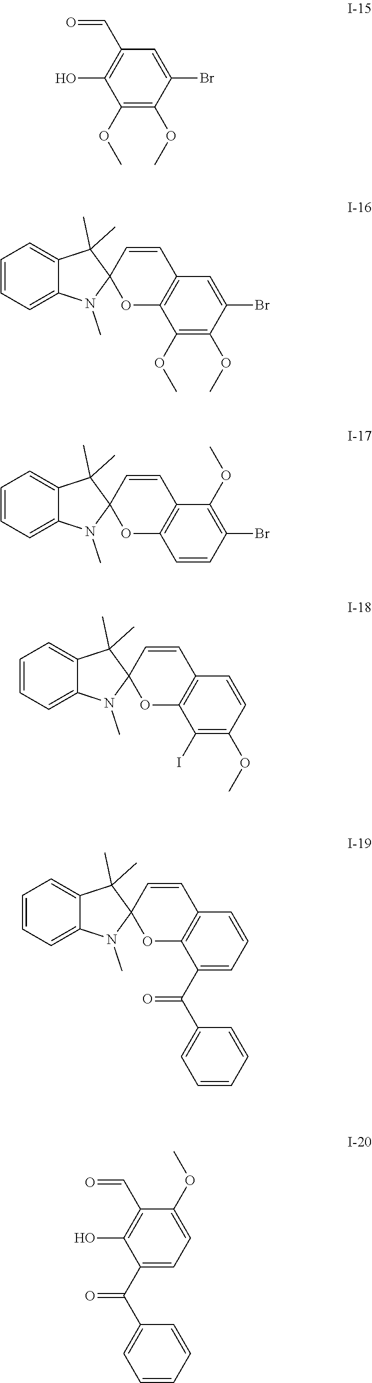 [SMILES: COc1c(Br)cc(C=O)c(O)c1OC ; COc1c(Br)cc2c(c1OC)OC1(C=C2)N(C)c2ccccc2C1(C)C ; COc1c(Br)ccc2c1C=CC1(O2)N(C)c2ccccc2C1(C)C ; COc1ccc2c(c1I)OC1(C=C2)N(C)c2ccccc2C1(C)C ; CN1c2ccccc2C(C)(C)C12C=Cc1cccc(C(=O)c3ccccc3)c1O2 ; COc1ccc(C(=O)c2ccccc2)c(O)c1C=O]

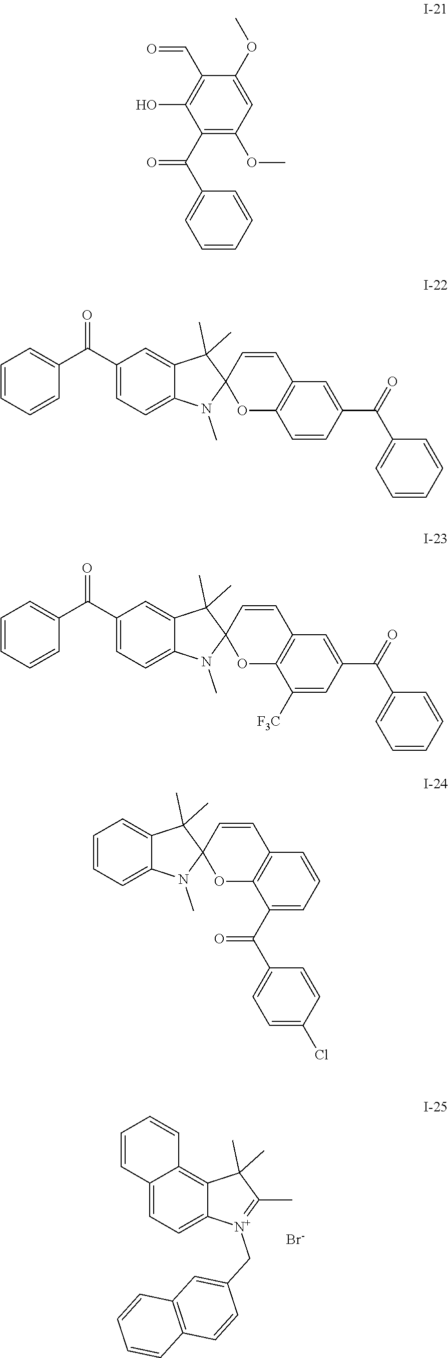 [SMILES: COc1cc(OC)c(C(=O)c2ccccc2)c(O)c1C=O ; CN1c2ccc(C(=O)c3ccccc3)cc2C(C)(C)C12C=Cc1cc(C(=O)c3ccccc3)ccc1O2 ; CN1c2ccc(C(=O)c3ccccc3)cc2C(C)(C)C12C=Cc1cc(C(=O)c3ccccc3)cc(C(F)(F)F)c1O2 ; CN1c2ccccc2C(C)(C)C12C=Cc1cccc(C(=O)c3ccc(Cl)cc3)c1O2 ; CC1=[N+](Cc2ccc3ccccc3c2)c2ccc3ccccc3c2C1(C)C ; [Br-]]

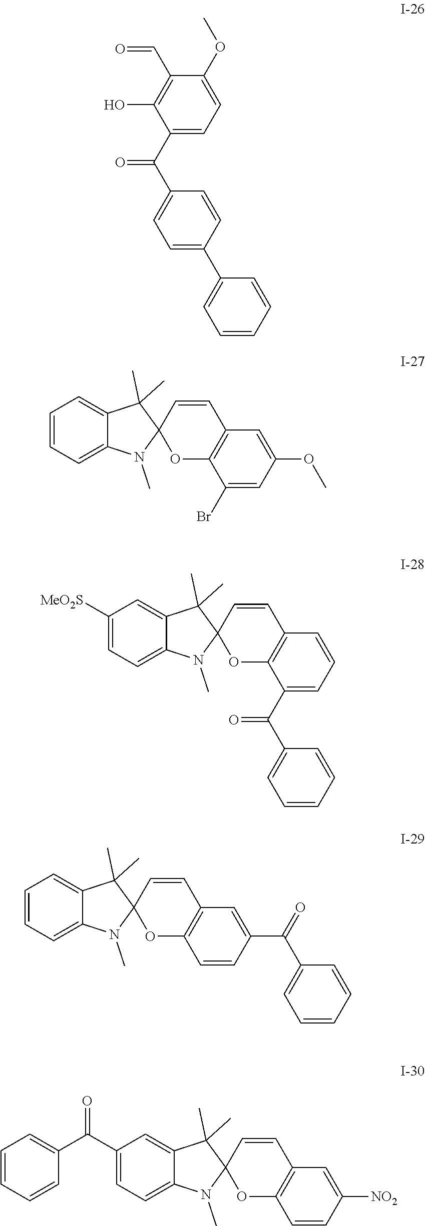 [SMILES: COc1ccc(C(=O)c2ccc(-c3ccccc3)cc2)c(O)c1C=O ; COc1cc(Br)c2c(c1)C=CC1(O2)N(C)c2ccccc2C1(C)C ; CN1c2ccc(S(C)(=O)=O)cc2C(C)(C)C12C=Cc1cccc(C(=O)c3ccccc3)c1O2 ; CN1c2ccccc2C(C)(C)C12C=Cc1cc(C(=O)c3ccccc3)ccc1O2 ; CN1c2ccc(C(=O)c3ccccc3)cc2C(C)(C)C12C=Cc1cc([N+](=O)[O-])ccc1O2]

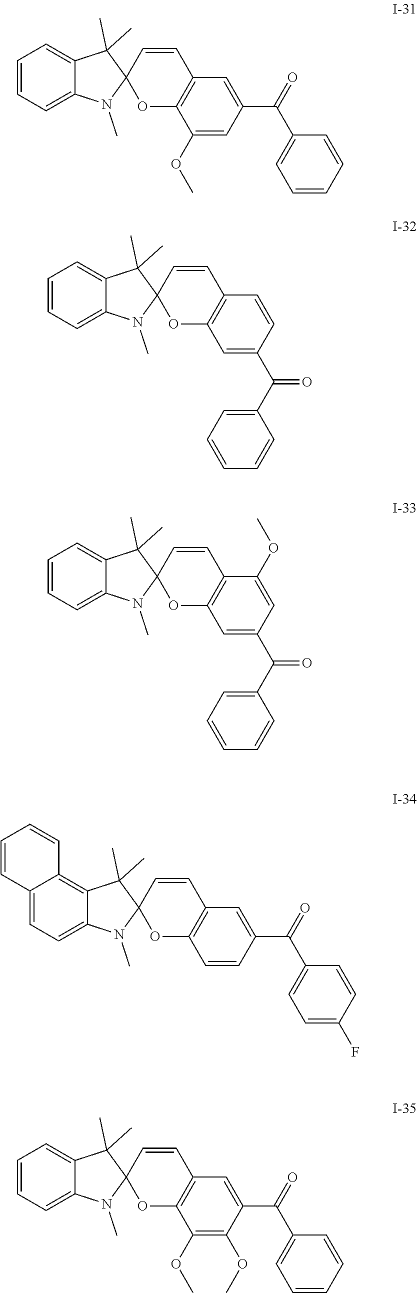 [SMILES: COc1cc(C(=O)c2ccccc2)cc2c1OC1(C=C2)N(C)c2ccccc2C1(C)C ; CN1c2ccccc2C(C)(C)C12C=Cc1ccc(C(=O)c3ccccc3)cc1O2 ; COc1cc(C(=O)c2ccccc2)cc2c1C=CC1(O2)N(C)c2ccccc2C1(C)C ; CN1c2ccc3ccccc3c2C(C)(C)C12C=Cc1cc(C(=O)c3ccc(F)cc3)ccc1O2 ; COc1c(C(=O)c2ccccc2)cc2c(c1OC)OC1(C=C2)N(C)c2ccccc2C1(C)C]

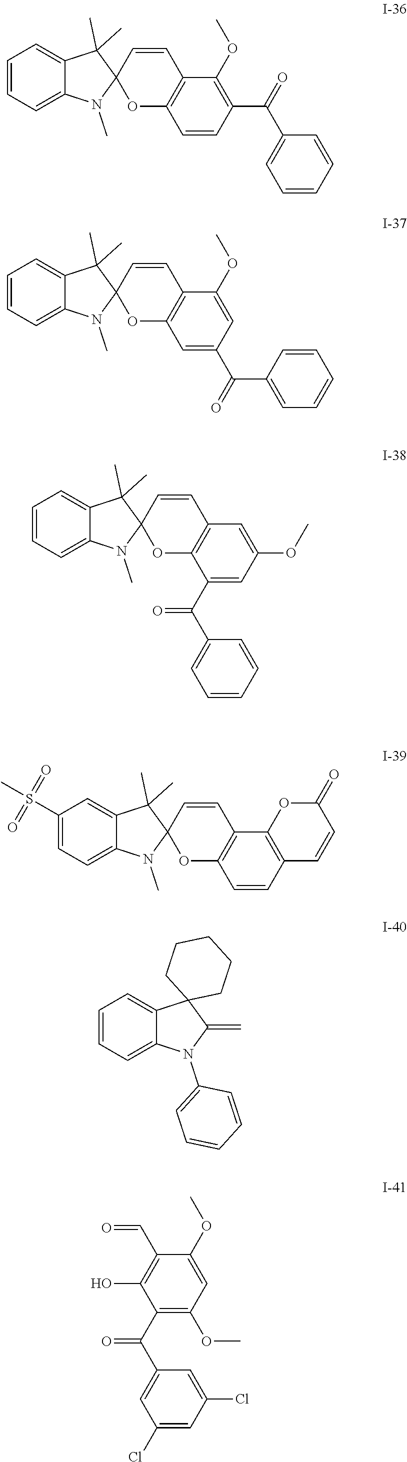

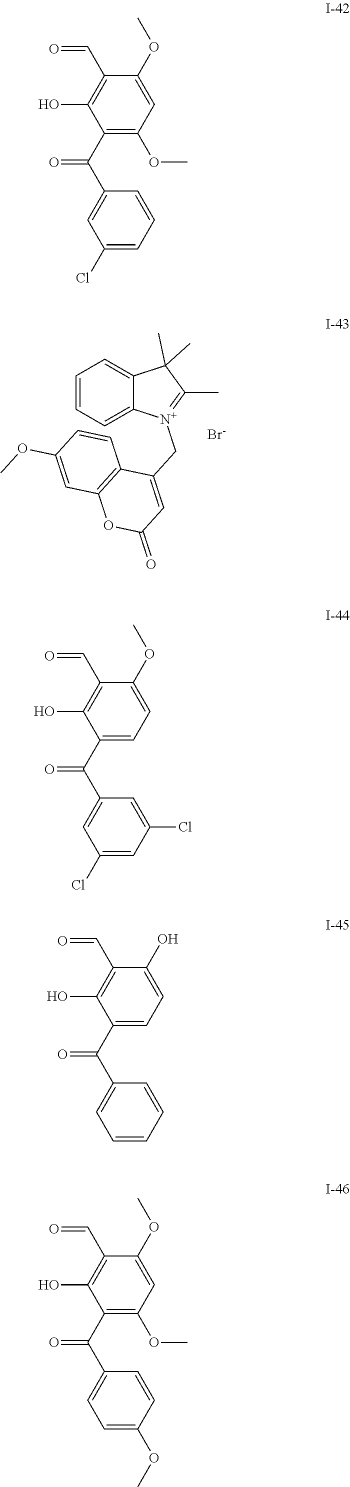

### Characterization

The following compounds have been characterized by ¹H NMR spectroscopy; all other compounds have been used without further purification:

I-1 ¹H NMR (500 MHz, Methylene Chloride-d₂) δ 7.75-7.72 (m, 1H), 7.70-7.66 (m, 2H), 7.64-7.58 (m, 2H), 7.55-7.52 (m, 3H), 6.28 (s, 2H), 3.14 (s, 3H), 1.66 (s, 6H).

I-2 ¹H NMR (500 MHz, Methylene Chloride-d₂) δ 7.94-7.79 (m, 3H), 7.67-7.48 (m, 4H), 6.48 (s, 2H), 3.17 (s, 3H), 1.68 (s, 6H).

I-3 ¹H NMR (500 MHz, Methylene Chloride-d₂) δ 7.84-7.79 (m, 2H), 7.78-7.75 (m, 2H), 7.68-7.59 (m, 4H), 7.59-7.55 (m, 1H), 7.53-7.47 (m, 2H), 7.47-7.43 (m, 2H), 6.20 (s, 2H), 1.69 (s, 6H).

I-4 ¹H NMR (500 MHz, Methylene Chloride-d₂) δ 7.65-7.57 (m, 3H), 7.57-7.51 (m, 1H), 6.44 (t, J=2.2 Hz, 1H), 6.43-6.40 (m, 2H), 5.94 (s, 2H), 3.75 (s, 6H), 3.09 (s, 3H), 1.67 (s, 6H).

I-5 ¹H NMR (500 MHz, Methylene Chloride-d₂) δ 8.19-8.13 (m, 1H), 8.11-8.05 (m, 2H), 7.83-7.77 (m, 1H), 7.76-7.72 (m, 1H), 7.72-7.68 (m, 3H), 7.54-7.48 (m, 2H), 6.39 (s, 2H), 3.22 (s, 3H), 1.91 (s, 6H).

I-6 ¹H NMR (500 MHz, Methylene Chloride-d₂) δ 8.22-8.18 (m, 1H), 8.14-8.10 (m, 2H), 7.88-7.70 (m, 4H), 7.65-7.58 (m, 1H), 7.53-7.46 (m, 2H), 7.45-7.35 (m, 2H), 6.23 (s, 2H), 3.11 (s, 3H), 2.95 (s, 3H), 1.60 (s, 6H).

I-7 ¹H NMR (500 MHz, Methylene Chloride-d₂) δ 8.06-8.03 (m, 1H), 8.01-7.99 (m, 1H), 7.89 (dd, J=8.2, 1.6 Hz, 1H), 7.78-7.75 (m, 2H), 7.72-7.69 (m, 2H), 7.68-7.64 (m, 2H), 7.55-7.48 (m, 3H), 6.31 (s, 3H), 2.94 (s, 3H), 1.59 (s, 6H).

I-8 ¹H NMR (400 MHz, Methylene Chloride-d₂) δ 7.77-7.68 (m, 3H), 7.61 (d, J=2.0 Hz, 1H), 7.55-7.45 (m, 3H), 6.27 (s, 2H), 3.13 (s, 3H), 1.69 (s, 6H).

I-9 ¹H NMR (400 MHz, Methylene Chloride-d₂) δ 7.96 (d, J=1.9 Hz, 1H), 7.66 (d, J=8.6 Hz, 1H), 7.59-7.51 (m, 1H), 7.43-7.36 (m, 3H), 7.30-7.24 (m, 2H), 6.01 (s, 2H), 3.12 (s, 3H), 2.14-1.81 (m, 7H), 1.81-1.47 (m, 3H).

I-10 ¹H NMR (400 MHz, Methylene Chloride-d₂) δ 8.05-7.97 (m, 1H), 7.68-7.63 (m, 1H), 7.61-7.55 (m, 2H), 7.43-7.37 (m, 3H), 7.31-7.26 (m, 2H), 6.01 (s, 2H), 3.11 (s, 3H), 2.16-1.87 (m, 7H), 1.74-1.53 (m, 3H).

I-11 ¹H NMR (400 MHz, Methylene Chloride-d₂) δ 8.02-7.95 (m, 1H), 7.53 (td, J=7.5, 1.3 Hz, 1H), 7.48 (td, J=7.8, 1.4 Hz, 1H), 7.43-7.38 (m, 1H), 7.25 (dd, J=8.1, 7.0 Hz, 1H), 7.12 (d, J=7.6 Hz, 2H), 6.06 (s, 2H), 2.88 (d, J=0.9 Hz, 3H), 2.33 (s, 6H), 2.12-1.84 (m, 7H), 1.81-1.49 (m, 3H).

I-12 ¹H NMR (400 MHz, Methylene Chloride-d₂) δ 8.05-7.99 (m, 1H), 7.72-7.65 (m, 3H), 7.63-7.57 (m, 2H), 7.50-7.44 (m, 2H), 6.27 (s, 2H), 3.14 (s, 3H), 2.16-1.85 (m, 7H), 1.77-1.52 (m, 3H).

I-13 ¹H NMR (500 MHz, Chloroform-d) δ 12.54 (s, 1H), 10.16 (s, 1H), 8.01-7.87 (m, 2H), 7.78-7.69 (m, 2H), 6.03 (s, 1H), 3.99 (s, 3H), 3.82 (s, 3H).

I-15 ¹H NMR (400 MHz, Chloroform-d) δ 11.18 (s, 1H), 9.73 (s, 1H), 7.49 (s, 1H), 4.05 (s, 3H), 3.93 (s, 3H).

I-16 ¹H NMR (300 MHz, Chloroform-d) δ 7.15 (td, J=7.7, 1.4 Hz, 1H), 7.07 (dd, J=7.3, 1.3 Hz, 1H), 6.99 (s, 1H), 6.84 (td, J=7.4, 1.0 Hz, 1H), 6.77 (d, J=10.3 Hz, 1H), 6.50 (d, J=7.7 Hz, 1H), 5.69 (d, J=10.2 Hz, 1H), 3.85 (s, 3H), 3.61 (s, 3H), 2.71 (s, 3H), 1.34 (s, 3H), 1.19 (s, 3H).

I-17 ¹H NMR (300 MHz, Acetonitrile-d₃) δ 7.27 (d, J=8.8 Hz, 1H), 7.22-7.05 (m, 3H), 6.82 (t, J=7.4 Hz, 1H), 6.56 (d, J=7.7 Hz, 1H), 6.37 (d, J=8.8 Hz, 1H), 5.85 (d, J=10.5 Hz, 1H), 3.84 (d, J=1.2 Hz, 3H), 2.69 (d, J=1.3 Hz, 3H), 1.25 (s, 3H), 1.14 (s, 3H).

I-18 ¹H NMR (300 MHz, Acetonitrile-d₃) δ 7.20-7.06 (m, 3H), 6.93-6.77 (m, 2H), 6.57 (d, J=7.7 Hz, 1H), 6.49 (d, J=8.4 Hz, 1H), 5.63 (d, J=10.2 Hz, 1H), 3.81 (s, 3H), 2.66 (s, 3H), 1.26 (s, 3H), 1.13 (s, 3H).

I-20 ¹H NMR (500 MHz, Chloroform-d) δ 12.71 (s, 1H), 10.41 (s, 1H), 7.86-7.71 (m, 3H), 7.62-7.52 (m, 1H), 7.46 (t, J=7.7 Hz, 2H), 6.51 (d, J=8.8 Hz, 1H), 3.99 (s, 3H).

I-21 ¹H NMR (500 MHz, Chloroform-d) δ 12.43 (s, 1H), 10.16 (s, 1H), 7.97-7.81 (m, 2H), 7.65-7.51 (m, 1H), 7.51-7.37 (m, 2H), 6.02 (s, 1H), 3.97 (s, 3H), 3.81 (s, 3H).

I-24 ¹H NMR (400 MHz, Methylene Chloride-d₂) δ 7.34-7.27 (m, 3H), 7.24 (dd, J=7.5, 1.7 Hz, 1H), 7.17-7.11 (m, 2H), 7.06 (td, J=7.6, 1.3 Hz, 1H), 6.97-6.88 (m, 2H), 6.83 (dd, J=6.7, 1.1 Hz, 1H), 6.70 (td, J=7.4, 1.0 Hz, 1H), 6.23 (dd, J=7.7, 0.8 Hz, 1H), 5.69 (d, J=10.3 Hz, 1H), 2.55 (s, 3H), 1.04 (s, 3H), 1.04 (s, 3H).

I25 ¹H NMR (400 MHz, Methylene Chloride-d₂) δ 8.21-8.14 (m, 1H), 8.09-8.03 (m, 2H), 7.90 (d, J=8.6 Hz, 1H), 7.88-7.75 (m, 5H), 7.69 (ddd, J=8.2, 6.9, 1.2 Hz, 1H), 7.57-7.48 (m, 2H), 7.43 (dd, J=8.5, 2.0 Hz, 1H), 6.32 (s, 2H), 3.25 (s, 3H), 1.93 (s, 7H).

I-26 ¹H NMR (400 MHz, Methylene Chloride-d₂) δ 12.66 (s, 1H), 10.41 (s, 1H), 8.21-8.13 (m, 1H), 7.89-7.83 (m, 1H), 7.78 (d, J=8.7 Hz, 1H), 7.73 (dd, J=8.6, 2.1 Hz, 1H), 7.72-7.69 (m, 1H), 7.69-7.65 (m, 2H), 7.53-7.45 (m, 2H), 7.44-7.36 (m, 1H), 6.58 (d, J=8.8 Hz, 1H), 3.99 (s, 3H).

I-31 ¹H NMR (400 MHz, Methylene Chloride-d₂) δ 7.78-7.72 (m, 2H), 7.63-7.56 (m, 1H), 7.54-7.45 (m, 2H), 7.34 (d, J=2.0 Hz, 1H), 7.23-7.16 (m, 2H), 7.08 (dd, J=7.2, 1.2 Hz, 1H), 6.92-6.83 (m, 2H), 6.55 (d, J=7.7 Hz, 1H), 5.76 (d, J=10.3 Hz, 1H), 3.79 (s, 3H), 2.77 (s, 3H), 1.30 (s, 3H), 1.16 (s, 3H).

I-33 ¹H NMR (300 MHz, Chloroform-d) δ 7.78-7.68 (m, 2H), 7.59-7.49 (m, 1H), 7.43 (dd, J=8.2, 6.7 Hz, 2H), 7.27 (dd, J=10.5, 0.7 Hz, 1H), 7.15 (td, J=7.6, 1.3 Hz, 1H), 7.05 (dd, J=7.2, 1.2 Hz, 1H), 6.94 (d, J=1.4 Hz, 1H), 6.82 (td, J=7.4, 1.0 Hz, 1H), 6.76-6.69 (m, 1H), 6.51 (d, J=7.7 Hz, 1H), 5.77 (d, J=10.5 Hz, 1H), 3.91 (s, 3H), 2.74 (s, 3H), 1.30 (s, 3H), 1.17 (s, 3H).

I-35 ¹H NMR (300 MHz, Acetonitrile-d₃) δ 7.79-7.69 (m, 2H), 7.65-7.57 (m, 1H), 7.48 (dd, J=8.2, 6.8 Hz, 2H), 7.21-7.10 (m, 2H), 6.98-6.90 (m, 2H), 6.89-6.80 (m, 1H), 6.59 (d, J=7.6 Hz, 1H), 5.79 (d, J=10.3 Hz, 1H), 3.59 (s, 3H), 3.57 (s, 3H), 2.74 (s, 3H), 1.34 (s, 3H), 1.17 (s, 3H).

I-36 ¹H NMR (300 MHz, Acetonitrile-d₃) δ 7.82-7.72 (m, 2H), 7.67-7.58 (m, 1H), 7.49 (dd, J=8.3, 7.0 Hz, 2H), 7.26-7.08 (m, 4H), 6.83 (td, J=7.4, 1.0 Hz, 1H), 6.58 (d, J=7.7 Hz, 1H), 6.48 (dd, J=8.5, 0.8 Hz, 1H), 5.87 (d, J=10.5 Hz, 1H), 3.62 (s, 3H), 2.75 (s, 3H), 1.30 (s, 3H), 1.16 (s, 3H).

I-37 ¹H NMR (300 MHz, Chloroform-d) δ 7.78-7.68 (m, 2H), 7.59-7.49 (m, 1H), 7.43 (dd, J=8.2, 6.7 Hz, 2H), 7.27 (d, J=10.2 Hz, 1H), 7.15 (td, J=7.6, 1.3 Hz, 1H), 7.05 (dd, J=7.2, 1.2 Hz, 1H), 6.94 (d, J=1.4 Hz, 1H), 6.82 (td, J=7.4, 1.0 Hz, 1H), 6.74-6.70 (m, 1H), 6.51 (d, J=7.7 Hz, 1H), 5.77 (d, J=10.5 Hz, 1H), 3.91 (s, 3H), 2.74 (s, 3H), 1.30 (s, 3H), 1.17 (s, 3H).

I-38 ¹H NMR (500 MHz, Chloroform-d) δ 7.46-7.40 (m, 2H), 7.37-7.32 (m, 1H), 7.21-7.15 (m, 2H), 6.99 (td, J=7.6, 1.3 Hz, 1H), 6.87 (d, J=3.0 Hz, 1H), 6.82 (d, J=10.3 Hz, 1H), 6.80-6.76 (m, 2H), 6.67 (td, J=7.3, 1.0 Hz, 1H), 6.18 (d, J=7.6 Hz, 1H), 5.69 (d, J=10.2 Hz, 1H), 3.80 (s, 3H), 2.50 (s, 3H), 1.04 (s, 3H), 0.98 (s, 3H).

I-39 ¹H NMR (500 MHz, Chloroform-d) δ 7.79 (dd, J=8.3, 1.9 Hz, 1H), 7.61 (d, J=9.4 Hz, 1H), 7.58 (d, J=1.9 Hz, 1H), 7.51 (d, J=10.4 Hz, 1H), 7.24 (d, J=8.5 Hz, 1H), 6.66 (d, J=8.5 Hz, 1H), 6.60 (d, J=8.2 Hz, 1H), 6.27 (d, J=9.4 Hz, 1H), 5.81 (d, J=10.5 Hz, 1H), 3.07 (s, 3H), 2.83 (s, 3H), 1.33 (s, 3H), 1.21 (s, 3H).

I-41 ¹H NMR (500 MHz, Chloroform-d) δ 12.52 (s, 1H), 10.16 (s, 1H), 7.69 (d, J=1.9 Hz, 2H), 7.53 (t, J=1.9 Hz, 1H), 6.03 (s, 1H), 3.99 (s, 3H), 3.83 (s, 3H).

I-42 ¹H NMR (500 MHz, Chloroform-d) δ 12.47 (s, 1H), 10.16 (s, 1H), 7.82 (t, J=1.9 Hz, 1H), 7.74 (dt, J=7.9, 1.3 Hz, 1H), 7.57-7.48 (m, 1H), 7.38 (t, J=7.8 Hz, 1H), 6.02 (s, 1H), 3.98 (s, 3H), 3.82 (s, 3H).

I-44 ¹H NMR (500 MHz, Chloroform-d) δ 12.66 (s, 1H), 10.38 (s, 1H), 7.78 (d, J=8.7 Hz, 1H), 7.61 (d, J=2.0 Hz, 2H), 7.53 (t, J=2.0 Hz, 1H), 6.55 (d, J=8.7 Hz, 1H), 4.01 (s, 3H).

I-45 ¹H NMR (500 MHz, Chloroform-d) δ 13.91 (s, 1H), 12.70 (s, 1H), 10.47 (s, 1H), 7.76 (d, J=9.1 Hz, 1H), 7.66-7.60 (m, 3H), 7.55-7.52 (m, 2H), 6.42 (d, J=9.1 Hz, 1H).

I-46 ¹H NMR (500 MHz, Chloroform-d) δ 12.39 (s, 1H), 10.16 (s, 1H), 7.89-7.81 (m, 2H), 6.91 (d, J=8.8 Hz, 2H), 6.02 (s, 1H), 3.96 (s, 3H), 3.85 (s, 3H), 3.81 (s, 3H).

B1 ¹H NMR (500 MHz, Methylene Chloride-d₂) δ 7.77 (s, 1H), 7.70 (s, 2H), 7.46 (d, J=7.8 Hz, 2H), 7.43 (t, J=7.6 Hz, 1H), 7.27 (t, J=7.6 Hz, 3H), 7.23 (d, J=7.4 Hz, 1H), 6.96 (t, J=7.6 Hz, 1H), 6.94-6.85 (m, 3H), 6.76 (t, J=7.4 Hz, 1H), 5.91 (d, J=7.7 Hz, 1H), 5.73 (d, J=10.3 Hz, 1H), 4.42 (d, J=17.0 Hz, 1H), 3.91 (d, J=17.0 Hz, 1H), 1.19 (s, 3H), 1.12 (s, 3H).

B2 ¹H NMR (500 MHz, Methylene Chloride-d₂) δ 7.79-7.71 (m, 6H), 7.65-7.61 (m, 2H), 7.60-7.56 (m, 2H), 7.49 (dd, J=8.3, 6.9 Hz, 4H), 7.46-7.43 (m, 2H), 7.14 (dd, J=7.3, 1.3 Hz, 1H), 7.07 (dd, J=7.7, 1.3 Hz, 1H), 6.95-6.90 (m, 1H), 6.87 (td, J=7.5, 1.0 Hz, 1H), 6.80 (d, J=8.3 Hz, 1H), 6.32 (dd, J=7.7, 0.9 Hz, 1H), 5.89 (d, J=10.3 Hz, 1H), 4.65 (d, J=17.0 Hz, 1H), 4.29 (d, J=17.0 Hz, 1H), 1.38 (s, 3H), 1.31 (s, 3H).

B3 ¹H NMR (500 MHz, Methylene Chloride-d₂) δ 8.06-8.00 (m, 2H), 7.76-7.70 (m, 3H), 7.63-7.60 (m, 2H), 7.60-7.55 (m, 2H), 7.51-7.46 (m, 2H), 7.44-7.38 (m, 2H), 7.00-6.95 (m, 1H), 6.79 (d, J=8.8 Hz, 1H), 6.31 (d, J=8.2 Hz, 1H), 5.89 (d, J=10.3 Hz, 1H), 4.64 (d, J=17.3 Hz, 1H), 4.39 (d, J=17.3 Hz, 1H), 1.38 (s, 3H), 1.33 (s, 3H).

B4 ¹H NMR (500 MHz, Methylene Chloride-d₂) δ 7.78 (s, 3H), 7.76-7.71 (m, 2H), 7.65-7.56 (m, 3H), 7.52-7.46 (m, 2H), 7.16 (dd, J=7.3, 1.3 Hz, 1H), 7.07 (td, J=7.7, 1.3 Hz, 1H), 6.94-6.87 (m, 2H), 6.78 (d, J=8.5 Hz, 1H), 6.26 (d, J=7.8 Hz, 1H), 5.82 (d, J=10.3 Hz, 1H), 4.64 (d, J=17.2 Hz, 1H), 4.34 (d, J=17.1 Hz, 1H), 1.38 (s, 3H), 1.30 (s, 3H).

B5 ¹H NMR (500 MHz, Methylene Chloride-d₂) δ 7.77-7.71 (m, 2H), 7.65-7.57 (m, 5H), 7.53-7.47 (m, 2H), 7.43 (d, J=7.8 Hz, 2H), 7.14 (dd, J=7.3, 1.2 Hz, 1H), 7.04 (td, J=7.7, 1.3 Hz, 1H), 6.94-6.85 (m, 2H), 6.77 (d, J=8.3 Hz, 1H), 6.23 (m, 1H), 5.83 (d, J=10.3 Hz, 1H), 4.61 (d, J=17.2 Hz, 1H), 4.25 (d, J=17.2 Hz, 1H), 1.37 (s, 3H), 1.30 (s, 3H).

B6 ¹H NMR (500 MHz, Methylene Chloride-d₂) δ 8.02-7.97 (m, 1H), 7.82-7.77 (m, 3H), 7.67-7.56 (m, 5H), 7.50-7.42 (m, 3H), 7.29-7.24 (m, 1H), 7.21-7.15 (m, 2H), 7.01-6.97 (m, 1H), 6.72 (d, J=8.4 Hz, 1H), 6.67 (d, J=8.6 Hz, 1H), 5.92 (d, J=10.3 Hz, 1H), 4.69 (d, J=17.4 Hz, 1H), 4.42 (d, J=17.4 Hz, 1H), 1.72 (s, 3H), 1.49 (s, 3H).

B7 ¹H NMR (500 MHz, Methylene Chloride-d₂) δ 7.78-7.72 (m, 5H), 7.68-7.61 (m, 4H), 7.61-7.57 (m, 2H), 7.52-7.47 (m, 5H), 7.43-7.38 (m, 2H), 7.01-6.95 (m, 1H), 6.80-6.76 (m, 1H), 6.44 (d, J=8.2 Hz, 1H), 5.85 (d, J=10.3 Hz, 1H), 4.72 (d, J=17.1 Hz, 1H), 4.41 (d, J=17.1 Hz, 1H), 3.02 (s, 3H), 1.55 (s, 3H), 1.43 (s, 3H).

B8 ¹H NMR (500 MHz, Chloroform-d) δ 7.47 (d, J=8.6 Hz, 1H), 7.35-7.30 (m, 2H), 7.29-7.20 (m, 3H), 7.09 (dd, J=8.4, 7.1 Hz, 2H), 6.94 (s, 1H), 6.52 (d, J=8.7 Hz, 1H), 6.20 (d, J=8.1 Hz, 1H), 5.57 (d, J=10.5 Hz, 1H), 3.93 (s, 3H), 2.58 (s, 3H), 1.02 (d, J=12.2 Hz, 6H).

B9 ¹H NMR (500 MHz, Chloroform-d) δ 7.62 (dd, J=8.3, 1.9 Hz, 1H), 7.43 (d, J=8.6 Hz, 1H), 7.41-7.37 (m, 2H), 7.33-7.25 (m, 3H), 7.19-7.14 (m, 2H), 6.52 (d, J=8.7 Hz, 1H), 6.24 (d, J=8.3 Hz, 1H), 5.57 (d, J=10.5 Hz, 1H), 4.62 (s, 2H), 3.93 (s, 3H), 2.63 (s, 3H), 1.06 (d, J=2.6 Hz, 6H).

B10 ¹H NMR (500 MHz, Methylene Chloride-d₂) δ 7.50-7.46 (m, 2H), 7.46-7.41 (m, 1H), 7.30-7.24 (m, 2H), 7.14 (d, J=10.5 Hz, 1H), 6.98 (td, J=7.6, 1.3 Hz, 1H), 6.87-6.82 (m, 1H), 6.68 (td, J=7.4, 1.0 Hz, 1H), 6.23-6.17 (m, 1H), 6.09 (s, 1H), 5.47 (d, J=10.5 Hz, 1H), 3.92 (s, 3H), 3.71 (s, 3H), 2.48 (s, 3H), 1.09-1.01 (m, 6H).

B11 ¹H NMR (500 MHz, Methylene Chloride-d₂) δ 7.40-7.36 (m, 2H), 7.34-7.30 (m, 2H), 7.22 (d, J=10.5 Hz, 1H), 7.20-7.15 (m, 2H), 7.00 (td, J=7.6, 1.3 Hz, 1H), 6.84-6.80 (m, 1H), 6.71-6.66 (m, 1H), 6.50 (d, J=8.6 Hz, 1H), 6.25-6.20 (m, 1H), 5.62 (d, J=10.5 Hz, 1H), 3.91 (s, 3H), 2.55 (s, 3H), 1.03 (d, J=3.4 Hz, 6H).

B12 ¹H NMR (500 MHz, Chloroform-d) δ 7.42 (d, J=8.6 Hz, 1H), 7.41-7.37 (m, 2H), 7.31-7.27 (m, 1H), 7.19-7.12 (m, 3H), 6.85 (td, J=7.6, 1.3 Hz, 1H), 6.79 (dd, J=7.2, 1.2 Hz, 1H), 6.67 (td, J=7.4, 0.9 Hz, 1H), 6.49 (d, J=8.6 Hz, 1H), 6.40 (d, J=2.3 Hz, 2H), 6.30 (t, J=2.3 Hz, 1H), 5.95 (d, J=7.6 Hz, 1H), 5.67 (d, J=10.5 Hz, 1H), 4.28 (d, J=16.6 Hz, 1H), 3.90 (s, 3H), 3.73 (s, 6H), 3.70 (d, J=16.6 Hz, 1H), 1.15 (s, 3H), 1.06 (s, 3H).

B13 ¹H NMR (500 MHz, Chloroform-d) δ 7.78-7.71 (m, 2H), 7.57-7.53 (m, 2H), 7.45 (d, J=8.5 Hz, 1H), 7.42 (d, J=8.6 Hz, 1H), 7.39-7.34 (m, 3H), 7.28-7.20 (m, 4H), 7.07-7.01 (m, 1H), 6.91 (dd, J=8.3, 7.1 Hz, 2H), 6.50 (d, J=8.6 Hz, 1H), 6.27 (d, J=8.6 Hz, 1H), 5.70 (d, J=10.6 Hz, 1H), 4.23 (dd, J=206.7, 17.4 Hz, 2H), 3.92 (s, 3H), 1.44 (s, 3H), 1.35 (s, 3H).

B14 ¹H NMR (500 MHz, Chloroform-d) δ 7.62 (d, J=8.7 Hz, 1H), 7.61-7.57 (m, 2H), 7.36-7.29 (m, 6H), 7.16 (d, J=10.5 Hz, 1H), 7.04 (td, J=7.4, 1.8 Hz, 1H), 6.86-6.78 (m, 2H), 6.56 (d, J=8.8 Hz, 1H), 5.91 (d, J=7.7 Hz, 1H), 5.59 (d, J=10.6 Hz, 1H), 4.39 (d, J=17.1 Hz, 1H), 3.93 (s, 3H), 3.80 (d, J=17.1 Hz, 1H), 1.13 (s, 3H), 1.04 (s, 3H).

B15 ¹H NMR (500 MHz, Chloroform-d) δ 7.57-7.53 (m, 2H), 7.45-7.39 (m, 3H), 7.38-7.33 (m, 1H), 7.30 (d, J=8.1 Hz, 2H), 7.24-7.18 (m, 3H), 6.82 (dd, J=8.2, 2.2 Hz, 1H), 6.77 (d, J=2.1 Hz, 1H), 6.51 (d, J=8.7 Hz, 1H), 5.74 (d, J=8.2 Hz, 1H), 5.60 (d, J=10.5 Hz, 1H), 4.36 (d, J=17.1 Hz, 1H), 3.91 (s, 3H), 3.83 (d, J=17.2 Hz, 1H), 1.16 (s, 3H), 1.08 (s, 3H).

B16 ¹H NMR (500 MHz, Chloroform-d) δ 7.48-7.44 (m, 2H), 7.41-7.36 (m, 1H), 7.23-7.18 (m, 2H), 7.14-7.10 (m, 1H), 7.09 (d, J=10.5 Hz, 1H), 6.96 (td, J=7.7, 1.2 Hz, 1H), 6.63 (td, J=7.4, 1.0 Hz, 1H), 6.14-6.09 (m, 1H), 6.05 (s, 1H), 5.47 (d, J=10.5 Hz, 1H), 3.91 (s, 3H), 3.76 (s, 3H), 2.41 (s, 3H), 1.71-1.15 (m, 10H).

B17 ¹H NMR (500 MHz, Chloroform-d) δ 7.53-7.48 (m, 2H), 7.46-7.39 (m, 1H), 7.28-7.22 (m, 5H), 7.21-7.14 (m, 5H), 7.06 (d, J=10.6 Hz, 1H), 6.81 (td, J=7.7, 1.2 Hz, 1H), 6.63 (td, J=7.5, 1.0 Hz, 1H), 6.06 (s, 1H), 5.85 (dd, J=7.8, 1.0 Hz, 1H), 5.57 (d, J=10.6 Hz, 1H), 4.29 (d, J=16.6 Hz, 1H), 3.90 (s, 3H), 3.78 (s, 3H), 3.58 (d, J=16.6 Hz, 1H), 1.76-1.62 (m, 4H), 1.62-1.54 (m, 2H), 1.54-1.44 (m, 2H), 1.44-1.35 (m, 1H), 1.30-1.19 (m, 1H).

B18 ¹H NMR (500 MHz, Chloroform-d) δ 7.40 (d, J=8.6 Hz, 1H), 7.38-7.32 (m, 2H), 7.30-7.26 (m, 1H), 7.17 (d, J=10.6 Hz, 1H), 7.12 (t, J=7.8 Hz, 3H), 6.98 (t, J=7.6 Hz, 1H), 6.64 (t, J=7.4 Hz, 1H), 6.48 (d, J=8.6 Hz, 1H), 6.14 (d, J=7.7 Hz, 1H), 5.61 (d, J=10.6 Hz, 1H), 3.92 (s, 3H), 2.51 (s, 3H), 1.75-1.12 (m, 10H).

B19 ¹H NMR (500 MHz, Chloroform-d) δ 7.54-7.50 (m, 2H), 7.47-7.42 (m, 1H), 7.31-7.23 (m, 4H), 7.21-7.14 (m, 3H), 7.11-7.05 (m, 2H), 6.77 (dd, J=8.3, 2.1 Hz, 1H), 6.07 (s, 1H), 5.75 (d, J=8.3 Hz, 1H), 5.54 (d, J=10.5 Hz, 1H), 4.29 (d, J=16.5 Hz, 1H), 3.90 (s, 3H), 3.79 (s, 3H), 3.57 (d, J=16.5 Hz, 1H), 1.77-0.79 (m, 10H).

B20 ¹H NMR (500 MHz, Chloroform-d) δ 7.59-7.53 (m, 2H), 7.44-7.39 (m, 1H), 7.26-7.18 (m, 4H), 7.15-7.09 (m, 1H), 7.03-6.98 (m, 2H), 6.94-6.88 (m, 3H), 6.76-6.71 (m, 1H), 6.24 (dd, J=8.2, 1.0 Hz, 1H), 5.99 (s, 1H), 5.44 (d, J=10.5 Hz, 1H), 3.84 (s, 3H), 3.71 (s, 3H), 1.16 (d, J=6.0 Hz, 6H).

B21 ¹H NMR (500 MHz, Chloroform-d) δ 7.56-7.49 (m, 1H), 7.47-7.44 (m, 1H), 7.43-7.36 (m, 1H), 7.25-7.18 (m, 1H), 7.12-7.04 (m, 1H), 7.00-6.93 (m, 1H), 6.81-6.72 (m, 1H), 6.68-6.62 (m, 1H), 6.20-6.02 (m, 2H), 5.54-5.43 (m, 1H), 3.93-3.86 (m, 3H), 3.78-3.71 (m, 3H), 2.47-2.37 (m, 3H), 1.49-1.18 (m, 3H), 1.13-1.07 (m, 3H), 0.97-0.93 (m, 3H).

B22 ¹H NMR (500 MHz, Chloroform-d) δ 7.54-7.47 (m, 4H), 7.47-7.42 (m, 1H), 7.30-7.23 (m, 4H), 7.18 (dd, J=7.4, 1.2 Hz, 1H), 7.07 (d, J=10.5 Hz, 1H), 6.84 (td, J=7.7, 1.2 Hz, 1H), 6.67 (td, J=7.5, 1.0 Hz, 1H), 6.06 (s, 1H), 5.75 (dd, J=7.9, 1.0 Hz, 1H), 5.50 (d, J=10.5 Hz, 1H), 4.29 (d, J=17.3 Hz, 1H), 3.90 (s, 3H), 3.77 (s, 3H), 3.69 (d, J=17.3 Hz, 1H), 1.80-0.76 (m, 10H).

B23 ¹H NMR (500 MHz, Chloroform-d) δ 7.52-7.47 (m, 2H), 7.45-7.39 (m, 1H), 7.28-7.23 (m, 2H), 7.20 (dd, J=7.4, 1.2 Hz, 1H), 7.03 (t, J=7.5 Hz, 1H), 6.95-6.88 (m, 3H), 6.72 (d, J=10.6 Hz, 1H), 6.65 (td, J=7.5, 0.9 Hz, 1H), 6.12 (d, J=7.8 Hz, 1H), 6.05 (s, 1H), 5.01 (d, J=10.6 Hz, 1H), 4.01 (dd, J=46.1, 14.5 Hz, 2H), 3.89 (s, 3H), 3.78 (s, 3H), 2.20 (s, 6H), 1.80-0.79 (m, 10H).

B24 ¹H NMR (500 MHz, Chloroform-d) δ 7.59-7.53 (m, 2H), 7.52-7.44 (m, 4H), 7.30 (d, J=8.1 Hz, 2H), 7.09 (d, J=10.5 Hz, 1H), 6.94 (td, J=7.6, 1.3 Hz, 1H), 6.84 (dd, J=7.2, 1.3 Hz, 1H), 6.75 (td, J=7.4, 0.9 Hz, 1H), 6.08 (s, 1H), 5.83 (d, J=7.7 Hz, 1H), 5.49 (d, J=10.5 Hz, 1H), 4.31 (d, J=17.0 Hz, 1H), 3.91 (s, 3H), 3.80 (s, 3H), 3.69 (d, J=17.0 Hz, 1H), 1.15 (s, 3H), 1.03 (s, 3H).

B25 ¹H NMR (500 MHz, Chloroform-d) δ 7.59 (dd, J=8.3, 2.0 Hz, 1H), 7.54-7.49 (m, 2H), 7.45-7.40 (m, 1H), 7.31 (d, J=2.0 Hz, 1H), 7.28-7.23 (m, 1H), 7.18 (d, J=10.5 Hz, 1H), 6.17 (d, J=8.3 Hz, 1H), 6.08 (s, 1H), 5.42 (d, J=10.5 Hz, 1H), 5.30 (s, 1H), 4.60 (s, 2H), 3.92 (s, 3H), 3.75 (s, 3H), 2.55 (s, 3H), 1.06 (d, J=1.5 Hz, 6H).

B26 ¹H NMR (500 MHz, Chloroform-d) δ 7.57-7.51 (m, 2H), 7.43-7.37 (m, 1H), 7.25-7.18 (m, 5H), 7.16-7.09 (m, 1H), 7.00-6.94 (m, 2H), 6.93-6.85 (m, 2H), 6.69 (td, J=7.4, 1.1 Hz, 1H), 6.18 (dd, J=7.8, 1.0 Hz, 1H), 5.99 (s, 1H), 5.45 (d, J=10.5 Hz, 1H), 3.84 (s, 3H), 3.73 (s, 3H), 1.80-0.80 (m, 10H).

B27 ¹H NMR (500 MHz, Chloroform-d) δ 7.57-7.51 (m, 2H), 7.43-7.38 (m, 3H), 7.35-7.30 (m, 3H), 7.21-7.15 (m, 3H), 6.89-6.83 (m, 2H), 6.72 (td, J=7.4, 0.9 Hz, 1H), 6.50 (d, J=8.6 Hz, 1H), 5.82 (d, J=7.6 Hz, 1H), 5.62 (d, J=10.5 Hz, 1H), 4.12 (dd, J=257.4, 17.2 Hz, 2H), 3.91 (s, 3H), 1.17 (s, 3H), 1.10 (s, 3H).

B28 ¹H NMR (500 MHz, Chloroform-d) δ 7.44 (d, J=8.6 Hz, 1H), 7.37-7.34 (m, 2H), 7.32-7.28 (m, 1H), 7.21 (d, J=10.5 Hz, 1H), 7.18-7.13 (m, 2H), 6.95 (dd, J=8.2, 2.1 Hz, 1H), 6.69 (d, J=2.1 Hz, 1H), 6.50 (d, J=8.6 Hz, 1H), 6.09 (d, J=8.2 Hz, 1H), 5.56 (d, J=10.5 Hz, 1H), 3.92 (s, 3H), 2.50 (s, 3H), 1.02 (s, 3H), 0.97 (s, 3H).

B29 ¹H NMR (300 MHz, Acetonitrile-d₃) δ 7.60-7.49 (m, 3H), 7.42-7.32 (m, 2H), 7.22 (d, J=8.4 Hz, 1H), 7.05-6.84 (m, 3H), 6.68 (td, J=7.4, 1.0 Hz, 1H), 6.60 (d, J=8.4 Hz, 1H), 6.29 (d, J=7.7 Hz, 1H), 5.61 (d, J=10.3 Hz, 1H), 3.65 (s, 3H), 2.53 (s, 3H), 1.07 (s, 3H), 1.05 (s, 3H).

B30 ¹H NMR (500 MHz, Chloroform-d) δ 7.51-7.45 (m, 2H), 7.44-7.39 (m, 1H), 7.24 (t, J=7.7 Hz, 2H), 7.10 (d, J=10.5 Hz, 1H), 7.03 (d, J=2.1 Hz, 1H), 6.92 (dd, J=8.2, 2.1 Hz, 1H), 6.05 (s, 1H), 6.03 (d, J=8.2 Hz, 1H), 5.44 (d, J=10.6 Hz, 1H), 3.91 (s, 3H), 3.76 (s, 3H), 2.40 (s, 3H), 1.68-1.11 (m, 10H).

B31 ¹H NMR (300 MHz, Chloroform-d) δ 8.05-7.37 (m, 17H), 6.97 (d, J=10.3 Hz, 1H), 6.27 (d, J=8.1 Hz, 1H), 5.92 (d, J=10.3 Hz, 1H), 4.79 (q, J=17.5 Hz, 2H), 1.42 (d, J=20.2 Hz, 6H).

B32 ¹H NMR (400 MHz, Methylene Chloride-d₂) δ 7.58-7.53 (m, 2H), 7.50-7.40 (m, 3H), 7.37-7.32 (m, 2H), 7.31-7.20 (m, 4H), 6.99-6.85 (m, 4H), 6.72 (td, J=7.4, 1.0 Hz, 1H), 5.87 (d, J=7.7 Hz, 1H), 5.73 (d, J=10.3 Hz, 1H), 4.15 (dd, J=201.8, 17.2 Hz, 2H), 1.19 (s, 3H), 1.13 (s, 3H).

B33 ¹H NMR (400 MHz, Methylene Chloride-d₂) δ 7.78 (s, 1H), 7.71 (s, 2H), 7.41-7.23 (m, 4H), 7.22-7.12 (m, 2H), 7.01-6.85 (m, 4H), 6.78 (td, J=7.4, 1.0 Hz, 1H), 6.00-5.91 (m, 1H), 5.71 (d, J=9.1 Hz, 1H), 4.16 (dd, J=208.4, 16.9 Hz, 2H), 1.18 (s, 3H), 1.11 (s, 3H).

B34 ¹H NMR (400 MHz, Methylene Chloride-d₂) δ 7.77 (s, 1H), 7.70 (s, 2H), 7.43-7.33 (m, 4H), 7.26-7.16 (m, 3H), 6.94-6.87 (m, 2H), 6.79-6.72 (m, 1H), 6.53 (d, J=8.7 Hz, 1H), 5.91 (dd, J=8.1, 0.9 Hz, 1H), 5.65 (d, J=10.5 Hz, 1H), 4.19 (dd, J=200.5, 17.0 Hz, 2H), 3.90 (s, 3H), 1.18 (s, 3H), 1.12 (s, 3H).

B35 ¹H NMR (400 MHz, Methylene Chloride-d₂) δ 7.66-7.59 (m, 2H), 7.57-7.45 (m, 8H), 7.43-7.36 (m, 2H), 7.37-7.31 (m, 2H), 7.20 (d, J=10.5 Hz, 1H), 6.91-6.83 (m, 1H), 6.73 (td, J=7.6, 1.4 Hz, 1H), 6.67 (td, J=7.4, 1.1 Hz, 1H), 6.54 (d, J=8.6 Hz, 1H), 5.85-5.76 (m, 1H), 5.66 (d, J=10.5 Hz, 1H), 4.51-3.88 (m, 5H), 1.17 (s, 3H), 1.15 (s, 3H).

B36 ¹H NMR (400 MHz, Methylene Chloride-d₂) δ 7.65-7.59 (m, 2H), 7.56-7.50 (m, 1H), 7.40-7.32 (m, 2H), 7.17 (d, J=10.5 Hz, 1H), 7.07 (td, J=7.6, 1.3 Hz, 1H), 6.80 (dd, J=7.2, 1.2 Hz, 1H), 6.72-6.66 (m, 1H), 6.49 (d, J=7.7 Hz, 1H), 6.11 (s, 1H), 5.43 (d, J=10.5 Hz, 1H), 3.94 (s, 3H), 3.67 (s, 3H), 3.65-3.37 (m, 2H), 3.28 (s, 9H), 2.17-1.88 (m, 2H), 1.37-1.18 (m, 2H), 1.03 (s, 3H), 0.91 (s, 3H).

B37 ¹H NMR (400 MHz, Methylene Chloride-d₂) δ 7.79-7.70 (m, 2H), 7.59 (d, J=8.5 Hz, 1H), 7.39-7.28 (m, 4H), 7.23-7.14 (m, 2H), 7.13-7.07 (m, 2H), 6.69 (d, J=8.6 Hz, 1H), 6.10 (s, 1H), 5.55 (d, J=10.5 Hz, 1H), 3.93 (s, 3H), 3.72 (s, 3H), 2.59 (s, 3H), 1.35 (s, 3H), 1.22 (s, 3H).

B38 ¹H NMR (500 MHz, Chloroform-d) δ 7.60 (dd, J=8.2, 2.0 Hz, 1H), 7.57-7.52 (m, 2H), 7.50-7.43 (m, 1H), 7.33 (d, J=1.9 Hz, 1H), 7.28 (t, J=7.8 Hz, 2H), 7.12 (d, J=8.4 Hz, 1H), 6.88 (d, J=10.3 Hz, 1H), 6.52 (d, J=8.5 Hz, 1H), 6.20 (d, J=8.3 Hz, 1H), 5.50 (d, J=10.3 Hz, 1H), 4.60 (s, 2H), 3.73 (s, 3H), 2.57 (s, 3H), 1.08 (s, 3H), 1.08 (s, 3H).

B39 ¹H NMR (400 MHz, Methylene Chloride-d₂) δ 8.03-7.97 (m, 1H), 7.87-7.80 (m, 2H), 7.79-7.74 (m, 3H), 7.74-7.71 (m, 2H), 7.64-7.56 (m, 4H), 7.52-7.42 (m, 6H), 7.28-7.22 (m, 1H), 6.97 (dd, J=10.4, 0.7 Hz, 1H), 6.80 (d, J=8.6 Hz, 1H), 6.74 (d, J=8.4 Hz, 1H), 6.01 (d, J=10.3 Hz, 1H), 4.74 (dd, J=113.9, 16.8 Hz, 2H), 1.74 (s, 3H), 1.53 (s, 3H).

B40 ¹H NMR (500 MHz, Chloroform-d) δ 7.45-7.41 (m, 2H), 7.37 (d, J=8.6 Hz, 1H), 7.27-7.20 (m, 3H), 7.10 (dd, J=8.4, 7.3 Hz, 3H), 7.08-7.00 (m, 3H), 6.93-6.87 (m, 2H), 6.76-6.69 (m, 1H), 6.42 (d, J=8.7 Hz, 1H), 6.34 (d, J=7.9 Hz, 1H), 5.54 (d, J=10.5 Hz, 1H), 3.85 (s, 3H), 1.13 (s, 3H), 1.13 (s, 3H).

B41 ¹H NMR (500 MHz, Chloroform-d) δ 7.53 (d, J=8.6 Hz, 1H), 7.43-7.37 (m, 2H), 7.30-7.26 (m, 1H), 7.24 (d, J=10.6 Hz, 1H), 7.17 (t, J=7.7 Hz, 2H), 6.83 (d, J=8.6 Hz, 1H), 6.52 (d, J=8.6 Hz, 2H), 5.47 (d, J=10.6 Hz, 1H), 3.92 (s, 3H), 2.95 (s, 3H), 1.20 (s, 3H), 1.05 (s, 3H).

B42 ¹H NMR (500 MHz, Chloroform-d) δ 7.68 (dd, J=8.2, 2.0 Hz, 1H), 7.55-7.48 (m, 4H), 7.35 (d, J=1.9 Hz, 1H), 7.17 (d, J=10.5 Hz, 1H), 6.19 (d, J=8.2 Hz, 1H), 6.09 (s, 1H), 5.42 (d, J=10.5 Hz, 1H), 4.81 (s, 2H), 3.93 (s, 3H), 3.78 (s, 3H), 2.54 (s, 3H), 1.06 (s, 3H), 1.05 (s, 3H).

B43 ¹H NMR (500 MHz, Chloroform-d) δ 7.50-7.45 (m, 4H), 7.09 (d, J=10.6 Hz, 1H), 7.07 (d, J=2.1 Hz, 1H), 7.03 (dd, J=8.2, 2.1 Hz, 1H), 6.06 (s, 1H), 6.03 (d, J=8.1 Hz, 1H), 5.44 (d, J=10.6 Hz, 1H), 3.92 (s, 3H), 3.80 (s, 3H), 2.38 (s, 3H), 1.70-1.12 (m, 10H).

B44 ¹H NMR (500 MHz, Chloroform-d) δ 7.63 (dd, J=8.2, 1.9 Hz, 1H), 7.39-7.33 (m, 4H), 7.18 (d, J=10.5 Hz, 1H), 6.21 (d, J=8.3 Hz, 1H), 6.09 (s, 1H), 5.44 (d, J=10.5 Hz, 1H), 4.71 (s, 2H), 3.94 (s, 3H), 3.79 (s, 3H), 2.61 (s, 3H), 1.10 (s, 3H), 1.06 (s, 3H).

B45 ¹H NMR (500 MHz, Chloroform-d) δ 7.60 (dd, J=8.2, 2.0 Hz, 1H), 7.44 (t, J=1.9 Hz, 1H), 7.41-7.32 (m, 3H), 7.21-7.15 (m, 2H), 6.18 (d, J=8.3 Hz, 1H), 6.09 (s, 1H), 5.43 (d, J=10.5 Hz, 1H), 4.68 (s, 2H), 3.93 (s, 3H), 3.77 (s, 3H), 2.57 (s, 3H), 1.08 (s, 3H), 1.06 (s, 3H).

B46 ¹H NMR (500 MHz, Chloroform-d) δ 7.74 (d, J=2.1 Hz, 1H), 7.70 (d, J=9.4 Hz, 2H), 7.60 (dd, J=8.6, 2.2 Hz, 1H), 7.56-7.52 (m, 1H), 7.44 (t, J=7.6 Hz, 2H), 7.23-7.17 (m, 2H), 6.94-6.84 (m, 5H), 6.72 (d, J=7.8 Hz, 1H), 5.24 (t, J=4.2 Hz, 1H), 4.51 (dd, J=333.1, 16.3 Hz, 2H), 3.88 (s, 3H), 2.52-2.44 (m, 2H), 1.48 (s, 3H), 1.20 (s, 3H).

B47 ¹H NMR (500 MHz, Chloroform-d) δ 7.35-7.30 (m, 3H), 7.13 (d, J=10.5 Hz, 1H), 6.96 (dd, J=8.2, 2.1 Hz, 1H), 6.78 (d, J=2.2 Hz, 1H), 6.09 (d, J=8.2 Hz, 1H), 6.07 (s, 1H), 5.43 (d, J=10.5 Hz, 1H), 3.92 (s, 3H), 3.79 (s, 3H), 2.49 (s, 3H), 1.05 (s, 3H), 1.04 (s, 3H).

B48 ¹H NMR (500 MHz, Chloroform-d) δ 7.35 (t, J=1.9 Hz, 1H), 7.32 (d, J=1.9 Hz, 2H), 7.14-7.08 (m, 2H), 6.98 (dd, J=8.2, 2.0 Hz, 1H), 6.12-6.01 (m, 2H), 5.46 (d, J=10.6 Hz, 1H), 3.92 (s, 3H), 3.79 (s, 3H), 2.47 (s, 3H), 1.84-1.11 (m, 10H).

B49 ¹H NMR (500 MHz, Chloroform-d) δ 7.54-7.47 (m, 2H), 7.12 (dd, J=7.4, 1.2 Hz, 1H), 7.10 (d, J=10.6 Hz, 1H), 6.96 (td, J=7.6, 1.1 Hz, 1H), 6.74-6.68 (m, 2H), 6.62 (td, J=7.4, 1.0 Hz, 1H), 6.15 (d, J=7.7 Hz, 1H), 6.04 (s, 1H), 5.47 (d, J=10.6 Hz, 1H), 3.90 (s, 3H), 3.84 (s, 3H), 3.75 (s, 3H), 2.48 (s, 3H), 1.77-1.17 (m, 10H).

B50 ¹H NMR (500 MHz, Chloroform-d) δ 7.73 (dd, J=8.2, 2.0 Hz, 1H), 7.57 (d, J=8.7 Hz, 1H), 7.40 (d, J=1.9 Hz, 1H), 7.32-7.28 (m, 2H), 7.26 (s, 1H), 7.23 (t, J=1.9 Hz, 1H), 6.60 (d, J=8.7 Hz, 1H), 6.36 (d, J=8.3 Hz, 1H), 5.60 (d, J=10.6 Hz, 1H), 4.79 (s, 2H), 3.98 (s, 3H), 2.76 (s, 3H), 1.12 (s, 3H), 1.08 (s, 3H).

B51 ¹H NMR (500 MHz, Chloroform-d) δ 7.62 (dd, J=8.3, 2.0 Hz, 1H), 7.49-7.37 (m, 8H), 7.34-7.29 (m, 3H), 7.16 (t, J=7.7 Hz, 2H), 6.59 (d, J=8.7 Hz, 1H), 6.23 (d, J=8.2 Hz, 1H), 5.56 (d, J=10.5 Hz, 1H), 5.17 (s, 2H), 4.64 (s, 2H), 2.64 (s, 3H), 1.07 (s, 3H), 1.05 (s, 3H).

B52 ¹H NMR (500 MHz, Chloroform-d) δ 7.43 (d, J=8.6 Hz, 1H), 7.35 (dd, J=8.2, 1.3 Hz, 2H), 7.32-7.28 (m, 1H), 7.19 (d, J=10.6 Hz, 1H), 7.18-7.13 (m, 2H), 7.03 (d, J=2.1 Hz, 1H), 6.94 (dd, J=8.2, 2.1 Hz, 1H), 6.49 (d, J=8.6 Hz, 1H), 6.06 (d, J=8.2 Hz, 1H), 5.58 (d, J=10.6 Hz, 1H), 3.92 (s, 3H), 2.49 (s, 3H), 1.68-1.12 (m, 10H).

### B. Preparation of Photopolymerizable Starting Materials

The dual color photoinitiator (DCPI) is dissolved in 1-Butyl-2-pyrrolidinone (NBP) and N-Methyldiethanolamine (MDEA), SR833S (CAS 42594-17-2), Urethane dimethacrylate (UDMA, CAS 72869-86-4), Rheobyk 7410CA (Provided by BYK), and Rheobyk 7420CA (Provided by BYK) are added. The mixture is homogenized in a Hauschild Speedmixer, filled into cuvettes or placed between glass plates according to FIG. 1. Cuvettes are centrifuged prior to analysis and printing to remove air bubbles. Table 2 gives an overview of compositions (formulations) for photopolymerizable starting materials (all values in g unless otherwise noted).

| Entry | DCPI [mg] | | NBP | MDEA | SR833S | 7410CA | 7420CA | UDMA | DR (l₁ [nm]) | |
|---|---|---|---|---|---|---|---|---|---|---|
| F1 | B7 | 4.3 | 0.4 | 1.0 | 0 | 0 | 0 | 19.0 | | |
| F2 | B10 | 3.9 | 0.4 | 1.0 | 0 | 0 | 0 | 19.0 | | |
| F3 | B1 | 4.2 | 0.4 | 1.0 | 0 | 0 | 0 | 19.0 | | |
| F4 | B3 | 4.3 | 0.4 | 1.0 | 0 | 0 | 0 | 19.0 | | |
| F5 | B11 | 3.6 | 0.4 | 1.0 | 0 | 0 | 0 | 19.0 | | |
| F6 | I-28 | 4.2 | 0.4 | 1.0 | 0 | 0 | 0 | 19.0 | | |
| F7 | I-30 | 4.6 | 0.4 | 1.0 | 0 | 0 | 0 | 19.0 | | |
| F8 | B15 | 5.1 | 0.2 | 1.0 | 3.0 | 0.15 | 0.15 | 16.1 | | |
| F9 | B21 | 4.2 | 0.2 | 1.0 | 3.0 | 0.15 | 0.15 | 16.7 | −0.038 | (375) |
| F10 | B52 | 5.8 | 0.2 | 1.0 | 3.0 | 0.15 | 0.15 | 15.8 | 0.092 | (375) |
| F11 | B23 | 5.4 | 0.2 | 1.0 | 3.0 | 0.15 | 0.15 | 15.8 | 0.021 | (375) |
| F12 | I-38 | 5.1 | 0.2 | 1.0 | 3.0 | 0.15 | 0.15 | 16.2 | 0.285 | (405) |
| F13 | B19 | 4.9 | 0.2 | 1.0 | 3.0 | 0.15 | 0.15 | 15.7 | 0.009 | (375) |
| F14 | B22 | 5.0 | 0.2 | 1.0 | 3.0 | 0.15 | 0.15 | 16.6 | −0.003 | (375) |
| F15 | B17 | 5.4 | 0.2 | 1.0 | 3.0 | 0.15 | 0.15 | 15.8 | 0.011 | (375) |
| F16 | B29 | 6.1 | 0.2 | 1.0 | 3.0 | 0.15 | 0.15 | 15.9 | 0.123 | (375) |
| F17 | B36 | 4.9 | 0.2 | 1.0 | 3.0 | 0.15 | 0.15 | 15.8 | −0.029 | (375) |
| F18 | B24 | 4.7 | 0.2 | 1.0 | 3.0 | 0.15 | 0.15 | 15.7 | 0.182 | (405) |
| F19 | B26 | 4.5 | 0.2 | 1.0 | 3.0 | 0.15 | 0.15 | 16.1 | −0.024 | (375) |
| F20 | B16 | 4.6 | 0.2 | 1.0 | 3.0 | 0.15 | 0.15 | 15.9 | 0.023 | (375) |
| F21 | B25 | 5.0 | 0.2 | 1.0 | 3.0 | 0.15 | 0.15 | 16.4 | 0.004 | (375) |
| F22 | B20 | 4.8 | 0.2 | 1.0 | 3.0 | 0.15 | 0.15 | 15.9 | 0.009 | (375) |
| F23 | B9 | 5.5 | 0.2 | 1.0 | 3.0 | 0.15 | 0.15 | 15.9 | 0.070 | (375) |
| F24 | B8 | 6.0 | 0.2 | 1.0 | 3.0 | 0.15 | 0.15 | 15.9 | 0.112 | (375) |
| F25 | I-19 | 5.2 | 0.2 | 1.0 | 3.0 | 0.15 | 0.15 | 15.7 | 0.290 | (375) |
| F26 | I-35 | 6.7 | 0.2 | 1.0 | 3.0 | 0.15 | 0.15 | 16.1 | 0.263 | (375) |
| F27 | I-37 | 6.4 | 0.2 | 1.0 | 3.0 | 0.15 | 0.15 | 15.6 | 0.321 | (405) |
| F28 | I-32 | 6.7 | 0.2 | 1.0 | 3.0 | 0.15 | 0.15 | 16.5 | 0.373 | (405) |
| F29 | B10 | 5.0 | 0.2 | 1.0 | 3.0 | 0.15 | 0.15 | 16.5 | 0.039 | (375) |
| F30 | B11 | 5.5 | 0.2 | 1.0 | 3.0 | 0.15 | 0.15 | 16.4 | 0.137 | (375) |
| F31 | I-29 | 4.8 | 0.2 | 1.0 | 3.0 | 0.15 | 0.15 | 16.0 | 0.421 | (375) |
| F32 | I-31 | 5.1 | 0.2 | 1.0 | 3.0 | 0.15 | 0.15 | 15.6 | 0.496 | (375) |
| F33 | I-33 | 5.4 | 0.2 | 1.0 | 3.0 | 0.15 | 0.15 | 15.8 | 0.274 | (405) |
| F34 | I-36 | 5.5 | 0.2 | 1.0 | 3.0 | 0.15 | 0.15 | 16.1 | 0.330 | (375) |
| F35 | B27 | 5.7 | 0.2 | 1.0 | 3.0 | 0.15 | 0.15 | 15.8 | 0.078 | (375) |
| F36* | B3 | 12.1 | 0.2 | 1.0 | — | — | — | 19.0 | | |
| F37* | I-34 | 9.6 | 0.2 | 1.0 | — | — | — | 21.4 | | |

*Containing 200 mg Aerosil 200

### C. Polymerization Experiments

An assembly according to FIG. 1 is placed in a Rapidshape RS PRO cure device for 20 min at highest intensity to polymerize the starting material. The assembly is irradiated for 20 min using a white light lamp (Optonica LED 100W ip65 cold white, article 5733, available from Optonica LED GmbH) and an absorption spectrum of the obtained polymer plate is measured using a Cary50 UV-vis Spectrophotometer. FIGS. 2a and 2b show the comparison of F3 and F6, where the material containing 1 (F3) shows better transmission in the near UV region and thus superior optical properties compared to the material containing I-28 (F6). It is thus evident that a dual color photoinitiator initiator comprising an electron withdrawing substituent in the R⁹ position shows better transparency compared to dual color photoinitiators initiators, where the electron withdrawing group is located in the R¹² position.

### D. Printing Tests

The following formulations have been used to manufacture plano convex lenses with the parameters as specified in the following Table 2 at a print speed of 2 mm/min. For the manufacture a Xube2 Device available from xolo GmbH has been used which represents an apparatus as described in FIGS. 7 and 8. A picture showing the printed lenses is given in FIG. 9.

| Formulation | l₁ [nm] | Intensity of l₁ | | l₂ [nm] | Intensity of l₂ |
|---|---|---|---|---|---|
| F13 | 375 | 13.5 | mJ/mm² | 480-630 | 390 mW/cm² |
| F18 | 405 | 20 | mJ/mm² | 480-630 | 390 mW/cm² |
| F20 | 375 | 11 | mJ/mm² | 480-630 | 390 mW/cm² |
| F23 | 375 | 9 | mJ/mm² | 480-630 | 390 mW/cm² |
| F35 | 375 | 4.5 | mJ/mm² | 480-630 | 390 mW/cm² |

The features disclosed in the foregoing description and in the dependent claims may, both separately and in any combination thereof, be material for realizing the aspects of the disclosure made in the independent claims, in diverse forms thereof.

1. A process for the manufacture of a spiropyran represented by the following formula (1): 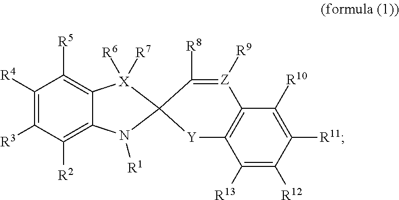
the process comprising the steps of
providing a precursor, wherein the precursor is a spiropyran represented by the following formula (2) 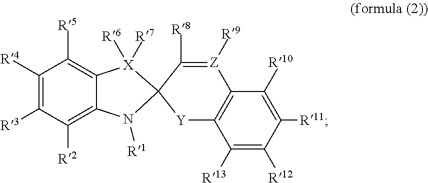
providing a reactant, wherein the reactant is
an indolenium salt represented by the following formula (3): 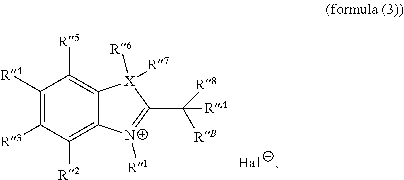
or the corresponding 2-methyleneindoline compound; or
a salicylaldehyde represented by the following formula (4): 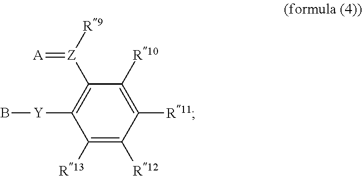
or
a spiropyran represented by the following formula 5: 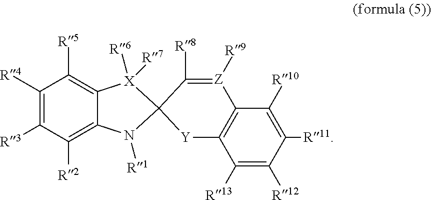
optionally pre-activating the precursor
providing a reaction mixture comprising the precursor or optionally a preactivated precursor, and the reactant to obtain said spiropyran of formula (1);
wherein the spiropyran, the precursor, and the reactant are different from each other;
wherein if the reactant is an indolenium salt of formula (3), the obtained spiropyran of formula (1) is represented by the following formula (1A): 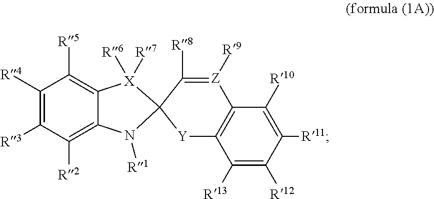
wherein R^{″1} to R^{″8} of formula (1A) are independently the same as R^{″1} to R^{″8} of formula (3) and R^{′9} to R^{′13} of formula (1A) are independently the same as R^{′9} to R^{′13} of formula (2);
wherein if the reactant is a salicylaldehyde of formula (4), the obtained spiropyran of formula (1) is represented by the following formula (1B): 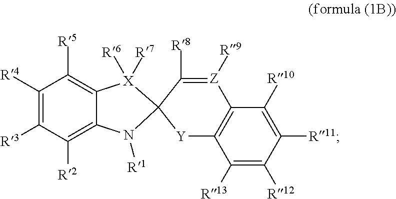
wherein R^{′1} to R^{′8} of formula (1B) are independently the same as R^{′1} to R^{′8} of formula (2) and R^{″9} to R^{″13} of formula (1B) are independently the same as R^{″9} to R^{″13} of formula (4);
wherein if the reactant is a spiropyran of formula (5),
the obtained spiropyran of formula (1) is represented by formula (1A), wherein R^{″1} to R^{″8} of formula (1A) are independently the same as R^{″1} to R^{″8} of formula (5) and R^{′9} to R^{′13} of formula (1A) are independently the same as R^{′9} to R^{′13} of formula (2), or
the obtained spiropyran of formula (1) is represented by formula (1B), wherein R^{′1} to R^{′8} of formula (1B) are independently the same as R^{′1} to R^{′8} of formula (2) and R^{″9} to R^{″13} of formula (1B) are independently the same as R^{″9} to R^{″13} of formula (5);
wherein X is selected from S, C, or N; if X is S, then R⁶, R⁷, R^{′6}, R^{′7}, R^{″6}, R^{″7} may not be present accordingly; if X is N, then R⁷, R^{′7}, R^{″7} may not be present accordingly;
wherein Y is selected from O, S, or N; where Y is N, the substituent contains the atoms necessary to complete a cyclic structure with R¹³ selected from the group consisting of benzimidazole, indoline, indole, dihydroquinoline, and tetrahydroquinoline;
wherein Z is selected from N or C;
wherein if present A is selected from O, S, or Se;
wherein if present B is selected from H or D;
wherein if present Hal- is a halogen anion or an anionic compound;
wherein if present R¹ to R¹³, R^{′1} to R^{′13}, and R^{″1} to R^{″13} are independently selected from the group consisting of H; D; halogen; NO₂; CN; OH; SH; CF₃; substituted or unsubstituted C₁-C₂₀-alkyl; substituted or unsubstituted C₃-C₂₀-cycloalkyl; substituted or unsubstituted C₆-C₄₈-aryl; substituted or unsubstituted C₂-C₄₂-heteroaryl; substituted or unsubstituted C₂-C₄₉-alkyl acyl; substituted or unsubstituted C₂-C₄₉-aryl acyl; substituted or unsubstituted C₁-C₂₀-alkoxy; substituted or unsubstituted C₆-C₄₈-aryloxy; NH₂; substituted or unsubstituted C₁-C₂₀-alkyl ester; substituted or unsubstituted C₆-C₄₈-aryl ester; substituted or unsubstituted C₁-C₂₀ alkyl amide; substituted or unsubstituted C₆-C₄₈-aryl amide; NR′₂; SiR′₃; —O—SiR′₃, wherein R′ is independently selected from the group consisting of substituted or unsubstituted C₁-C₂₀-alkyl and substituted or unsubstituted C₆-C₄₈-aryl, two R′ may form a ring structure; substituted or unsubstituted carboxylic acids and salts thereof; substituted or unsubstituted sulfonic acids and salts thereof; substituted or unsubstituted sulfonic esters; substituted or unsubstituted sulfonic amides; formyl; ether, thioether; carbonate; carbonate ester; sulfates; boronic acids; boronic esters; phosphonic acids; phosphonic esters; phosphines; phosphates; peroxycarbonic acids; thiocarbonic acids; sulfinic acids; sulfinic esters; sulfonates; thiolesters, sulfoxides; sulfones; alkylsulfones; hydrazides; thioaldehydes; ketones; thioketones; oximes; hydrazines; nitroso; azo; diazo; diazonium; isocyanides; cyanate; isocyanate; thiocyanate; isothiocyanate; hydroperoxide; peroxide; acetals; ketal; orthoester; orthocarbonate esters; ammonium; imines; imides; azide; nitrate; isonitrile; nitrosoxy; substituted or unsubstituted carbamates; substituted or unsubstituted ethers; substituted or unsubstituted polyether carbamates; substituted or unsubstituted arylazo; substituted or unsubstituted C₂-C₂₀-alkynyl and substituted or unsubstituted C₂-C₂₀-alkenyl;
wherein the one or more substituents if present in one or more of R¹ to R¹³, are independently selected from the group consisting of D; halogen; NO₂; CN, C₂-C₄₉-alkyl acyl; substituted or unsubstituted C₁-C₂₀-alkoxy; substituted or unsubstituted C₆-C₄₈-aryloxy; substituted or unsubstituted C₂-C₄₉-aryl acyl; (meth)acrylate; tosyl; sulfonic acid or salts thereof; carboxylic acid or salts thereof; boronic acid or salts thereof; phosphonic acid or salts thereof; NR′₃⁺, wherein R′ is independently selected from the group consisting of H, D, substituted or unsubstituted C₁-C₂₀-alkyl and substituted or unsubstituted C₆-C₄₈-aryl, two R′ may form a ring structure; NH₂; and OH;
wherein if present two adjacent groups of R² to R⁵, R^{′2} to R^{′5}, R^{″2} to R^{″5}, R¹⁰ to R¹³; R^{′10} to R^{′13} and R^{″10} to R^{″13} may be independently linked to each other to form a fused ring structure; and
wherein if present R^{″A} to R^{″B} are independently selected from H and D.

2. The process according to claim 1, wherein in formula (1) at least one of R² to R⁵ and R¹⁰ to R¹³ is a substituent selected from one of the following structures: 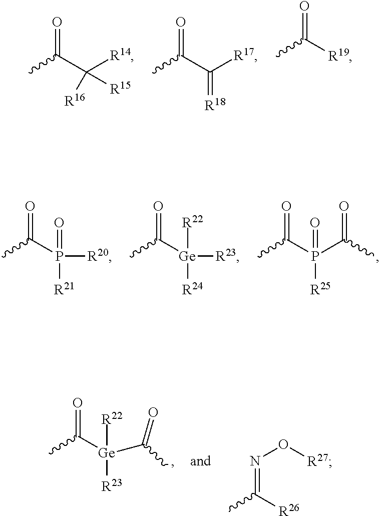
wherein R¹⁴ to R²⁷ are independently selected from the group consisting of H; D; halogen; NO₂; CN; OH; SH; substituted or unsubstituted C₁-C₂₀-alkyl; substituted or unsubstituted C₃-C₂₀-cycloalkyl; substituted or unsubstituted C₆-C₄₈-aryl; substituted or unsubstituted C₂-C₄₂-heteroaryl; substituted or unsubstituted C₂-C₄₉-alkyl acyl; substituted or unsubstituted C₂-C₄₉-aryl acyl; substituted or unsubstituted C₁-C₂₀-alkoxy; substituted or unsubstituted C₆-C₄₈-aryloxy; NH₂; substituted or unsubstituted C₁-C₂₀-alkyl ester; substituted or unsubstituted C₆-C₄₈-aryl ester; substituted or unsubstituted C₁-C₂₀ alkyl amide; substituted or unsubstituted C₆-C₄₈-aryl amide; NR′₂; SiR′₃; —O—SiR′₃, wherein R′ is independently selected from the group consisting of substituted or unsubstituted C₁-C₂₀-alkyl and substituted or unsubstituted C₆-C₄₈-aryl, two R′ may form a ring structure; substituted or unsubstituted carboxylic acids and salts thereof; substituted or unsubstituted sulfonic acids and salts thereof; substituted or unsubstituted sulfonic esters; substituted or unsubstituted sulfonic amides; formyl; ether; thioether; carbonate; carbonate ester; sulfates; boronic acids; boronic esters; phosphonic acids; phosphonic esters; phosphines; phosphates; peroxycarbonic acids; thiocarbonic acids; sulfinic acids; sulfinic esters; sulfonates; thiolesters, sulfoxides; sulfones; alkylsulfones; hydrazides; thioaldehydes; ketones; thioketones; oximes; hydrazines; nitroso; azo; diazo; diazonium; isocyanides; cyanate; isocyanate; thiocyanate; isothiocyanate; hydroperoxide; peroxide; acetals; ketal; orthoester; orthocarbonate esters; ammonium; imines; imides; azide; nitrate; isonitrile; nitrosoxy; substituted or unsubstituted carbamates; substituted or unsubstituted ethers; substituted or unsubstituted polyether carbamates; substituted or unsubstituted arylazo; substituted or unsubstituted C₂-C₂₀-alkynyl and substituted or unsubstituted C₂-C₂₀-alkenyl; wherein the one or more substituents, if present in one or more of R¹⁴-R²⁷, are independently selected from the group consisting of D; halogen; NO₂; CN; C₂-C₄₉-alkyl acyl; substituted or unsubstituted C₁-C₂₀-alkoxy; substituted or unsubstituted C₆-C₄₈-aryloxy; substituted or unsubstituted C₂-C₄₉-aryl acyl; (meth)acrylate; tosyl; sulfonic acid or salts thereof; carboxylic acid or salts thereof; boronic acid or salts thereof; phosphonic acid or salts thereof; NR′₃⁺, wherein R′ is independently selected from the group consisting of H, D, substituted or unsubstituted C₁-C₂₀-alkyl and substituted or unsubstituted C₆-C₄₈-aryl, two R′ may form a ring structure; NH₂; and OH; and R¹⁵ and R¹⁶ may be linked to each other to form a unsubstituted or substituted ring structure;
and/or
wherein in formula (1) at least one of R² to R⁵ and R¹⁰ to R¹³ is a substituent selected from the group consisting of chlorine; bromine; iodine; formyl; carbonate; carbonate ester; ester; amide; CF₃; substituted or unsubstituted C₂-C₄₉-alkyl acyl; substituted or unsubstituted C₂-C₄₉-aryl acyl; ketone; acyl; acetyl; oxime; aldehyde, NO₂; CN; (meth)acrylate; sulfone; alkylsulfones; sulfonamide; SO₂Me; SO₂NH₂; and tosyl;
and/or
wherein in formula (1) at least one of R² to R⁵ and R¹⁰ to R¹³ is a substituent selected from the group consisting of sulfonic acid or salts thereof; carboxylic acid or salts thereof; boronic acid or salts thereof; phosphonic acid or salts thereof; NR′₃⁺, wherein R′ is independently selected from the group consisting of H, D, substituted or unsubstituted C₁-C₂₀-alkyl and substituted or unsubstituted C₆-C₄₈-aryl, two R′ may form a ring structure; substituted or unsubstituted sulfonic esters; substituted or unsubstituted sulfonic amides; phosphonic esters; phosphines; phosphates; sulfinic acids; sulfinic esters; sulfonates; sulfoxides; sulfones; alkylsulfones; oximes; isocyanides; cyanate; isocyanate; thiocyanate; isothiocyanate; ammonium; substituted or unsubstituted carbamates; (meth)acrylate; tosyl; NH₂ and OH;
and/or
wherein in formula (1) R¹ is selected from the group consisting of H, D, substituted or unsubstituted C₁-C₆-alkyl, —CH₂—CH₂—OH, —CH₂—COOH, —CH₂—CH₂—COOH, —CH₂—CH₂—CH₂—NMe₃⁺, —CH₂—CH₂—CH₂—SO₃⁻, phenyl and benzyl,
and/or
the substituent on any of R¹⁴ to R²⁷ may contain the atoms necessary to complete a cyclic structure with one of R⁵-R⁸ or R¹⁰-R¹³.

3. The process according to claim 1, wherein in formula (2) at least one of R^{′2} to R^{′5} and R^{′10} to R^{′13} is a substituent selected from the group consisting of electron withdrawing substituents, acetyl, benzoyl, CN, CF₃ or one of the following structures: 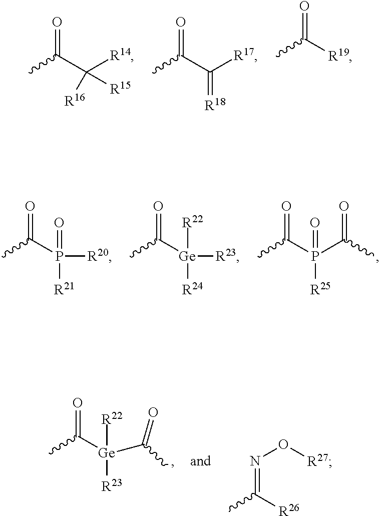
wherein R¹⁴ to R²⁷ are defined as in claim 2, or alternatively
wherein in formula (2) at least one of R^{′2} to R^{′5} is a substituent selected from the group consisting of electron donating substituents, alkoxy, methoxy, or one of the following structures: 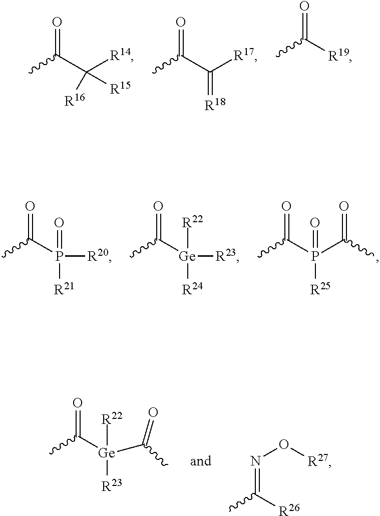
wherein R¹⁴ to R²⁷ are defined as in claim 2;
and/or
wherein at least one of R^{′10} to R^{′13} is CF₃;
and/or
wherein R^{′8} is alkyl.

4. The process according to claim 1, wherein prior to providing the reaction mixture, the precursor is preactivated with a nucleophile to obtain a preactivated precursor.

5. The process according to claim 1, wherein the reactant is the indolenium salt of formula (3): 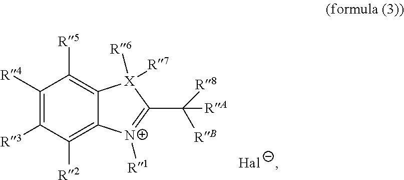
or the corresponding 2-methyleneindoline compound;
wherein X, Hal, R^{″A}, R^{″B} and R^{″1} to R^{″8} are substituents as defined in claim 1;
and/or
the amount of electron withdrawing substituents in R^{″2} to R^{″5} of formula (3) is higher than the amount of electron withdrawing substituents in R^{′2} to R^{′5} of formula (2);
and/or
wherein the substituent in R^{″4} of formula (3) is stronger electron withdrawing group than the substituent in R^{′4} of formula (2),
and/or
wherein the substituent in R^{″4} of formula (3) is chosen from the group consisting of carbonyl;
chlorine; bromine; iodine; formyl; carbonate; carbonate ester; ester; amide; CF₃; substituted or unsubstituted C₂-C₄₉-alkyl acyl; substituted or unsubstituted C₂-C₄₉-aryl acyl; ketone; acyl; acetyl; benzoyl; oxime; aldehyde, NO₂; CN; (meth)acrylate; sulfones; alkylsulfones; sulfonamides; SO₂Me; SO₂NH₂.

6. The process according to claim 1, wherein the reaction mixture further comprises a catalytic amount of a base and/or
the reaction mixture comprises a solvent, wherein the obtained spiropyran has a lower solubility compared to the precursor in said solvent;
and/or
wherein the reaction mixture further comprises a scavenger and said scavenger is able to bind to the obtained spiropyran, to bind to an obtained side product, or to deactivate an obtained side product in the reaction mixture.

7. The process according to claim 1, wherein the reactant is a salicylaldehyde of formula (4): 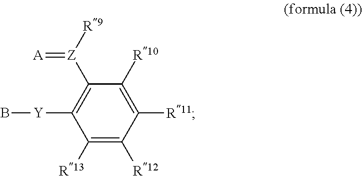
wherein A, B, Z, Y, and R^{″9} to R^{″13} are substituents as defined in claim 1, wherein R^{″10} to R^{″13} are independently selected from the group consisting of H; D; halogen; tosyl; formyl; carbonate; carbonate ester; ester; amide; CF₃; substituted or unsubstituted C₂-C₄₉-alkyl acyl; substituted or unsubstituted C₂-C₄₉-aryl acyl; ketone; acyl; acetyl; benzoyl; oxime; aldehyde, NO₂; CN, (meth)acrylate; sulfone; alkylsulfone; sulfonamide; SO₂Me; SO₂NH₂; sulfonic acid or salts thereof; carboxylic acid or salts thereof; boronic acid or salts thereof; phosphonic acid or salts thereof; NR′₃⁺, wherein R′ is independently selected from the group consisting of H, D, substituted or unsubstituted C₁-C₂₀-alkyl and substituted or unsubstituted C₆-C₄₈-aryl, two R′ may form a ring structure; substituted or unsubstituted sulfonic esters; substituted or unsubstituted sulfonic amides; phosphonic esters; phosphines; phosphates; sulfinic acids; sulfinic esters; sulfonates; sulfoxides; sulfones; alkylsulfones; oximes; isocyanides; cyanate; isocyanate; thiocyanate; isothiocyanate; ammonium; substituted or unsubstituted carbamates; NH₂; OH; substituted or unsubstituted C₁-C₂₀-alkyl; substituted or unsubstituted C₃-C₂₀-cycloalkyl; substituted or unsubstituted C₆-C₄₈-aryl; substituted or unsubstituted C₁-C₂₀-alkoxy; substituted or unsubstituted C₆-C₄₈-aryloxy; substituted or unsubstituted C₁-C₂₀-alkyl ester; substituted or unsubstituted C₆-C₄₈-aryl ester; SiR′₃, —O—SiR′₃ wherein R′ is independently selected from the group consisting of substituted or unsubstituted C₁-C₂₀-alkyl and substituted or unsubstituted C₆-C₄₈-aryl, two R′ may form a ring structure, and or the reactant is a spiropyran of formula (5): 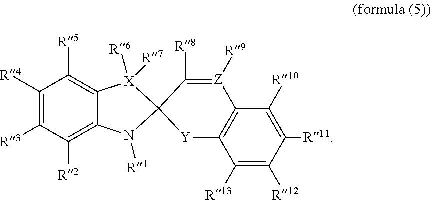
wherein the reaction mixture may comprise a further catalys.

8. A spiropyran represented by the following formula 1: 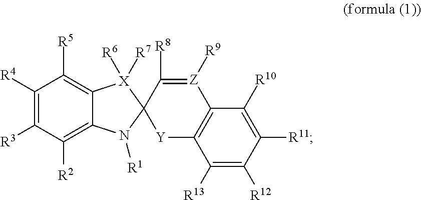
wherein X is selected from S, C, or N; if X is S, then R⁶, R⁷ may not be present accordingly; if X is N, then R⁷ may not be present accordingly,
wherein Y is selected from O, S, or N; where Y is N, the substituent contains the atoms necessary to complete a cyclic structure with R¹³ selected from the group consisting of benzimidazole, indoline, indole, dihydroquinoline, and tetrahydroquinoline;
wherein Z is selected from N or CR⁹;
wherein R¹ to R¹³ are independently selected from the group consisting of H; D; halogen; NO₂; CN; OH; SH; substituted or unsubstituted C₁-C₂₀-alkyl; substituted or unsubstituted C₃-C₂₀-cycloalkyl; substituted or unsubstituted C₆-C₄₈-aryl; substituted or unsubstituted C₂-C₄₂-heteroaryl; substituted or unsubstituted C₂-C₄₉-alkyl acyl; substituted or unsubstituted C₂-C₄₉-aryl acyl; substituted or unsubstituted C₁-C₂₀-alkoxy; substituted or unsubstituted C₆-C₄₈-aryloxy; NH₂; substituted or unsubstituted C₁-C₂₀-alkyl ester; substituted or unsubstituted C₆-C₄₈-aryl ester; substituted or unsubstituted C₁-C₂₀ alkyl amide; substituted or unsubstituted C₆-C₄₈-aryl amide; NR′₂; SiR′₃; —O—SiR′₃, wherein R′ is independently selected from the group consisting of substituted or unsubstituted C₁-C₂₀-alkyl and substituted or unsubstituted C₆-C₄₈-aryl, two R′ may form a ring structure; substituted or unsubstituted carboxylic acids and salts thereof; substituted or unsubstituted sulfonic acids and salts thereof; substituted or unsubstituted sulfonic esters; substituted or unsubstituted sulfonic amides; formyl; ether; thioether; carbonate; carbonate ester; sulfates; boronic acids; boronic esters; phosphonic acids; phosphonic esters; phosphines; phosphates; peroxycarbonic acids; thiocarbonic acids; sulfinic acids; sulfinic esters; sulfonates; thiolesters, sulfoxides; sulfones; alkylsulfones; hydrazides; thioaldehydes; ketones; thioketones; oximes; hydrazines; nitroso; azo; diazo; diazonium; isocyanides; cyanate; isocyanate; thiocyanate; isothiocyanate; hydroperoxide; peroxide; acetals; ketal; orthoester; orthocarbonate esters; ammonium; imines; imides; sulfonium salts; iodonium salts; diazo; azide; nitrate; isonitrile; nitrosoxy; substituted or unsubstituted thiocarbamates; substituted or unsubstituted carbamates; substituted or unsubstituted ethers; substituted or unsubstituted polyether carbamates; substituted or unsubstituted arylazo; substituted or unsubstituted C₂-C₂₀-alkynyl and substituted or unsubstituted C₂-C₂₀-alkenyl; wherein two adjacent groups of may be linked to each other to form a fused ring structure;
wherein the one or more substituents, if present in one or more of R¹ to R¹³, are independently selected from the group consisting of D; halogen; NO₂; CN, C₂-C₄₉-alkyl acyl; substituted or unsubstituted C₁-C₂₀-alkoxy; substituted or unsubstituted C₆-C₄₈-aryloxy; substituted or unsubstituted C₂-C₄₉-aryl acyl; (meth)acrylate; tosyl; sulfonic acid or salts thereof; carboxylic acid or salts thereof; boronic acid or salts thereof; phosphonic acid or salts thereof; NR′₃, wherein R′ is independently selected from the group consisting of H, D, substituted or unsubstituted C₁-C₂₀-alkyl and substituted or unsubstituted C₆-C₄₈-aryl, two R′ may form a ring structure; NH₂; and OH;
wherein if present two adjacent groups of R¹⁰ to R¹³, and R² to R⁵ may be independently linked to each other to form a fused ring structure;
and
wherein at least one substituent for R² to R⁵ and R¹⁰ to R¹³ is selected from the group consisting of carbonyl; chlorine; bromine; iodine; formyl; carbonate; carbonate ester; ester; amide; CF₃; substituted or unsubstituted C₂-C₄₉-alkyl acyl; substituted or unsubstituted C₂-C₄₉-aryl acyl; ketone; acyl; acetyl; benzoyl; oxime; aldehyde, NO₂; CN; (meth)acrylate; sulfones; alkylsulfones; sulfonamides; SO₂Me; SO₂NH₂; methoxy; and tosyl;
or is selected from one of the following formulae: 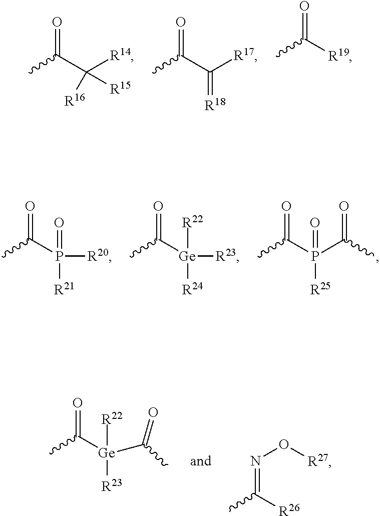
wherein R¹⁴ to R²⁷ are independently selected from the group consisting of H; D; halogen; NO₂; CN; OH; SH; CF₃; benzoyl; substituted or unsubstituted C₁-C₂₀-alkyl; substituted or unsubstituted C₃-C₂₀-cycloalkyl; substituted or unsubstituted C₆-C₄₈-aryl; substituted or unsubstituted C₂-C₄₂-heteroaryl; substituted or unsubstituted C₂-C₄₉-alkyl acyl; substituted or unsubstituted C₂-C₄₉-aryl acyl; substituted or unsubstituted C₁-C₂₀-alkoxy; substituted or unsubstituted C₆-C₄₈-aryloxy, and NH₂; substituted or unsubstituted C₁-C₂₀-alkyl ester; substituted or unsubstituted C₆-C₄₈-aryl ester; substituted or unsubstituted C₁-C₂₀ alkyl amide; substituted or unsubstituted C₆-C₄₈-aryl amide; NR′₂, SiR′₃, —O—SiR′₃ wherein R′ is independently selected from the group consisting of substituted or unsubstituted C₁-C₂₀-alkyl and substituted or unsubstituted C₆-C₄₈-aryl, two R′ may form a ring structure; substituted or unsubstituted carboxylic acids and salts thereof; substituted or unsubstituted sulfonic acids and salts thereof; substituted or unsubstituted sulfonic esters; substituted or unsubstituted sulfonic amides; formyl; ether, thioether; carbonate; carbonate ester; sulfates; boronic acids; boronic esters; phosphonic acids; phosphonic esters; phosphines; phosphates; peroxycarbonic acids; thiocarbonic acids; sulfinic acids; sulfinic esters; sulfonates; thiolesters, sulfoxides; sulfones; alkylsulfones; hydrazides; thioaldehydes; ketones; thioketones; oximes; hydrazines; nitroso; azo; diazo; diazonium; isocyanides; cyanate; isocyanate; thiocyanate; isothiocyanate; hydroperoxide; peroxide; acetals; ketal; orthoester; orthocarbonate esters; ammonium; imines; imides; azide; nitrate; isonitrile; nitrosoxy; substituted or unsubstituted carbamates; substituted or unsubstituted ethers; substituted or unsubstituted polyether carbamates; substituted or unsubstituted arylazo; substituted or unsubstituted C₂-C₂₀-alkynyl and substituted or unsubstituted C₂-C₂₀-alkenyl;
wherein the one or more substituents, if present in one or more of R¹⁴-R²⁷, are independently selected from the group consisting of D; halogen; NO₂; CN, C₂-C₄₉-alkyl acyl; substituted or unsubstituted C₁-C₂₀-alkoxy; substituted or unsubstituted C₆-C₄₈-aryloxy; substituted or unsubstituted C₂-C₄₉-aryl acyl; (meth)acrylate; tosyl; sulfonic acid or salts thereof, carboxylic acid or salts thereof, boronic acid or salts thereof, phosphonic acid or salts thereof, NR′₃⁺, wherein R′ is independently selected from the group consisting of H, D, substituted or unsubstituted C₁-C₂₀-alkyl and substituted or unsubstituted C₆-C₄₈-aryl, two R′ may form a ring structure; NH₂; and OH; and R¹⁵ and R¹⁶ may be linked to each other to form a unsubstituted or substituted ring structure,
and
wherein at least one other substituent for R² to R⁵ and R¹⁰ to R¹³ is selected from the group consisting of chlorine; bromine; iodine; formyl; carbonate; carbonate ester; ester; amide; CF₃; substituted or unsubstituted C₂-C₄₉-alkyl acyl; substituted or unsubstituted C₂-C₄₉-aryl acyl; ketone; acyl; oxime; aldehyde, NO₂; CN; (meth)acrylate; sulfones; alkylsulfones; sulfonamides; SO₂Me; SO₂NH₂; methoxy; and tosyl;
and/or
wherein at least one other substituent for R² to R⁵ and R¹⁰ to R¹³ is selected from sulfonic acid or salts thereof; carboxylic acid or salts thereof; boronic acid or salts thereof; phosphonic acid or salts thereof; NR′₃⁺, wherein R′ is independently selected from the group consisting of H, D, substituted or unsubstituted C₁-C₂₀-alkyl and substituted or unsubstituted C₆-C₄₈-aryl, two R′ may form a ring structure; substituted or unsubstituted sulfonic esters; substituted or unsubstituted sulfonic amides; phosphonic esters; phosphines; phosphates; sulfinic acids; sulfinic esters; sulfonates; sulfoxides; sulfones; alkylsulfones; oximes; isocyanides; cyanate; isocyanate; thiocyanate; isothiocyanate; ammonium; substituted or unsubstituted carbamates; (meth)acrylate; tosyl; NH₂; and OH
and/or
the substituent on any of R¹⁴ to R²⁷ may contain the atoms necessary to complete a cyclic structure with one of R⁵-R⁸ or R¹⁰-R¹³;
and/or
wherein
R¹³ is 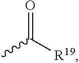
and/or
two or more of R² to R⁵ and R¹⁰ to R¹³ are electron withdrawing groups,
and/or
R¹ is selected from the group consisting of H, D, substituted or unsubstituted C₁-C₆-alkyl, —CH₂—CH₂—OH, —CH₂—COOH, —CH₂—CH₂—COOH, —CH₂—CH₂—CH₂—NMe₃⁺, —CH₂—CH₂—CH₂—SO₃⁻, phenyl and benzyl.

9. A spiropyran according to claim 8, wherein R¹³ is 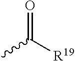
and R¹⁹ is substituted or unsubstituted C₆-C₄₈-aryl.

10. The spiropyran according to claim 9, wherein at least one of R² to R⁵ is selected from the group consisting of chlorine; bromine; iodine; formyl; carbonate; carbonate ester; ester; amide; CF₃; substituted or unsubstituted C₂-C₄₉-alkyl acyl; substituted or unsubstituted C₂-C₄₉-aryl acyl; ketone; acyl; oxime; aldehyde, NO₂; CN; (meth)acrylate; sulfones; alkylsulfones; sulfonamides; SO₂Me; SO₂NH₂; methoxy; and tosyl.

11. A spiropyran according to claim 8, wherein at least one of R¹⁰-R¹³ is a substituted arylacyl and contains an electron withdrawing group as a substituent.

12. A process for locally polymerizing a starting material by using a spiropyran according to claim 8 as a photoinitiator and irradiating the spiropyran with light of least one wavelength.

13. A process for locally polymerizing a starting material by dual color photopolymerization, comprising:
providing a polymerizable starting material containing photoinitiator molecules wherein the photoinitiator molecule is a spiropyran according to claim 8, which can be converted by sequential optical excitation into a reactive state in which the photoinitiator molecules locally trigger polymerization of the starting material; and
photopolymerizing the starting material in a local volume by irradiating light of a first wavelength and light of a second wavelength, different from the first wavelength, into the local volume, whereby in the local volume
the photoinitiator molecules are converted, due to the absorption of the light of the first wavelength, from an initial state in which the photoinitiator molecules substantially do not absorb the light of the second wavelength, into an intermediate state with changed optical properties compared to the initial state, such that the photoinitiator molecules in the intermediate state absorb the light of the second wavelength; and
the photoinitiator molecules are transferred from the intermediate state to the reactive state due to the absorption of the light of the second wavelength, which triggers the polymerization locally.

14. A process for the formation of shaped body by dual color photopolymerization, comprising:
providing a polymerizable starting material containing photoinitiator molecules wherein the photoinitiator molecule is a spiropyran according to claim 8, which can be converted by sequential optical excitation into a reactive state in which the photoinitiator molecules locally trigger polymerization of the starting material; and
photopolymerizing the starting material in a local volume by irradiating light of a first wavelength and light of a second wavelength, different from the first wavelength, into the local volume, whereby in the local volume
the photoinitiator molecules are converted, due to the absorption of the light of the first wavelength, from an initial state in which the photoinitiator molecules substantially do not absorb the light of the second wavelength, into an intermediate state with changed optical properties compared to the initial state, such that the photoinitiator molecules in the intermediate state absorb the light of the second wavelength; and
the photoinitiator molecules are transferred from the intermediate state to the reactive state due to the absorption of the light of the second wavelength, which triggers the polymerization locally; and
the photoinitiator molecules may be transferred from the intermediate state to the initial state spontaneously in a thermal reaction.

15. A process for locally polymerizing a starting material by dual color photopolymerization and forming a shaped body, comprising:
providing a container which is at least partially filled with a polymerizable starting material containing photoinitiator molecules, which can be converted by sequential optical excitation into a reactive state in which the photoinitiator molecules locally trigger polymerization of the starting material; and
photopolymerizing the starting material in a container by irradiating light of a first wavelength and light of a second wavelength, different from the first wavelength, into the container, whereby in the local volume
the photoinitiator molecules are converted, due to the absorption of the light of the first wavelength, from an initial state in which the photoinitiator molecules substantially do not absorb the light of the second wavelength, into an intermediate state with changed optical properties compared to the initial state, such that the photoinitiator molecules in the intermediate state absorb the light of the second wavelength; and
the photoinitiator molecules are transferred from the intermediate state to the reactive state due to the absorption of the light of the second wavelength, which triggers the polymerization locally to form the shaped body; wherein
the initial state of the photoinitiator in the photopolymerizable material has an extinction coefficient at the first wavelength in the range of 2500 to 10 L mol⁻¹ cm⁻¹.

16. A process for locally polymerizing a starting material by dual color photopolymerization, comprising:
providing a polymerizable starting material containing photoinitiator molecules which can be converted by sequential optical excitation into a reactive state in which the photoinitiator molecules locally trigger polymerization of the starting material; and
photopolymerizing the starting material in a local volume by irradiating light of a first wavelength and light of a second wavelength, different from the first wavelength, into the local volume, whereby in the local volume
the photoinitiator molecules are converted, due to the absorption of the light of the first wavelength, from an initial state, into an intermediate state with changed optical properties compared to the initial state, such that the photoinitiator molecules in the intermediate state absorb the light of the second wavelength; and
the photoinitiator molecules are transferred from the intermediate state to the reactive state due to the absorption of the light of the second wavelength, which triggers the polymerization locally; wherein the photoinitiator molecule comprises the following structural motif (BI) 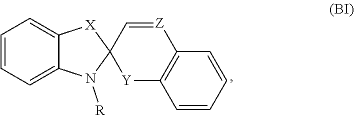
wherein
X is selected from the group consisting of S, CR^{S}₂, and NR^{S};
Y is selected from the group consisting of O, S, and NR^{S};
Z is selected from the group consisting of N and CR^{S′}; and
R^{S} are independently selected.

17. The process according to claim 16, wherein the structural motif (BI) is the structural motif (BII) 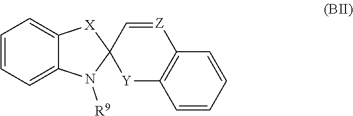
wherein
X is selected from the group consisting of S, CR^{S}₂, and NR^{S};
Y is selected from the group consisting of O, S, and NR^{S};
Z is selected from the group consisting of N and CR^{S′};
R^{S} are independently selected; and
R⁹ is electron withdrawing alkyl aryl or electron withdrawing alkyl heteroaryl.

18. The process according to claim 16, wherein R⁹ is-CH₂-(electron withdrawing C₅-C₄₈-aryl);
or
wherein R⁹ is-CH₂-(electron withdrawing phenyl).

19. The process according to claim 17, wherein the electron withdrawing aryl is substituted aryl, wherein the aryl is substituted with one or more electron withdrawing group(s).

20. The process according to claim 19, wherein the electron withdrawing group is selected from the group consisting of F, Cl, Br, I, NO₂, CN, carbonyl, carbonate, carbonate ester, partially fluorinated C₁-C₂₀ alkyl, partially fluorinated C₅-C₄₈ aryl, perfluorinated C₁ to C₂₀ alkyl, perfluorinated C₅-C₄₈ aryl, C₂-C₄₉-alkyl acyl, C₅-C₄₈-aryl acyl, substituted C₂-C₄₉-alkyl acyl, substituted C₅-C₄₈-aryl acyl, substituted or unsubstituted (meth)acrylate, C₁-C₂₀ alkylsulfone, C₅-C₄₈-aryl sulfone, sulfone, SO₂Me, SO₂NH₂, methoxy, C₂-C₄₉-alkoxy carbonyl, C₂-C₄₉-aryloxy carbonyl, formyl, tosyl, mesyl, C₂-C₂₀ alkyl amide, and C₅-C₄₈-aryl amide.

21. The process according to claim 16, wherein the structural motif (BI) or (BII) is the structural motif (BIII) 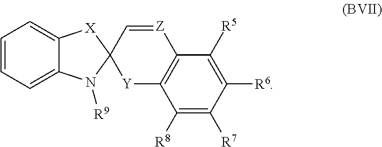
and at least one of R⁵ to R⁸ is an electron withdrawing group;
or
wherein the structural motif (BI), (BII) or (BIII) is the structural motif (BIV) 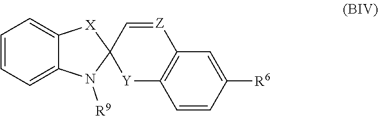
and R⁶ is an electron withdrawing group;
or
wherein the structural motif (BI), (BII) or (BIII) is the structural motif (BV) 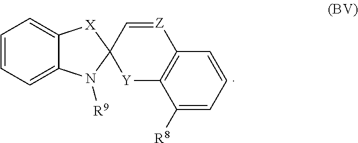
and R⁸ is an electron withdrawing group.

22. The process according to claim 21, wherein at least one of R⁵ and R⁷ is an electron donating group, preferably selected from the group consisting of substituted or unsubstituted C₁-C₂₀-alkoxy, preferably methoxy, C₅-C₄₈-aryloxy, and NR″₂, wherein R″ is independently selected from the group consisting of substituted or unsubstituted C₁-C₂₀-alkyl and substituted or unsubstituted C₅-C₄₈-aryl, and two R″ may form a ring structure.

23. The process according to claim 16, wherein the structural motif (BI) or (BII) is the structural motif (BVII) 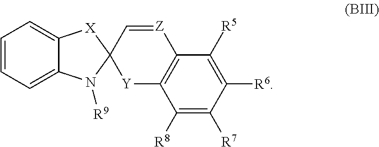
at least one of R⁵ and R⁷ is independently an electron donating group; and
R₈ is an electron withdrawing group.

24. The process according to claim 16, wherein the compound comprising the structural motif (BI) is selected from the following compounds

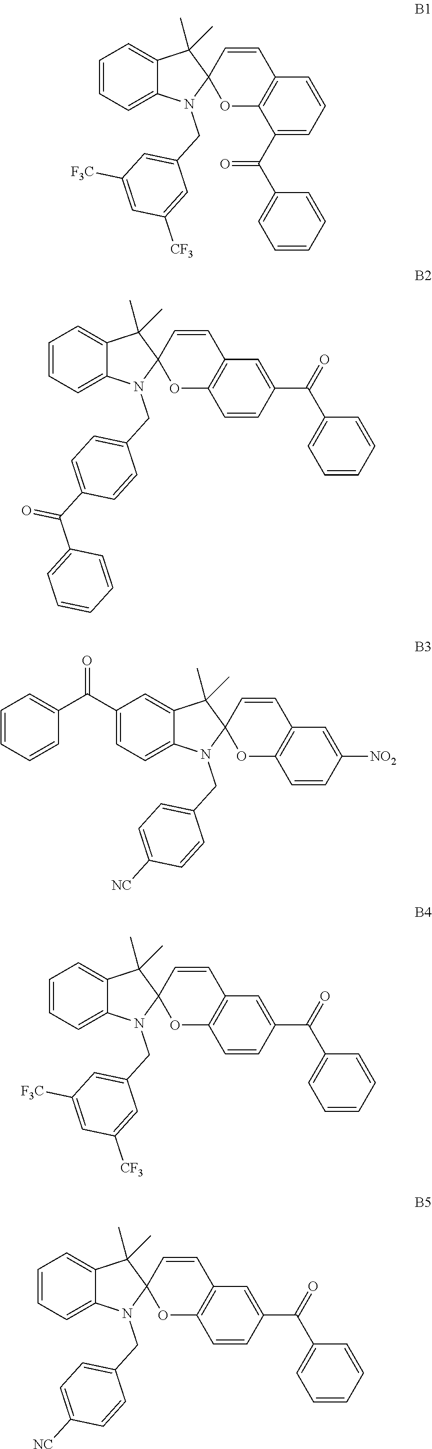 [SMILES: CC1(C)c2ccccc2N(Cc2cc(C(F)(F)F)cc(C(F)(F)F)c2)C12C=Cc1cccc(C(=O)c3ccccc3)c1O2 ; CC1(C)c2ccccc2N(Cc2ccc(C(=O)c3ccccc3)cc2)C12C=Cc1cc(C(=O)c3ccccc3)ccc1O2 ; CC1(C)c2cc(C(=O)c3ccccc3)ccc2N(Cc2ccc(C#N)cc2)C12C=Cc1cc([N+](=O)[O-])ccc1O2 ; CC1(C)c2ccccc2N(Cc2cc(C(F)(F)F)cc(C(F)(F)F)c2)C12C=Cc1cc(C(=O)c3ccccc3)ccc1O2 ; CC1(C)c2ccccc2N(Cc2ccc(C#N)cc2)C12C=Cc1cc(C(=O)c3ccccc3)ccc1O2]

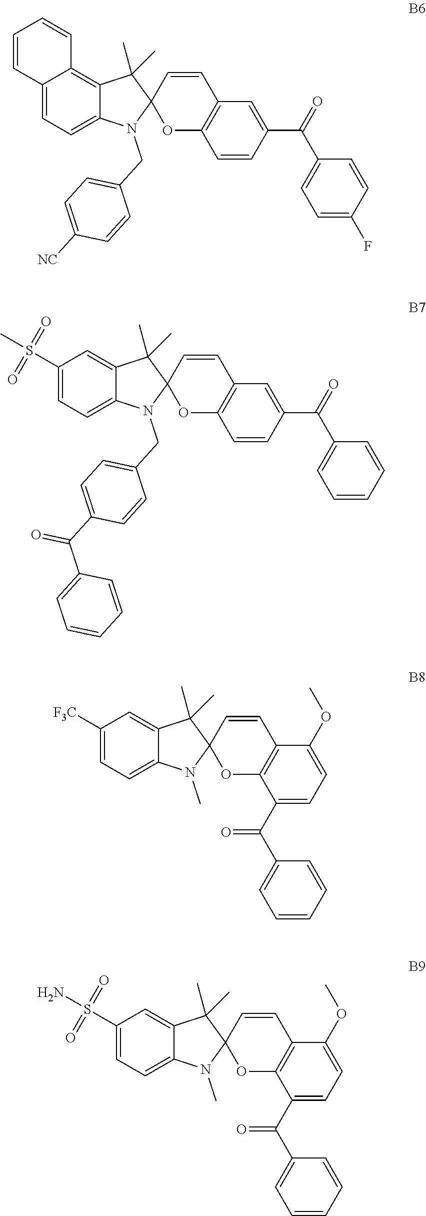 [SMILES: CC1(C)c2c(ccc3ccccc23)N(Cc2ccc(C#N)cc2)C12C=Cc1cc(C(=O)c3ccc(F)cc3)ccc1O2 ; CC1(C)c2cc(S(C)(=O)=O)ccc2N(Cc2ccc(C(=O)c3ccccc3)cc2)C12C=Cc1cc(C(=O)c3ccccc3)ccc1O2 ; COc1ccc(C(=O)c2ccccc2)c2c1C=CC1(O2)N(C)c2ccc(C(F)(F)F)cc2C1(C)C ; COc1ccc(C(=O)c2ccccc2)c2c1C=CC1(O2)N(C)c2ccc(S(N)(=O)=O)cc2C1(C)C]

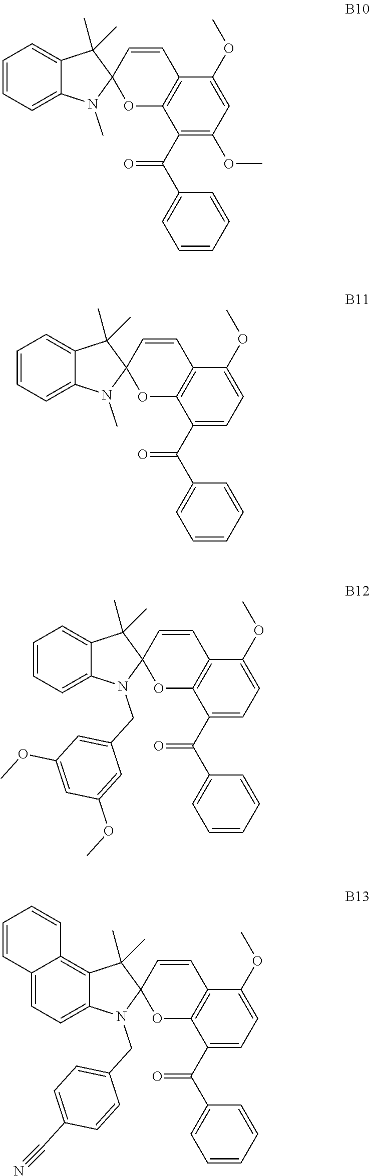

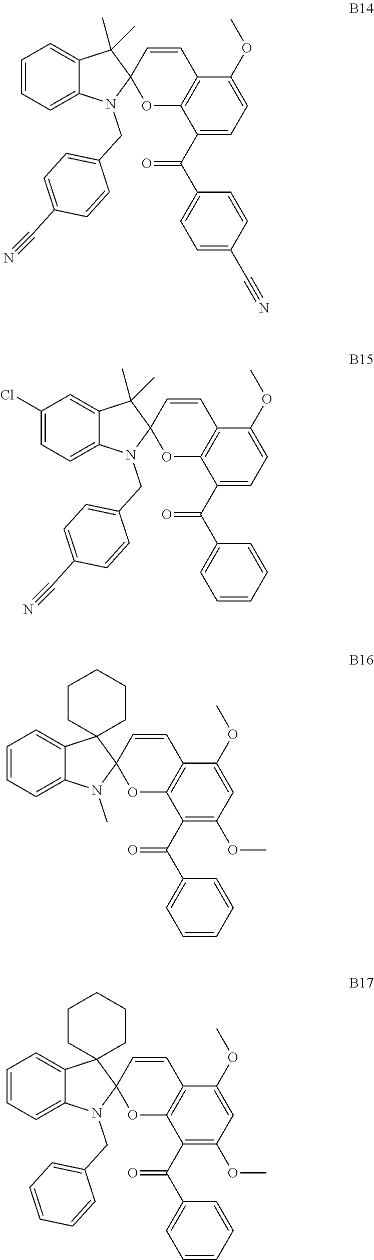 [SMILES: COc1ccc(C(=O)c2ccc(C#N)cc2)c2c1C=CC1(O2)N(Cc2ccc(C#N)cc2)c2ccccc2C1(C)C ; COc1ccc(C(=O)c2ccccc2)c2c1C=CC1(O2)N(Cc2ccc(C#N)cc2)c2ccc(Cl)cc2C1(C)C ; COc1cc(OC)c(C(=O)c2ccccc2)c2c1C=CC1(O2)N(C)c2ccccc2C12CCCCC2 ; COc1cc(OC)c(C(=O)c2ccccc2)c2c1C=CC1(O2)N(Cc2ccccc2)c2ccccc2C12CCCCC2]

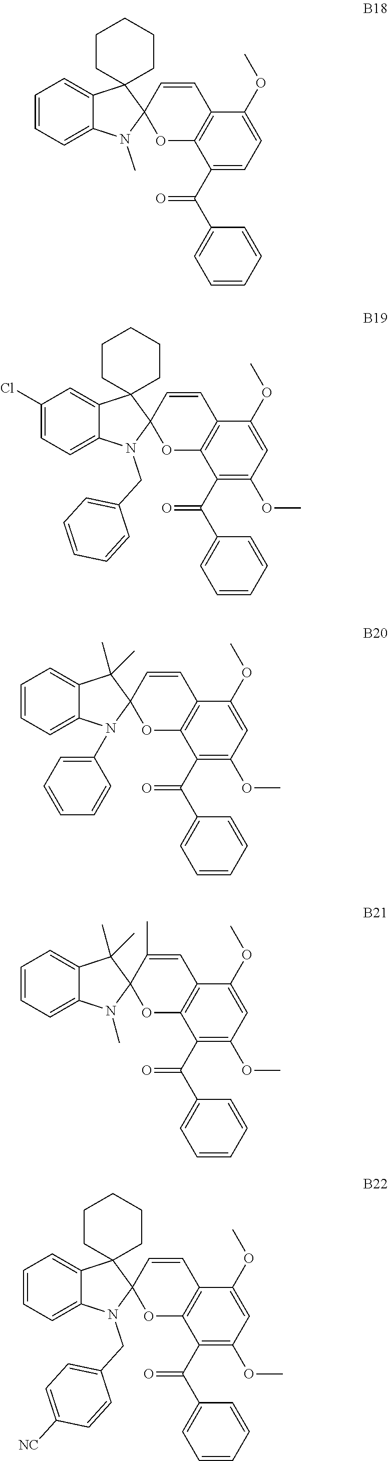 [SMILES: COc1ccc(C(=O)c2ccccc2)c2c1C=CC1(O2)N(C)c2ccccc2C12CCCCC2 ; COc1cc(OC)c(C(=O)c2ccccc2)c2c1C=CC1(O2)N(Cc2ccccc2)c2ccc(Cl)cc2C12CCCCC2 ; COc1cc(OC)c(C(=O)c2ccccc2)c2c1C=CC1(O2)N(c2ccccc2)c2ccccc2C1(C)C ; COc1cc(OC)c(C(=O)c2ccccc2)c2c1C=C(C)C1(O2)N(C)c2ccccc2C1(C)C ; COc1cc(OC)c(C(=O)c2ccccc2)c2c1C=CC1(O2)N(Cc2ccc(C#N)cc2)c2ccccc2C12CCCCC2]

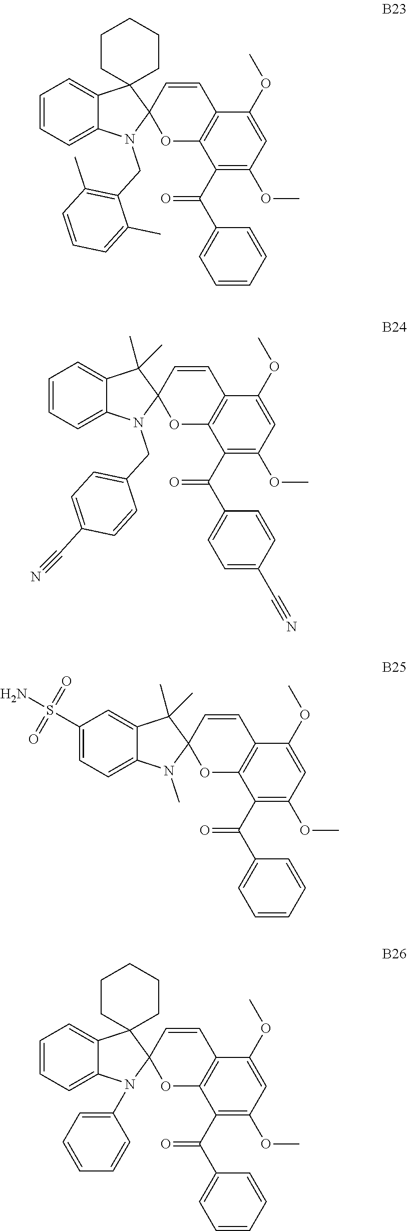 [SMILES: COc1cc(OC)c(C(=O)c2ccccc2)c2c1C=CC1(O2)N(Cc2c(C)cccc2C)c2ccccc2C12CCCCC2 ; COc1cc(OC)c(C(=O)c2ccc(C#N)cc2)c2c1C=CC1(O2)N(Cc2ccc(C#N)cc2)c2ccccc2C1(C)C ; COc1cc(OC)c(C(=O)c2ccccc2)c2c1C=CC1(O2)N(C)c2ccc(S(N)(=O)=O)cc2C1(C)C ; COc1cc(OC)c(C(=O)c2ccccc2)c2c1C=CC1(O2)N(c2ccccc2)c2ccccc2C12CCCCC2]

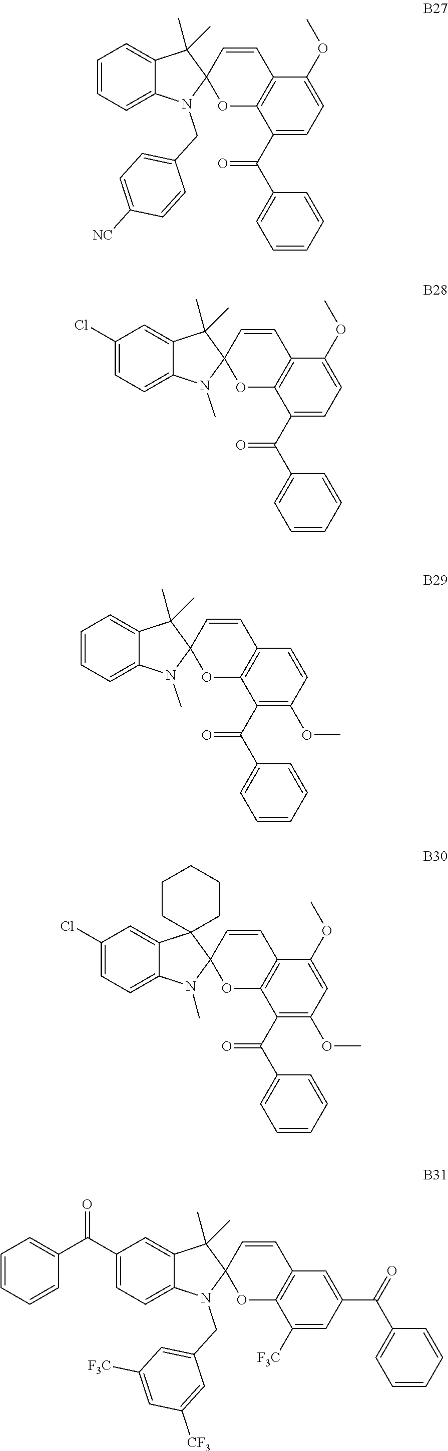 [SMILES: COc1ccc(C(=O)c2ccccc2)c2c1C=CC1(O2)N(Cc2ccc(C#N)cc2)c2ccccc2C1(C)C ; COc1ccc(C(=O)c2ccccc2)c2c1C=CC1(O2)N(C)c2ccc(Cl)cc2C1(C)C ; COc1ccc2c(c1C(=O)c1ccccc1)OC1(C=C2)N(C)c2ccccc2C1(C)C ; COc1cc(OC)c(C(=O)c2ccccc2)c2c1C=CC1(O2)N(C)c2ccc(Cl)cc2C12CCCCC2 ; CC1(C)c2cc(C(=O)c3ccccc3)ccc2N(Cc2cc(C(F)(F)F)cc(C(F)(F)F)c2)C12C=Cc1cc(C(=O)c3ccccc3)cc(C(F)(F)F)c1O2]

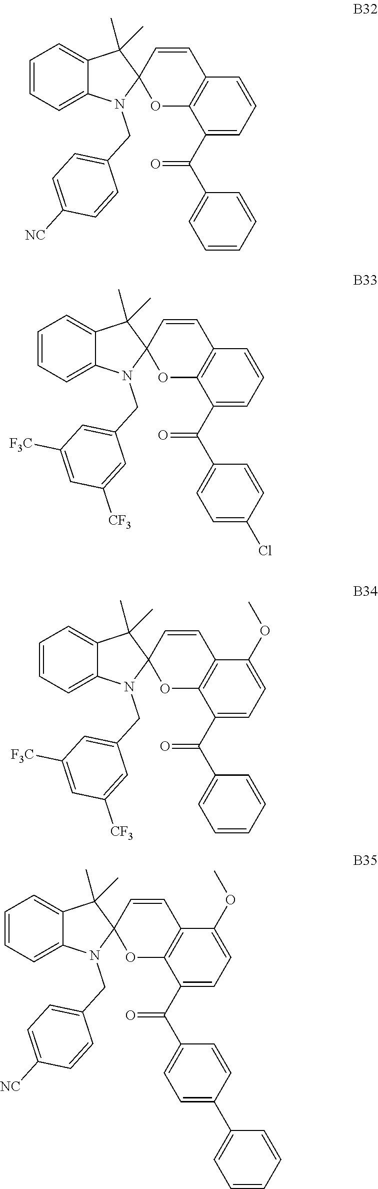

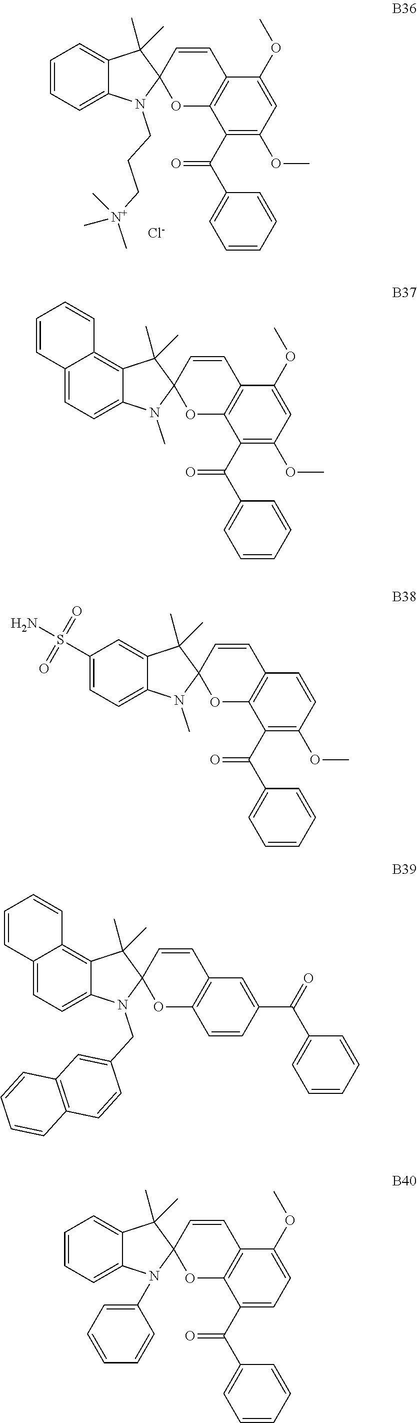 [SMILES: COc1cc(OC)c(C(=O)c2ccccc2)c2c1C=CC1(O2)N(CCC[N+](C)(C)C)c2ccccc2C1(C)C ; [Cl-] ; COc1cc(OC)c(C(=O)c2ccccc2)c2c1C=CC1(O2)N(C)c2ccc3ccccc3c2C1(C)C ; COc1ccc2c(c1C(=O)c1ccccc1)OC1(C=C2)N(C)c2ccc(S(N)(=O)=O)cc2C1(C)C ; CC1(C)c2c(ccc3ccccc23)N(Cc2ccc3ccccc3c2)C12C=Cc1cc(C(=O)c3ccccc3)ccc1O2 ; COc1ccc(C(=O)c2ccccc2)c2c1C=CC1(O2)N(c2ccccc2)c2ccccc2C1(C)C]

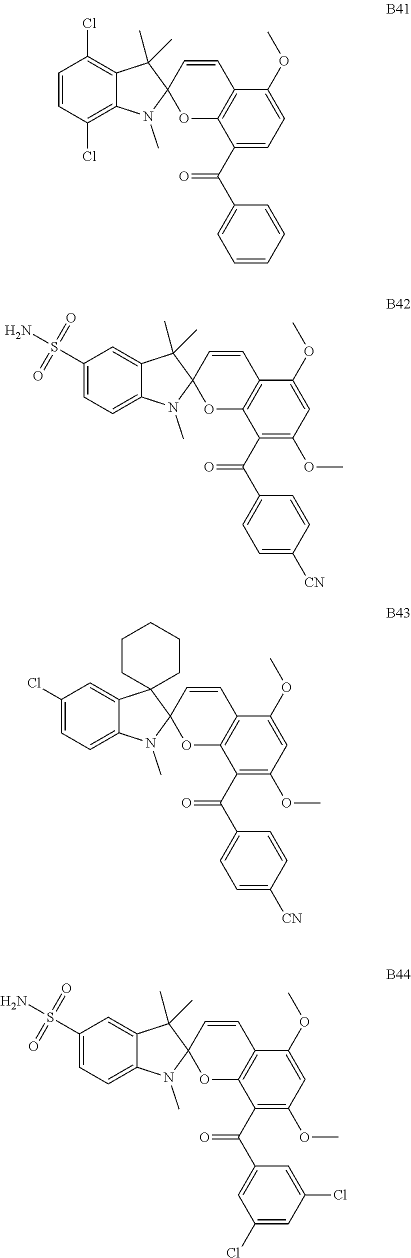 [SMILES: COc1ccc(C(=O)c2ccccc2)c2c1C=CC1(O2)N(C)c2c(Cl)ccc(Cl)c2C1(C)C ; COc1cc(OC)c(C(=O)c2ccc(C#N)cc2)c2c1C=CC1(O2)N(C)c2ccc(S(N)(=O)=O)cc2C1(C)C ; COc1cc(OC)c(C(=O)c2ccc(C#N)cc2)c2c1C=CC1(O2)N(C)c2ccc(Cl)cc2C12CCCCC2 ; COc1cc(OC)c(C(=O)c2cc(Cl)cc(Cl)c2)c2c1C=CC1(O2)N(C)c2ccc(S(N)(=O)=O)cc2C1(C)C]

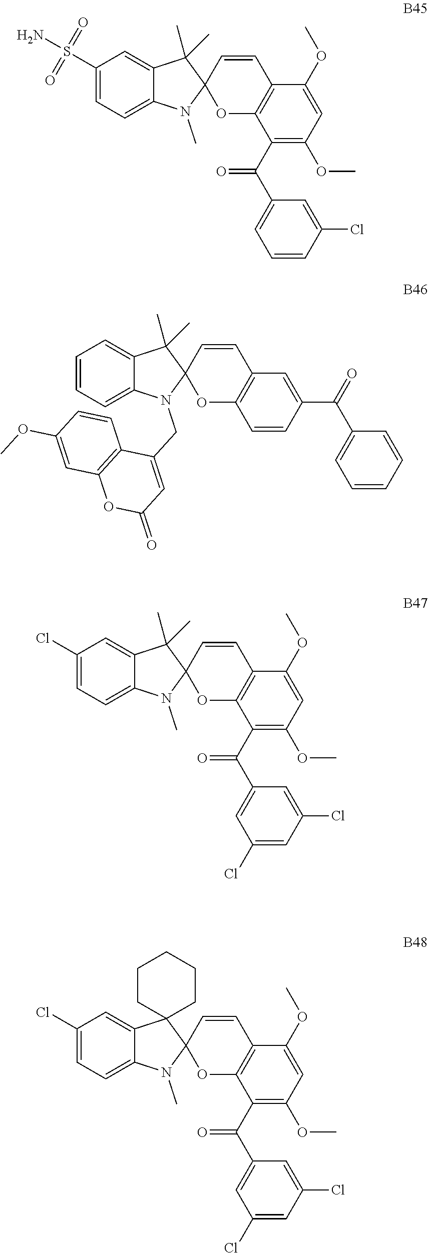 [SMILES: COc1cc(OC)c(C(=O)c2cccc(Cl)c2)c2c1C=CC1(O2)N(C)c2ccc(S(N)(=O)=O)cc2C1(C)C ; COc1ccc2c(CN3c4ccccc4C(C)(C)C34C=Cc3cc(C(=O)c5ccccc5)ccc3O4)cc(=O)oc2c1 ; COc1cc(OC)c(C(=O)c2cc(Cl)cc(Cl)c2)c2c1C=CC1(O2)N(C)c2ccc(Cl)cc2C1(C)C ; COc1cc(OC)c(C(=O)c2cc(Cl)cc(Cl)c2)c2c1C=CC1(O2)N(C)c2ccc(Cl)cc2C12CCCCC2]

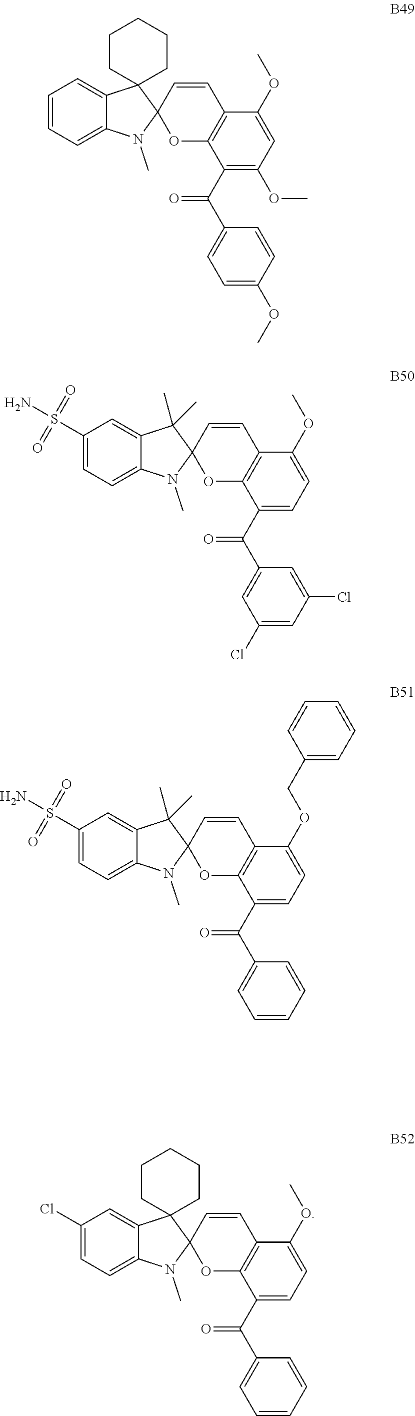 [SMILES: COc1ccc(C(=O)c2c(OC)cc(OC)c3c2OC2(C=C3)N(C)c3ccccc3C23CCCCC3)cc1 ; COc1ccc(C(=O)c2cc(Cl)cc(Cl)c2)c2c1C=CC1(O2)N(C)c2ccc(S(N)(=O)=O)cc2C1(C)C ; CN1c2ccc(S(N)(=O)=O)cc2C(C)(C)C12C=Cc1c(OCc3ccccc3)ccc(C(=O)c3ccccc3)c1O2 ; COc1ccc(C(=O)c2ccccc2)c2c1C=CC1(O2)N(C)c2ccc(Cl)cc2C12CCCCC2]

25. A process for locally polymerizing a starting material by dual color photopolymerization, comprising:
providing a polymerizable starting material containing photoinitiator molecules which can be converted by sequential optical excitation into a reactive state in which the photoinitiator molecules locally trigger polymerization of the starting material; and
photopolymerizing the starting material in a local volume by irradiating light of a first wavelength and light of a second wavelength, different from the first wavelength, into the local volume, whereby in the local volume
the photoinitiator molecules are converted, due to the absorption of the light of the first wavelength, from an initial state, into an intermediate state with changed optical properties compared to the initial state, such that the photoinitiator molecules in the intermediate state absorb the light of the second wavelength; and
the photoinitiator molecules are transferred from the intermediate state to the reactive state due to the absorption of the light of the second wavelength, which triggers the polymerization locally;
wherein the polymerizable starting material has a Difference Ratio DR of 0.2 or less
$$DR = \frac{{Abs}_{dif , UV , irr}}{{Abs}_{dif , vis , irr}}$$
wherein
Abs_{dif,vis,irr} is the absorption of the polymerizable starting material at the absorption maximum of the difference spectrum at the second wavelength measured as set forth in the description; and
Abs_{dif,UV,irr} is the absorption of the polymerizable starting material at the absorption maximum of the difference spectrum at the first wavelength measured as set forth in the description.
